(19) United States
(12) Patent Application Publication
(10) Pub. No.: US 2016/0272997 A1
(43) Pub. Date: Sep. 22, 2016

(54) STEM CANKER TOLERANT SOYBEANS AND METHODS OF USE

(71) Applicant: PIONEER HI-BRED INTERNATIONAL, INC., Johnsoton, IA (US)

(72) Inventors: Carlos Da Rosa, Johnston, IA (US); Mark J. Hood, Johnston, IA (US); Luis C. Prado, Johnston, IA (US); Sally A. Santiago-Parton, Johnston, IA (US); Joshua M. Shendelman, Johnston, IA (US); Robert J. Suttner, Johnston, IA (US); John B. Woodward, Johnston, IA (US); Yanwen Xiong, Johnston, IA (US)

(73) Assignee: PIONEER HI-BRED INTERNATIONAL, INC., Johnston, IA (US)

(21) Appl. No.: 15/031,674

(22) PCT Filed: Oct. 23, 2014
(86) PCT No.: PCT/US14/61931

§ 371 Date: Apr. 22, 2016

Related U.S. Application Data
(60) Provisional application No. 61/895,702, filed on Oct. 25, 2013.

(51) Int. Cl. C12N 15/82; C12Q 1/68

(52) U.S. Cl. CPC C12N 15/8282; C12N 15/8274; C12N 15/8275; C12N 15/8277; C12N 15/8278; C12N 15/8273; C12N 15/8271; C12N 15/8279; C12N 15/8261; C12N 15/8247; C12N 15/8251; C12N 15/8286; C12N 15/8283; C12N 15/8285; C12Q 1/6895; C12Q 2600/156; C12Q 2600/158; C12Q 2600/13

(57) ABSTRACT

Soybean plants, germplasm and seed comprising at least one native locus conferring improved stem canker tolerance, molecular markers useful for identifying and, optionally, selecting soybean plants displaying tolerance, improved tolerance, or susceptibility to stem canker, and methods of their use are provided. Also provided are isolated polynucleotides, probes, kits, systems, and the like, useful for carrying out the methods described herein.

### REFERENCE TO SEQUENCE LISTING SUBMITTED ELECTRONICALLY

The official copy of the sequence listing is submitted electronically via EFS-Web as an ASCII formatted sequence listing with a file named “4374-WO-PCT_seqlist_ST25.txt” created on Oct. 10, 2013, and having a size of 573 kilobytes and is filed concurrently with the specification. The sequence listing contained in this ASCII formatted document is part of the specification and is herein incorporated by reference in its entirety.

### FIELD OF THE INVENTION

This invention relates to stem canker resistant soybean plants, molecular markers, and methods.

### BACKGROUND

Soybeans (Glycine max (L.) Merr.) are a major cash crop and investment commodity in North America and elsewhere. Soybean oil is one of the most widely used edible oils, and soybeans are used worldwide both in animal feed and in human food production. Additionally, soybean utilization is expanding to industrial, manufacturing, and pharmaceutical applications. Stem canker is a widely recognized fungal disease that can cause serious yield losses of up to about 50% in soybean. Stem canker has been divided into Northern stem canker and Southern stem canker based on two different pathogenic varieties of the fungus Diaporthe phaseolorum. Soybean varieties resistant to at least one variety of stem canker provide efficient and effective disease control and crop management options to provide the best possible combination of flexibility and economy.

There is need for methods and compositions to identify and/or select soybean plants and germplasm with improved tolerance to fungal pathogens, improved genetic markers for identifying plants possessing tolerance or susceptibility to fungal pathogens, including agents that cause stem canker such as Diaporthe phaseolorum var. meridionalis.

### SUMMARY

Soybean plants, germplasm and seed comprising at least one native locus conferring improved stem canker tolerance, molecular markers useful for identifying and, optionally, selecting soybean plants displaying tolerance, improved tolerance, or susceptibility to stem canker, and methods of their use are provided. Also provided are isolated polynucleotides, probes, kits, systems, and the like, useful for carrying out the methods described herein.

### BRIEF DESCRIPTION OF DRAWINGS

FIG. 1 summarizes the genetic positions of loci associated with tolerance to stem canker.

### SUMMARY OF THE SEQUENCES

SEQ ID NOs:1-786 comprise polynucleotide sequences of regions of the soybean genome, each capable of being used as a probe or primer, either alone or in combination, for the detection of a marker locus associated with stem canker tolerance in soybean, and/or encoding polypeptides which are also provided as SEQ ID NOs:787-799. In certain examples, Primer1 and Primer2 are used as allele specific primers and Probe1 and Probe2 are used as allele probes. The SEQ ID NOs provided in the “Region” column of the table below are each a genomic DNA region encompassing the respective marker locus. In some examples, the primers and/or probes detect the polymorphism on based on a polynucleotide complementary to the genomic region provided here. It is to be understood that the sequences provided are sufficient for one of skill in the art to detect a locus associated with stem canker tolerance in soybean regardless of the orientation (forward, or reverse) of the strand used for detection.

TABLE 1

| | | SEQ ID NO | | | | |
|---|---|---|---|---|---|---|
| Locus | Allele (R/S) | Region | Probe1 | Probe2 | Primer1 | Primer2 |
| Gm14: 1706695 | A/T | 1 | — | — | — | — |
| Gm14: 1723656 | C/T | 2 | — | — | — | — |
| Gm14: 1723831 | G/A | 3 | — | — | — | — |
| Gm14: 1724319 | G/A | 4 | — | — | — | — |
| Gm14: 1738483 | G/A | 5 | — | — | — | — |
| Gm14: 1739929 | A/G | 6 | — | — | — | — |
| Gm14: 1850843 | A/G | 7 | — | — | — | — |
| Gm14: 1853044 | T/G | 8 | — | — | — | — |
| Gm14: 1863371 | G/A | 9 | — | — | — | — |
| Gm14: 1863436 | G/A | 10 | — | — | — | — |
| Gm14: 1863868 | C/T | 11 | — | — | — | — |
| Gm14: 1914305 | C/T | 12 | — | — | — | — |
| Gm14: 1936105 | T/C | 13 | — | — | — | — |
| Gm14: 1939210 | A/G | 14 | — | — | — | — |
| Gm14: 1940046 | C/T | 15 | — | — | — | — |
| Gm14: 1942461 | C/T | 16 | — | — | — | — |
| Gm14: 1942681 | T/C | 17 | — | — | — | — |
| Gm14: 1700735 | T/C | 18 | — | — | — | — |
| Gm14: 1702258 | G/A | 19 | — | — | — | — |
| Gm14: 1715352 | C/T | 20 | — | — | — | — |
| Gm14: 1718565 | A/C | 21 | — | — | — | — |
| Gm14: 1722078 | C/T | 22 | — | — | — | — |
| Gm14: 1722192 | T/C | 23 | — | — | — | — |
| Gm14: 1723045 | G/C | 24 | — | — | — | — |
| Gm14: 1724087 | A/G | 25 | — | — | — | — |
| Gm14: 1724245 | A/G | 26 | — | — | — | — |
| Gm14: 1724558 | T/A | 27 | — | — | — | — |
| Gm14: 1724603 | T/C | 28 | — | — | — | — |
| Gm14: 1725318 | A/G | 29 | — | — | — | — |
| Gm14: 1726899 | A/T | 30 | — | — | — | — |
| Gm14: 1738054 | T/G | 31 | — | — | — | — |
| Gm14: 1784545 | A/G | 32 | — | — | — | — |
| Gm14: 1785276 | C/T | 33 | — | — | — | — |
| Gm14: 1793750 | C/T | 34 | — | — | — | — |
| Gm14: 1842226 | T/C | 35 | — | — | — | — |
| Gm14: 1850134 | T/C | 36 | — | — | — | — |
| Gm14: 1850654 | A/G | 37 | — | — | — | — |
| Gm14: 1850808 | C/T | 38 | — | — | — | — |
| Gm14: 1851180 | C/T | 39 | — | — | — | — |
| Gm14: 1851369 | G/C | 40 | — | — | — | — |
| Gm14: 1851467 | A/G | 41 | — | — | — | — |
| Gm14: 1851793 | C/T | 42 | — | — | — | — |
| Gm14: 1852388 | G/T | 43 | — | — | — | — |
| Gm14: 1852579 | C/T | 44 | — | — | — | — |
| Gm14: 1855401 | A/T | 45 | — | — | — | — |
| Gm14: 1867651 | A/G | 46 | — | — | — | — |
| Gm14: 1867913 | A/G | 47 | — | — | — | — |
| Gm14: 1867927 | C/G | 48 | — | — | — | — |
| Gm14: 1910547 | C/A | 49 | — | — | — | — |
| Gm14: 1910746 | T/A | 50 | — | — | — | — |
| Gm14: 1910787 | C/G | 51 | — | — | — | — |
| Gm14: 1911679 | A/T | 52 | — | — | — | — |
| Gm14: 1916729 | A/G | 53 | — | — | — | — |
| Gm14: 1916742 | T/A | 54 | — | — | — | — |
| Gm14: 1919121 | T/C | 55 | — | — | — | — |
| Gm14: 1934770 | C/T | 56 | — | — | — | — |
| Gm14: 1935561 | C/T | 57 | — | — | — | — |
| Gm14: 1935695 | G/A | 58 | — | — | — | — |
| Gm14: 1935949 | A/G | 59 | — | — | — | — |
| Gm14: 1936112 | T/A | 60 | — | — | — | — |
| Gm14: 1936619 | A/G | 61 | — | — | — | — |
| Gm14: 1936750 | T/A | 62 | — | — | — | — |
| Gm14: 1936804 | A/G | 63 | — | — | — | — |
| Gm14: 1936827 | G/A | 64 | — | — | — | — |
| Gm14: 1937070 | T/A | 65 | — | — | — | — |
| Gm14: 1937136 | C/T | 66 | — | — | — | — |
| Gm14: 1937495 | C/A | 67 | — | — | — | — |
| Gm14: 1938061 | A/G | 68 | — | — | — | — |
| Gm14: 1940079 | C/T | 69 | — | — | — | — |
| Gm14: 1940534 | T/C | 70 | — | — | — | — |
| Gm14: 1940728 | G/A | 71 | — | — | — | — |
| Gm14: 1940825 | C/T | 72 | — | — | — | — |
| Gm14: 1941047 | G/A | 73 | — | — | — | — |
| Gm14: 1941105 | G/T | 74 | — | — | — | — |
| Gm14: 1944642 | A/T | 75 | — | — | — | — |
| Gm14: 1700746 | T/C | 76 | — | — | — | — |
| Gm14: 1706616 | G/A | 77 | — | — | — | — |
| Gm14: 1706705 | G/A | 78 | — | — | — | — |
| Gm14: 1706782 | A/G | 79 | — | — | — | — |
| Gm14: 1708836 | C/T | 80 | — | — | — | — |
| Gm14: 1709816 | T/A | 81 | — | — | — | — |
| Gm14: 1712174 | T/A | 82 | — | — | — | — |
| Gm14: 1717270 | A/G | 83 | — | — | — | — |
| Gm14: 1722544 | C/T | 84 | — | — | — | — |
| Gm14: 1723756 | T/A | 85 | — | — | — | — |
| Gm14: 1725153 | T/C | 86 | — | — | — | — |
| Gm14: 1725269 | C/A | 87 | — | — | — | — |
| Gm14: 1726477 | G/C | 88 | — | — | — | — |
| Gm14: 1731690 | C/T | 89 | — | — | — | — |
| Gm14: 1738538 | T/C | 90 | — | — | — | — |
| Gm14: 1739491 | T/G | 91 | — | — | — | — |
| Gm14: 1739824 | G/C | 92 | — | — | — | — |
| Gm14: 1785457 | G/C | 93 | — | — | — | — |
| Gm14: 1787260 | T/G | 94 | — | — | — | — |
| Gm14: 1796497 | T/G | 95 | — | — | — | — |
| Gm14: 1840314 | G/T | 96 | — | — | — | — |
| Gm14: 1842837 | A/G | 97 | — | — | — | — |
| Gm14: 1850412 | C/A | 98 | — | — | — | — |
| Gm14: 1850433 | T/A | 99 | — | — | — | — |
| Gm14: 1850907 | T/A | 100 | — | — | — | — |
| Gm14: 1851535 | T/A | 101 | — | — | — | — |
| Gm14: 1852210 | A/G | 102 | — | — | — | — |
| Gm14: 1859255 | C/T | 103 | — | — | — | — |
| Gm14: 1860411 | G/A | 104 | — | — | — | — |
| Gm14: 1909564 | T/C | 105 | — | — | — | — |
| Gm14: 1910968 | G/T | 106 | — | — | — | — |
| Gm14: 1935936 | A/G | 107 | — | — | — | — |
| Gm14: 1936214 | A/G | 108 | — | — | — | — |
| Gm14: 1936876 | G/A | 109 | — | — | — | — |
| Gm14: 1939030 | C/T | 110 | — | — | — | — |
| Gm14: 1939031 | T/A | 111 | — | — | — | — |
| Gm14: 1939081 | A/G | 112 | — | — | — | — |
| Gm14: 1940392 | A/C | 113 | — | — | — | — |
| Gm14: 1941517 | A/G | 114 | — | — | — | — |
| Gm14: 1989692 | G/A | 115 | — | — | — | — |
| Gm14: 1989717 | C/T | 116 | — | — | — | — |
| Gm14: 1992951 | T/A | 117 | — | — | — | — |
| Gm14: 2003498 | A/G | 118 | — | — | — | — |
| Gm14: 2004242 | A/G | 119 | — | — | — | — |
| Gm14: 2004409 | T/C | 120 | — | — | — | — |
| Gm14: 2006356 | A/T | 121 | — | — | — | — |
| Gm14: 1698254 | A/G | 122 | — | — | — | — |
| Gm14: 1698262 | T/C | 123 | — | — | — | — |
| Gm14: 1698269 | C/T | 124 | — | — | — | — |
| Gm14: 1701170 | T/A | 125 | — | — | — | — |
| Gm14: 1701174 | G/A | 126 | — | — | — | — |
| Gm14: 1701472 | T/A | 127 | — | — | — | — |
| Gm14: 1701825 | G/T | 128 | — | — | — | — |
| Gm14: 1702477 | A/T | 129 | — | — | — | — |
| Gm14: 1702552 | G/A | 130 | — | — | — | — |
| Gm14: 1702622 | T/C | 131 | — | — | — | — |
| Gm14: 1704204 | C/A | 132 | — | — | — | — |
| Gm14: 1705803 | A/G | 133 | — | — | — | — |
| Gm14: 1706201 | T/G | 134 | — | — | — | — |
| Gm14: 1706361 | C/T | 135 | — | — | — | — |
| Gm14: 1706585 | A/G | 136 | — | — | — | — |
| Gm14: 1706915 | T/C | 137 | — | — | — | — |
| Gm14: 1708746 | A/G | 138 | — | — | — | — |
| Gm14: 1713290 | A/T | 139 | — | — | — | — |
| Gm14: 1718056 | A/G | 140 | — | — | — | — |
| Gm14: 1718944 | A/G | 141 | — | — | — | — |
| Gm14: 1719373 | T/C | 142 | — | — | — | — |
| Gm14: 1723066 | A/C | 143 | — | — | — | — |
| Gm14: 1726434 | T/A | 144 | — | — | — | — |
| Gm14: 1726867 | G/A | 145 | — | — | — | — |
| Gm14: 1727235 | A/T | 146 | — | — | — | — |
| Gm14: 1728343 | G/A | 147 | — | — | — | — |
| Gm14: 1730044 | A/G | 148 | — | — | — | — |
| Gm14: 1731518 | T/G | 149 | — | — | — | — |
| Gm14: 1736053 | C/T | 150 | — | — | — | — |
| Gm14: 1738509 | C/T | 151 | — | — | — | — |
| Gm14: 1738914 | A/G | 152 | — | — | — | — |
| Gm14: 1739468 | C/T | 153 | — | — | — | — |
| Gm14: 1739718 | C/T | 154 | — | — | — | — |
| Gm14: 1739772 | T/G | 155 | — | — | — | — |
| Gm14: 1749899 | C/T | 156 | — | — | — | — |
| Gm14: 1785516 | A/G | 157 | — | — | — | — |
| Gm14: 1785559 | G/C | 158 | — | — | — | — |
| Gm14: 1786034 | T/G | 159 | — | — | — | — |
| Gm14: 1786259 | A/C | 160 | — | — | — | — |
| Gm14: 1793453 | A/G | 161 | — | — | — | — |
| Gm14: 1796469 | A/C | 162 | — | — | — | — |
| Gm14: 1797188 | A/G | 163 | — | — | — | — |
| Gm14: 1798987 | G/A | 164 | — | — | — | — |
| Gm14: 1800055 | A/G | 165 | — | — | — | — |
| Gm14: 1842374 | T/A | 166 | — | — | — | — |
| Gm14: 1850066 | A/G | 167 | — | — | — | — |
| Gm14: 1850741 | A/G | 168 | — | — | — | — |
| Gm14: 1850928 | C/T | 169 | — | — | — | — |
| Gm14: 1852469 | T/A | 170 | — | — | — | — |
| Gm14: 1854015 | G/A | 171 | — | — | — | — |
| Gm14: 1854023 | A/C | 172 | — | — | — | — |
| Gm14: 1859810 | T/A | 173 | — | — | — | — |
| Gm14: 1860271 | G/A | 174 | — | — | — | — |
| Gm14: 1862541 | G/A | 175 | — | — | — | — |
| Gm14: 1867332 | A/T | 176 | — | — | — | — |
| Gm14: 1868414 | C/A | 177 | — | — | — | — |
| Gm14: 1919937 | C/T | 178 | — | — | — | — |
| Gm14: 1935444 | T/C | 179 | — | — | — | — |
| Gm14: 1936433 | T/C | 180 | — | — | — | — |
| Gm14: 1937170 | G/A | 181 | — | — | — | — |
| Gm14: 1937923 | T/A | 182 | — | — | — | — |
| Gm14: 1938818 | T/C | 183 | — | — | — | — |
| Gm14: 1939343 | T/C | 184 | — | — | — | — |
| Gm14: 1939364 | T/C | 185 | — | — | — | — |
| Gm14: 1939647 | A/G | 186 | — | — | — | — |
| Gm14: 1939662 | T/A | 187 | — | — | — | — |
| Gm14: 1939746 | C/T | 188 | — | — | — | — |
| Gm14: 1939749 | G/A | 189 | — | — | — | — |
| Gm14: 1940207 | A/T | 190 | — | — | — | — |
| Gm14: 1940573 | A/G | 191 | — | — | — | — |
| Gm14: 1941546 | T/A | 192 | — | — | — | — |
| Gm14: 1945962 | A/T | 193 | — | — | — | — |
| Gm14: 1946355 | A/G | 194 | — | — | — | — |
| Gm14: 1990996 | A/G | 195 | — | — | — | — |
| Gm14: 1991421 | C/T | 196 | — | — | — | — |
| Gm14: 2001506 | T/C | 197 | — | — | — | — |
| Gm14: 2001882 | T/C | 198 | — | — | — | — |
| Gm14: 2003462 | G/A | 199 | — | — | — | — |
| Gm14: 2003672 | T/C | 200 | — | — | — | — |
| Gm14: 1697331 | C/T | 201 | — | — | — | — |
| Gm14: 1698694 | G/A | 202 | — | — | — | — |
| Gm14: 1700508 | C/T | 203 | — | — | — | — |
| Gm14: 1701739 | G/C | 204 | — | — | — | — |
| Gm14: 1701858 | G/T | 205 | — | — | — | — |
| Gm14: 1702110 | A/T | 206 | — | — | — | — |
| Gm14: 1702378 | C/T | 207 | — | — | — | — |
| Gm14: 1706884 | T/C | 208 | — | — | — | — |
| Gm14: 1706973 | T/C | 209 | — | — | — | — |
| Gm14: 1708902 | G/C | 210 | — | — | — | — |
| Gm14: 1711952 | T/A | 211 | — | — | — | — |
| Gm14: 1712050 | C/T | 212 | — | — | — | — |
| Gm14: 1712345 | A/C | 213 | — | — | — | — |
| Gm14: 1717261 | G/T | 214 | — | — | — | — |
| Gm14: 1722228 | T/C | 215 | — | — | — | — |
| Gm14: 1722277 | C/T | 216 | — | — | — | — |
| Gm14: 1723101 | C/T | 217 | — | — | — | — |
| Gm14: 1724793 | T/A | 218 | — | — | — | — |
| Gm14: 1725740 | G/A | 219 | — | — | — | — |
| Gm14: 1726334 | T/A | 220 | — | — | — | — |
| Gm14: 1727170 | T/C | 221 | — | — | — | — |
| Gm14: 1727625 | T/C | 222 | — | — | — | — |
| Gm14: 1730199 | C/T | 223 | — | — | — | — |
| Gm14: 1736329 | G/C | 224 | — | — | — | — |
| Gm14: 1736463 | G/A | 225 | — | — | — | — |
| Gm14: 1736832 | T/C | 226 | — | — | — | — |
| Gm14: 1737771 | G/C | 227 | — | — | — | — |
| Gm14: 1738824 | G/A | 228 | — | — | — | — |
| Gm14: 1738960 | A/C | 229 | — | — | — | — |
| Gm14: 1743818 | G/A | 230 | — | — | — | — |
| Gm14: 1756757 | G/T | 231 | — | — | — | — |
| Gm14: 1760506 | C/T | 232 | — | — | — | — |
| Gm14: 1778710 | T/A | 233 | — | — | — | — |
| Gm14: 1783716 | T/C | 234 | — | — | — | — |
| Gm14: 1784221 | T/A | 235 | — | — | — | — |
| Gm14: 1786098 | G/A | 236 | — | — | — | — |
| Gm14: 1797150 | A/T | 237 | — | — | — | — |
| Gm14: 1797155 | G/A | 238 | — | — | — | — |
| Gm14: 1799977 | T/G | 239 | — | — | — | — |
| Gm14: 1811618 | G/T | 240 | — | — | — | — |
| Gm14: 1812051 | C/G | 241 | — | — | — | — |
| Gm14: 1824464 | A/G | 242 | — | — | — | — |
| Gm14: 1839360 | T/A | 243 | — | — | — | — |
| Gm14: 1843408 | G/C | 244 | — | — | — | — |
| Gm14: 1850104 | T/C | 245 | — | — | — | — |
| Gm14: 1851115 | A/G | 246 | — | — | — | — |
| Gm14: 1851116 | G/A | 247 | — | — | — | — |
| Gm14: 1855960 | G/T | 248 | — | — | — | — |
| Gm14: 1858300 | C/T | 249 | — | — | — | — |
| Gm14: 1860243 | A/G | 250 | — | — | — | — |
| Gm14: 1860675 | C/A | 251 | — | — | — | — |
| Gm14: 1866981 | C/T | 252 | — | — | — | — |
| Gm14: 1869210 | C/A | 253 | — | — | — | — |
| Gm14: 1872625 | A/G | 254 | — | — | — | — |
| Gm14: 1902929 | C/T | 255 | — | — | — | — |
| Gm14: 1913544 | T/C | 256 | — | — | — | — |
| Gm14: 1914780 | T/C | 257 | — | — | — | — |
| Gm14: 1935408 | C/T | 258 | — | — | — | — |
| Gm14: 1936381 | C/T | 259 | — | — | — | — |
| Gm14: 1936425 | A/G | 260 | — | — | — | — |
| Gm14: 1939383 | G/A | 261 | — | — | — | — |
| Gm14: 1988977 | T/A | 262 | — | — | — | — |
| Gm14: 1989052 | A/T | 263 | — | — | — | — |
| Gm14: 1990632 | A/T | 264 | — | — | — | — |
| Gm14: 1990775 | A/G | 265 | — | — | — | — |
| Gm14: 1991550 | G/A | 266 | — | — | — | — |
| Gm14: 1999220 | C/T | 267 | — | — | — | — |
| Gm14: 2003981 | G/A | 268 | — | — | — | — |
| Gm14: 1697620 | G/T | 269 | — | — | — | — |
| Gm14: 1697978 | A/T | 270 | — | — | — | — |
| Gm14: 1699410 | A/G | 271 | — | — | — | — |
| Gm14: 1700636 | G/C | 272 | — | — | — | — |
| Gm14: 1701683 | C/T | 273 | — | — | — | — |
| Gm14: 1701935 | T/C | 274 | — | — | — | — |
| Gm14: 1702661 | T/C | 275 | — | — | — | — |
| Gm14: 1702755 | A/T | 276 | — | — | — | — |
| Gm14: 1704651 | C/T | 277 | — | — | — | — |
| Gm14: 1704664 | C/T | 278 | — | — | — | — |
| Gm14: 1712038 | A/G | 279 | — | — | — | — |
| Gm14: 1712320 | A/C | 280 | — | — | — | — |
| Gm14: 1712713 | A/G | 281 | — | — | — | — |
| Gm14: 1712865 | T/C | 282 | — | — | — | — |
| Gm14: 1712866 | A/T | 283 | — | — | — | — |
| Gm14: 1713999 | T/C | 284 | — | — | — | — |
| Gm14: 1719022 | T/G | 285 | — | — | — | — |
| Gm14: 1728216 | C/T | 286 | — | — | — | — |
| Gm14: 1731767 | T/C | 287 | — | — | — | — |
| Gm14: 1735093 | C/A | 288 | — | — | — | — |
| Gm14: 1735102 | T/A | 289 | — | — | — | — |
| Gm14: 1736945 | A/G | 290 | — | — | — | — |
| Gm14: 1743592 | G/A | 291 | — | — | — | — |
| Gm14: 1748064 | C/T | 292 | — | — | — | — |
| Gm14: 1749126 | C/A | 293 | — | — | — | — |
| Gm14: 1754686 | G/A | 294 | — | — | — | — |
| Gm14: 1757996 | C/G | 295 | — | — | — | — |
| Gm14: 1760483 | C/T | 296 | — | — | — | — |
| Gm14: 1783231 | T/A | 297 | — | — | — | — |
| Gm14: 1783538 | G/A | 298 | — | — | — | — |
| Gm14: 1783756 | T/G | 299 | — | — | — | — |
| Gm14: 1786013 | G/A | 300 | — | — | — | — |
| Gm14: 1787773 | A/G | 301 | — | — | — | — |
| Gm14: 1792218 | C/T | 302 | — | — | — | — |
| Gm14: 1799327 | A/T | 303 | — | — | — | — |
| Gm14: 1799390 | C/T | 304 | — | — | — | — |
| Gm14: 1799540 | T/C | 305 | — | — | — | — |
| Gm14: 1800012 | G/T | 306 | — | — | — | — |
| Gm14: 1800319 | T/G | 307 | — | — | — | — |
| Gm14: 1811113 | T/G | 308 | — | — | — | — |
| Gm14: 1811977 | G/A | 309 | — | — | — | — |
| Gm14: 1824284 | C/T | 310 | — | — | — | — |
| Gm14: 1837872 | C/T | 311 | — | — | — | — |
| Gm14: 1837888 | G/A | 312 | — | — | — | — |
| Gm14: 1851947 | G/A | 313 | — | — | — | — |
| Gm14: 1858277 | G/C | 314 | — | — | — | — |
| Gm14: 1861083 | A/T | 315 | — | — | — | — |
| Gm14: 1887021 | T/G | 316 | — | — | — | — |
| Gm14: 1903293 | C/T | 317 | — | — | — | — |
| Gm14: 1910033 | T/C | 318 | — | — | — | — |
| Gm14: 1919915 | A/G | 319 | — | — | — | — |
| Gm14: 1934737 | A/G | 320 | — | — | — | — |
| Gm14: 1946125 | A/G | 321 | — | — | — | — |
| Gm14: 1958086 | T/C | 322 | — | — | — | — |
| Gm14: 1986602 | T/C | 323 | — | — | — | — |
| Gm14: 1987892 | A/G | 324 | — | — | — | — |
| Gm14: 1988001 | G/A | 325 | — | — | — | — |
| Gm14: 1989951 | A/C | 326 | — | — | — | — |
| Gm14: 1991474 | A/C | 327 | — | — | — | — |
| Gm14: 1997517 | C/A | 328 | — | — | — | — |
| Gm14: 2006319 | C/G | 329 | — | — | — | — |
| Gm14: 2017162 | C/A | 330 | — | — | — | — |
| Gm14: 2019929 | A/G | 331 | — | — | — | — |
| Gm14: 1701692 | C/T | 332 | — | — | — | — |
| Gm14: 1702315 | A/T | 333 | — | — | — | — |
| Gm14: 1702956 | C/G | 334 | — | — | — | — |
| Gm14: 1703980 | A/C | 335 | — | — | — | — |
| Gm14: 1706523 | A/G | 336 | — | — | — | — |
| Gm14: 1711087 | C/A | 337 | — | — | — | — |
| Gm14: 1712377 | C/A | 338 | — | — | — | — |
| Gm14: 1714965 | G/A | 339 | — | — | — | — |
| Gm14: 1727031 | G/A | 340 | — | — | — | — |
| Gm14: 1727851 | A/T | 341 | — | — | — | — |
| Gm14: 1728741 | G/A | 342 | — | — | — | — |
| Gm14: 1729293 | A/G | 343 | — | — | — | — |
| Gm14: 1730480 | A/G | 344 | — | — | — | — |
| Gm14: 1730798 | A/T | 345 | — | — | — | — |
| Gm14: 1731840 | G/A | 346 | — | — | — | — |
| Gm14: 1732232 | G/A | 347 | — | — | — | — |
| Gm14: 1732358 | G/A | 348 | — | — | — | — |
| Gm14: 1732363 | T/G | 349 | — | — | — | — |
| Gm14: 1732403 | T/C | 350 | — | — | — | — |
| Gm14: 1735755 | T/C | 351 | — | — | — | — |
| Gm14: 1735945 | T/C | 352 | — | — | — | — |
| Gm14: 1743425 | A/G | 353 | — | — | — | — |
| Gm14: 1744356 | T/C | 354 | — | — | — | — |
| Gm14: 1746573 | T/C | 355 | — | — | — | — |
| Gm14: 1748959 | A/C | 356 | — | — | — | — |
| Gm14: 1750564 | G/C | 357 | — | — | — | — |
| Gm14: 1753875 | G/A | 358 | — | — | — | — |
| Gm14: 1754007 | A/G | 359 | — | — | — | — |
| Gm14: 1755671 | C/T | 360 | — | — | — | — |
| Gm14: 1756046 | G/T | 361 | — | — | — | — |
| Gm14: 1760299 | T/G | 362 | — | — | — | — |
| Gm14: 1760329 | A/G | 363 | — | — | — | — |
| Gm14: 1761362 | G/T | 364 | — | — | — | — |
| Gm14: 1766162 | T/G | 365 | — | — | — | — |
| Gm14: 1780165 | G/T | 366 | — | — | — | — |
| Gm14: 1780408 | G/A | 367 | — | — | — | — |
| Gm14: 1782983 | C/A | 368 | — | — | — | — |
| Gm14: 1789475 | G/T | 369 | — | — | — | — |
| Gm14: 1799494 | C/G | 370 | — | — | — | — |
| Gm14: 1799548 | C/T | 371 | — | — | — | — |
| Gm14: 1799571 | A/G | 372 | — | — | — | — |
| Gm14: 1811318 | A/T | 373 | — | — | — | — |
| Gm14: 1825691 | T/C | 374 | — | — | — | — |
| Gm14: 1834023 | G/A | 375 | — | — | — | — |
| Gm14: 1841281 | G/A | 376 | — | — | — | — |
| Gm14: 1846850 | T/C | 377 | — | — | — | — |
| Gm14: 1887108 | G/A | 378 | — | — | — | — |
| Gm14: 1887251 | T/C | 379 | — | — | — | — |
| Gm14: 1935369 | C/T | 380 | — | — | — | — |
| Gm14: 1969791 | A/G | 381 | — | — | — | — |
| Gm14: 1981574 | G/C | 382 | — | — | — | — |
| Gm14: 1981644 | G/A | 383 | — | — | — | — |
| Gm14: 1983907 | G/T | 384 | — | — | — | — |
| Gm14: 1986614 | C/G | 385 | — | — | — | — |
| Gm14: 1986675 | G/A | 386 | — | — | — | — |
| Gm14: 1987587 | A/T | 387 | — | — | — | — |
| Gm14: 1988422 | T/G | 388 | — | — | — | — |
| Gm14: 1988424 | C/T | 389 | — | — | — | — |
| Gm14: 1988520 | T/C | 390 | — | — | — | — |
| Gm14: 1988558 | G/A | 391 | — | — | — | — |
| Gm14: 1989403 | G/T | 392 | — | — | — | — |
| Gm14: 1991457 | C/T | 393 | — | — | — | — |
| Gm14: 1991910 | T/G | 394 | — | — | — | — |
| Gm14: 1992830 | T/C | 395 | — | — | — | — |
| Gm14: 1993931 | T/G | 396 | — | — | — | — |
| Gm14: 1997124 | A/G | 397 | — | — | — | — |
| Gm14: 1998879 | C/T | 398 | — | — | — | — |
| Gm14: 2004939 | G/A | 399 | — | — | — | — |
| Gm14: 2005896 | C/T | 400 | — | — | — | — |
| Gm14: 1698816 | A/G | 401 | — | — | — | — |
| Gm14: 1701991 | G/A | 402 | — | — | — | — |
| Gm14: 1710294 | G/A | 403 | — | — | — | — |
| Gm14: 1710303 | C/G | 404 | — | — | — | — |
| Gm14: 1711007 | G/T | 405 | — | — | — | — |
| Gm14: 1711575 | C/T | 406 | — | — | — | — |
| Gm14: 1713816 | G/C | 407 | — | — | — | — |
| Gm14: 1728293 | G/T | 408 | — | — | — | — |
| Gm14: 1729743 | A/G | 409 | — | — | — | — |
| Gm14: 1730281 | A/G | 410 | — | — | — | — |
| Gm14: 1730795 | G/T | 411 | — | — | — | — |
| Gm14: 1732205 | G/C | 412 | — | — | — | — |
| Gm14: 1744432 | A/G | 413 | — | — | — | — |
| Gm14: 1751350 | G/A | 414 | — | — | — | — |
| Gm14: 1755398 | T/A | 415 | — | — | — | — |
| Gm14: 1756188 | G/A | 416 | — | — | — | — |
| Gm14: 1761081 | A/G | 417 | — | — | — | — |
| Gm14: 1761387 | A/G | 418 | — | — | — | — |
| Gm14: 1762867 | A/G | 419 | — | — | — | — |
| Gm14: 1765302 | G/A | 420 | — | — | — | — |
| Gm14: 1777323 | T/A | 421 | — | — | — | — |
| Gm14: 1779323 | A/G | 422 | — | — | — | — |
| Gm14: 1780397 | A/T | 423 | — | — | — | — |
| Gm14: 1800359 | C/T | 424 | — | — | — | — |
| Gm14: 1803234 | A/C | 425 | — | — | — | — |
| Gm14: 1811158 | A/G | 426 | — | — | — | — |
| Gm14: 1825653 | G/A | 427 | — | — | — | — |
| Gm14: 1869500 | G/A | 428 | — | — | — | — |
| Gm14: 1876026 | T/C | 429 | — | — | — | — |
| Gm14: 1876675 | G/A | 430 | — | — | — | — |
| Gm14: 1876844 | G/A | 431 | — | — | — | — |
| Gm14: 1876847 | G/T | 432 | — | — | — | — |
| Gm14: 1879162 | A/T | 433 | — | — | — | — |
| Gm14: 1884603 | T/C | 434 | — | — | — | — |
| Gm14: 1904675 | A/G | 435 | — | — | — | — |
| Gm14: 1944708 | T/C | 436 | — | — | — | — |
| Gm14: 1954056 | C/T | 437 | — | — | — | — |
| Gm14: 1954062 | T/G | 438 | — | — | — | — |
| Gm14: 1967108 | A/G | 439 | — | — | — | — |
| Gm14: 1972107 | C/A | 440 | — | — | — | — |
| Gm14: 2006215 | C/T | 441 | — | — | — | — |
| Gm14: 2011465 | G/C | 442 | — | — | — | — |
| Gm14: 1704689 | G/A | 443 | — | — | — | — |
| Gm14: 1711569 | G/A | 444 | — | — | — | — |
| Gm14: 1713323 | A/C | 445 | — | — | — | — |
| Gm14: 1728052 | G/A | 446 | — | — | — | — |
| Gm14: 1729537 | T/C | 447 | — | — | — | — |
| Gm14: 1732261 | T/A | 448 | — | — | — | — |
| Gm14: 1732593 | A/G | 449 | — | — | — | — |
| Gm14: 1735227 | A/C | 450 | — | — | — | — |
| Gm14: 1744452 | A/G | 451 | — | — | — | — |
| Gm14: 1754156 | A/G | 452 | — | — | — | — |
| Gm14: 1755877 | A/G | 453 | — | — | — | — |
| Gm14: 1756355 | C/A | 454 | — | — | — | — |
| Gm14: 1759702 | A/G | 455 | — | — | — | — |
| Gm14: 1780316 | A/G | 456 | — | — | — | — |
| Gm14: 1801585 | G/T | 457 | — | — | — | — |
| Gm14: 1810099 | A/G | 458 | — | — | — | — |
| Gm14: 1810126 | A/G | 459 | — | — | — | — |
| Gm14: 1810421 | C/T | 460 | — | — | — | — |
| Gm14: 1823776 | C/G | 461 | — | — | — | — |
| Gm14: 1875278 | T/A | 462 | — | — | — | — |
| Gm14: 1875874 | T/G | 463 | — | — | — | — |
| Gm14: 1902961 | G/A | 464 | — | — | — | — |
| Gm14: 1910054 | A/T | 465 | — | — | — | — |
| Gm14: 1949216 | T/A | 466 | — | — | — | — |
| Gm14: 1957357 | G/C | 467 | — | — | — | — |
| Gm14: 1957358 | A/T | 468 | — | — | — | — |
| Gm14: 1960447 | A/T | 469 | — | — | — | — |
| Gm14: 1980728 | C/T | 470 | — | — | — | — |
| Gm14: 1984129 | G/C | 471 | — | — | — | — |
| Gm14: 1992331 | C/T | 472 | — | — | — | — |
| Gm14: 1993147 | T/G | 473 | — | — | — | — |
| Gm14: 1995593 | G/A | 474 | — | — | — | — |
| Gm14: 1995600 | T/C | 475 | — | — | — | — |
| Gm14: 1996952 | G/T | 476 | — | — | — | — |
| Gm14: 1997412 | G/C | 477 | — | — | — | — |
| Gm14: 2004591 | T/A | 478 | — | — | — | — |
| Gm14: 1645762 | G/A | 479 | — | — | — | — |
| Gm14: 1713775 | T/C | 480 | — | — | — | — |
| Gm14: 1716505 | C/A | 481 | — | — | — | — |
| Gm14: 1729186 | A/G | 482 | — | — | — | — |
| Gm14: 1729798 | A/C | 483 | — | — | — | — |
| Gm14: 1732529 | A/G | 484 | — | — | — | — |
| Gm14: 1753922 | T/G | 485 | — | — | — | — |
| Gm14: 1755572 | A/C | 486 | — | — | — | — |
| Gm14: 1759619 | C/T | 487 | — | — | — | — |
| Gm14: 1759635 | C/T | 488 | — | — | — | — |
| Gm14: 1760144 | A/T | 489 | — | — | — | — |
| Gm14: 1761168 | T/G | 490 | — | — | — | — |
| Gm14: 1771612 | C/A | 491 | — | — | — | — |
| Gm14: 1777105 | T/A | 492 | — | — | — | — |
| Gm14: 1778517 | T/C | 493 | — | — | — | — |
| Gm14: 1779288 | T/C | 494 | — | — | — | — |
| Gm14: 1779568 | A/G | 495 | — | — | — | — |
| Gm14: 1804682 | A/C | 496 | — | — | — | — |
| Gm14: 1810135 | A/G | 497 | — | — | — | — |
| Gm14: 1825225 | G/T | 498 | — | — | — | — |
| Gm14: 1828925 | G/T | 499 | — | — | — | — |
| Gm14: 1846994 | G/C | 500 | — | — | — | — |
| Gm14: 1848374 | A/T | 501 | — | — | — | — |
| Gm14: 1874941 | T/C | 502 | — | — | — | — |
| Gm14: 1875261 | G/A | 503 | — | — | — | — |
| Gm14: 1875470 | A/G | 504 | — | — | — | — |
| Gm14: 1879189 | C/T | 505 | — | — | — | — |
| Gm14: 1881168 | G/C | 506 | — | — | — | — |
| Gm14: 1923548 | A/T | 507 | — | — | — | — |
| Gm14: 1923935 | T/C | 508 | — | — | — | — |
| Gm14: 1955048 | G/A | 509 | — | — | — | — |
| Gm14: 1955118 | C/A | 510 | — | — | — | — |
| Gm14: 1955144 | T/C | 511 | — | — | — | — |
| Gm14: 1959610 | T/G | 512 | — | — | — | — |
| Gm14: 1960454 | A/C | 513 | — | — | — | — |
| Gm14: 1967167 | T/G | 514 | — | — | — | — |
| Gm14: 1970908 | C/T | 515 | — | — | — | — |
| Gm14: 1976442 | G/T | 516 | — | — | — | — |
| Gm14: 1981936 | C/A | 517 | — | — | — | — |
| Gm14: 1982632 | C/T | 518 | — | — | — | — |
| Gm14: 1982756 | C/T | 519 | — | — | — | — |
| Gm14: 1984276 | G/T | 520 | — | — | — | — |
| Gm14: 1994907 | A/T | 521 | — | — | — | — |
| Gm14: 2013156 | G/T | 522 | — | — | — | — |
| Gm14: 2019411 | A/G | 523 | — | — | — | — |
| Gm14: 1711379 | G/A | 524 | — | — | — | — |
| Gm14: 1713140 | T/C | 525 | — | — | — | — |
| Gm14: 1727997 | G/A | 526 | — | — | — | — |
| Gm14: 1744286 | A/G | 527 | — | — | — | — |
| Gm14: 1757864 | T/C | 528 | — | — | — | — |
| Gm14: 1759363 | C/T | 529 | — | — | — | — |
| Gm14: 1765933 | A/G | 530 | — | — | — | — |
| Gm14: 1767525 | C/T | 531 | — | — | — | — |
| Gm14: 1767582 | C/G | 532 | — | — | — | — |
| Gm14: 1768845 | C/T | 533 | — | — | — | — |
| Gm14: 1775564 | T/A | 534 | — | — | — | — |
| Gm14: 1778106 | G/A | 535 | — | — | — | — |
| Gm14: 1778621 | T/C | 536 | — | — | — | — |
| Gm14: 1780281 | T/C | 537 | — | — | — | — |
| Gm14: 1780494 | G/A | 538 | — | — | — | — |
| Gm14: 1805372 | C/T | 539 | — | — | — | — |
| Gm14: 1809090 | A/T | 540 | — | — | — | — |
| Gm14: 1810313 | G/A | 541 | — | — | — | — |
| Gm14: 1812109 | T/A | 542 | — | — | — | — |
| Gm14: 1812114 | A/T | 543 | — | — | — | — |
| Gm14: 1824651 | A/G | 544 | — | — | — | — |
| Gm14: 1875656 | A/G | 545 | — | — | — | — |
| Gm14: 1877489 | G/T | 546 | — | — | — | — |
| Gm14: 1878068 | T/C | 547 | — | — | — | — |
| Gm14: 1903061 | G/A | 548 | — | — | — | — |
| Gm14: 1950278 | A/G | 549 | — | — | — | — |
| Gm14: 1950577 | C/G | 550 | — | — | — | — |
| Gm14: 1950969 | G/A | 551 | — | — | — | — |
| Gm14: 1970110 | T/A | 552 | — | — | — | — |
| Gm14: 1982411 | C/T | 553 | — | — | — | — |
| Gm14: 1983489 | T/C | 554 | — | — | — | — |
| Gm14: 1983507 | T/C | 555 | — | — | — | — |
| Gm14: 1983593 | T/C | 556 | — | — | — | — |
| Gm14: 1993730 | G/A | 557 | — | — | — | — |
| Gm14: 1993788 | T/G | 558 | — | — | — | — |
| Gm14: 1993806 | A/G | 559 | — | — | — | — |
| Gm14: 2006229 | C/T | 560 | — | — | — | — |
| Gm14: 2015853 | C/A | 561 | — | — | — | — |
| Gm14: 2359579 | G/A | 562 | — | — | — | — |
| Gm14: 1579650 | G/A | 563 | — | — | — | — |
| Gm14: 1764334 | T/C | 564 | — | — | — | — |
| Gm14: 1764392 | G/A | 565 | — | — | — | — |
| Gm14: 1764403 | T/C | 566 | — | — | — | — |
| Gm14: 1767878 | C/T | 567 | — | — | — | — |
| Gm14: 1805366 | T/G | 568 | — | — | — | — |
| Gm14: 1825173 | A/G | 569 | — | — | — | — |
| Gm14: 1879313 | C/T | 570 | — | — | — | — |
| Gm14: 1886238 | G/C | 571 | — | — | — | — |
| Gm14: 1899408 | G/T | 572 | — | — | — | — |
| Gm14: 1960137 | G/A | 573 | — | — | — | — |
| Gm14: 1971009 | G/C | 574 | — | — | — | — |
| Gm14: 1983348 | T/G | 575 | — | — | — | — |
| Gm14: 2011607 | C/A | 576 | — | — | — | — |
| Gm14: 2012105 | C/T | 577 | — | — | — | — |
| Gm14: 2340746 | A/C | 578 | — | — | — | — |
| Gm14: 1767671 | C/A | 579 | — | — | — | — |
| Gm14: 1771502 | A/G | 580 | — | — | — | — |
| Gm14: 1771578 | A/C | 581 | — | — | — | — |
| Gm14: 1803995 | G/A | 582 | — | — | — | — |
| Gm14: 1805322 | G/A | 583 | — | — | — | — |
| Gm14: 1809988 | C/T | 584 | — | — | — | — |
| Gm14: 1825310 | C/T | 585 | — | — | — | — |
| Gm14: 1888310 | G/A | 586 | — | — | — | — |
| Gm14: 1889307 | A/T | 587 | — | — | — | — |
| Gm14: 1957202 | A/G | 588 | — | — | — | — |
| Gm14: 1981029 | A/C | 589 | — | — | — | — |
| Gm14: 1981073 | A/T | 590 | — | — | — | — |
| Gm14: 1981255 | T/C | 591 | — | — | — | — |
| Gm14: 1992432 | C/G | 592 | — | — | — | — |
| Gm14: 2010834 | C/T | 593 | — | — | — | — |
| Gm14: 1759440 | C/T | 594 | — | — | — | — |
| Gm14: 1775298 | A/T | 595 | — | — | — | — |
| Gm14: 1775605 | C/T | 596 | — | — | — | — |
| Gm14: 1804116 | C/T | 597 | — | — | — | — |
| Gm14: 1804868 | T/C | 598 | — | — | — | — |
| Gm14: 1806330 | C/T | 599 | — | — | — | — |
| Gm14: 1806377 | T/C | 600 | — | — | — | — |
| Gm14: 1889158 | T/C | 601 | — | — | — | — |
| Gm14: 1899428 | T/A | 602 | — | — | — | — |
| Gm14: 1926218 | T/A | 603 | — | — | — | — |
| Gm14: 1928522 | C/T | 604 | — | — | — | — |
| Gm14: 1957206 | T/C | 605 | — | — | — | — |
| Gm14: 1959033 | A/T | 606 | — | — | — | — |
| Gm14: 1969518 | C/T | 607 | — | — | — | — |
| Gm14: 1975722 | A/G | 608 | — | — | — | — |
| Gm14: 1981293 | C/T | 609 | — | — | — | — |
| Gm14: 1983189 | T/A | 610 | — | — | — | — |
| Gm14: 2013786 | T/C | 611 | — | — | — | — |
| Gm14: 2014487 | G/A | 612 | — | — | — | — |
| Gm14: 2191538 | A/C | 613 | — | — | — | — |
| Gm14: 2191541 | C/T | 614 | — | — | — | — |
| Gm14: 2191563 | T/C | 615 | — | — | — | — |
| Gm14: 1711534 | C/A | 616 | — | — | — | — |
| Gm14: 1773993 | C/A | 617 | — | — | — | — |
| Gm14: 1808347 | G/A | 618 | — | — | — | — |
| Gm14: 1808348 | C/A | 619 | — | — | — | — |
| Gm14: 1812356 | T/A | 620 | — | — | — | — |
| Gm14: 1812358 | C/A | 621 | — | — | — | — |
| Gm14: 1874583 | G/T | 622 | — | — | — | — |
| Gm14: 1971096 | C/T | 623 | — | — | — | — |
| Gm14: 1975723 | G/A | 624 | — | — | — | — |
| Gm14: 1976101 | G/C | 625 | — | — | — | — |
| Gm14: 1998648 | T/A | 626 | — | — | — | — |
| Gm14: 2006929 | T/G | 627 | — | — | — | — |
| Gm14: 2013850 | T/A | 628 | — | — | — | — |
| Gm14: 2014479 | T/A | 629 | — | — | — | — |
| Gm14: 2014481 | T/A | 630 | — | — | — | — |
| Gm14: 2101794 | A/T | 631 | — | — | — | — |
| Gm14: 766045 | A/T | 632 | — | — | — | — |
| Gm14: 1801323 | T/A | 633 | — | — | — | — |
| Gm14: 1805026 | A/T | 634 | — | — | — | — |
| Gm14: 1879302 | G/A | 635 | — | — | — | — |
| Gm14: 1975774 | T/C | 636 | — | — | — | — |
| Gm14: 2014429 | G/A | 637 | — | — | — | — |
| Gm14: 2191662 | G/A | 638 | — | — | — | — |
| Gm14: 584976 | C/T | 639 | — | — | — | — |
| Gm14: 2007005 | C/A | 640 | — | — | — | — |
| Gm14: 1650177 | A/C | 641 | — | — | — | — |
| Gm14: 1899046 | A/G | 642 | — | — | — | — |
| Gm14: 806705 | C/T | 643 | — | — | — | — |
| Gm14: 1976057 | G/A | 644 | — | — | — | — |
| Gm14: 1889837 | A/T | 645 | — | — | — | — |
| Gm14: 1727084 | C/G | 646 | 695 | 696 | 697 | 698 |
| S08231-3 | | | | | | |
| Gm14: 1755250 | C/T | 647 | 699 | 700 | 701 | 702 |
| S08255-3 | | | | | | |
| S03188-1 | A/C | 648 | 703 | 704 | 705 | 706 |
| S04492-1 | A/T | 649 | 707 | 708 | 709 | 710 |
| S08256-1 | A/G | 650 | 711 | 712 | 713 | 714 |
| S08257-1 | T/C | 651 | 715 | 716 | 717 | 718 |
| S08231-4 | T/C | 652 | 719 | 720 | 721 | 722 |
| S08241-1 | T/C | 653 | 723 | 724 | 725 | 726 |
| S08251-4 | C/A | 654 | 727 | 728 | 729 | 730 |
| S08251-2 | G/A | 655 | 731 | 732 | 733 | 734 |
| S08255-4 | A/C | 656 | 735 | 736 | 737 | 738 |
| S13721-2 | T/C | 657 | 739 | 740 | 741 | 742 |
| S01591-1 | A/G | 658 | 743 | 744 | 745 | 746 |
| S13722-1 | G/A | 659 | 747 | 748 | 749 | 750 |
| S02987-1 | G/C | 660 | 751 | 752 | 753 | 754 |
| S00802-1 | G/C | 661 | 755 | 756 | 757 | 758 |
| S00777-1 | A/T | 662 | 759 | 760 | 761 | 762 |
| S01799-1 | C/G | 663 | 763 | 764 | 765 | 766 |
| S00288-1 | A/C | 664 | 767 | 768 | 769 | 770 |
| S03923-1 | A/G | 665 | 771 | 772 | 773 | 774 |
| S00341-1 | T/C | 666 | 775 | 776 | 777 | 778 |
| S01718-1 | T/A | 667 | 779 | 780 | 781 | 782 |
| S04785-1 | T/C | 668 | 783 | 784 | 785 | 786 |

| Locus | Region | CDS | Protein |
|---|---|---|---|
| Glyma14g02740.1 | 669 | 670 | 787 |
| Glyma14g02750.1 | 671 | 672 | 788 |
| Glyma14g02780.1 | 673 | 674 | 789 |
| Glyma14g02800.1 | 675 | 676 | 790 |
| Glyma14g02820.1 | 677 | 678 | 791 |
| Glyma14g02920.1 | 679 | 680 | 792 |
| Glyma14g03010.1 | 681 | 682 | 793 |
| Glyma14g03030.1 | 683 | 684 | 794 |
| Glyma14g03050.1 | 685 | 686 | 795 |
| Glyma14g03060.1 | 687 | 688 | 796 |
| Glyma14g03100.1 | 689 | 690 | 797 |
| Glyma14g02710.1 | 691 | 692 | 798 |
| Glyma14g02730.1 | 693 | 694 | 799 |

### DETAILED DESCRIPTION

Methods for identifying a soybean plant or germplasm having tolerance, improved tolerance, or susceptibility to stem canker, are provided, the methods comprising detecting at least one allele of one or more marker loci associated with stem canker tolerance.

In some examples, the method involves identifying a soybean plant, germplasm or seed comprising at least one marker locus associated with tolerance to stem canker, in its genome, the method comprising isolating nucleic acids from the plant, germplasm or seed, and detecting at least one allele of one or more marker locus that is associated with stem canker resistance.

In some examples, the method involves detecting a single marker locus. In other examples, the method involves detecting two marker loci to provide a haplotype or marker profile for the plant or germplasm. In other examples, the method involves detecting two marker loci on different linkage groups or chromosomes to provide a marker profile for the plant or germplasm. In some examples, at least one marker locus is identified using methods of amplifying the marker locus or a portion thereof and detecting the marker amplicon produced.

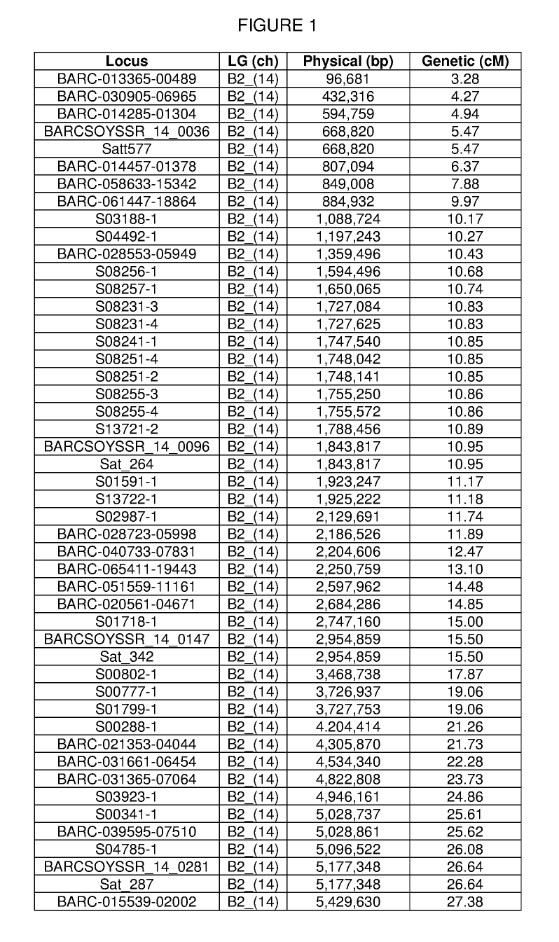

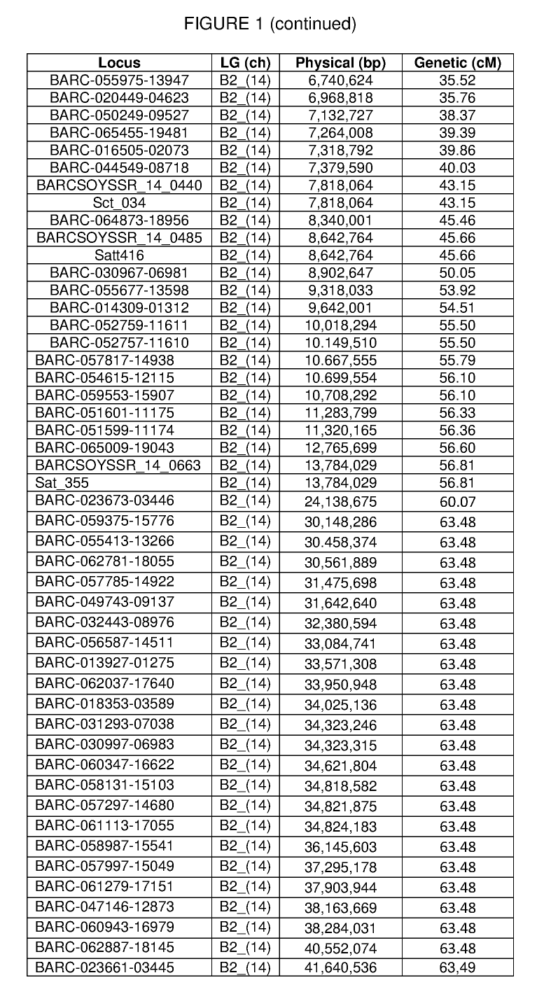

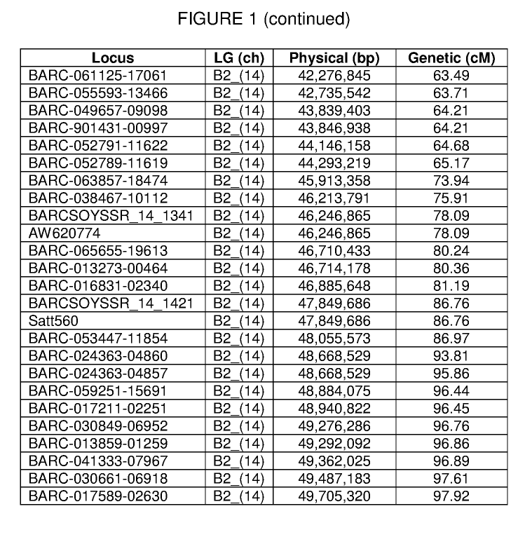

In some examples, the method comprises detecting an interval comprising at least one polymorphism associated with tolerance to stem canker. In some examples the interval is flanked by and includes BARC-013365-00489 and BARCSOYSSR_14_0281 on LG B2 (ch14). In some examples the interval is flanked by and includes BARC-013365-00489 and S04785-1 on LG B2 (ch14). In some examples the interval is flanked by and includes BARCSOYSSR_14_0036 and BARCSOYSSR_14_0281 on LG B2 (ch14). In some examples the interval is flanked by and includes positions S03188-1 and S04785-1 on LG B2 (ch14). In some examples the interval is flanked by and includes S03188-1 and S02987-1 on LG B2 (ch14). In some examples the interval is flanked by and includes positions Gm14:584976 and Gm14:2359579 on chromosome 14 (LG B2). In some examples the interval is flanked by and includes positions Gm14:1697331 and Gm14:2019929 on chromosome 14 (LG B2). In some examples the interval is flanked by and includes positions Gm14:1706695 and Gm14:1942681 on chromosome 14 (LG B2). In some examples the interval is flanked by and includes any loci, marker, polymorphism, and/or position disclosed in FIG. 1 and/or any Table or Example provided herein. In some examples the interval is an approximately 30 cM region comprising at least one locus selected from the group consisting of S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14). In some examples the interval is an approximately 20 cM region comprising at least one locus selected from the group consisting of S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14). In some examples the interval is an approximately 10 cM region comprising at least one locus selected from the group consisting of S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14). In some examples the interval is an approximately 5 cM region comprising at least one locus selected from the group consisting of S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14). In some examples the interval is an approximately 30 cM region comprising at least one locus selected from the group consisting of Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, and Glyma14g02730.1 on chromosome 14 (LG B2). In some examples the interval is an approximately 20 cM region comprising at least one locus selected from the group consisting of Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, and Glyma14g02730.1 on chromosome 14 (LG B2). In some examples the interval is an approximately 10 cM region comprising at least one locus selected from the group consisting of Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, and Glyma14g02730.1 on chromosome 14 (LG B2). In some examples the interval is an approximately 5 cM region comprising at least one locus selected from the group consisting of Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, and Glyma14g02730.1 on chromosome 14 (LG B2). In some examples, the interval is detected using a marker linked to the interval. In other examples, the marker is closely linked to the interval. In other examples the marker is in the interval.

In some examples, one or more marker locus is selected from the group consisting S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14), a marker locus linked or closely linked to any one or more of the marker loci, a marker locus in any one or more of FIG. 1 or Tables 1-13, and any combination thereof.

In some examples, the method or composition detects one or more nucleotide polymorphisms associated with stem canker resistance, wherein the polymorphism is associated with a polynucleotide selected from the group consisting of Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, and Glyma14g02730.1. In some examples the polymorphism is linked to a polynucleotide selected from the group consisting of Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, and Glyma14g02730.1. In some examples the polymorphism is closely linked to a polynucleotide selected from the group consisting of Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, and Glyma14g02730.1. In some examples the polymorphism is in a polynucleotide selected from the group consisting of Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, and Glyma14g02730.1. In some examples the polymorphism produces a non-synonymous codon change at one or more positions in a polynucleotide selected from the group consisting of Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, and Glyma14g02730.1.

In some examples, the method or composition detects one or more nucleotide polymorphisms associated with stem canker resistance, wherein the polymorphism is at a position selected from the group consisting of Gm14:1088724, Gm14:1197243, Gm14:1594889, Gm14:1650065, Gm14:1727084, Gm14:1727625, Gm14:1747540, Gm14:1748042, Gm14:1748141, Gm14:1755250, Gm14:1755572, Gm14:1788456, Gm14:1923247, Gm14:1925222, Gm14:2129691, Gm14:3468738, Gm14:3726937, Gm14:3727753, Gm14:4204414, Gm14:4946161, Gm14:5028737, Gm14:5096522, Gm14:1706695, Gm14:1723656, Gm14:1723831, Gm14:1724319, Gm14:1738483, Gm14:1739929, Gm14:1850843, Gm14:1853044, Gm14:1863371, Gm14:1863436, Gm14:1863868, Gm14:1914305, Gm14:1936105, Gm14:1939210, Gm14:1940046, Gm14:1942461, Gm14:1942681, Gm14:1700735, Gm14:1702258, Gm14:1715352, Gm14:1718565, Gm14:1722078, Gm14:1722192, Gm14:1723045, Gm14:1724087, Gm14:1724245, Gm14:1724558, Gm14:1724603, Gm14:1725318, Gm14:1726899, Gm14:1738054, Gm14:1784545, Gm14:1785276, Gm14:1793750, Gm14:1842226, Gm14:1850134, Gm14:1850654, Gm14:1850808, Gm14:1851180, Gm14:1851369, Gm14:1851467, Gm14:1851793, Gm14:1852388, Gm14:1852579, Gm14:1855401, Gm14:1867651, Gm14:1867913, Gm14:1867927, Gm14:1910547, Gm14:1910746, Gm14:1910787, Gm14:1911679, Gm14:1916729, Gm14:1916742, Gm14:1919121, Gm14:1934770, Gm14:1935561, Gm14:1935695, Gm14:1935949, Gm14:1936112, Gm14:1936619, Gm14:1936750, Gm14:1936804, Gm14:1936827, Gm14:1937070, Gm14:1937136, Gm14:1937495, Gm14:1938061, Gm14:1940079, Gm14:1940534, Gm14:1940728, Gm14:1940825, Gm14:1941047, Gm14:1941105, Gm14:1944642, Gm14:1700746, Gm14:1706616, Gm14:1706705, Gm14:1706782, Gm14:1708836, Gm14:1709816, Gm14:1712174, Gm14:1717270, Gm14:1722544, Gm14:1723756, Gm14:1725153, Gm14:1725269, Gm14:1726477, Gm14:1731690, Gm14:1738538, Gm14:1739491, Gm14:1739824, Gm14:1785457, Gm14:1787260, Gm14:1796497, Gm14:1840314, Gm14:1842837, Gm14:1850412, Gm14:1850433, Gm14:1850907, Gm14:1851535, Gm14:1852210, Gm14:1859255, Gm14:1860411, Gm14:1909564, Gm14:1910968, Gm14:1935936, Gm14:1936214, Gm14:1936876, Gm14:1939030, Gm14:1939031, Gm14:1939081, Gm14:1940392, Gm14:1941517, Gm14:1989692, Gm14:1989717, Gm14:1992951, Gm14:2003498, Gm14:2004242, Gm14:2004409, Gm14:2006356, Gm14:1698254, Gm14:1698262, Gm14:1698269, Gm14:1701170, Gm14:1701174, Gm14:1701472, Gm14:1701825, Gm14:1702477, Gm14:1702552, Gm14:1702622, Gm14:1704204, Gm14:1705803, Gm14:1706201, Gm14:1706361, Gm14:1706585, Gm14:1706915, Gm14:1708746, Gm14:1713290, Gm14:1718056, Gm14:1718944, Gm14:1719373, Gm14:1723066, Gm14:1726434, Gm14:1726867, Gm14:1727235, Gm14:1728343, Gm14:1730044, Gm14:1731518, Gm14:1736053, Gm14:1738509, Gm14:1738914, Gm14:1739468, Gm14:1739718, Gm14:1739772, Gm14:1749899, Gm14:1785516, Gm14:1785559, Gm14:1786034, Gm14:1786259, Gm14:1793453, Gm14:1796469, Gm14:1797188, Gm14:1798987, Gm14:1800055, Gm14:1842374, Gm14:1850066, Gm14:1850741, Gm14:1850928, Gm14:1852469, Gm14:1854015, Gm14:1854023, Gm14:1859810, Gm14:1860271, Gm14:1862541, Gm14:1867332, Gm14:1868414, Gm14:1919937, Gm14:1935444, Gm14:1936433, Gm14:1937170, Gm14:1937923, Gm14:1938818, Gm14:1939343, Gm14:1939364, Gm14:1939647, Gm14:1939662, Gm14:1939746, Gm14:1939749, Gm14:1940207, Gm14:1940573, Gm14:1941546, Gm14:1945962, Gm14:1946355, Gm14:1990996, Gm14:1991421, Gm14:2001506, Gm14:2001882, Gm14:2003462, Gm14:2003672, Gm14:1697331, Gm14:1698694, Gm14:1700508, Gm14:1701739, Gm14:1701858, Gm14:1702110, Gm14:1702378, Gm14:1706884, Gm14:1706973, Gm14:1708902, Gm14:1711952, Gm14:1712050, Gm14:1712345, Gm14:1717261, Gm14:1722228, Gm14:1722277, Gm14:1723101, Gm14:1724793, Gm14:1725740, Gm14:1726334, Gm14:1727170, Gm14:1730199, Gm14:1736329, Gm14:1736463, Gm14:1736832, Gm14:1737771, Gm14:1738824, Gm14:1738960, Gm14:1743818, Gm14:1756757, Gm14:1760506, Gm14:1778710, Gm14:1783716, Gm14:1784221, Gm14:1786098, Gm14:1797150, Gm14:1797155, Gm14:1799977, Gm14:1811618, Gm14:1812051, Gm14:1824464, Gm14:1839360, Gm14:1843408, Gm14:1850104, Gm14:1851115, Gm14:1851116, Gm14:1855960, Gm14:1858300, Gm14:1860243, Gm14:1860675, Gm14:1866981, Gm14:1869210, Gm14:1872625, Gm14:1902929, Gm14:1913544, Gm14:1914780, Gm14:1935408, Gm14:1936381, Gm14:1936425, Gm14:1939383, Gm14:1988977, Gm14:1989052, Gm14:1990632, Gm14:1990775, Gm14:1991550, Gm14:1999220, Gm14:2003981, Gm14:1697620, Gm14:1697978, Gm14:1699410, Gm14:1700636, Gm14:1701683, Gm14:1701935, Gm14:1702661, Gm14:1702755, Gm14:1704651, Gm14:1704664, Gm14:1712038, Gm14:1712320, Gm14:1712713, Gm14:1712865, Gm14:1712866, Gm14:1713999, Gm14:1719022, Gm14:1728216, Gm14:1731767, Gm14:1735093, Gm14:1735102, Gm14:1736945, Gm14:1743592, Gm14:1748064, Gm14:1749126, Gm14:1754686, Gm14:1757996, Gm14:1760483, Gm14:1783231, Gm14:1783538, Gm14:1783756, Gm14:1786013, Gm14:1787773, Gm14:1792218, Gm14:1799327, Gm14:1799390, Gm14:1799540, Gm14:1800012, Gm14:1800319, Gm14:1811113, Gm14:1811977, Gm14:1824284, Gm14:1837872, Gm14:1837888, Gm14:1851947, Gm14:1858277, Gm14:1861083, Gm14:1887021, Gm14:1903293, Gm14:1910033, Gm14:1919915, Gm14:1934737, Gm14:1946125, Gm14:1958086, Gm14:1986602, Gm14:1987892, Gm14:1988001, Gm14:1989951, Gm14:1991474, Gm14:1997517, Gm14:2006319, Gm14:2017162, Gm14:2019929, Gm14:1701692, Gm14:1702315, Gm14:1702956, Gm14:1703980, Gm14:1706523, Gm14:1711087, Gm14:1712377, Gm14:1714965, Gm14:1727031, Gm14:1727851, Gm14:1728741, Gm14:1729293, Gm14:1730480, Gm14:1730798, Gm14:1731840, Gm14:1732232, Gm14:1732358, Gm14:1732363, Gm14:1732403, Gm14:1735755, Gm14:1735945, Gm14:1743425, Gm14:1744356, Gm14:1746573, Gm14:1748959, Gm14:1750564, Gm14:1753875, Gm14:1754007, Gm14:1755671, Gm14:1756046, Gm14:1760299, Gm14:1760329, Gm14:1761362, Gm14:1766162, Gm14:1780165, Gm14:1780408, Gm14:1782983, Gm14:1789475, Gm14:1799494, Gm14:1799548, Gm14:1799571, Gm14:1811318, Gm14:1825691, Gm14:1834023, Gm14:1841281, Gm14:1846850, Gm14:1887108, Gm14:1887251, Gm14:1935369, Gm14:1969791, Gm14:1981574, Gm14:1981644, Gm14:1983907, Gm14:1986614, Gm14:1986675, Gm14:1987587, Gm14:1988422, Gm14:1988424, Gm14:1988520, Gm14:1988558, Gm14:1989403, Gm14:1991457, Gm14:1991910, Gm14:1992830, Gm14:1993931, Gm14:1997124, Gm14:1998879, Gm14:2004939, Gm14:2005896, Gm14:1698816, Gm14:1701991, Gm14:1710294, Gm14:1710303, Gm14:1711007, Gm14:1711575, Gm14:1713816, Gm14:1728293, Gm14:1729743, Gm14:1730281, Gm14:1730795, Gm14:1732205, Gm14:1744432, Gm14:1751350, Gm14:1755398, Gm14:1756188, Gm14:1761081, Gm14:1761387, Gm14:1762867, Gm14:1765302, Gm14:1777323, Gm14:1779323, Gm14:1780397, Gm14:1800359, Gm14:1803234, Gm14:1811158, Gm14:1825653, Gm14:1869500, Gm14:1876026, Gm14:1876675, Gm14:1876844, Gm14:1876847, Gm14:1879162, Gm14:1884603, Gm14:1904675, Gm14:1944708, Gm14:1954056, Gm14:1954062, Gm14:1967108, Gm14:1972107, Gm14:2006215, Gm14:2011465, Gm14:1704689, Gm14:1711569, Gm14:1713323, Gm14:1728052, Gm14:1729537, Gm14:1732261, Gm14:1732593, Gm14:1735227, Gm14:1744452, Gm14:1754156, Gm14:1755877, Gm14:1756355, Gm14:1759702, Gm14:1780316, Gm14:1801585, Gm14:1810099, Gm14:1810126, Gm14:1810421, Gm14:1823776, Gm14:1875278, Gm14:1875874, Gm14:1902961, Gm14:1910054, Gm14:1949216, Gm14:1957357, Gm14:1957358, Gm14:1960447, Gm14:1980728, Gm14:1984129, Gm14:1992331, Gm14:1993147, Gm14:1995593, Gm14:1995600, Gm14:1996952, Gm14:1997412, Gm14:2004591, Gm14:1645762, Gm14:1713775, Gm14:1716505, Gm14:1729186, Gm14:1729798, Gm14:1732529, Gm14:1753922, Gm14:1759619, Gm14:1759635, Gm14:1760144, Gm14:1761168, Gm14:1771612, Gm14:1777105, Gm14:1778517, Gm14:1779288, Gm14:1779568, Gm14:1804682, Gm14:1810135, Gm14:1825225, Gm14:1828925, Gm14:1846994, Gm14:1848374, Gm14:1874941, Gm14:1875261, Gm14:1875470, Gm14:1879189, Gm14:1881168, Gm14:1923548, Gm14:1923935, Gm14:1955048, Gm14:1955118, Gm14:1955144, Gm14:1959610, Gm14:1960454, Gm14:1967167, Gm14:1970908, Gm14:1976442, Gm14:1981936, Gm14:1982632, Gm14:1982756, Gm14:1984276, Gm14:1994907, Gm14:2013156, Gm14:2019411, Gm14:1711379, Gm14:1713140, Gm14:1727997, Gm14:1744286, Gm14:1757864, Gm14:1759363, Gm14:1765933, Gm14:1767525, Gm14:1767582, Gm14:1768845, Gm14:1775564, Gm14:1778106, Gm14:1778621, Gm14:1780281, Gm14:1780494, Gm14:1805372, Gm14:1809090, Gm14:1810313, Gm14:1812109, Gm14:1812114, Gm14:1824651, Gm14:1875656, Gm14:1877489, Gm14:1878068, Gm14:1903061, Gm14:1950278, Gm14:1950577, Gm14:1950969, Gm14:1970110, Gm14:1982411, Gm14:1983489, Gm14:1983507, Gm14:1983593, Gm14:1993730, Gm14:1993788, Gm14:1993806, Gm14:2006229, Gm14:2015853, Gm14:2359579, Gm14:1579650, Gm14:1764334, Gm14:1764392, Gm14:1764403, Gm14:1767878, Gm14:1805366, Gm14:1825173, Gm14:1879313, Gm14:1886238, Gm14:1899408, Gm14:1960137, Gm14:1971009, Gm14:1983348, Gm14:2011607, Gm14:2012105, Gm14:2340746, Gm14:1767671, Gm14:1771502, Gm14:1771578, Gm14:1803995, Gm14:1805322, Gm14:1809988, Gm14:1825310, Gm14:1888310, Gm14:1889307, Gm14:1957202, Gm14:1981029, Gm14:1981073, Gm14:1981255, Gm14:1992432, Gm14:2010834, Gm14:1759440, Gm14:1775298, Gm14:1775605, Gm14:1804116, Gm14:1804868, Gm14:1806330, Gm14:1806377, Gm14:1889158, Gm14:1899428, Gm14:1926218, Gm14:1928522, Gm14:1957206, Gm14:1959033, Gm14:1969518, Gm14:1975722, Gm14:1981293, Gm14:1983189, Gm14:2013786, Gm14:2014487, Gm14:2191538, Gm14:2191541, Gm14:2191563, Gm14:1711534, Gm14:1773993, Gm14:1808347, Gm14:1808348, Gm14:1812356, Gm14:1812358, Gm14:1874583, Gm14:1971096, Gm14:1975723, Gm14:1976101, Gm14:1998648, Gm14:2006929, Gm14:2013850, Gm14:2014479, Gm14:2014481, Gm14:2101794, Gm14:766045, Gm14:1801323, Gm14:1805026, Gm14:1879302, Gm14:1975774, Gm14:2014429, Gm14:2191662, Gm14:584976, Gm14:2007005, Gm14:1650177, Gm14:1899046, Gm14:806705, Gm14:1976057, Gm14:2747160, and Gm14:1889837, and any combination thereof.

In some examples, one or more marker locus is detected using a marker selected from the group consisting of S03188-1-A, S04492-1-A, S08256-1-Q1, S08257-1-Q1, S08231-3-01, S08231-4-01, S08241-1-06, S08251-4-07, S08251-2-010, S08255-3-03, S08255-4-Q1, S13721-2-01, 501591-1-A, S13722-1-01, S02987-1-A, S00802-1-A, S00777-1-A, S01799-1-A, S00288-1-A, S03923-1-A, S00341-1-A, S01718-1-A, and S04785-1-A on LG B2 (ch 14), and any combination thereof.

In some examples, at least one favorable allele associated with stem canker resistance is selected from the group consisting of allele A of S03188-1, allele A of S04492-1, allele A of S08256-1, allele T of S08257-1, allele C of S08231-3, allele T of S08231-4, allele T of S08241-1, allele C of S08251-4, allele G of S08251-2, allele C of S08255-3, allele A of S08255-4, allele T of S13721-2, allele A of S01591-1, allele G of S13722-1, allele G of S02987-1, allele G of S00802-1, allele A of S00777-1, allele C of S01799-1, allele A of S00288-1, allele A of S03923-1, allele T of S00341-1, allele T of S01718-1, and allele T of S04785-1 on LG B2 (ch 14), and any combination thereof. In some examples the method comprises detecting at least one favorable allele. In other examples, the method comprises detecting more than one favorable allele, up to and including all of the favorable alleles.

In some examples, at least one favorable allele associated with stem canker resistance is selected from the group consisting of allele A of Gm14:1088724, allele A of Gm14:1197243, allele A of Gm14:1594889, allele T of Gm14:1650065, allele C of Gm14:1727084, allele T of Gm14:1727625, allele T of Gm14:1747540, allele C of Gm14:1748042, allele G of Gm14:1748141, allele C of Gm14:1755250, allele A of Gm14:1755572, allele T of Gm14:1788456, allele A of Gm14:1923247, allele G of Gm14:1925222, allele G of Gm14:2129691, allele G of Gm14:3468738, allele A of Gm14:3726937, allele C of Gm14:3727753, allele A of Gm14:4204414, allele A of Gm14:4946161, allele T of Gm14:5028737, allele T of Gm14:5096522, allele A of Gm14:1706695, allele C of Gm14:1723656, allele G of Gm14:1723831, allele G of Gm14:1724319, allele G of Gm14:1738483, allele A of Gm14:1739929, allele A of Gm14:1850843, allele T of Gm14:1853044, allele G of Gm14:1863371, allele G of Gm14:1863436, allele C of Gm14:1863868, allele C of Gm14:1914305, allele T of Gm14:1936105, allele A of Gm14:1939210, allele C of Gm14:1940046, allele C of Gm14:1942461, allele T of Gm14:1942681, allele T of Gm14:1700735, allele G of Gm14:1702258, allele C of Gm14:1715352, allele A of Gm14:1718565, allele C of Gm14:1722078, allele T of Gm14:1722192, allele G of Gm14:1723045, allele A of Gm14:1724087, allele A of Gm14:1724245, allele T of Gm14:1724558, allele T of Gm14:1724603, allele A of Gm14:1725318, allele A of Gm14:1726899, allele T of Gm14:1738054, allele A of Gm14:1784545, allele C of Gm14:1785276, allele C of Gm14:1793750, allele T of Gm14:1842226, allele T of Gm14:1850134, allele A of Gm14:1850654, allele C of Gm14:1850808, allele C of Gm14:1851180, allele G of Gm14:1851369, allele A of Gm14:1851467, allele C of Gm14:1851793, allele G of Gm14:1852388, allele C of Gm14:1852579, allele A of Gm14:1855401, allele A of Gm14:1867651, allele A of Gm14:1867913, allele C of Gm14:1867927, allele C of Gm14:1910547, allele T of Gm14:1910746, allele C of Gm14:1910787, allele A of Gm14:1911679, allele A of Gm14:1916729, allele T of Gm14:1916742, allele T of Gm14:1919121, allele C of Gm14:1934770, allele C of Gm14:1935561, allele G of Gm14:1935695, allele A of Gm14:1935949, allele T of Gm14:1936112, allele A of Gm14:1936619, allele T of Gm14:1936750, allele A of Gm14:1936804, allele G of Gm14:1936827, allele T of Gm14:1937070, allele C of Gm14:1937136, allele C of Gm14:1937495, allele A of Gm14:1938061, allele C of Gm14:1940079, allele T of Gm14:1940534, allele G of Gm14:1940728, allele C of Gm14:1940825, allele G of Gm14:1941047, allele G of Gm14:1941105, allele A of Gm14:1944642, allele T of Gm14:1700746, allele G of Gm14:1706616, allele G of Gm14:1706705, allele A of Gm14:1706782, allele C of Gm14:1708836, allele T of Gm14:1709816, allele T of Gm14:1712174, allele A of Gm14:1717270, allele C of Gm14:1722544, allele T of Gm14:1723756, allele T of Gm14:1725153, allele C of Gm14:1725269, allele G of Gm14:1726477, allele C of Gm14:1731690, allele T of Gm14:1738538, allele T of Gm14:1739491, allele G of Gm14:1739824, allele G of Gm14:1785457, allele T of Gm14:1787260, allele T of Gm14:1796497, allele G of Gm14:1840314, allele A of Gm14:1842837, allele C of Gm14:1850412, allele T of Gm14:1850433, allele T of Gm14:1850907, allele T of Gm14:1851535, allele A of Gm14:1852210, allele C of Gm14:1859255, allele G of Gm14:1860411, allele T of Gm14:1909564, allele G of Gm14:1910968, allele A of Gm14:1935936, allele A of Gm14:1936214, allele G of Gm14:1936876, allele C of Gm14:1939030, allele T of Gm14:1939031, allele A of Gm14:1939081, allele A of Gm14:1940392, allele A of Gm14:1941517, allele G of Gm14:1989692, allele C of Gm14:1989717, allele T of Gm14:1992951, allele A of Gm14:2003498, allele A of Gm14:2004242, allele T of Gm14:2004409, allele A of Gm14:2006356, allele A of Gm14:1698254, allele T of Gm14:1698262, allele C of Gm14:1698269, allele T of Gm14:1701170, allele G of Gm14:1701174, allele T of Gm14:1701472, allele G of Gm14:1701825, allele A of Gm14:1702477, allele G of Gm14:1702552, allele T of Gm14:1702622, allele C of Gm14:1704204, allele A of Gm14:1705803, allele T of Gm14:1706201, allele C of Gm14:1706361, allele A of Gm14:1706585, allele T of Gm14:1706915, allele A of Gm14:1708746, allele A of Gm14:1713290, allele A of Gm14:1718056, allele A of Gm14:1718944, allele T of Gm14:1719373, allele A of Gm14:1723066, allele T of Gm14:1726434, allele G of Gm14:1726867, allele A of Gm14:1727235, allele G of Gm14:1728343, allele A of Gm14:1730044, allele T of Gm14:1731518, allele C of Gm14:1736053, allele C of Gm14:1738509, allele A of Gm14:1738914, allele C of Gm14:1739468, allele C of Gm14:1739718, allele T of Gm14:1739772, allele C of Gm14:1749899, allele A of Gm14:1785516, allele G of Gm14:1785559, allele T of Gm14:1786034, allele A of Gm14:1786259, allele A of Gm14:1793453, allele A of Gm14:1796469, allele A of Gm14:1797188, allele G of Gm14:1798987, allele A of Gm14:1800055, allele T of Gm14:1842374, allele A of Gm14:1850066, allele A of Gm14:1850741, allele C of Gm14:1850928, allele T of Gm14:1852469, allele G of Gm14:1854015, allele A of Gm14:1854023, allele T of Gm14:1859810, allele G of Gm14:1860271, allele G of Gm14:1862541, allele A of Gm14:1867332, allele C of Gm14:1868414, allele C of Gm14:1919937, allele T of Gm14:1935444, allele T of Gm14:1936433, allele G of Gm14:1937170, allele T of Gm14:1937923, allele T of Gm14:1938818, allele T of Gm14:1939343, allele T of Gm14:1939364, allele A of Gm14:1939647, allele T of Gm14:1939662, allele C of Gm14:1939746, allele G of Gm14:1939749, allele A of Gm14:1940207, allele A of Gm14:1940573, allele T of Gm14:1941546, allele A of Gm14:1945962, allele A of Gm14:1946355, allele A of Gm14:1990996, allele C of Gm14:1991421, allele T of Gm14:2001506, allele T of Gm14:2001882, allele G of Gm14:2003462, allele T of Gm14:2003672, allele C of Gm14:1697331, allele G of Gm14:1698694, allele C of Gm14:1700508, allele G of Gm14:1701739, allele G of Gm14:1701858, allele A of Gm14:1702110, allele C of Gm14:1702378, allele T of Gm14:1706884, allele T of Gm14:1706973, allele G of Gm14:1708902, allele T of Gm14:1711952, allele C of Gm14:1712050, allele A of Gm14:1712345, allele G of Gm14:1717261, allele T of Gm14:1722228, allele C of Gm14:1722277, allele C of Gm14:1723101, allele T of Gm14:1724793, allele G of Gm14:1725740, allele T of Gm14:1726334, allele T of Gm14:1727170, allele C of Gm14:1730199, allele G of Gm14:1736329, allele G of Gm14:1736463, allele T of Gm14:1736832, allele G of Gm14:1737771, allele G of Gm14:1738824, allele A of Gm14:1738960, allele G of Gm14:1743818, allele G of Gm14:1756757, allele C of Gm14:1760506, allele T of Gm14:1778710, allele T of Gm14:1783716, allele T of Gm14:1784221, allele G of Gm14:1786098, allele A of Gm14:1797150, allele G of Gm14:1797155, allele T of Gm14:1799977, allele G of Gm14:1811618, allele C of Gm14:1812051, allele A of Gm14:1824464, allele T of Gm14:1839360, allele G of Gm14:1843408, allele T of Gm14:1850104, allele A of Gm14:1851115, allele G of Gm14:1851116, allele G of Gm14:1855960, allele C of Gm14:1858300, allele A of Gm14:1860243, allele C of Gm14:1860675, allele C of Gm14:1866981, allele C of Gm14:1869210, allele A of Gm14:1872625, allele C of Gm14:1902929, allele T of Gm14:1913544, allele T of Gm14:1914780, allele C of Gm14:1935408, allele C of Gm14:1936381, allele A of Gm14:1936425, allele G of Gm14:1939383, allele T of Gm14:1988977, allele A of Gm14:1989052, allele A of Gm14:1990632, allele A of Gm14:1990775, allele G of Gm14:1991550, allele C of Gm14:1999220, allele G of Gm14:2003981, allele G of Gm14:1697620, allele A of Gm14:1697978, allele A of Gm14:1699410, allele G of Gm14:1700636, allele C of Gm14:1701683, allele T of Gm14:1701935, allele T of Gm14:1702661, allele A of Gm14:1702755, allele C of Gm14:1704651, allele C of Gm14:1704664, allele A of Gm14:1712038, allele A of Gm14:1712320, allele A of Gm14:1712713, allele T of Gm14:1712865, allele A of Gm14:1712866, allele T of Gm14:1713999, allele T of Gm14:1719022, allele C of Gm14:1728216, allele T of Gm14:1731767, allele C of Gm14:1735093, allele T of Gm14:1735102, allele A of Gm14:1736945, allele G of Gm14:1743592, allele C of Gm14:1748064, allele C of Gm14:1749126, allele G of Gm14:1754686, allele C of Gm14:1757996, allele C of Gm14:1760483, allele T of Gm14:1783231, allele G of Gm14:1783538, allele T of Gm14:1783756, allele G of Gm14:1786013, allele A of Gm14:1787773, allele C of Gm14:1792218, allele A of Gm14:1799327, allele C of Gm14:1799390, allele T of Gm14:1799540, allele G of Gm14:1800012, allele T of Gm14:1800319, allele T of Gm14:1811113, allele G of Gm14:1811977, allele C of Gm14:1824284, allele C of Gm14:1837872, allele G of Gm14:1837888, allele G of Gm14:1851947, allele G of Gm14:1858277, allele A of Gm14:1861083, allele T of Gm14:1887021, allele C of Gm14:1903293, allele T of Gm14:1910033, allele A of Gm14:1919915, allele A of Gm14:1934737, allele A of Gm14:1946125, allele T of Gm14:1958086, allele T of Gm14:1986602, allele A of Gm14:1987892, allele G of Gm14:1988001, allele A of Gm14:1989951, allele A of Gm14:1991474, allele C of Gm14:1997517, allele C of Gm14:2006319, allele C of Gm14:2017162, allele A of Gm14:2019929, allele C of Gm14:1701692, allele A of Gm14:1702315, allele C of Gm14:1702956, allele A of Gm14:1703980, allele A of Gm14:1706523, allele C of Gm14:1711087, allele C of Gm14:1712377, allele G of Gm14:1714965, allele G of Gm14:1727031, allele A of Gm14:1727851, allele G of Gm14:1728741, allele A of Gm14:1729293, allele A of Gm14:1730480, allele A of Gm14:1730798, allele G of Gm14:1731840, allele G of Gm14:1732232, allele G of Gm14:1732358, allele T of Gm14:1732363, allele T of Gm14:1732403, allele T of Gm14:1735755, allele T of Gm14:1735945, allele A of Gm14:1743425, allele T of Gm14:1744356, allele T of Gm14:1746573, allele A of Gm14:1748959, allele G of Gm14:1750564, allele G of Gm14:1753875, allele A of Gm14:1754007, allele C of Gm14:1755671, allele G of Gm14:1756046, allele T of Gm14:1760299, allele A of Gm14:1760329, allele G of Gm14:1761362, allele T of Gm14:1766162, allele G of Gm14:1780165, allele G of Gm14:1780408, allele C of Gm14:1782983, allele G of Gm14:1789475, allele C of Gm14:1799494, allele C of Gm14:1799548, allele A of Gm14:1799571, allele A of Gm14:1811318, allele T of Gm14:1825691, allele G of Gm14:1834023, allele G of Gm14:1841281, allele T of Gm14:1846850, allele G of Gm14:1887108, allele T of Gm14:1887251, allele C of Gm14:1935369, allele A of Gm14:1969791, allele G of Gm14:1981574, allele G of Gm14:1981644, allele G of Gm14:1983907, allele C of Gm14:1986614, allele G of Gm14:1986675, allele A of Gm14:1987587, allele T of Gm14:1988422, allele C of Gm14:1988424, allele T of Gm14:1988520, allele G of Gm14:1988558, allele G of Gm14:1989403, allele C of Gm14:1991457, allele T of Gm14:1991910, allele T of Gm14:1992830, allele T of Gm14:1993931, allele A of Gm14:1997124, allele C of Gm14:1998879, allele G of Gm14:2004939, allele C of Gm14:2005896, allele A of Gm14:1698816, allele G of Gm14:1701991, allele G of Gm14:1710294, allele C of Gm14:1710303, allele G of Gm14:1711007, allele C of Gm14:1711575, allele G of Gm14:1713816, allele G of Gm14:1728293, allele A of Gm14:1729743, allele A of Gm14:1730281, allele G of Gm14:1730795, allele G of Gm14:1732205, allele A of Gm14:1744432, allele G of Gm14:1751350, allele T of Gm14:1755398, allele G of Gm14:1756188, allele A of Gm14:1761081, allele A of Gm14:1761387, allele A of Gm14:1762867, allele G of Gm14:1765302, allele T of Gm14:1777323, allele A of Gm14:1779323, allele A of Gm14:1780397, allele C of Gm14:1800359, allele A of Gm14:1803234, allele A of Gm14:1811158, allele G of Gm14:1825653, allele G of Gm14:1869500, allele T of Gm14:1876026, allele G of Gm14:1876675, allele G of Gm14:1876844, allele G of Gm14:1876847, allele A of Gm14:1879162, allele T of Gm14:1884603, allele A of Gm14:1904675, allele T of Gm14:1944708, allele C of Gm14:1954056, allele T of Gm14:1954062, allele A of Gm14:1967108, allele C of Gm14:1972107, allele C of Gm14:2006215, allele G of Gm14:2011465, allele G of Gm14:1704689, allele G of Gm14:1711569, allele A of Gm14:1713323, allele G of Gm14:1728052, allele T of Gm14:1729537, allele T of Gm14:1732261, allele A of Gm14:1732593, allele A of Gm14:1735227, allele A of Gm14:1744452, allele A of Gm14:1754156, allele A of Gm14:1755877, allele C of Gm14:1756355, allele A of Gm14:1759702, allele A of Gm14:1780316, allele G of Gm14:1801585, allele A of Gm14:1810099, allele A of Gm14:1810126, allele C of Gm14:1810421, allele C of Gm14:1823776, allele T of Gm14:1875278, allele T of Gm14:1875874, allele G of Gm14:1902961, allele A of Gm14:1910054, allele T of Gm14:1949216, allele G of Gm14:1957357, allele A of Gm14:1957358, allele A of Gm14:1960447, allele C of Gm14:1980728, allele G of Gm14:1984129, allele C of Gm14:1992331, allele T of Gm14:1993147, allele G of Gm14:1995593, allele T of Gm14:1995600, allele G of Gm14:1996952, allele G of Gm14:1997412, allele T of Gm14:2004591, allele G of Gm14:1645762, allele T of Gm14:1713775, allele C of Gm14:1716505, allele A of Gm14:1729186, allele A of Gm14:1729798, allele A of Gm14:1732529, allele T of Gm14:1753922, allele C of Gm14:1759619, allele C of Gm14:1759635, allele A of Gm14:1760144, allele T of Gm14:1761168, allele C of Gm14:1771612, allele T of Gm14:1777105, allele T of Gm14:1778517, allele T of Gm14:1779288, allele A of Gm14:1779568, allele A of Gm14:1804682, allele A of Gm14:1810135, allele G of Gm14:1825225, allele G of Gm14:1828925, allele G of Gm14:1846994, allele A of Gm14:1848374, allele T of Gm14:1874941, allele G of Gm14:1875261, allele A of Gm14:1875470, allele C of Gm14:1879189, allele G of Gm14:1881168, allele A of Gm14:1923548, allele T of Gm14:1923935, allele G of Gm14:1955048, allele C of Gm14:1955118, allele T of Gm14:1955144, allele T of Gm14:1959610, allele A of Gm14:1960454, allele T of Gm14:1967167, allele C of Gm14:1970908, allele G of Gm14:1976442, allele C of Gm14:1981936, allele C of Gm14:1982632, allele C of Gm14:1982756, allele G of Gm14:1984276, allele A of Gm14:1994907, allele G of Gm14:2013156, allele A of Gm14:2019411, allele G of Gm14:1711379, allele T of Gm14:1713140, allele G of Gm14:1727997, allele A of Gm14:1744286, allele T of Gm14:1757864, allele C of Gm14:1759363, allele A of Gm14:1765933, allele C of Gm14:1767525, allele C of Gm14:1767582, allele C of Gm14:1768845, allele T of Gm14:1775564, allele G of Gm14:1778106, allele T of Gm14:1778621, allele T of Gm14:1780281, allele G of Gm14:1780494, allele C of Gm14:1805372, allele A of Gm14:1809090, allele G of Gm14:1810313, allele T of Gm14:1812109, allele A of Gm14:1812114, allele A of Gm14:1824651, allele A of Gm14:1875656, allele G of Gm14:1877489, allele T of Gm14:1878068, allele G of Gm14:1903061, allele A of Gm14:1950278, allele C of Gm14:1950577, allele G of Gm14:1950969, allele T of Gm14:1970110, allele C of Gm14:1982411, allele T of Gm14:1983489, allele T of Gm14:1983507, allele T of Gm14:1983593, allele G of Gm14:1993730, allele T of Gm14:1993788, allele A of Gm14:1993806, allele C of Gm14:2006229, allele C of Gm14:2015853, allele G of Gm14:2359579, allele G of Gm14:1579650, allele T of Gm14:1764334, allele G of Gm14:1764392, allele T of Gm14:1764403, allele C of Gm14:1767878, allele T of Gm14:1805366, allele A of Gm14:1825173, allele C of Gm14:1879313, allele G of Gm14:1886238, allele G of Gm14:1899408, allele G of Gm14:1960137, allele G of Gm14:1971009, allele T of Gm14:1983348, allele C of Gm14:2011607, allele C of Gm14:2012105, allele A of Gm14:2340746, allele C of Gm14:1767671, allele A of Gm14:1771502, allele A of Gm14:1771578, allele G of Gm14:1803995, allele G of Gm14:1805322, allele C of Gm14:1809988, allele C of Gm14:1825310, allele G of Gm14:1888310, allele A of Gm14:1889307, allele A of Gm14:1957202, allele A of Gm14:1981029, allele A of Gm14:1981073, allele T of Gm14:1981255, allele C of Gm14:1992432, allele C of Gm14:2010834, allele C of Gm14:1759440, allele A of Gm14:1775298, allele C of Gm14:1775605, allele C of Gm14:1804116, allele T of Gm14:1804868, allele C of Gm14:1806330, allele T of Gm14:1806377, allele T of Gm14:1889158, allele T of Gm14:1899428, allele T of Gm14:1926218, allele C of Gm14:1928522, allele T of Gm14:1957206, allele A of Gm14:1959033, allele C of Gm14:1969518, allele A of Gm14:1975722, allele C of Gm14:1981293, allele T of Gm14:1983189, allele T of Gm14:2013786, allele G of Gm14:2014487, allele A of Gm14:2191538, allele C of Gm14:2191541, allele T of Gm14:2191563, allele C of Gm14:1711534, allele C of Gm14:1773993, allele G of Gm14:1808347, allele C of Gm14:1808348, allele T of Gm14:1812356, allele C of Gm14:1812358, allele G of Gm14:1874583, allele C of Gm14:1971096, allele G of Gm14:1975723, allele G of Gm14:1976101, allele T of Gm14:1998648, allele T of Gm14:2006929, allele T of Gm14:2013850, allele T of Gm14:2014479, allele T of Gm14:2014481, allele A of Gm14:2101794, allele A of Gm14:766045, allele T of Gm14:1801323, allele A of Gm14:1805026, allele G of Gm14:1879302, allele T of Gm14:1975774, allele G of Gm14:2014429, allele G of Gm14:2191662, allele C of Gm14:584976, allele C of Gm14:2007005, allele A of Gm14:1650177, allele A of Gm14:1899046, allele C of Gm14:806705, allele G of Gm14:1976057, allele T of Gm14:2747160, and allele A of Gm14:1889837, and any combination thereof. In some examples the method comprises detecting at least one favorable allele. In other examples, the method comprises detecting more than one favorable allele, up to and including all of the favorable alleles.

In some examples, the one or more alleles are favorable alleles that positively correlate with tolerance or improved tolerance to stem canker. In other examples, the one or more alleles are disfavored alleles that positively correlate with susceptibility or increased susceptibility to stem canker. In some examples, at least one allele is a favorable allele that positively correlates with improved stem canker resistance when compared to a soybean plant lacking the favorable allele.

Marker loci, haplotypes and marker profiles associated with tolerance or improved tolerance to stem canker, are provided. Further provided are genomic loci that are associated with soybean tolerance or improved tolerance to stem canker. In certain examples, soybean plants or germplasm are identified that have at least one favorable allele, marker locus, haplotype or marker profile that positively correlates with tolerance or improved tolerance to stem canker. However, it is useful for exclusionary purposes during breeding to identify alleles, marker loci, haplotypes, or marker profiles that negatively correlate with tolerance, for example, to eliminate such plants or germplasm from subsequent rounds of breeding.

In one example, marker loci useful for identifying a first soybean plant or first soybean germplasm that displays tolerance or improved tolerance to stem canker are associated with an interval from about 0 cM to about 30 cM on linkage group B2. In some examples the interval is from about 2, 5, 10, 15, 20, 25, 30, 35, 40, 45, 50, 55, 60 or more cM on linkage group B2. In some examples, the interval associated with tolerance or improved tolerance to stem canker is flanked by and includes BARC-013365-00489 and BARCSOYSSR_14_0281 on linkage group B2. In some examples, the interval associated with tolerance or improved tolerance to stem canker is flanked by and includes BARC-013365-00489 and S04785-1 on linkage group B2. In some examples, the interval associated with tolerance or improved tolerance to stem canker is flanked by and includes BARCSOYSSR_14_0036 and BARCSOYSSR_14_0281 on linkage group B2. In other examples, the interval is flanked by and includes S03188-1 and S04785-1 on linkage group B2. In some examples, the interval associated with tolerance or improved tolerance to stem canker is flanked by and includes S03188-1 and S02987-1 on linkage group B2. In other examples, the interval is flanked by and includes nucleotide positions Gm14:584976 and Gm14:2359579. In other examples, the interval is flanked by and includes nucleotide positions Gm14:1697331 and Gm14:2019929. In other examples, the interval is flanked by and includes nucleotide positions Gm14:1706695 and Gm14:1942681. In some examples, the interval comprises at least one or more loci selected from the group consisting of S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14). In some examples the interval comprises one or more loci identified and provided in FIG. 1, or any one of Tables 1-13, or a marker closely linked thereto on linkage group B2.

Kits for characterizing a soybean plant, germplasm or seed are also provided. In some examples a kit comprises primers and/or probes for detecting one or more markers for one or more polynucleotides associated with stem canker tolerance, and instructions for using the primers and/or probes to detect the one or more marker loci and for correlating the detected marker loci with predicted tolerance to stem canker. In some examples the kit comprises at least one primer and/or probe which has a heterologous label that facilitates detection of at least one of a locus, marker, allele, sequence, and/or polymorphism of interest. In some examples, one or more marker loci are selected from the group consisting of S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14), and markers closely linked thereto. In some examples, the primers or probes comprise one or more of SEQ ID NOs: 1-786. In some examples the kit further comprises a buffer or other reagent. In some examples, the kit can include one or more primers or probes for detecting one or more markers for another trait of interest. In some examples, the trait of interest is a transgene. In some example the trait of interest is a native trait.

Isolated polynucleotides are also provided. In one example, an isolated polynucleotide for detecting a marker locus associated with stem canker tolerance is provided. In some examples the isolated polynucleotide comprises at least one heterologous label that facilitates detection of at least one of a locus, marker, allele, sequence, and/or polymorphism of interest. In some examples isolated polynucleotides include a polynucleotide that detects a polymorphism at a locus selected from the group consisting of S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14). In some examples isolated polynucleotides include a polynucleotide that detects a polymorphism selected from the group consisting of Gm14:1088724, Gm14:1197243, Gm14:1594889, Gm14:1650065, Gm14:1727084, Gm14:1727625, Gm14:1747540, Gm14:1748042, Gm14:1748141, Gm14:1755250, Gm14:1755572, Gm14:1788456, Gm14:1923247, Gm14:1925222, Gm14:2129691, Gm14:3468738, Gm14:3726937, Gm14:3727753, Gm14:4204414, Gm14:4946161, Gm14:5028737, Gm14:5096522, Gm14:1706695, Gm14:1723656, Gm14:1723831, Gm14:1724319, Gm14:1738483, Gm14:1739929, Gm14:1850843, Gm14:1853044, Gm14:1863371, Gm14:1863436, Gm14:1863868, Gm14:1914305, Gm14:1936105, Gm14:1939210, Gm14:1940046, Gm14:1942461, Gm14:1942681, Gm14:1700735, Gm14:1702258, Gm14:1715352, Gm14:1718565, Gm14:1722078, Gm14:1722192, Gm14:1723045, Gm14:1724087, Gm14:1724245, Gm14:1724558, Gm14:1724603, Gm14:1725318, Gm14:1726899, Gm14:1738054, Gm14:1784545, Gm14:1785276, Gm14:1793750, Gm14:1842226, Gm14:1850134, Gm14:1850654, Gm14:1850808, Gm14:1851180, Gm14:1851369, Gm14:1851467, Gm14:1851793, Gm14:1852388, Gm14:1852579, Gm14:1855401, Gm14:1867651, Gm14:1867913, Gm14:1867927, Gm14:1910547, Gm14:1910746, Gm14:1910787, Gm14:1911679, Gm14:1916729, Gm14:1916742, Gm14:1919121, Gm14:1934770, Gm14:1935561, Gm14:1935695, Gm14:1935949, Gm14:1936112, Gm14:1936619, Gm14:1936750, Gm14:1936804, Gm14:1936827, Gm14:1937070, Gm14:1937136, Gm14:1937495, Gm14:1938061, Gm14:1940079, Gm14:1940534, Gm14:1940728, Gm14:1940825, Gm14:1941047, Gm14:1941105, Gm14:1944642, Gm14:1700746, Gm14:1706616, Gm14:1706705, Gm14:1706782, Gm14:1708836, Gm14:1709816, Gm14:1712174, Gm14:1717270, Gm14:1722544, Gm14:1723756, Gm14:1725153, Gm14:1725269, Gm14:1726477, Gm14:1731690, Gm14:1738538, Gm14:1739491, Gm14:1739824, Gm14:1785457, Gm14:1787260, Gm14:1796497, Gm14:1840314, Gm14:1842837, Gm14:1850412, Gm14:1850433, Gm14:1850907, Gm14:1851535, Gm14:1852210, Gm14:1859255, Gm14:1860411, Gm14:1909564, Gm14:1910968, Gm14:1935936, Gm14:1936214, Gm14:1936876, Gm14:1939030, Gm14:1939031, Gm14:1939081, Gm14:1940392, Gm14:1941517, Gm14:1989692, Gm14:1989717, Gm14:1992951, Gm14:2003498, Gm14:2004242, Gm14:2004409, Gm14:2006356, Gm14:1698254, Gm14:1698262, Gm14:1698269, Gm14:1701170, Gm14:1701174, Gm14:1701472, Gm14:1701825, Gm14:1702477, Gm14:1702552, Gm14:1702622, Gm14:1704204, Gm14:1705803, Gm14:1706201, Gm14:1706361, Gm14:1706585, Gm14:1706915, Gm14:1708746, Gm14:1713290, Gm14:1718056, Gm14:1718944, Gm14:1719373, Gm14:1723066, Gm14:1726434, Gm14:1726867, Gm14:1727235, Gm14:1728343, Gm14:1730044, Gm14:1731518, Gm14:1736053, Gm14:1738509, Gm14:1738914, Gm14:1739468, Gm14:1739718, Gm14:1739772, Gm14:1749899, Gm14:1785516, Gm14:1785559, Gm14:1786034, Gm14:1786259, Gm14:1793453, Gm14:1796469, Gm14:1797188, Gm14:1798987, Gm14:1800055, Gm14:1842374, Gm14:1850066, Gm14:1850741, Gm14:1850928, Gm14:1852469, Gm14:1854015, Gm14:1854023, Gm14:1859810, Gm14:1860271, Gm14:1862541, Gm14:1867332, Gm14:1868414, Gm14:1919937, Gm14:1935444, Gm14:1936433, Gm14:1937170, Gm14:1937923, Gm14:1938818, Gm14:1939343, Gm14:1939364, Gm14:1939647, Gm14:1939662, Gm14:1939746, Gm14:1939749, Gm14:1940207, Gm14:1940573, Gm14:1941546, Gm14:1945962, Gm14:1946355, Gm14:1990996, Gm14:1991421, Gm14:2001506, Gm14:2001882, Gm14:2003462, Gm14:2003672, Gm14:1697331, Gm14:1698694, Gm14:1700508, Gm14:1701739, Gm14:1701858, Gm14:1702110, Gm14:1702378, Gm14:1706884, Gm14:1706973, Gm14:1708902, Gm14:1711952, Gm14:1712050, Gm14:1712345, Gm14:1717261, Gm14:1722228, Gm14:1722277, Gm14:1723101, Gm14:1724793, Gm14:1725740, Gm14:1726334, Gm14:1727170, Gm14:1730199, Gm14:1736329, Gm14:1736463, Gm14:1736832, Gm14:1737771, Gm14:1738824, Gm14:1738960, Gm14:1743818, Gm14:1756757, Gm14:1760506, Gm14:1778710, Gm14:1783716, Gm14:1784221, Gm14:1786098, Gm14:1797150, Gm14:1797155, Gm14:1799977, Gm14:1811618, Gm14:1812051, Gm14:1824464, Gm14:1839360, Gm14:1843408, Gm14:1850104, Gm14:1851115, Gm14:1851116, Gm14:1855960, Gm14:1858300, Gm14:1860243, Gm14:1860675, Gm14:1866981, Gm14:1869210, Gm14:1872625, Gm14:1902929, Gm14:1913544, Gm14:1914780, Gm14:1935408, Gm14:1936381, Gm14:1936425, Gm14:1939383, Gm14:1988977, Gm14:1989052, Gm14:1990632, Gm14:1990775, Gm14:1991550, Gm14:1999220, Gm14:2003981, Gm14:1697620, Gm14:1697978, Gm14:1699410, Gm14:1700636, Gm14:1701683, Gm14:1701935, Gm14:1702661, Gm14:1702755, Gm14:1704651, Gm14:1704664, Gm14:1712038, Gm14:1712320, Gm14:1712713, Gm14:1712865, Gm14:1712866, Gm14:1713999, Gm14:1719022, Gm14:1728216, Gm14:1731767, Gm14:1735093, Gm14:1735102, Gm14:1736945, Gm14:1743592, Gm14:1748064, Gm14:1749126, Gm14:1754686, Gm14:1757996, Gm14:1760483, Gm14:1783231, Gm14:1783538, Gm14:1783756, Gm14:1786013, Gm14:1787773, Gm14:1792218, Gm14:1799327, Gm14:1799390, Gm14:1799540, Gm14:1800012, Gm14:1800319, Gm14:1811113, Gm14:1811977, Gm14:1824284, Gm14:1837872, Gm14:1837888, Gm14:1851947, Gm14:1858277, Gm14:1861083, Gm14:1887021, Gm14:1903293, Gm14:1910033, Gm14:1919915, Gm14:1934737, Gm14:1946125, Gm14:1958086, Gm14:1986602, Gm14:1987892, Gm14:1988001, Gm14:1989951, Gm14:1991474, Gm14:1997517, Gm14:2006319, Gm14:2017162, Gm14:2019929, Gm14:1701692, Gm14:1702315, Gm14:1702956, Gm14:1703980, Gm14:1706523, Gm14:1711087, Gm14:1712377, Gm14:1714965, Gm14:1727031, Gm14:1727851, Gm14:1728741, Gm14:1729293, Gm14:1730480, Gm14:1730798, Gm14:1731840, Gm14:1732232, Gm14:1732358, Gm14:1732363, Gm14:1732403, Gm14:1735755, Gm14:1735945, Gm14:1743425, Gm14:1744356, Gm14:1746573, Gm14:1748959, Gm14:1750564, Gm14:1753875, Gm14:1754007, Gm14:1755671, Gm14:1756046, Gm14:1760299, Gm14:1760329, Gm14:1761362, Gm14:1766162, Gm14:1780165, Gm14:1780408, Gm14:1782983, Gm14:1789475, Gm14:1799494, Gm14:1799548, Gm14:1799571, Gm14:1811318, Gm14:1825691, Gm14:1834023, Gm14:1841281, Gm14:1846850, Gm14:1887108, Gm14:1887251, Gm14:1935369, Gm14:1969791, Gm14:1981574, Gm14:1981644, Gm14:1983907, Gm14:1986614, Gm14:1986675, Gm14:1987587, Gm14:1988422, Gm14:1988424, Gm14:1988520, Gm14:1988558, Gm14:1989403, Gm14:1991457, Gm14:1991910, Gm14:1992830, Gm14:1993931, Gm14:1997124, Gm14:1998879, Gm14:2004939, Gm14:2005896, Gm14:1698816, Gm14:1701991, Gm14:1710294, Gm14:1710303, Gm14:1711007, Gm14:1711575, Gm14:1713816, Gm14:1728293, Gm14:1729743, Gm14:1730281, Gm14:1730795, Gm14:1732205, Gm14:1744432, Gm14:1751350, Gm14:1755398, Gm14:1756188, Gm14:1761081, Gm14:1761387, Gm14:1762867, Gm14:1765302, Gm14:1777323, Gm14:1779323, Gm14:1780397, Gm14:1800359, Gm14:1803234, Gm14:1811158, Gm14:1825653, Gm14:1869500, Gm14:1876026, Gm14:1876675, Gm14:1876844, Gm14:1876847, Gm14:1879162, Gm14:1884603, Gm14:1904675, Gm14:1944708, Gm14:1954056, Gm14:1954062, Gm14:1967108, Gm14:1972107, Gm14:2006215, Gm14:2011465, Gm14:1704689, Gm14:1711569, Gm14:1713323, Gm14:1728052, Gm14:1729537, Gm14:1732261, Gm14:1732593, Gm14:1735227, Gm14:1744452, Gm14:1754156, Gm14:1755877, Gm14:1756355, Gm14:1759702, Gm14:1780316, Gm14:1801585, Gm14:1810099, Gm14:1810126, Gm14:1810421, Gm14:1823776, Gm14:1875278, Gm14:1875874, Gm14:1902961, Gm14:1910054, Gm14:1949216, Gm14:1957357, Gm14:1957358, Gm14:1960447, Gm14:1980728, Gm14:1984129, Gm14:1992331, Gm14:1993147, Gm14:1995593, Gm14:1995600, Gm14:1996952, Gm14:1997412, Gm14:2004591, Gm14:1645762, Gm14:1713775, Gm14:1716505, Gm14:1729186, Gm14:1729798, Gm14:1732529, Gm14:1753922, Gm14:1759619, Gm14:1759635, Gm14:1760144, Gm14:1761168, Gm14:1771612, Gm14:1777105, Gm14:1778517, Gm14:1779288, Gm14:1779568, Gm14:1804682, Gm14:1810135, Gm14:1825225, Gm14:1828925, Gm14:1846994, Gm14:1848374, Gm14:1874941, Gm14:1875261, Gm14:1875470, Gm14:1879189, Gm14:1881168, Gm14:1923548, Gm14:1923935, Gm14:1955048, Gm14:1955118, Gm14:1955144, Gm14:1959610, Gm14:1960454, Gm14:1967167, Gm14:1970908, Gm14:1976442, Gm14:1981936, Gm14:1982632, Gm14:1982756, Gm14:1984276, Gm14:1994907, Gm14:2013156, Gm14:2019411, Gm14:1711379, Gm14:1713140, Gm14:1727997, Gm14:1744286, Gm14:1757864, Gm14:1759363, Gm14:1765933, Gm14:1767525, Gm14:1767582, Gm14:1768845, Gm14:1775564, Gm14:1778106, Gm14:1778621, Gm14:1780281, Gm14:1780494, Gm14:1805372, Gm14:1809090, Gm14:1810313, Gm14:1812109, Gm14:1812114, Gm14:1824651, Gm14:1875656, Gm14:1877489, Gm14:1878068, Gm14:1903061, Gm14:1950278, Gm14:1950577, Gm14:1950969, Gm14:1970110, Gm14:1982411, Gm14:1983489, Gm14:1983507, Gm14:1983593, Gm14:1993730, Gm14:1993788, Gm14:1993806, Gm14:2006229, Gm14:2015853, Gm14:2359579, Gm14:1579650, Gm14:1764334, Gm14:1764392, Gm14:1764403, Gm14:1767878, Gm14:1805366, Gm14:1825173, Gm14:1879313, Gm14:1886238, Gm14:1899408, Gm14:1960137, Gm14:1971009, Gm14:1983348, Gm14:2011607, Gm14:2012105, Gm14:2340746, Gm14:1767671, Gm14:1771502, Gm14:1771578, Gm14:1803995, Gm14:1805322, Gm14:1809988, Gm14:1825310, Gm14:1888310, Gm14:1889307, Gm14:1957202, Gm14:1981029, Gm14:1981073, Gm14:1981255, Gm14:1992432, Gm14:2010834, Gm14:1759440, Gm14:1775298, Gm14:1775605, Gm14:1804116, Gm14:1804868, Gm14:1806330, Gm14:1806377, Gm14:1889158, Gm14:1899428, Gm14:1926218, Gm14:1928522, Gm14:1957206, Gm14:1959033, Gm14:1969518, Gm14:1975722, Gm14:1981293, Gm14:1983189, Gm14:2013786, Gm14:2014487, Gm14:2191538, Gm14:2191541, Gm14:2191563, Gm14:1711534, Gm14:1773993, Gm14:1808347, Gm14:1808348, Gm14:1812356, Gm14:1812358, Gm14:1874583, Gm14:1971096, Gm14:1975723, Gm14:1976101, Gm14:1998648, Gm14:2006929, Gm14:2013850, Gm14:2014479, Gm14:2014481, Gm14:2101794, Gm14:766045, Gm14:1801323, Gm14:1805026, Gm14:1879302, Gm14:1975774, Gm14:2014429, Gm14:2191662, Gm14:584976, Gm14:2007005, Gm14:1650177, Gm14:1899046, Gm14:806705, Gm14:1976057, Gm14:2747160, and Gm14:1889837. In some examples isolated polynucleotides include a polynucleotide that detects a polymorphism in a polynucleotide selected from the group consisting of Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, and Glyma14g02730.1. In some examples, the polynucleotide comprises a nucleotide sequence selected from the group consisting of SEQ ID NOs: 1-786.

A soybean plant, germplasm, plant part, or seed comprising at least one marker locus in its genome which confers improved stem canker resistance is provided. In some examples, the soybean plant, germplasm, plant part, or seed comprising said at least one marker locus in its genome which confers improved stem canker resistance is an elite soybean variety. In some examples the soybean plant, germplasm, plant part, or seed comprises an interval on LG B2 as described herein. In some examples soybean plant, germplasm, plant part, or seed comprises at least one marker locus selected from the group consisting of S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, 500341-1, S01718-1, and S04785-1 on LG B2 (ch 14). In some examples the soybean plant, germplasm, plant part, or seed comprises at least one marker locus having a polymorphism selected from the group consisting of Gm14:1088724, Gm14:1197243, Gm14:1594889, Gm14:1650065, Gm14:1727084, Gm14:1727625, Gm14:1747540, Gm14:1748042, Gm14:1748141, Gm14:1755250, Gm14:1755572, Gm14:1788456, Gm14:1923247, Gm14:1925222, Gm14:2129691, Gm14:3468738, Gm14:3726937, Gm14:3727753, Gm14:4204414, Gm14:4946161, Gm14:5028737, Gm14:5096522, Gm14:1706695, Gm14:1723656, Gm14:1723831, Gm14:1724319, Gm14:1738483, Gm14:1739929, Gm14:1850843, Gm14:1853044, Gm14:1863371, Gm14:1863436, Gm14:1863868, Gm14:1914305, Gm14:1936105, Gm14:1939210, Gm14:1940046, Gm14:1942461, Gm14:1942681, Gm14:1700735, Gm14:1702258, Gm14:1715352, Gm14:1718565, Gm14:1722078, Gm14:1722192, Gm14:1723045, Gm14:1724087, Gm14:1724245, Gm14:1724558, Gm14:1724603, Gm14:1725318, Gm14:1726899, Gm14:1738054, Gm14:1784545, Gm14:1785276, Gm14:1793750, Gm14:1842226, Gm14:1850134, Gm14:1850654, Gm14:1850808, Gm14:1851180, Gm14:1851369, Gm14:1851467, Gm14:1851793, Gm14:1852388, Gm14:1852579, Gm14:1855401, Gm14:1867651, Gm14:1867913, Gm14:1867927, Gm14:1910547, Gm14:1910746, Gm14:1910787, Gm14:1911679, Gm14:1916729, Gm14:1916742, Gm14:1919121, Gm14:1934770, Gm14:1935561, Gm14:1935695, Gm14:1935949, Gm14:1936112, Gm14:1936619, Gm14:1936750, Gm14:1936804, Gm14:1936827, Gm14:1937070, Gm14:1937136, Gm14:1937495, Gm14:1938061, Gm14:1940079, Gm14:1940534, Gm14:1940728, Gm14:1940825, Gm14:1941047, Gm14:1941105, Gm14:1944642, Gm14:1700746, Gm14:1706616, Gm14:1706705, Gm14:1706782, Gm14:1708836, Gm14:1709816, Gm14:1712174, Gm14:1717270, Gm14:1722544, Gm14:1723756, Gm14:1725153, Gm14:1725269, Gm14:1726477, Gm14:1731690, Gm14:1738538, Gm14:1739491, Gm14:1739824, Gm14:1785457, Gm14:1787260, Gm14:1796497, Gm14:1840314, Gm14:1842837, Gm14:1850412, Gm14:1850433, Gm14:1850907, Gm14:1851535, Gm14:1852210, Gm14:1859255, Gm14:1860411, Gm14:1909564, Gm14:1910968, Gm14:1935936, Gm14:1936214, Gm14:1936876, Gm14:1939030, Gm14:1939031, Gm14:1939081, Gm14:1940392, Gm14:1941517, Gm14:1989692, Gm14:1989717, Gm14:1992951, Gm14:2003498, Gm14:2004242, Gm14:2004409, Gm14:2006356, Gm14:1698254, Gm14:1698262, Gm14:1698269, Gm14:1701170, Gm14:1701174, Gm14:1701472, Gm14:1701825, Gm14:1702477, Gm14:1702552, Gm14:1702622, Gm14:1704204, Gm14:1705803, Gm14:1706201, Gm14:1706361, Gm14:1706585, Gm14:1706915, Gm14:1708746, Gm14:1713290, Gm14:1718056, Gm14:1718944, Gm14:1719373, Gm14:1723066, Gm14:1726434, Gm14:1726867, Gm14:1727235, Gm14:1728343, Gm14:1730044, Gm14:1731518, Gm14:1736053, Gm14:1738509, Gm14:1738914, Gm14:1739468, Gm14:1739718, Gm14:1739772, Gm14:1749899, Gm14:1785516, Gm14:1785559, Gm14:1786034, Gm14:1786259, Gm14:1793453, Gm14:1796469, Gm14:1797188, Gm14:1798987, Gm14:1800055, Gm14:1842374, Gm14:1850066, Gm14:1850741, Gm14:1850928, Gm14:1852469, Gm14:1854015, Gm14:1854023, Gm14:1859810, Gm14:1860271, Gm14:1862541, Gm14:1867332, Gm14:1868414, Gm14:1919937, Gm14:1935444, Gm14:1936433, Gm14:1937170, Gm14:1937923, Gm14:1938818, Gm14:1939343, Gm14:1939364, Gm14:1939647, Gm14:1939662, Gm14:1939746, Gm14:1939749, Gm14:1940207, Gm14:1940573, Gm14:1941546, Gm14:1945962, Gm14:1946355, Gm14:1990996, Gm14:1991421, Gm14:2001506, Gm14:2001882, Gm14:2003462, Gm14:2003672, Gm14:1697331, Gm14:1698694, Gm14:1700508, Gm14:1701739, Gm14:1701858, Gm14:1702110, Gm14:1702378, Gm14:1706884, Gm14:1706973, Gm14:1708902, Gm14:1711952, Gm14:1712050, Gm14:1712345, Gm14:1717261, Gm14:1722228, Gm14:1722277, Gm14:1723101, Gm14:1724793, Gm14:1725740, Gm14:1726334, Gm14:1727170, Gm14:1730199, Gm14:1736329, Gm14:1736463, Gm14:1736832, Gm14:1737771, Gm14:1738824, Gm14:1738960, Gm14:1743818, Gm14:1756757, Gm14:1760506, Gm14:1778710, Gm14:1783716, Gm14:1784221, Gm14:1786098, Gm14:1797150, Gm14:1797155, Gm14:1799977, Gm14:1811618, Gm14:1812051, Gm14:1824464, Gm14:1839360, Gm14:1843408, Gm14:1850104, Gm14:1851115, Gm14:1851116, Gm14:1855960, Gm14:1858300, Gm14:1860243, Gm14:1860675, Gm14:1866981, Gm14:1869210, Gm14:1872625, Gm14:1902929, Gm14:1913544, Gm14:1914780, Gm14:1935408, Gm14:1936381, Gm14:1936425, Gm14:1939383, Gm14:1988977, Gm14:1989052, Gm14:1990632, Gm14:1990775, Gm14:1991550, Gm14:1999220, Gm14:2003981, Gm14:1697620, Gm14:1697978, Gm14:1699410, Gm14:1700636, Gm14:1701683, Gm14:1701935, Gm14:1702661, Gm14:1702755, Gm14:1704651, Gm14:1704664, Gm14:1712038, Gm14:1712320, Gm14:1712713, Gm14:1712865, Gm14:1712866, Gm14:1713999, Gm14:1719022, Gm14:1728216, Gm14:1731767, Gm14:1735093, Gm14:1735102, Gm14:1736945, Gm14:1743592, Gm14:1748064, Gm14:1749126, Gm14:1754686, Gm14:1757996, Gm14:1760483, Gm14:1783231, Gm14:1783538, Gm14:1783756, Gm14:1786013, Gm14:1787773, Gm14:1792218, Gm14:1799327, Gm14:1799390, Gm14:1799540, Gm14:1800012, Gm14:1800319, Gm14:1811113, Gm14:1811977, Gm14:1824284, Gm14:1837872, Gm14:1837888, Gm14:1851947, Gm14:1858277, Gm14:1861083, Gm14:1887021, Gm14:1903293, Gm14:1910033, Gm14:1919915, Gm14:1934737, Gm14:1946125, Gm14:1958086, Gm14:1986602, Gm14:1987892, Gm14:1988001, Gm14:1989951, Gm14:1991474, Gm14:1997517, Gm14:2006319, Gm14:2017162, Gm14:2019929, Gm14:1701692, Gm14:1702315, Gm14:1702956, Gm14:1703980, Gm14:1706523, Gm14:1711087, Gm14:1712377, Gm14:1714965, Gm14:1727031, Gm14:1727851, Gm14:1728741, Gm14:1729293, Gm14:1730480, Gm14:1730798, Gm14:1731840, Gm14:1732232, Gm14:1732358, Gm14:1732363, Gm14:1732403, Gm14:1735755, Gm14:1735945, Gm14:1743425, Gm14:1744356, Gm14:1746573, Gm14:1748959, Gm14:1750564, Gm14:1753875, Gm14:1754007, Gm14:1755671, Gm14:1756046, Gm14:1760299, Gm14:1760329, Gm14:1761362, Gm14:1766162, Gm14:1780165, Gm14:1780408, Gm14:1782983, Gm14:1789475, Gm14:1799494, Gm14:1799548, Gm14:1799571, Gm14:1811318, Gm14:1825691, Gm14:1834023, Gm14:1841281, Gm14:1846850, Gm14:1887108, Gm14:1887251, Gm14:1935369, Gm14:1969791, Gm14:1981574, Gm14:1981644, Gm14:1983907, Gm14:1986614, Gm14:1986675, Gm14:1987587, Gm14:1988422, Gm14:1988424, Gm14:1988520, Gm14:1988558, Gm14:1989403, Gm14:1991457, Gm14:1991910, Gm14:1992830, Gm14:1993931, Gm14:1997124, Gm14:1998879, Gm14:2004939, Gm14:2005896, Gm14:1698816, Gm14:1701991, Gm14:1710294, Gm14:1710303, Gm14:1711007, Gm14:1711575, Gm14:1713816, Gm14:1728293, Gm14:1729743, Gm14:1730281, Gm14:1730795, Gm14:1732205, Gm14:1744432, Gm14:1751350, Gm14:1755398, Gm14:1756188, Gm14:1761081, Gm14:1761387, Gm14:1762867, Gm14:1765302, Gm14:1777323, Gm14:1779323, Gm14:1780397, Gm14:1800359, Gm14:1803234, Gm14:1811158, Gm14:1825653, Gm14:1869500, Gm14:1876026, Gm14:1876675, Gm14:1876844, Gm14:1876847, Gm14:1879162, Gm14:1884603, Gm14:1904675, Gm14:1944708, Gm14:1954056, Gm14:1954062, Gm14:1967108, Gm14:1972107, Gm14:2006215, Gm14:2011465, Gm14:1704689, Gm14:1711569, Gm14:1713323, Gm14:1728052, Gm14:1729537, Gm14:1732261, Gm14:1732593, Gm14:1735227, Gm14:1744452, Gm14:1754156, Gm14:1755877, Gm14:1756355, Gm14:1759702, Gm14:1780316, Gm14:1801585, Gm14:1810099, Gm14:1810126, Gm14:1810421, Gm14:1823776, Gm14:1875278, Gm14:1875874, Gm14:1902961, Gm14:1910054, Gm14:1949216, Gm14:1957357, Gm14:1957358, Gm14:1960447, Gm14:1980728, Gm14:1984129, Gm14:1992331, Gm14:1993147, Gm14:1995593, Gm14:1995600, Gm14:1996952, Gm14:1997412, Gm14:2004591, Gm14:1645762, Gm14:1713775, Gm14:1716505, Gm14:1729186, Gm14:1729798, Gm14:1732529, Gm14:1753922, Gm14:1759619, Gm14:1759635, Gm14:1760144, Gm14:1761168, Gm14:1771612, Gm14:1777105, Gm14:1778517, Gm14:1779288, Gm14:1779568, Gm14:1804682, Gm14:1810135, Gm14:1825225, Gm14:1828925, Gm14:1846994, Gm14:1848374, Gm14:1874941, Gm14:1875261, Gm14:1875470, Gm14:1879189, Gm14:1881168, Gm14:1923548, Gm14:1923935, Gm14:1955048, Gm14:1955118, Gm14:1955144, Gm14:1959610, Gm14:1960454, Gm14:1967167, Gm14:1970908, Gm14:1976442, Gm14:1981936, Gm14:1982632, Gm14:1982756, Gm14:1984276, Gm14:1994907, Gm14:2013156, Gm14:2019411, Gm14:1711379, Gm14:1713140, Gm14:1727997, Gm14:1744286, Gm14:1757864, Gm14:1759363, Gm14:1765933, Gm14:1767525, Gm14:1767582, Gm14:1768845, Gm14:1775564, Gm14:1778106, Gm14:1778621, Gm14:1780281, Gm14:1780494, Gm14:1805372, Gm14:1809090, Gm14:1810313, Gm14:1812109, Gm14:1812114, Gm14:1824651, Gm14:1875656, Gm14:1877489, Gm14:1878068, Gm14:1903061, Gm14:1950278, Gm14:1950577, Gm14:1950969, Gm14:1970110, Gm14:1982411, Gm14:1983489, Gm14:1983507, Gm14:1983593, Gm14:1993730, Gm14:1993788, Gm14:1993806, Gm14:2006229, Gm14:2015853, Gm14:2359579, Gm14:1579650, Gm14:1764334, Gm14:1764392, Gm14:1764403, Gm14:1767878, Gm14:1805366, Gm14:1825173, Gm14:1879313, Gm14:1886238, Gm14:1899408, Gm14:1960137, Gm14:1971009, Gm14:1983348, Gm14:2011607, Gm14:2012105, Gm14:2340746, Gm14:1767671, Gm14:1771502, Gm14:1771578, Gm14:1803995, Gm14:1805322, Gm14:1809988, Gm14:1825310, Gm14:1888310, Gm14:1889307, Gm14:1957202, Gm14:1981029, Gm14:1981073, Gm14:1981255, Gm14:1992432, Gm14:2010834, Gm14:1759440, Gm14:1775298, Gm14:1775605, Gm14:1804116, Gm14:1804868, Gm14:1806330, Gm14:1806377, Gm14:1889158, Gm14:1899428, Gm14:1926218, Gm14:1928522, Gm14:1957206, Gm14:1959033, Gm14:1969518, Gm14:1975722, Gm14:1981293, Gm14:1983189, Gm14:2013786, Gm14:2014487, Gm14:2191538, Gm14:2191541, Gm14:2191563, Gm14:1711534, Gm14:1773993, Gm14:1808347, Gm14:1808348, Gm14:1812356, Gm14:1812358, Gm14:1874583, Gm14:1971096, Gm14:1975723, Gm14:1976101, Gm14:1998648, Gm14:2006929, Gm14:2013850, Gm14:2014479, Gm14:2014481, Gm14:2101794, Gm14:766045, Gm14:1801323, Gm14:1805026, Gm14:1879302, Gm14:1975774, Gm14:2014429, Gm14:2191662, Gm14:584976, Gm14:2007005, Gm14:1650177, Gm14:1899046, Gm14:806705, Gm14:1976057, Gm14:2747160, and Gm14:1889837. In some examples the soybean plant, germplasm, plant part, or seed comprises at least one marker locus comprising a polynucleotide selected from the group consisting of Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, and Glyma14g02730.1. In some examples the marker locus comprises a polynucleotide encoding a non-synonymous codon change in a polynucleotide selected from the group consisting of Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, and Glyma14g02730.1. In some examples, the soybean plant, germplasm, plant part, or seed further comprises resistance to a herbicidal formulation comprising a compound selected from the group consisting of a metribuzin, a hydroxyphenylpyruvatedioxygenase inhibitor, a phosphanoglycine (including but not limited to a glyphosate), a sulfonylurea, a sulfonamide, an imidazolinone, a bialaphos, a phosphinothricin, a mesotrione, an isoxaflutole, an azafenidin, a butafenacil, a sulfosate, a glufosinate, a dicamba, a 2,4-D, and a protox inhibitor. In some examples, resistance to the herbicidal formulation is conferred by a transgene. In some examples, the plant or germplasm further comprises a trait selected from the group consisting of drought tolerance, stress tolerance, disease resistance, herbicide resistance, enhanced yield, modified oil, modified protein, tolerance to chlorotic conditions, and insect resistance, or any combination thereof. In some examples, the trait is selected from the group consisting of brown stem rot resistance, charcoal rot drought complex resistance, Fusarium resistance, Phytophthora resistance, stem canker resistance, sudden death syndrome resistance, Sclerotinia resistance, Cercospora resistance, Soybean Mosaic Virus resistance, carlavirus resistance, anthracnose resistance, target spot resistance, frogeye leaf spot resistance, soybean cyst nematode resistance, root knot nematode resistance, rust resistance, high oleic content, low linolenic content, aphid resistance, stink bug resistance, and iron chlorosis deficiency tolerance, or any combination thereof. In some examples, one or more of the traits is conferred by one or more transgenes, by one or more native loci, or any combination thereof.

In another example a method of producing a cleaned soybean seed is provided, the method comprising cleaning a soybean seed comprising at least one marker locus in its genome which confers improved stem canker resistance is provided. In some examples said one or more loci is selected from the group consisting of S03188-1, S04492-1, S08256-1, S08257-1, S08231-3, S08231-4, S08241-1, S08251-4, S08251-2, S08255-3, S08255-4, S13721-2, 501591-1, S13722-1, S02987-1, S00802-1, S00777-1, S01799-1, S00288-1, S03923-1, S00341-1, S01718-1, and S04785-1 on LG B2 (ch 14), and/or Gm14:1088724, Gm14:1197243, Gm14:1594889, Gm14:1650065, Gm14:1727084, Gm14:1727625, Gm14:1747540, Gm14:1748042, Gm14:1748141, Gm14:1755250, Gm14:1755572, Gm14:1788456, Gm14:1923247, Gm14:1925222, Gm14:2129691, Gm14:3468738, Gm14:3726937, Gm14:3727753, Gm14:4204414, Gm14:4946161, Gm14:5028737, Gm14:5096522, Gm14:1706695, Gm14:1723656, Gm14:1723831, Gm14:1724319, Gm14:1738483, Gm14:1739929, Gm14:1850843, Gm14:1853044, Gm14:1863371, Gm14:1863436, Gm14:1863868, Gm14:1914305, Gm14:1936105, Gm14:1939210, Gm14:1940046, Gm14:1942461, Gm14:1942681, Gm14:1700735, Gm14:1702258, Gm14:1715352, Gm14:1718565, Gm14:1722078, Gm14:1722192, Gm14:1723045, Gm14:1724087, Gm14:1724245, Gm14:1724558, Gm14:1724603, Gm14:1725318, Gm14:1726899, Gm14:1738054, Gm14:1784545, Gm14:1785276, Gm14:1793750, Gm14:1842226, Gm14:1850134, Gm14:1850654, Gm14:1850808, Gm14:1851180, Gm14:1851369, Gm14:1851467, Gm14:1851793, Gm14:1852388, Gm14:1852579, Gm14:1855401, Gm14:1867651, Gm14:1867913, Gm14:1867927, Gm14:1910547, Gm14:1910746, Gm14:1910787, Gm14:1911679, Gm14:1916729, Gm14:1916742, Gm14:1919121, Gm14:1934770, Gm14:1935561, Gm14:1935695, Gm14:1935949, Gm14:1936112, Gm14:1936619, Gm14:1936750, Gm14:1936804, Gm14:1936827, Gm14:1937070, Gm14:1937136, Gm14:1937495, Gm14:1938061, Gm14:1940079, Gm14:1940534, Gm14:1940728, Gm14:1940825, Gm14:1941047, Gm14:1941105, Gm14:1944642, Gm14:1700746, Gm14:1706616, Gm14:1706705, Gm14:1706782, Gm14:1708836, Gm14:1709816, Gm14:1712174, Gm14:1717270, Gm14:1722544, Gm14:1723756, Gm14:1725153, Gm14:1725269, Gm14:1726477, Gm14:1731690, Gm14:1738538, Gm14:1739491, Gm14:1739824, Gm14:1785457, Gm14:1787260, Gm14:1796497, Gm14:1840314, Gm14:1842837, Gm14:1850412, Gm14:1850433, Gm14:1850907, Gm14:1851535, Gm14:1852210, Gm14:1859255, Gm14:1860411, Gm14:1909564, Gm14:1910968, Gm14:1935936, Gm14:1936214, Gm14:1936876, Gm14:1939030, Gm14:1939031, Gm14:1939081, Gm14:1940392, Gm14:1941517, Gm14:1989692, Gm14:1989717, Gm14:1992951, Gm14:2003498, Gm14:2004242, Gm14:2004409, Gm14:2006356, Gm14:1698254, Gm14:1698262, Gm14:1698269, Gm14:1701170, Gm14:1701174, Gm14:1701472, Gm14:1701825, Gm14:1702477, Gm14:1702552, Gm14:1702622, Gm14:1704204, Gm14:1705803, Gm14:1706201, Gm14:1706361, Gm14:1706585, Gm14:1706915, Gm14:1708746, Gm14:1713290, Gm14:1718056, Gm14:1718944, Gm14:1719373, Gm14:1723066, Gm14:1726434, Gm14:1726867, Gm14:1727235, Gm14:1728343, Gm14:1730044, Gm14:1731518, Gm14:1736053, Gm14:1738509, Gm14:1738914, Gm14:1739468, Gm14:1739718, Gm14:1739772, Gm14:1749899, Gm14:1785516, Gm14:1785559, Gm14:1786034, Gm14:1786259, Gm14:1793453, Gm14:1796469, Gm14:1797188, Gm14:1798987, Gm14:1800055, Gm14:1842374, Gm14:1850066, Gm14:1850741, Gm14:1850928, Gm14:1852469, Gm14:1854015, Gm14:1854023, Gm14:1859810, Gm14:1860271, Gm14:1862541, Gm14:1867332, Gm14:1868414, Gm14:1919937, Gm14:1935444, Gm14:1936433, Gm14:1937170, Gm14:1937923, Gm14:1938818, Gm14:1939343, Gm14:1939364, Gm14:1939647, Gm14:1939662, Gm14:1939746, Gm14:1939749, Gm14:1940207, Gm14:1940573, Gm14:1941546, Gm14:1945962, Gm14:1946355, Gm14:1990996, Gm14:1991421, Gm14:2001506, Gm14:2001882, Gm14:2003462, Gm14:2003672, Gm14:1697331, Gm14:1698694, Gm14:1700508, Gm14:1701739, Gm14:1701858, Gm14:1702110, Gm14:1702378, Gm14:1706884, Gm14:1706973, Gm14:1708902, Gm14:1711952, Gm14:1712050, Gm14:1712345, Gm14:1717261, Gm14:1722228, Gm14:1722277, Gm14:1723101, Gm14:1724793, Gm14:1725740, Gm14:1726334, Gm14:1727170, Gm14:1730199, Gm14:1736329, Gm14:1736463, Gm14:1736832, Gm14:1737771, Gm14:1738824, Gm14:1738960, Gm14:1743818, Gm14:1756757, Gm14:1760506, Gm14:1778710, Gm14:1783716, Gm14:1784221, Gm14:1786098, Gm14:1797150, Gm14:1797155, Gm14:1799977, Gm14:1811618, Gm14:1812051, Gm14:1824464, Gm14:1839360, Gm14:1843408, Gm14:1850104, Gm14:1851115, Gm14:1851116, Gm14:1855960, Gm14:1858300, Gm14:1860243, Gm14:1860675, Gm14:1866981, Gm14:1869210, Gm14:1872625, Gm14:1902929, Gm14:1913544, Gm14:1914780, Gm14:1935408, Gm14:1936381, Gm14:1936425, Gm14:1939383, Gm14:1988977, Gm14:1989052, Gm14:1990632, Gm14:1990775, Gm14:1991550, Gm14:1999220, Gm14:2003981, Gm14:1697620, Gm14:1697978, Gm14:1699410, Gm14:1700636, Gm14:1701683, Gm14:1701935, Gm14:1702661, Gm14:1702755, Gm14:1704651, Gm14:1704664, Gm14:1712038, Gm14:1712320, Gm14:1712713, Gm14:1712865, Gm14:1712866, Gm14:1713999, Gm14:1719022, Gm14:1728216, Gm14:1731767, Gm14:1735093, Gm14:1735102, Gm14:1736945, Gm14:1743592, Gm14:1748064, Gm14:1749126, Gm14:1754686, Gm14:1757996, Gm14:1760483, Gm14:1783231, Gm14:1783538, Gm14:1783756, Gm14:1786013, Gm14:1787773, Gm14:1792218, Gm14:1799327, Gm14:1799390, Gm14:1799540, Gm14:1800012, Gm14:1800319, Gm14:1811113, Gm14:1811977, Gm14:1824284, Gm14:1837872, Gm14:1837888, Gm14:1851947, Gm14:1858277, Gm14:1861083, Gm14:1887021, Gm14:1903293, Gm14:1910033, Gm14:1919915, Gm14:1934737, Gm14:1946125, Gm14:1958086, Gm14:1986602, Gm14:1987892, Gm14:1988001, Gm14:1989951, Gm14:1991474, Gm14:1997517, Gm14:2006319, Gm14:2017162, Gm14:2019929, Gm14:1701692, Gm14:1702315, Gm14:1702956, Gm14:1703980, Gm14:1706523, Gm14:1711087, Gm14:1712377, Gm14:1714965, Gm14:1727031, Gm14:1727851, Gm14:1728741, Gm14:1729293, Gm14:1730480, Gm14:1730798, Gm14:1731840, Gm14:1732232, Gm14:1732358, Gm14:1732363, Gm14:1732403, Gm14:1735755, Gm14:1735945, Gm14:1743425, Gm14:1744356, Gm14:1746573, Gm14:1748959, Gm14:1750564, Gm14:1753875, Gm14:1754007, Gm14:1755671, Gm14:1756046, Gm14:1760299, Gm14:1760329, Gm14:1761362, Gm14:1766162, Gm14:1780165, Gm14:1780408, Gm14:1782983, Gm14:1789475, Gm14:1799494, Gm14:1799548, Gm14:1799571, Gm14:1811318, Gm14:1825691, Gm14:1834023, Gm14:1841281, Gm14:1846850, Gm14:1887108, Gm14:1887251, Gm14:1935369, Gm14:1969791, Gm14:1981574, Gm14:1981644, Gm14:1983907, Gm14:1986614, Gm14:1986675, Gm14:1987587, Gm14:1988422, Gm14:1988424, Gm14:1988520, Gm14:1988558, Gm14:1989403, Gm14:1991457, Gm14:1991910, Gm14:1992830, Gm14:1993931, Gm14:1997124, Gm14:1998879, Gm14:2004939, Gm14:2005896, Gm14:1698816, Gm14:1701991, Gm14:1710294, Gm14:1710303, Gm14:1711007, Gm14:1711575, Gm14:1713816, Gm14:1728293, Gm14:1729743, Gm14:1730281, Gm14:1730795, Gm14:1732205, Gm14:1744432, Gm14:1751350, Gm14:1755398, Gm14:1756188, Gm14:1761081, Gm14:1761387, Gm14:1762867, Gm14:1765302, Gm14:1777323, Gm14:1779323, Gm14:1780397, Gm14:1800359, Gm14:1803234, Gm14:1811158, Gm14:1825653, Gm14:1869500, Gm14:1876026, Gm14:1876675, Gm14:1876844, Gm14:1876847, Gm14:1879162, Gm14:1884603, Gm14:1904675, Gm14:1944708, Gm14:1954056, Gm14:1954062, Gm14:1967108, Gm14:1972107, Gm14:2006215, Gm14:2011465, Gm14:1704689, Gm14:1711569, Gm14:1713323, Gm14:1728052, Gm14:1729537, Gm14:1732261, Gm14:1732593, Gm14:1735227, Gm14:1744452, Gm14:1754156, Gm14:1755877, Gm14:1756355, Gm14:1759702, Gm14:1780316, Gm14:1801585, Gm14:1810099, Gm14:1810126, Gm14:1810421, Gm14:1823776, Gm14:1875278, Gm14:1875874, Gm14:1902961, Gm14:1910054, Gm14:1949216, Gm14:1957357, Gm14:1957358, Gm14:1960447, Gm14:1980728, Gm14:1984129, Gm14:1992331, Gm14:1993147, Gm14:1995593, Gm14:1995600, Gm14:1996952, Gm14:1997412, Gm14:2004591, Gm14:1645762, Gm14:1713775, Gm14:1716505, Gm14:1729186, Gm14:1729798, Gm14:1732529, Gm14:1753922, Gm14:1759619, Gm14:1759635, Gm14:1760144, Gm14:1761168, Gm14:1771612, Gm14:1777105, Gm14:1778517, Gm14:1779288, Gm14:1779568, Gm14:1804682, Gm14:1810135, Gm14:1825225, Gm14:1828925, Gm14:1846994, Gm14:1848374, Gm14:1874941, Gm14:1875261, Gm14:1875470, Gm14:1879189, Gm14:1881168, Gm14:1923548, Gm14:1923935, Gm14:1955048, Gm14:1955118, Gm14:1955144, Gm14:1959610, Gm14:1960454, Gm14:1967167, Gm14:1970908, Gm14:1976442, Gm14:1981936, Gm14:1982632, Gm14:1982756, Gm14:1984276, Gm14:1994907, Gm14:2013156, Gm14:2019411, Gm14:1711379, Gm14:1713140, Gm14:1727997, Gm14:1744286, Gm14:1757864, Gm14:1759363, Gm14:1765933, Gm14:1767525, Gm14:1767582, Gm14:1768845, Gm14:1775564, Gm14:1778106, Gm14:1778621, Gm14:1780281, Gm14:1780494, Gm14:1805372, Gm14:1809090, Gm14:1810313, Gm14:1812109, Gm14:1812114, Gm14:1824651, Gm14:1875656, Gm14:1877489, Gm14:1878068, Gm14:1903061, Gm14:1950278, Gm14:1950577, Gm14:1950969, Gm14:1970110, Gm14:1982411, Gm14:1983489, Gm14:1983507, Gm14:1983593, Gm14:1993730, Gm14:1993788, Gm14:1993806, Gm14:2006229, Gm14:2015853, Gm14:2359579, Gm14:1579650, Gm14:1764334, Gm14:1764392, Gm14:1764403, Gm14:1767878, Gm14:1805366, Gm14:1825173, Gm14:1879313, Gm14:1886238, Gm14:1899408, Gm14:1960137, Gm14:1971009, Gm14:1983348, Gm14:2011607, Gm14:2012105, Gm14:2340746, Gm14:1767671, Gm14:1771502, Gm14:1771578, Gm14:1803995, Gm14:1805322, Gm14:1809988, Gm14:1825310, Gm14:1888310, Gm14:1889307, Gm14:1957202, Gm14:1981029, Gm14:1981073, Gm14:1981255, Gm14:1992432, Gm14:2010834, Gm14:1759440, Gm14:1775298, Gm14:1775605, Gm14:1804116, Gm14:1804868, Gm14:1806330, Gm14:1806377, Gm14:1889158, Gm14:1899428, Gm14:1926218, Gm14:1928522, Gm14:1957206, Gm14:1959033, Gm14:1969518, Gm14:1975722, Gm14:1981293, Gm14:1983189, Gm14:2013786, Gm14:2014487, Gm14:2191538, Gm14:2191541, Gm14:2191563, Gm14:1711534, Gm14:1773993, Gm14:1808347, Gm14:1808348, Gm14:1812356, Gm14:1812358, Gm14:1874583, Gm14:1971096, Gm14:1975723, Gm14:1976101, Gm14:1998648, Gm14:2006929, Gm14:2013850, Gm14:2014479, Gm14:2014481, Gm14:2101794, Gm14:766045, Gm14:1801323, Gm14:1805026, Gm14:1879302, Gm14:1975774, Gm14:2014429, Gm14:2191662, Gm14:584976, Gm14:2007005, Gm14:1650177, Gm14:1899046, Gm14:806705, Gm14:1976057, Gm14:2747160, and Gm14:1889837, wherein said seed or plant produced therefrom has improved stem canker resistance when compared to a soybean plant or germplasm lacking said one or more loci in its genome. In some examples, the seed or plant produced therefrom comprises a haplotype or marker profile comprising at least two marker loci selected from the group consisting of S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14), and/or Gm14:1088724, Gm14:1197243, Gm14:1594889, Gm14:1650065, Gm14:1727084, Gm14:1727625, Gm14:1747540, Gm14:1748042, Gm14:1748141, Gm14:1755250, Gm14:1755572, Gm14:1788456, Gm14:1923247, Gm14:1925222, Gm14:2129691, Gm14:3468738, Gm14:3726937, Gm14:3727753, Gm14:4204414, Gm14:4946161, Gm14:5028737, Gm14:5096522, Gm14:1706695, Gm14:1723656, Gm14:1723831, Gm14:1724319, Gm14:1738483, Gm14:1739929, Gm14:1850843, Gm14:1853044, Gm14:1863371, Gm14:1863436, Gm14:1863868, Gm14:1914305, Gm14:1936105, Gm14:1939210, Gm14:1940046, Gm14:1942461, Gm14:1942681, Gm14:1700735, Gm14:1702258, Gm14:1715352, Gm14:1718565, Gm14:1722078, Gm14:1722192, Gm14:1723045, Gm14:1724087, Gm14:1724245, Gm14:1724558, Gm14:1724603, Gm14:1725318, Gm14:1726899, Gm14:1738054, Gm14:1784545, Gm14:1785276, Gm14:1793750, Gm14:1842226, Gm14:1850134, Gm14:1850654, Gm14:1850808, Gm14:1851180, Gm14:1851369, Gm14:1851467, Gm14:1851793, Gm14:1852388, Gm14:1852579, Gm14:1855401, Gm14:1867651, Gm14:1867913, Gm14:1867927, Gm14:1910547, Gm14:1910746, Gm14:1910787, Gm14:1911679, Gm14:1916729, Gm14:1916742, Gm14:1919121, Gm14:1934770, Gm14:1935561, Gm14:1935695, Gm14:1935949, Gm14:1936112, Gm14:1936619, Gm14:1936750, Gm14:1936804, Gm14:1936827, Gm14:1937070, Gm14:1937136, Gm14:1937495, Gm14:1938061, Gm14:1940079, Gm14:1940534, Gm14:1940728, Gm14:1940825, Gm14:1941047, Gm14:1941105, Gm14:1944642, Gm14:1700746, Gm14:1706616, Gm14:1706705, Gm14:1706782, Gm14:1708836, Gm14:1709816, Gm14:1712174, Gm14:1717270, Gm14:1722544, Gm14:1723756, Gm14:1725153, Gm14:1725269, Gm14:1726477, Gm14:1731690, Gm14:1738538, Gm14:1739491, Gm14:1739824, Gm14:1785457, Gm14:1787260, Gm14:1796497, Gm14:1840314, Gm14:1842837, Gm14:1850412, Gm14:1850433, Gm14:1850907, Gm14:1851535, Gm14:1852210, Gm14:1859255, Gm14:1860411, Gm14:1909564, Gm14:1910968, Gm14:1935936, Gm14:1936214, Gm14:1936876, Gm14:1939030, Gm14:1939031, Gm14:1939081, Gm14:1940392, Gm14:1941517, Gm14:1989692, Gm14:1989717, Gm14:1992951, Gm14:2003498, Gm14:2004242, Gm14:2004409, Gm14:2006356, Gm14:1698254, Gm14:1698262, Gm14:1698269, Gm14:1701170, Gm14:1701174, Gm14:1701472, Gm14:1701825, Gm14:1702477, Gm14:1702552, Gm14:1702622, Gm14:1704204, Gm14:1705803, Gm14:1706201, Gm14:1706361, Gm14:1706585, Gm14:1706915, Gm14:1708746, Gm14:1713290, Gm14:1718056, Gm14:1718944, Gm14:1719373, Gm14:1723066, Gm14:1726434, Gm14:1726867, Gm14:1727235, Gm14:1728343, Gm14:1730044, Gm14:1731518, Gm14:1736053, Gm14:1738509, Gm14:1738914, Gm14:1739468, Gm14:1739718, Gm14:1739772, Gm14:1749899, Gm14:1785516, Gm14:1785559, Gm14:1786034, Gm14:1786259, Gm14:1793453, Gm14:1796469, Gm14:1797188, Gm14:1798987, Gm14:1800055, Gm14:1842374, Gm14:1850066, Gm14:1850741, Gm14:1850928, Gm14:1852469, Gm14:1854015, Gm14:1854023, Gm14:1859810, Gm14:1860271, Gm14:1862541, Gm14:1867332, Gm14:1868414, Gm14:1919937, Gm14:1935444, Gm14:1936433, Gm14:1937170, Gm14:1937923, Gm14:1938818, Gm14:1939343, Gm14:1939364, Gm14:1939647, Gm14:1939662, Gm14:1939746, Gm14:1939749, Gm14:1940207, Gm14:1940573, Gm14:1941546, Gm14:1945962, Gm14:1946355, Gm14:1990996, Gm14:1991421, Gm14:2001506, Gm14:2001882, Gm14:2003462, Gm14:2003672, Gm14:1697331, Gm14:1698694, Gm14:1700508, Gm14:1701739, Gm14:1701858, Gm14:1702110, Gm14:1702378, Gm14:1706884, Gm14:1706973, Gm14:1708902, Gm14:1711952, Gm14:1712050, Gm14:1712345, Gm14:1717261, Gm14:1722228, Gm14:1722277, Gm14:1723101, Gm14:1724793, Gm14:1725740, Gm14:1726334, Gm14:1727170, Gm14:1730199, Gm14:1736329, Gm14:1736463, Gm14:1736832, Gm14:1737771, Gm14:1738824, Gm14:1738960, Gm14:1743818, Gm14:1756757, Gm14:1760506, Gm14:1778710, Gm14:1783716, Gm14:1784221, Gm14:1786098, Gm14:1797150, Gm14:1797155, Gm14:1799977, Gm14:1811618, Gm14:1812051, Gm14:1824464, Gm14:1839360, Gm14:1843408, Gm14:1850104, Gm14:1851115, Gm14:1851116, Gm14:1855960, Gm14:1858300, Gm14:1860243, Gm14:1860675, Gm14:1866981, Gm14:1869210, Gm14:1872625, Gm14:1902929, Gm14:1913544, Gm14:1914780, Gm14:1935408, Gm14:1936381, Gm14:1936425, Gm14:1939383, Gm14:1988977, Gm14:1989052, Gm14:1990632, Gm14:1990775, Gm14:1991550, Gm14:1999220, Gm14:2003981, Gm14:1697620, Gm14:1697978, Gm14:1699410, Gm14:1700636, Gm14:1701683, Gm14:1701935, Gm14:1702661, Gm14:1702755, Gm14:1704651, Gm14:1704664, Gm14:1712038, Gm14:1712320, Gm14:1712713, Gm14:1712865, Gm14:1712866, Gm14:1713999, Gm14:1719022, Gm14:1728216, Gm14:1731767, Gm14:1735093, Gm14:1735102, Gm14:1736945, Gm14:1743592, Gm14:1748064, Gm14:1749126, Gm14:1754686, Gm14:1757996, Gm14:1760483, Gm14:1783231, Gm14:1783538, Gm14:1783756, Gm14:1786013, Gm14:1787773, Gm14:1792218, Gm14:1799327, Gm14:1799390, Gm14:1799540, Gm14:1800012, Gm14:1800319, Gm14:1811113, Gm14:1811977, Gm14:1824284, Gm14:1837872, Gm14:1837888, Gm14:1851947, Gm14:1858277, Gm14:1861083, Gm14:1887021, Gm14:1903293, Gm14:1910033, Gm14:1919915, Gm14:1934737, Gm14:1946125, Gm14:1958086, Gm14:1986602, Gm14:1987892, Gm14:1988001, Gm14:1989951, Gm14:1991474, Gm14:1997517, Gm14:2006319, Gm14:2017162, Gm14:2019929, Gm14:1701692, Gm14:1702315, Gm14:1702956, Gm14:1703980, Gm14:1706523, Gm14:1711087, Gm14:1712377, Gm14:1714965, Gm14:1727031, Gm14:1727851, Gm14:1728741, Gm14:1729293, Gm14:1730480, Gm14:1730798, Gm14:1731840, Gm14:1732232, Gm14:1732358, Gm14:1732363, Gm14:1732403, Gm14:1735755, Gm14:1735945, Gm14:1743425, Gm14:1744356, Gm14:1746573, Gm14:1748959, Gm14:1750564, Gm14:1753875, Gm14:1754007, Gm14:1755671, Gm14:1756046, Gm14:1760299, Gm14:1760329, Gm14:1761362, Gm14:1766162, Gm14:1780165, Gm14:1780408, Gm14:1782983, Gm14:1789475, Gm14:1799494, Gm14:1799548, Gm14:1799571, Gm14:1811318, Gm14:1825691, Gm14:1834023, Gm14:1841281, Gm14:1846850, Gm14:1887108, Gm14:1887251, Gm14:1935369, Gm14:1969791, Gm14:1981574, Gm14:1981644, Gm14:1983907, Gm14:1986614, Gm14:1986675, Gm14:1987587, Gm14:1988422, Gm14:1988424, Gm14:1988520, Gm14:1988558, Gm14:1989403, Gm14:1991457, Gm14:1991910, Gm14:1992830, Gm14:1993931, Gm14:1997124, Gm14:1998879, Gm14:2004939, Gm14:2005896, Gm14:1698816, Gm14:1701991, Gm14:1710294, Gm14:1710303, Gm14:1711007, Gm14:1711575, Gm14:1713816, Gm14:1728293, Gm14:1729743, Gm14:1730281, Gm14:1730795, Gm14:1732205, Gm14:1744432, Gm14:1751350, Gm14:1755398, Gm14:1756188, Gm14:1761081, Gm14:1761387, Gm14:1762867, Gm14:1765302, Gm14:1777323, Gm14:1779323, Gm14:1780397, Gm14:1800359, Gm14:1803234, Gm14:1811158, Gm14:1825653, Gm14:1869500, Gm14:1876026, Gm14:1876675, Gm14:1876844, Gm14:1876847, Gm14:1879162, Gm14:1884603, Gm14:1904675, Gm14:1944708, Gm14:1954056, Gm14:1954062, Gm14:1967108, Gm14:1972107, Gm14:2006215, Gm14:2011465, Gm14:1704689, Gm14:1711569, Gm14:1713323, Gm14:1728052, Gm14:1729537, Gm14:1732261, Gm14:1732593, Gm14:1735227, Gm14:1744452, Gm14:1754156, Gm14:1755877, Gm14:1756355, Gm14:1759702, Gm14:1780316, Gm14:1801585, Gm14:1810099, Gm14:1810126, Gm14:1810421, Gm14:1823776, Gm14:1875278, Gm14:1875874, Gm14:1902961, Gm14:1910054, Gm14:1949216, Gm14:1957357, Gm14:1957358, Gm14:1960447, Gm14:1980728, Gm14:1984129, Gm14:1992331, Gm14:1993147, Gm14:1995593, Gm14:1995600, Gm14:1996952, Gm14:1997412, Gm14:2004591, Gm14:1645762, Gm14:1713775, Gm14:1716505, Gm14:1729186, Gm14:1729798, Gm14:1732529, Gm14:1753922, Gm14:1759619, Gm14:1759635, Gm14:1760144, Gm14:1761168, Gm14:1771612, Gm14:1777105, Gm14:1778517, Gm14:1779288, Gm14:1779568, Gm14:1804682, Gm14:1810135, Gm14:1825225, Gm14:1828925, Gm14:1846994, Gm14:1848374, Gm14:1874941, Gm14:1875261, Gm14:1875470, Gm14:1879189, Gm14:1881168, Gm14:1923548, Gm14:1923935, Gm14:1955048, Gm14:1955118, Gm14:1955144, Gm14:1959610, Gm14:1960454, Gm14:1967167, Gm14:1970908, Gm14:1976442, Gm14:1981936, Gm14:1982632, Gm14:1982756, Gm14:1984276, Gm14:1994907, Gm14:2013156, Gm14:2019411, Gm14:1711379, Gm14:1713140, Gm14:1727997, Gm14:1744286, Gm14:1757864, Gm14:1759363, Gm14:1765933, Gm14:1767525, Gm14:1767582, Gm14:1768845, Gm14:1775564, Gm14:1778106, Gm14:1778621, Gm14:1780281, Gm14:1780494, Gm14:1805372, Gm14:1809090, Gm14:1810313, Gm14:1812109, Gm14:1812114, Gm14:1824651, Gm14:1875656, Gm14:1877489, Gm14:1878068, Gm14:1903061, Gm14:1950278, Gm14:1950577, Gm14:1950969, Gm14:1970110, Gm14:1982411, Gm14:1983489, Gm14:1983507, Gm14:1983593, Gm14:1993730, Gm14:1993788, Gm14:1993806, Gm14:2006229, Gm14:2015853, Gm14:2359579, Gm14:1579650, Gm14:1764334, Gm14:1764392, Gm14:1764403, Gm14:1767878, Gm14:1805366, Gm14:1825173, Gm14:1879313, Gm14:1886238, Gm14:1899408, Gm14:1960137, Gm14:1971009, Gm14:1983348, Gm14:2011607, Gm14:2012105, Gm14:2340746, Gm14:1767671, Gm14:1771502, Gm14:1771578, Gm14:1803995, Gm14:1805322, Gm14:1809988, Gm14:1825310, Gm14:1888310, Gm14:1889307, Gm14:1957202, Gm14:1981029, Gm14:1981073, Gm14:1981255, Gm14:1992432, Gm14:2010834, Gm14:1759440, Gm14:1775298, Gm14:1775605, Gm14:1804116, Gm14:1804868, Gm14:1806330, Gm14:1806377, Gm14:1889158, Gm14:1899428, Gm14:1926218, Gm14:1928522, Gm14:1957206, Gm14:1959033, Gm14:1969518, Gm14:1975722, Gm14:1981293, Gm14:1983189, Gm14:2013786, Gm14:2014487, Gm14:2191538, Gm14:2191541, Gm14:2191563, Gm14:1711534, Gm14:1773993, Gm14:1808347, Gm14:1808348, Gm14:1812356, Gm14:1812358, Gm14:1874583, Gm14:1971096, Gm14:1975723, Gm14:1976101, Gm14:1998648, Gm14:2006929, Gm14:2013850, Gm14:2014479, Gm14:2014481, Gm14:2101794, Gm14:766045, Gm14:1801323, Gm14:1805026, Gm14:1879302, Gm14:1975774, Gm14:2014429, Gm14:2191662, Gm14:584976, Gm14:2007005, Gm14:1650177, Gm14:1899046, Gm14:806705, Gm14:1976057, Gm14:2747160, and Gm14:1889837. In some examples, the cleaned soybean seed has enhanced yield characteristics when compared to a soybean seed which has not been cleaned. Cleaned soybean seed produced by the methods are also provided.

In another example a method of producing a treated soybean seed is provided, the method comprising treating a soybean seed comprising at least one marker locus in its genome which confers improved stem canker resistance is provided. In some examples said one or more loci is selected from the group consisting of S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14), wherein said seed or plant produced therefrom has improved stem canker resistance when compared to a soybean plant or germplasm said one or more loci in its genome. In some examples, the seed or plant produced therefrom comprises a haplotype or marker profile comprising at least two marker loci selected from the group consisting of S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14). In some examples, the seed treatment comprises a fungicide, an insecticide, or any combination thereof. In some examples the seed treatment comprises trifloxystrobin, metalaxyl, imidacloprid, Bacillus spp., and any combination thereof. In some examples the seed treatment comprises picoxystrobin, penthiopyrad, cyantraniliprole, chlorantraniliprole, and any combination thereof. In some examples, the seed treatment improves seed germination under normal and/or stress environments, early stand count, vigor, yield, root formation, nodulation, and any combination thereof when compared to a soybean seed which has not been treated. In some examples seed treatment reduces seed dust levels, insect damage, pathogen establishment and/or damage, plant virus infection and/or damage, and any combination thereof. Treated soybean seed produced by the methods are also provided.

In certain examples, detecting comprises amplifying the marker locus or a portion of the marker locus and detecting the resulting amplified marker amplicon. In particular examples, the amplifying comprises: 1) admixing an amplification primer or amplification primer pair and, optionally at least one nucleic acid probe, with a nucleic acid isolated from the first soybean plant or germplasm, wherein the primer or primer pair and optional probe is complementary or partially complementary to at least a portion of the marker locus and is capable of initiating DNA polymerization by a DNA polymerase using the soybean nucleic acid as a template; and 2) extending the primer or primer pair in a DNA polymerization reaction comprising a DNA polymerase and a template nucleic acid to generate at least one amplicon. In some examples detecting is accomplished by using at least one primer or probe comprising a heterologous detectable label. In particular examples, the detection comprises real time PCR analysis.

The methods can be used to aid in the selection of breeding plants, lines, and populations containing tolerance to stem canker for use in introgression of this trait into elite soybean germplasm, or germplasm of proven genetic superiority suitable for variety release. Also provided is a method for introgressing a soybean QTL, marker, marker profile, and/or haplotype associated with stem canker tolerance into non-tolerant or less tolerant soybean germplasm. According to the method, markers, marker profiles, and/or haplotypes are used to select soybean plants containing the improved tolerance trait. Plants so selected can be used in a soybean breeding program. Through the process of introgression, the QTL, marker, marker profile, and/or haplotype associated with an improved stem canker tolerance is introduced from plants identified using marker-assisted selection (MAS) to other plants. According to the method, agronomically desirable plants and seeds can be produced containing the QTL, marker, marker profile, and/or haplotype associated with a stem canker tolerance from germplasm containing the QTL, marker, marker profile, and/or haplotype.

Also provided herein is a method for producing a soybean plant adapted for conferring improved stem canker tolerance. First, donor soybean plants for a parental line containing one or more tolerance QTL, marker, haplotype, and/or marker profile are selected. According to the method, selection can be accomplished via MAS as explained herein. Selected plant material may represent, among others, an inbred line, a hybrid line, a heterogeneous population of soybean plants, or an individual plant. According to techniques well known in the art of plant breeding, this donor parental line is crossed with a second parental line. In some examples, the second parental line is a high yielding line. This cross produces a segregating plant population composed of genetically heterogeneous plants. Plants of the segregating plant population are screened for one or more of the tolerance QTL, marker, haplotype, and/or marker profile. Further breeding may include, among other techniques, additional crosses with other lines, with hybrids, backcrossing, or self-crossing. The result is a line of soybean plants that has improved tolerance to stem canker and optionally also has other desirable traits from one or more other soybean lines.

Soybean plants, germplasm, seeds, tissue cultures, variants and mutants having improved stem canker tolerance produced by the foregoing methods are provided. Also provided are isolated nucleic acids, kits, and systems useful for the identification and selection methods disclosed herein.

It is to be understood that this invention is not limited to particular embodiments, which can, of course, vary. It is also to be understood that the terminology used herein is for the purpose of describing particular embodiments only, and is not intended to be limiting. Further, all publications referred to herein are incorporated by reference for the purpose cited to the same extent as if each was specifically and individually indicated to be incorporated by reference herein.

### DEFINITIONS

As used in this specification and the appended claims, terms in the singular and the singular forms “a,” “an,” and “the,” for example, include plural referents unless the content clearly dictates otherwise. Thus, for example, reference to “plant,” “the plant,” or “a plant” also includes a plurality of plants; also, depending on the context, use of the term “plant” can also include genetically similar or identical progeny of that plant; use of the term “a nucleic acid” optionally includes, as a practical matter, many copies of that nucleic acid molecule; similarly, the term “probe” optionally (and typically) encompasses many similar or identical probe molecules.

As used herein, the terms “comprises,” “comprising,” “includes,” “including,” “has,” “having,” “contains”, “containing,” “characterized by” or any other variation thereof, are intended to cover a non-exclusive inclusion, subject to any limitation explicitly indicated. For example, a composition, mixture, process, method, article, or apparatus that comprises a list of elements is not necessarily limited to only those elements but may include other elements not expressly listed or inherent to such composition, mixture, process, method, article, or apparatus.

The transitional phrase “consisting of” excludes any element, step, or ingredient not specified. In a claim, such would close the claim to the inclusion of materials other than those recited except for impurities ordinarily associated therewith. When the phrase “consisting of” appears in a clause of the body of a claim, rather than immediately following the preamble, it limits only the element set forth in that clause; other elements are not excluded from the claim as a whole. The transitional phrase “consisting essentially of” is used to define a composition, method or apparatus that includes materials, steps, features, components, or elements, in addition to those literally disclosed, provided that these additional materials, steps, features, components, or elements do not materially affect the basic and novel characteristic(s) of the claimed invention.

Certain definitions used in the specification and claims are provided below. In order to provide a clear and consistent understanding of the specification and claims, including the scope to be given such terms, the following definitions are provided:

“Allele” means any of one or more alternative forms of a genetic sequence. In a diploid cell or organism, the two alleles of a given sequence typically occupy corresponding loci on a pair of homologous chromosomes. With regard to a SNP marker, allele refers to the specific nucleotide base present at that SNP locus in that individual plant. A favorable allele is an allele correlated with the preferred phenotype. A favorable allele is typically denoted as a nucleotide variant on one strand at a specified position of a polynucleotide, but clearly includes the nucleotide at the corresponding position on the complementary strand of the polynucleotide. For example, a favorable allele “T” at position 10 of polynucleotide X includes the “A” at the corresponding position of the other strand of polynucleotide X based nucleotide base pairing.

The term “amplifying” in the context of nucleic acid amplification is any process whereby additional copies of a selected nucleic acid (or a transcribed form thereof) are produced. An “amplicon” is an amplified nucleic acid, e.g., a nucleic acid that is produced by amplifying a template nucleic acid by any available amplification method.

“Backcrossing” is a process in which a breeder crosses a progeny variety back to one of the parental genotypes one or more times.

The term “chromosome segment” designates a contiguous linear span of genomic DNA that resides in planta on a single chromosome. “Chromosome interval” refers to a chromosome segment defined by specific flanking marker loci.

“Cultivar” and “variety” are used synonymously and mean a group of plants within a species (e.g., Glycine max) that share certain genetic traits that separate them from other possible varieties within that species. Soybean cultivars are inbred lines produced after several generations of self-pollinations. Individuals within a soybean cultivar are homogeneous, nearly genetically identical, with most loci in the homozygous state.

An “elite line” is an agronomically superior line that has resulted from many cycles of breeding and selection for superior agronomic performance. Numerous elite lines are available and known to those of skill in the art of soybean breeding.

An “elite population” is an assortment of elite individuals or lines that can be used to represent the state of the art in terms of agronomically superior genotypes of a given crop species, such as soybean.

An “exotic soybean strain” or an “exotic soybean germplasm” is a strain or germplasm derived from a soybean not belonging to an available elite soybean line or strain of germplasm. In the context of a cross between two soybean plants or strains of germplasm, an exotic germplasm is not closely related by descent to the elite germplasm with which it is crossed. Most commonly, the exotic germplasm is not derived from any known elite line of soybean, but rather is selected to introduce novel genetic elements (typically novel alleles) into a breeding program.

A “genetic map” is a description of genetic association or linkage relationships among loci on one or more chromosomes (or linkage groups) within a given species, generally depicted in a diagrammatic or tabular form.

“Genotype” refers to the genetic constitution of a cell or organism.

“Germplasm” means the genetic material that comprises the physical foundation of the hereditary qualities of an organism. As used herein, germplasm includes seeds and living tissue from which new plants may be grown; or, another plant part, such as leaf, stem, pollen, or cells, that may be cultured into a whole plant. Germplasm resources provide sources of genetic traits used by plant breeders to improve commercial cultivars.

“Stem canker resistance” and “stem canker tolerance” are used interchangeably to classify plants that when exposed to or inoculated with a stem canker pathogen will show reduced damage or symptoms as compared to an appropriate control plant treated under substantially identical conditions.

An individual is “homozygous” if the individual has only one type of allele at a given locus (e.g., a diploid individual has a copy of the same allele at a locus for each of two homologous chromosomes). An individual is “heterozygous” if more than one allele type is present at a given locus (e.g., a diploid individual with one copy each of two different alleles). The term “homogeneity” indicates that members of a group have the same genotype at one or more specific loci. In contrast, the term “heterogeneity” is used to indicate that individuals within the group differ in genotype at one or more specific loci.

“Introgression” means the entry or introduction of a gene, a transgene, a QTL, a marker, a haplotype, a marker profile, a trait, a trait locus, or a chromosomal segment from the genome of one plant into the genome of another plant.

The terms “label” and “detectable label” refer to a molecule capable of detection. A detectable label can also include a combination of a reporter and a quencher, such as are employed in FRET probes or TaqMan™ probes. The term “reporter” refers to a substance or a portion thereof which is capable of exhibiting a detectable signal, which signal can be suppressed by a quencher. The detectable signal of the reporter is, e.g., fluorescence in the detectable range. The term “quencher” refers to a substance or portion thereof which is capable of suppressing, reducing, inhibiting, etc., the detectable signal produced by the reporter. As used herein, the terms “quenching” and “fluorescence energy transfer” refer to the process whereby, when a reporter and a quencher are in close proximity, and the reporter is excited by an energy source, a substantial portion of the energy of the excited state nonradiatively transfers to the quencher where it either dissipates nonradiatively or is emitted at a different emission wavelength than that of the reporter.

A “line” or “strain” is a group of individuals of identical parentage that are generally inbred to some degree and that are generally homozygous and homogeneous at most loci (isogenic or near isogenic). A “subline” refers to an inbred subset of descendents that are genetically distinct from other similarly inbred subsets descended from the same progenitor. Traditionally, a subline has been derived by inbreeding the seed from an individual soybean plant selected at the F3 to F5 generation until the residual segregating loci are homozygous (fixed) across most or all loci. Commercial soybean varieties (or lines) are typically produced by aggregating (bulking) the self-pollinated progeny of a single F3 to F5 plant from a controlled cross between 2 genetically different parents. While the variety typically appears uniform, the self-pollinating variety derived from the selected plant eventually (e.g., F8) becomes a mixture of homozygous plants that can vary in genotype at any locus that was heterozygous in the originally selected F3 to F5 plant. Marker-based sublines that differ from each other based on qualitative polymorphism at the DNA level at one or more specific marker loci are derived by genotyping a sample of seed derived from individual self-pollinated progeny derived from a selected F3-F5 plant. The seed sample can be genotyped directly as seed, or as plant tissue grown from such a seed sample. Optionally, seed sharing a common genotype at the specified locus (or loci) are bulked providing a subline that is genetically homogenous at identified loci important for a trait of interest (e.g., yield, tolerance, etc.).

“Linkage” refers to the tendency for alleles to segregate together more often than expected by chance if their transmission was independent. Typically, linkage refers to loci on the same chromosome that do not segregate independently during meiosis. Genetic recombination occurs with an assumed random frequency over the entire genome. Genetic maps are constructed by measuring the frequency of recombination between pairs of traits or markers, the lower the frequency of recombination, the greater the degree of linkage. A 1/100 probability of recombination per generation is defined as a map distance of 1.0 centiMorgan (1.0 cM).

With regard to physical position on a chromosome, closely linked markers can be separated, for example, by about 1 megabase (Mb; 1 million nucleotides), about 500 kilobases (Kb; 1000 nucleotides), about 400 Kb, about 300 Kb, about 200 Kb, about 100 Kb, about 50 Kb, about 25 Kb, about 10 Kb, about 5 Kb, about 4 Kb, about 3 Kb, about 2 Kb, about 1 Kb, about 500 nucleotides, about 250 nucleotides, or less.

When referring to the relationship between two genetic elements, such as a genetic element contributing to tolerance and a proximal marker, “coupling” phase linkage indicates the state where the “favorable” allele at the tolerance locus is physically associated on the same chromosome strand as the “favorable” allele of the respective linked marker locus. In coupling phase, both favorable alleles are inherited together by progeny that inherit that chromosome strand. In “repulsion” phase linkage, the “favorable” allele at the locus of interest (e.g., a QTL for tolerance) is physically linked with an “unfavorable” allele at the proximal marker locus, and the two “favorable” alleles are not inherited together (i.e., the two loci are “out of phase” with each other).

“Linkage disequilibrium” refers to cases wherein alleles tend to remain together when segregating from parents to offspring, with a greater frequency than expected from their individual frequencies. Linkage disequilibrium indicates a non-random association of alleles.

“Linkage group” refers to traits or markers that generally co-segregate. A linkage group generally corresponds to a chromosomal region containing genetic material that encodes the traits or markers.

“Locus” is a defined segment of DNA.

A “map location,” a “map position,” or, “relative map position” is an assigned location on a genetic map relative to associated genetic markers where a specified marker can be found within a given species. Map positions are generally provided in centimorgans (cM), unless otherwise indicated, genetic positions provided are based on the Glycine max consensus map v 4.0 as provided by Hyten et al. (2010) Crop Sci 50:960-968. A “physical position” or “physical location” is the position, typically in nucleotide bases, of a particular nucleotide, such as a SNP nucleotide, on the chromosome. Unless otherwise indicated, the physical position within the soybean genome provided is based on the Glyma 1.0 genome sequence described in Schmutz et al. (2010) Nature 463:178-183, available from the Phytozome website (phytozome-dot-net/soybean), and includes the corresponding coordinates in future revisions of the soybean genome assembly.

“Mapping” is the process of defining the linkage relationships of loci through the use of genetic markers, populations segregating for the markers, and standard genetic principles of recombination frequency.

“Marker” or “molecular marker” is a term used to denote a nucleic acid or amino acid sequence that is sufficiently unique to characterize a specific locus on the genome. Any detectable polymorphic trait can be used as a marker so long as it is inherited differentially. Preferably, the marker also exhibits linkage disequilibrium with a phenotypic trait of interest.

“Marker assisted selection” refers to the process of selecting a desired trait or traits in a plant or plants by detecting one or more nucleic acids from the plant, where the nucleic acid is linked to the desired trait, and then selecting the plant or germplasm possessing those one or more nucleic acids.

“Marker profile” denotes a combination of particular alleles present within a particular plant's genome at two or more marker loci which are not necessarily linked, including but not limited to instances when two or more loci are on two or more different linkage groups. In certain other examples a plant's marker profile comprises one or more haplotypes. In some examples, the marker profile encompasses two or more loci for the same trait, such as stem canker resistance. In other examples, the marker profile encompasses two or more loci associated with two or more traits of interest, such as stem canker resistance and a second trait of interest.

“Haplotype” refers to a combination of particular alleles present within a particular plant's genome at two or more marker loci, for instance at two or more loci on a particular linkage group or chromosome. A haplotype can include 2, 3, 4, 5, 6, 7, 8, 9, 10, 11, 12, 13, 14, 15, 16, 17, 18, 19, 20, or more marker loci used to define a haplotype for a particular plant.

“Maturity Group” is an agreed-on industry division of groups of varieties, based on the zones in which they are adapted primarily according to day length and/or latitude. Soybean varieties are grouped into 13 maturity groups, depending on the climate and latitude for which they are adapted. Soybean maturities are divided into relative maturity groups (denoted as 000, 00, 0, I, II, III, IV, V, VI, VII, VIII, IX, X, or 000, 00, 0, 1, 2, 3, 4, 5, 6, 7, 8, 9, 10). These maturity groups are given numbers, with numbers 000, 00, 0 and 1 typically being adapted to Canada and the northern United States, groups VII, VIII and IX being grown in the southern regions, and Group X is tropical. Within a maturity group are sub-groups. A sub-group is a tenth of a relative maturity group (for example 1.3 would indicate a group 1 and subgroup 3). Within narrow comparisons, the difference of a tenth of a relative maturity group equates very roughly to a day difference in maturity at harvest.

A “mixed defined plant population” refers to a plant population containing many different families and lines of plants. Typically, the defined plant population exhibits a quantitative variability for a phenotype that is of interest. “Multiple plant families” refers to different families of related plants within a population.

The term “plant” includes reference to an immature or mature whole plant, including a plant from which seed or grain or anthers have been removed. Seed or an embryo that will produce the plant is also considered to be the plant.

“Plant parts” means any portion or piece of a plant, including leaves, stems, buds, roots, root tips, anthers, seed, grain, embryo, pollen, ovules, flowers, cotyledons, hypocotyls, pods, flowers, shoots, stalks, tissues, tissue cultures, cells, and the like.

“Polymorphism” means a change or difference between two related nucleic acids. A “nucleotide polymorphism” refers to a nucleotide that is different in one sequence when compared to a related sequence when the two nucleic acids are aligned for maximal correspondence. Polymorphism is inclusive of one or more nucleotide changes such as substitutions, deletions, and additions.

“Polynucleotide,” “polynucleotide sequence,” “nucleic acid sequence,” “nucleic acid fragment,” and “oligonucleotide” are used interchangeably herein to indicate a polymer of nucleotides that is single- or multi-stranded, that optionally contains synthetic, non-natural, or altered RNA or DNA nucleotide bases. A DNA polynucleotide may be comprised of one or more strands of cDNA, genomic DNA, synthetic DNA, or mixtures thereof.

“Primer” refers to an oligonucleotide which is capable of acting as a point of initiation of nucleic acid synthesis or replication along a complementary strand when placed under conditions in which synthesis of a complementary strand is catalyzed by a polymerase. Typically, primers are about 10 to 30 nucleotides in length, but longer or shorter sequences can be employed. Primers may be provided in double-stranded form, though the single-stranded form is more typically used. A primer can further contain a detectable label, for example a 5′ end label.

“Probe” refers to an oligonucleotide that is complementary (though not necessarily fully complementary) to a polynucleotide of interest and forms a duplexed structure by hybridization with at least one strand of the polynucleotide of interest. Typically, probes are oligonucleotides from 10 to 50 nucleotides in length, but longer or shorter sequences can be employed. A probe can further contain a detectable label.

“Quantitative trait locus” or “QTL” refer to the genetic elements controlling a quantitative trait.

“Recombination frequency” is the frequency of a crossing over event (recombination) between two genetic loci. Recombination frequency can be observed by following the segregation of markers and/or traits during meiosis.

“Tolerance,” “improved tolerance,” “resistance,” and “improved resistance” are used interchangeably herein and refer to any type of increase in resistance or tolerance, or any type of decrease in susceptibility. A “tolerant plant” or “tolerant plant variety” need not possess absolute or complete tolerance. Instead, a “tolerant plant,” “tolerant plant variety,” or a plant or plant variety with “improved tolerance” will have a level of resistance or tolerance which is higher than that of a comparable susceptible or less tolerant plant or variety.

“Self crossing” or “self pollination” or “selfing” is a process through which a breeder crosses a plant with itself; for example, a second generation hybrid F2 with itself to yield progeny designated F2:3.

“SNP” or “single nucleotide polymorphism” means a sequence variation that occurs when a single nucleotide (A, T, C, or G) in the genome sequence is altered or variable. “SNP markers” exist when SNPs are mapped to sites on the soybean genome.

The term “yield” refers to the productivity per unit area of a particular plant product of commercial value. For example, yield of soybean is commonly measured in bushels of seed per acre or metric tons of seed per hectare per season. Yield is affected by both genetic and environmental factors. Yield is the final culmination of all agronomic traits.

An “isolated” or “purified” polynucleotide or polypeptide, or biologically active portion thereof, is substantially or essentially free from components that normally accompany or interact with the polynucleotide or polypeptide as found in its naturally occurring environment. Typically, an “isolated” polynucleotide is free of sequences (optimally protein encoding sequences) that naturally flank the polynucleotide (i.e., sequences located at the 5′ and 3′ ends of the polynucleotide) in the genomic DNA of the organism from which the polynucleotide is derived. For example, the isolated polynucleotide can contain less than about 5 kb, 4 kb, 3 kb, 2 kb, 1 kb, 0.5 kb, or 0.1 kb of nucleotide sequence that naturally flank the polynucleotide in genomic DNA of the cell from which the polynucleotide is derived. A polypeptide that is substantially free of cellular material includes preparations of polypeptides having less than about 30%, 20%, 10%, 5%, or 1% (by dry weight) of contaminating protein, culture media, or other chemical components.

Stem Canker is a fungal disease of soybeans characterized by two distinct disease isolates, Southern Stem Canker (Diaporthe phaseolorum var. meridionalis) and Northern Stem Canker (Diaporthe phaseolorum var. caulivora). The southern isolate is considered endemic to the entire United States southern soybean growing region. After its first occurrence in the United States in 1973, Southern Stem Canker caused widespread crop damage during the early 1980's, with losses up to 100% in severely affected fields. Currently, there does not appear to be any overlap in the geographical regions impacted by northern versus southern pathogen isolates. Stem canker is now a worldwide problem, with occurrences in at least the U.S., Europe, Brazil and Argentina. Stem canker infection occurs during the early vegetative growth period (V1 to V5). Like many other fungal scorch diseases, leaf symptoms generally don't appear until the late reproductive growth stages (R3 to R6). Stem canker symptoms begin as reddish brown lesions on the stem, generally centered on a leaf node and concentrated on one side of the stem. As the disease progresses, cankers will enlarge longitudinally, turn dark brown to black in color, become slightly sunken and eventually completely girdle stems. At this point, the free flow of nutrients and water is disrupted in the plant. Cankers, which coalesce, may be confused with stem discoloration caused by Phytophthora. However, stem canker usually forms higher on the plant than does Phytophthora. Severe stem canker can result in premature plant death. Foliage of diseased plants initially exhibits interveinal yellowing. This is followed by tissue death between the veins. Eventually, leaves die and usually remain attached to leaf stems (petioles).

As the external lesions increase in size, the internal pith area of the stem is slowly degraded. In some cases, the external symptoms do not accurately reflect the amount of lesion growth and internal damage to the stem. Some instances of stem canker can result in plant lodging. Upon entering reproductive growth, plants can begin to exhibit interveinal chlorotic and necrotic leaf symptoms. These leaf symptoms are caused in part by a phytotoxin that is exuded by the pathogen in the stem. Upon plant death, the leaves generally remain attached to the stem. Damage due to stem canker primarily results from premature plant death, along with reductions in seed number and seed size.

Losses from Southern Stem Canker can be minimized by use of resistant cultivars and delayed planting of problem fields. Conventional tillage can also offer some benefits due to the fact that the pathogen over-winters in crop debris, which can serve as a source of infection for subsequent growing seasons. The pathogen can also be associated with seed. No seed treatments are suggested for the control of stem canker. Production practices that reduce the chances of stress conditions during the growing season can help mitigate yield reductions from stem canker. Additionally, foliar-applied fungicides are not recommended stem canker control. The suggested timing for using foliar-applied fungicides to control other diseases appears to have little effect on reducing stem canker. Use of varieties having resistance to stem canker is the best option to protect against crop loss.

A soybean plant, germplasm, plant part, or seed further comprising resistance to a herbicidal formulation is provided. For example, the herbicidal formulation can comprise a compound selected from the group consisting of a metribuzin, glyphosate, a hydroxyphenylpyruvatedioxygenase (HPPD) inhibitor, a sulfonamide, a sulfonylurea, an imidazolinone, a bialaphos, a phosphinothricin, a mesotrione, an isoxaflutole, an azafenidin, a butafenacil, a sulfosate, a glufosinate, a dicamba, a 2,4-D, and a protox inhibitor. In some examples, resistance to an herbicidal formulation is conferred by a transgene. In other examples, resistance to an herbicide or herbicidal formulation is conferred as a naturally occurring (native) trait.

Glyphosate resistance can be conferred from genes including but not limited to EPSPS, GAT, GOX, and the like, such as described in U.S. Pat. Nos. 6,248,876; 5,627,061; 5,804,425; 5,633,435; 5,145,783; 4,971,908; 5,312,910; 5,188,642; 4,940,835; 5,866,775; 6,225,114; 6,130,366; 5,310,667; 4,535,060; 4,769,061; 5,633,448; 5,510,471; RE36,449; RE37,287 E; 5,491,288; 5,776,760; 5,463,175; 8,044,261; 7,527,955; 7,666,643; 7,998,703; 7,951,995; 7,968,770; 8,088,972, 7,863,503; and US20030083480; WO 97/04103; WO 00/66746; WO 01/66704; and WO 00/66747, which are each incorporated herein by reference in their entireties for all purposes. Additionally, glyphosate tolerant plants can be generated through the selection of naturally occurring mutations that impart tolerance to glyphosate.

HPPD resistance can be conferred by genes including exemplary sequences disclosed in U.S. Pat. Nos. 6,245,968; 6,268,549; and 6,069,115; and WO 99/23886, which are each incorporated herein by reference in their entireties for all purposes. Mutant hydroxyphenylpyruvatedioxygenases having this activity are also known. For further examples see US20110185444 and US20110185445.

Resistance to auxins, such as 2,4-D or dicamba, can be provided by polynucleotides as described, for example, in WO2005/107437, US20070220629, and U.S. Pat. No. 7,838,733 and in Herman et al. (2005) J. Biol. Chem. 280:24759-24767, each which is herein incorporated by reference.

Resistance to PPO-inhibiting herbicides can be provided as described in U.S. Pat. Nos. 6,288,306; 6,282,837; and 5,767,373; and WO 01/12825, each of which is herein incorporated by reference. Plants containing such polynucleotides can exhibit improved tolerance to any of a variety of herbicides which target the protox enzyme. Resistance can also be conferred as described in US20100186131; US20110185444; US20100024080, each of which is herein incorporated by reference.

The development of plants containing an exogenous phosphinothricin acetyltransferase which confers resistance to glufosinate, bialaphos, or phosphinothricin is described, for example, in U.S. Pat. Nos. 5,969,213; 5,489,520; 5,550,318; 5,874,265; 5,919,675; 5,561,236; 5,648,477; 5,646,024; 6,177,616; and 5,879,903, which are each incorporated herein by reference in their entireties for all purposes. Mutant phosphinothricin acetyltransferase having this activity are also known in the art.

In some examples, the plant or germplasm further comprises a trait selected from the group consisting of drought tolerance, stress tolerance, disease resistance, herbicide resistance, enhanced yield, modified oil, modified protein, tolerance to chlorotic conditions, and insect resistance, or any combination thereof. In some examples, the trait is selected from the group consisting of brown stem rot resistance, charcoal rot drought complex resistance, Fusarium resistance, Phytophthora resistance, stem canker resistance, sudden death syndrome resistance, Sclerotinia resistance, Cercospora resistance, anthracnose resistance, target spot resistance, frogeye leaf spot resistance, soybean cyst nematode resistance, root knot nematode resistance, rust resistance, high oleic content, low linolenic content, aphid resistance, stink bug resistance, and iron chlorosis deficiency tolerance, or any combination thereof. In some examples, one or more of the traits is conferred by one or more transgenes, by one or more native loci, or any combination thereof. Examples of markers and loci conferring improved iron chlorosis deficiency tolerance are disclosed in US20110258743, U.S. Pat. No. 7,582,806, and U.S. Pat. No. 7,977,533, each of which is herein incorporated by reference. Various disease resistance loci and markers are disclosed, for example, in WO1999031964, U.S. Pat. No. 5,948,953, U.S. Pat. No. 5,689,035, US20090170112, US20090172829, US20090172830, US20110271409, US20110145953, U.S. Pat. No. 7,642,403, U.S. Pat. No. 7,919,675, US20110131677, U.S. Pat. No. 7,767,882, U.S. Pat. No. 7,910,799, US20080263720, U.S. Pat. No. 7,507,874, US20040034890, US20110055960, US20110185448, US20110191893, US20120017339, U.S. Pat. No. 7,250,552, U.S. Pat. No. 7,595,432, U.S. Pat. No. 7,790,949, U.S. Pat. No. 7,956,239, U.S. Pat. No. 7,968,763, each of which is herein incorporated by reference. Markers and loci conferring improved yield are provided, for example, in U.S. Pat. No. 7,973,212 and WO2000018963, each of which is herein incorporated by reference. Markers and loci conferring improved resistance to insects are disclosed in, for example, US20090049565, U.S. Pat. No. 7,781,648, US20100263085, U.S. Pat. No. 7,928,286, U.S. Pat. No. 7,994,389, and WO2011116131, each of which is herein incorporated by reference. Markers and loci for modified soybean oil content or composition are disclosed in, for example, US20120028255 and US20110277173, each of which is herein incorporated by reference. Methods and compositions to modified soybean oil content are described in, for example, WO2008147935, U.S. Pat. No. 8,119,860; U.S. Pat. No. 8,119,784; U.S. Pat. No. 8,101,189; U.S. Pat. No. 8,058,517; U.S. Pat. No. 8,049,062; U.S. Pat. No. 8,124,845; U.S. Pat. No. 7,790,959; U.S. Pat. No. 7,531,718; U.S. Pat. No. 7,504,563; and U.S. Pat. No. 6,949,698, each of which is herein incorporated by reference. Markers and loci conferring tolerance to nematodes are disclosed in, for example, US20090064354, US20090100537, US20110083234, US20060225150, US20110083224, U.S. Pat. No. 5,491,081, U.S. Pat. No. 6,162,967, U.S. Pat. No. 6,538,175, U.S. Pat. No. 7,872,171, U.S. Pat. No. 6,096,944, and U.S. Pat. No. 6,300,541, each of which is herein incorporated by reference. Resistance to nematodes may be conferred using a transgenic approach as described, for example, in U.S. Pat. No. 6,284,948 and U.S. Pat. No. 6,228,992, each of which is herein incorporated by reference. Plant phenotypes can be modified using isopentyl transferase polynucleotides as described, for example, in U.S. Pat. No. 7,553,951 and U.S. Pat. No. 7,893,236, each of which is herein incorporated by reference.

Soybean plants, germplasm, cells, or seed may be evaluated by any method to determine the presence of a polynucleotide and/or polypeptide associated with tolerance to stem canker. Methods include phenotypic evaluations, genotypic evaluations, or combinations thereof. The progeny plants may be evaluated in subsequent generations for stem canker resistance, and other desirable traits. Resistance to stem canker may be evaluated by exposing plants, cells, or seed to one or more appropriate stem canker pathogens and evaluating injury. Genotypic evaluation of the plants, germplasm, cells or seeds includes using techniques such as isozyme electrophoresis, restriction fragment length polymorphisms (RFLPs), randomly amplified polymorphic DNAs (RAPDs), arbitrarily primed polymerase chain reaction (AP-PCR), DNA amplification fingerprinting (DAF), sequence characterized amplified regions (SCARs), amplified fragment length polymorphisms (AFLPs), simple sequence repeats (SSRs), single nucleotide polymorphisms (SNPs), insertions or deletions (indels), sequencing, northern blots, southern blots, marker profiles, and the like.

Provided are markers, marker combinations, haplotypes, and/or marker profiles associated with tolerance of soybean plants to stem canker, as well as related primers and/or probes and methods for the use of any of the foregoing for identifying and/or selecting soybean plants with improved tolerance to stem canker. A method for determining the presence or absence of at least one allele of a particular marker or combination of markers associated with tolerance to stem canker comprises analyzing genomic DNA from a soybean plant or germplasm to determine if at least one, or a plurality, of such markers is present or absent and if present, and determining the allelic form of the marker(s). In some examples a plurality of markers on a single linkage group are investigated, and the markers present in the particular plant or germplasm can be used to determine a haplotype for that plant/germplasm. In other examples a plurality of markers on distinct linkage groups are investigated, and the markers present in the particular plant or germplasm can be used to determine a marker profile for that plant or germplasm.

Soybean seeds, plants, and plant parts comprising a polynucleotide associated with stem canker tolerance may be cleaned and/or treated. The resulting seeds, plants, or plant parts produced by the cleaning and/or treating process(es) may exhibit enhanced yield characteristics. Enhanced yield characteristics can include one or more of the following: increased germination efficiency under normal and/or stress conditions, improved plant physiology, growth and/or development, such as water use efficiency, water retention efficiency, improved nitrogen use, enhanced carbon assimilation, improved photosynthesis, and accelerated maturation, and improved disease and/or pathogen tolerance. Yield characteristics can furthermore include enhanced plant architecture (under stress and non-stress conditions), including but not limited to early flowering, flowering control for hybrid seed production, seedling vigor, plant size, internode number and distance, root growth, seed size, fruit size, pod size, pod or ear number, seed number per pod or ear, seed mass, enhanced seed filling, reduced seed dispersal, reduced pod dehiscence and lodging resistance. Further yield characteristics include seed composition, such as carbohydrate content, protein content, oil content and composition, nutritional value, reduction in anti-nutritional compounds, improved processability and better storage stability.

Cleaning a seed or seed cleaning refers to the removal of impurities and debris material from the harvested seed. Material to be removed from the seed includes but is not limited to soil, and plant waste, pebbles, weed seeds, broken soybean seeds, fungi, bacteria, insect material, including insect eggs, larvae, and parts thereof, and any other pests that exist with the harvested crop. The terms cleaning a seed or seed cleaning also refer to the removal of any debris or low quality, infested, or infected seeds and seeds of different species that are foreign to the sample.

Treating a seed or applying a treatment to a seed refers to the application of a composition to a seed as a coating or otherwise. The composition may be applied to the seed in a seed treatment at any time from harvesting of the seed to sowing of the seed. The composition may be applied using methods including but not limited to mixing in a container, mechanical application, tumbling, spraying, misting, and immersion. Thus, the composition may be applied as a powder, a crystalline, a ready-to-use, a slurry, a mist, and/or a soak. For a general discussion of techniques used to apply fungicides to seeds, see “Seed Treatment,” 2d ed., (1986), edited by K A Jeffs (chapter 9), herein incorporated by reference in its entirety. The composition to be used as a seed treatment can comprise one or more of a pesticide, a fungicide, an insecticide, a nematicide, an antimicrobial, an inoculant, a growth promoter, a polymer, a flow agent, a coating, or any combination thereof. General classes or family of seed treatment agents include triazoles, anilides, pyrazoles, carboxamides, succinate dehydrogenase inhibitors (SDHI), triazolinthiones, strobilurins, amides, and anthranilic diamides. In some examples, the seed treatment comprises trifloxystrobin, azoxystrobin, metalaxyl, metalaxyl-m, mefenoxam, fludioxinil, imidacloprid, thiamethoxam, thiabendazole, ipconazole, penflufen, sedaxane, prothioconazole, picoxystrobin, penthiopyrad, pyraclastrobin, xemium, Rhizobia spp., Bradyrhizobium spp. (e.g., B. japonicum), Bacillus spp. (e.g., B. firmus, B. pumilus, B. subtilus), lipo-chitooligosaccharide, clothianidin, cyantraniliprole, chlorantraniliprole, abamectin, and any combination thereof. In some examples the seed treatment comprises trifloxystrobin, metalaxyl, imidacloprid, Bacillus spp., and any combination thereof. In some examples the seed treatment comprises picoxystrobin, penthiopyrad, cyantraniliprole, chlorantraniliprole, and any combination thereof. In some examples, the seed treatment improves seed germination under normal and/or stress environments, early stand count, vigor, yield, root formation, nodulation, and any combination thereof. In some examples seed treatment reduces seed dust levels, insect damage, pathogen establishment and/or damage, plant virus infection and/or damage, and any combination thereof.

Genetic elements or genes located on a single chromosome segment are physically linked. In some examples, the two loci are located in close proximity such that recombination between homologous chromosome pairs does not occur between the two loci during meiosis with high frequency, e.g., such that linked loci co-segregate at least about 90% of the time, e.g., 91%, 92%, 93%, 94%, 95%, 96%, 97%, 98%, 99%, 99.5%, 99.75%, or more of the time. The genetic elements located within a chromosome segment are also genetically linked, typically within a genetic recombination distance of less than or equal to 50 centimorgans (cM), e.g., about 49, 40, 30, 20, 10, 9, 8, 7, 6, 5, 4, 3, 2, 1, 0.75, 0.5, or 0.25 cM or less. That is, two genetic elements within a single chromosome segment undergo recombination during meiosis with each other at a frequency of less than or equal to about 50%, e.g., about 49%, 40%, 30%, 20%, 10%, 9%, 8%, 7%, 6%, 5%, 4%, 3%, 2%, 1%, 0.75%, 0.5%, or 0.25% or less. Closely linked markers display a cross over frequency with a given marker of about 10% or less (the given marker is within about 10 cM of a closely linked marker). Put another way, closely linked loci co-segregate at least about 90% of the time.

In certain examples, plants or germplasm are identified that have at least one favorable allele, marker, marker profile, and/or haplotype that positively correlate with tolerance or improved tolerance. However, in other examples, it is useful to identify alleles, markers, marker profiles, and/or haplotypes that negatively correlate with tolerance, for example to eliminate such plants or germplasm from subsequent rounds of breeding.

Any marker associated with an stem canker tolerance locus or QTL is useful, including but not limited to, for example, a locus on LG B2. Further, any suitable type of marker can be used, including Restriction Fragment Length Polymorphisms (RFLPs), Single Sequence Repeats (SSRs), Target Region Amplification Polymorphisms (TRAPs), Isozyme Electrophoresis, Randomly Amplified Polymorphic DNAs (RAPDs), Arbitrarily Primed Polymerase Chain Reaction (AP-PCR), DNA Amplification Fingerprinting (DAF), Sequence Characterized Amplified Regions (SCARs), Amplified Fragment Length Polymorphisms (AFLPs), and Single Nucleotide Polymorphisms (SNPs). Additionally, other types of molecular markers known in the art or phenotypic traits may also be used in the methods.

Markers that map closer to an stem canker tolerance QTL are generally preferred over markers that map farther from such a QTL. Marker loci are especially useful when they are closely linked to an stem canker tolerance QTL. Thus, in one example, marker loci display an inter-locus cross-over frequency of about 10% or less, about 9% or less, about 8% or less, about 7% or less, about 6% or less, about 5% or less, about 4% or less, about 3% or less, about 2% or less, about 1% or less, about 0.75% or less, about 0.5% or less, or about 0.25% or less with a stem canker tolerance QTL to which they are linked. Thus, the loci are separated from the QTL to which they are linked by about 10 cM, 9 cM, 8 cM, 7 cM, 6 cM, 5 cM, 4 cM, 3 cM, 2 cM, 1 cM, 0.75 cM, 0.5 cM, or 0.25 cM or less. In certain examples, multiple marker loci that collectively make up a haplotype are investigated, for instance 2, 3, 4, 5, 6, 7, 8, 9, 10, 11, 12, 13, 14, 15, 16, 17, 18, 19, 20, 21, 22, 23, 24, 25, 26, 27, 28, 29, 30, or more marker loci.

Both chromosome number and linkage group identifiers have been used to describe soybean genome based on genetic mapping data, physical mapping data, and sequencing data and assemblies. Linkage group lengths in cM are based on the Soybean Consensus Map 3.0 produced by Perry Cregan's group at the USDA-ARS Soybean Genomics and Improvement Lab. The 11 initial linkage group to chromosome number assignments were made by Ted Hymowitz's group (Zou et al. (2003) Theor Appl Genet 107:745-750 and citations therein). The remaining 9 were given chromosome numbers in decreasing order of linkage group genetic length. Based on this system, linkage group C1 is chromosome 4 (Gm04), and linkage group C2 is chromosome 6 (Gm06). The soybean chromosome number to linkage group assignments can be found at Soybase (see, e.g., soybase.org/LG2Xsome.php).

Large numbers of soybean genetic markers have been mapped and linkage groups created, for example as described in Cregan et al., “An Integrated Genetic Linkage Map of the Soybean Genome” (1999) Crop Sci 39:1464-90, and Choi et al., “A Soybean Transcript Map: Gene Distribution, Haplotype and Single-Nucleotide Polymorphism Analysis” (2007) Genetics 176:685-96. Many soybean markers are publicly available at the USDA affiliated soybase website (www.soybase.org). All markers are used to define a specific locus on the soybean genome. Each marker is therefore an indicator of a specific segment of DNA, having a unique nucleotide sequence. The map positions provide a measure of the relative positions of particular markers with respect to one another. When a trait is stated to be linked to a given marker it will be understood that the actual DNA segment whose sequence affects the trait generally co-segregates with the marker. More precise and definite localization of a trait can be obtained if markers are identified on both sides of the trait. By measuring the appearance of the marker(s) in progeny of crosses, the existence of the trait can be detected by relatively simple molecular tests without actually evaluating the appearance of the trait itself, which can be difficult and time-consuming because the actual evaluation of the trait requires growing plants to a stage and/or under environmental conditions where the trait can be expressed. Molecular markers have been widely used to determine genetic composition in soybeans.

Favorable genotypes associated with at least trait of interest may be identified by one or more methodologies. In some examples one or more markers are used, including but not limited to AFLPs, RFLPs, ASH, SSRs, SNPs, indels, padlock probes, molecular inversion probes, microarrays, sequencing, and the like. In some methods, a target nucleic acid is amplified prior to hybridization with a probe. In other cases, the target nucleic acid is not amplified prior to hybridization, such as methods using molecular inversion probes (see, for example Hardenbol et al. (2003) Nat Biotech 21:673-678). In some examples, the genotype related to a specific trait is monitored, while in other examples, a genome-wide evaluation including but not limited to one or more of marker panels, library screens, association studies, microarrays, gene chips, expression studies, or sequencing such as whole-genome resequencing and genotyping-by-sequencing (GBS) may be used. In some examples, no target-specific probe is needed, for example by using sequencing technologies, including but not limited to next-generation sequencing methods (see, for example, Metzker (2010) Nat Rev Genet 11:31-46; and, Egan et al. (2012) Am J Bot 99:175-185) such as sequencing by synthesis (e.g., Roche 454 pyrosequencing, Illumina Genome Analyzer, and Ion Torrent PGM or Proton systems), sequencing by ligation (e.g., SOLiD from Applied Biosystems, and Polnator system from Azco Biotech), and single molecule sequencing (SMS or third-generation sequencing) which eliminate template amplification (e.g., Helicos system, and PacBio RS system from Pacific BioSciences). Further technologies include optical sequencing systems (e.g., Starlight from Life Technologies), and nanopore sequencing (e.g., GridION from Oxford Nanopore Technologies). Each of these may be coupled with one or more enrichment strategies for organellar or nuclear genomes in order to reduce the complexity of the genome under investigation via PCR, hybridization, restriction enzyme (see, e.g., Elshire et al. (2011) PLoS ONE 6:e19379), and expression methods. In some examples, no reference genome sequence is needed in order to complete the analysis.

In some examples, markers within 1 cM, 5 cM, 10 cM, 15 cM, or 30 cM of any one or more of SEQ ID NOs: 1-786 are provided. Similarly, one or more markers mapped within 1, 5, 10, 20 and 30 cM or less from the markers provided can be used for the selection or introgression of the region associated with a stem canker tolerance phenotype. In other examples, any marker that is linked with any one or more of SEQ ID NOs: 1-786 and associated with a stem canker tolerance phenotype is provided. In other examples, markers provided include a substantially a nucleic acid molecule within 5 kb, 10 kb, 20 kb, 30 kb, 100 kb, 500 kb, 1,000 kb, 10,000 kb, 25,000 kb, or 50,000 kb of a marker selected from the group consisting of SEQ ID NOs:1-786.

In addition to the markers discussed herein, information regarding useful soybean markers can be found, for example, on the USDA's Soybase website, available at www.soybase.org. One of skill in the art will recognize that the identification of favorable marker alleles may be germplasm-specific. One of skill will also recognize that methods for identifying the favorable alleles are routine and well known in the art, and furthermore, that the identification and use of such favorable alleles is well within the scope of the invention.

The use of marker assisted selection (MAS) to select a soybean plant or germplasm based upon detection of a particular marker or haplotype of interest is provided. For instance, in certain examples, a soybean plant or germplasm possessing a certain predetermined favorable marker allele, marker profile, or haplotype will be selected via MAS. Using MAS, soybean plants or germplasm can be selected for markers or marker alleles that positively correlate with tolerance, without actually raising soybean and measuring for tolerance (or, contrawise, soybean plants can be selected against if they possess markers that negatively correlate with tolerance). MAS is a powerful tool to select for desired phenotypes and for introgressing desired traits into cultivars of soybean (e.g., introgressing desired traits into elite lines). MAS is easily adapted to high throughput molecular analysis methods that can quickly screen large numbers of plant or germplasm genetic material for the markers of interest and is much more cost effective than raising and observing plants for visible traits.

In some examples, molecular markers are detected using a suitable amplification-based detection method. Typical amplification methods include various polymerase based replication methods, including the polymerase chain reaction (PCR), ligase mediated methods, such as the ligase chain reaction (LCR), and RNA polymerase based amplification (e.g., by transcription) methods. In these types of methods, nucleic acid primers are typically hybridized to the conserved regions flanking the polymorphic marker region. In certain methods, nucleic acid probes that bind to the amplified region are also employed. In general, synthetic methods for making oligonucleotides, including primers and probes, are well known in the art. For example, oligonucleotides can be synthesized chemically according to the solid phase phosphoramidite triester method described by Beaucage & Caruthers (1981) Tetrahedron Letts 22:1859-1862, e.g., using a commercially available automated synthesizer, e.g., as described in Needham-VanDevanter, et al. (1984) Nucl Acids Res 12:6159-6168. Oligonucleotides, including modified oligonucleotides, can also be ordered from a variety of commercial sources known to persons of skill in the art.

It will be appreciated that suitable primers and probes to be used can be designed using any suitable method. It is not intended that the invention be limited to any particular primer, primer pair, or probe. For example, primers can be designed using any suitable software program, such as LASERGENE® or Primer3.

It is not intended that the primers be limited to generating an amplicon of any particular size. For example, the primers used to amplify the marker loci and alleles herein are not limited to amplifying the entire region of the relevant locus. In some examples, marker amplification produces an amplicon at least 20 nucleotides in length, at least 50 nucleotides in length, at least 100 nucleotides in length, at least 200 nucleotides in length, at least 300 nucleotides in length, at least 400 nucleotides in length, at least 500 nucleotides in length, at least 1000 nucleotides in length, at least 2000 nucleotides in length, or greater than 2000 nucleotides in length. PCR, RT-PCR, and LCR are common amplification and amplification-detection methods for amplifying nucleic acids of interest (e.g., those comprising marker loci), facilitating detection of the markers. Details regarding the use of these and other amplification methods are well known in the art and can be found in any of a variety of standard texts. Details for these techniques can also be found in numerous journal and patent references, such as Mullis, et al. (1987) U.S. Pat. No. 4,683,202; Arnheim & Levinson (1990) C&EN 68:36-47; Kwoh et al. (1989) Proc Natl Acad Sci USA 86:1173; Guatelli et al. (1990) Proc Natl Acad Sci USA 87:1874; Lomell et al. (1989) J Clin Chem 35:1826; Landegren et al. (1988) Science 241:1077-1080; Van Brunt (1990) Biotechnology 8:291-294; Wu & Wallace (1989) Gene 4:560; Barringer et al. (1990) Gene 89:117; and Sooknanan & Malek (1995) Biotechnology 13:563-564.

Such nucleic acid amplification techniques can be applied to amplify and/or detect nucleic acids of interest, such as nucleic acids comprising marker loci. Amplification primers for amplifying useful marker loci and suitable probes to detect useful marker loci or to genotype alleles, such as SNP alleles, are provided. However, one of skill will immediately recognize that other primer and probe sequences could also be used. For instance primers to either side of the given primers can be used in place of the given primers, so long as the primers can amplify a region that includes the allele to be detected, as can primers and probes directed to other marker loci. Further, it will be appreciated that the precise probe to be used for detection can vary, e.g., any probe that can identify the region of a marker amplicon to be detected can be substituted for those examples provided herein, and the configuration of the amplification primers and detection probes can, of course, vary. Thus, the compositions and methods are not limited to the primers and probes specifically recited herein.

In certain examples, probes will possess a detectable label. Any suitable label can be used with a probe. Detectable labels suitable for use with nucleic acid probes include, for example, any composition detectable by spectroscopic, radioisotopic, photochemical, biochemical, immunochemical, electrical, optical, or chemical means. Useful labels include biotin for staining with labeled streptavidin conjugate, magnetic beads, fluorescent dyes, radiolabels, enzymes, and colorimetric labels. Other labels include ligands, which bind to antibodies labeled with fluorophores, chemiluminescent agents, and enzymes. A probe can also constitute radiolabelled PCR primers that are used to generate a radiolabelled amplicon. Labeling strategies for nucleic acids and corresponding detection strategies can be found, e.g., in Haugland (1996) Handbook of Fluorescent Probes and Research Chemicals (6th Ed.), Molecular Probes, Inc. (Eugene, Oreg.); or in Haugland (2001) Handbook of Fluorescent Probes and Research Chemicals (8th Ed.), Molecular Probes, Inc. (Eugene, Oreg.).

Detectable labels may also include reporter-quencher pairs, such as are employed in Molecular Beacon and TaqMan™ probes. The reporter may be a fluorescent organic dye modified with a suitable linking group for attachment to the oligonucleotide, such as to the terminal 3′ carbon or terminal 5′ carbon. The quencher may also be an organic dye, which may or may not be fluorescent. Generally, whether the quencher is fluorescent or simply releases the transferred energy from the reporter by non-radiative decay, the absorption band of the quencher should at least substantially overlap the fluorescent emission band of the reporter to optimize the quenching. Non-fluorescent quenchers or dark quenchers typically function by absorbing energy from excited reporters, but do not release the energy radiatively.

Selection of appropriate reporter-quencher pairs for particular probes may be undertaken in accordance with known techniques. Fluorescent and dark quenchers and their relevant optical properties from which exemplary reporter-quencher pairs may be selected are listed and described, for example, in Berlman, Handbook of Fluorescence Spectra of Aromatic Molecules (2nd Ed.), Academic Press, New York, 1971, the content of which is incorporated herein by reference. Examples of modifying reporters and quenchers for covalent attachment via common reactive groups that can be added to an oligonucleotide in the present invention may be found, for example, in Haugland (2001) Handbook of Fluorescent Probes and Research Chemicals (8th Ed.), Molecular Probes, Inc. (Eugene, Oreg.), the content of which is incorporated herein by reference.

In certain examples, reporter-quencher pairs are selected from xanthene dyes including fluorescein and rhodamine dyes. Many suitable forms of these compounds are available commercially with substituents on the phenyl groups, which can be used as the site for bonding or as the bonding functionality for attachment to an oligonucleotide. Another useful group of fluorescent compounds for use as reporters are the naphthylamines, having an amino group in the alpha or beta position. Included among such naphthylamino compounds are 1-dimethylaminonaphthyl-5 sulfonate, 1-anilino-8-naphthalene sulfonate and 2-p-touidinyl-6-naphthalene sulfonate. Other dyes include 3-phenyl-7-isocyanatocoumarin; acridines such as 9-isothiocyanatoacridine; N-(p-(2-benzoxazolyl)phenyl)maleimide; benzoxadiazoles; stilbenes; pyrenes and the like. In certain other examples, the reporters and quenchers are selected from fluorescein and rhodamine dyes. These dyes and appropriate linking methodologies for attachment to oligonucleotides are well known in the art.

Suitable examples of reporters may be selected from dyes such as SYBR green, 5-carboxyfluorescein (5-FAM™ available from Applied Biosystems (Foster City, Calif., USA), 6-carboxyfluorescein (6-FAM™), tetrachloro-6-carboxyfluorescein (TET), 2,7-dimethoxy-4,5-dichloro-6-carboxyfluorescein, hexachloro-6-carboxyfluorescein (HEX), 6-carboxy-2′,4,7,7′-tetrachlorofluorescein (6-TET™ available from Applied Biosystems), carboxy-X-rhodamine (ROX), 6-carboxy-4′,5′-dichloro-2′,7′-dimethoxyfluorescein (6-JOE™ available from Applied Biosystems), VIC™ dye products available from Molecular Probes, Inc., NED™ dye products available from available from Applied Biosystems, and the like. Suitable examples of quenchers may be selected from 6-carboxy-tetramethyl-rhodamine, 4-(4-dimethylaminophenylazo) benzoic acid (DABYL), tetramethylrhodamine (TAMRA), BHQ-0™, BHQ-1™, BHQ-2™, and BHQ-3™, each of which are available from Biosearch Technologies, Inc. (Novato, Calif., USA), QSY-7™, QSY-9™, QSY-21™ and Q5Y-35™, each of which are available from Molecular Probes, Inc., and the like.

In one aspect, real time PCR or LCR is performed on the amplification mixtures described herein, e.g., using molecular beacons or TAQMAN™ probes. A molecular beacon (MB) is an oligonucleotide which, under appropriate hybridization conditions, self-hybridizes to form a stem and loop structure. The MB has a label and a quencher at the termini of the oligonucleotide; thus, under conditions that permit intra-molecular hybridization, the label is typically quenched (or at least altered in its fluorescence) by the quencher. Under conditions where the MB does not display intra-molecular hybridization (e.g., when bound to a target nucleic acid, such as to a region of an amplicon during amplification), the MB label is unquenched. Details regarding standard methods of making and using MBs are well established in the literature and MBs are available from a number of commercial reagent sources. See also, e.g., Leone, et al., (1995) Nucl Acids Res 26:2150-2155; Tyagi & Kramer (1996) Nature Biotechnol 14:303-308; Blok & Kramer (1997) Mol Cell Probes 11:187-194; Hsuih et al. (1997) J Clin Microbiol 34:501-507; Kostrikis et al. (1998) Science 279:1228-1229; Sokol et al. (1998) Proc Natl Acad Sci USA 95:11538-11543; Tyagi et al. (1998) Nature Biotechnol 16:49-53; Bonnet et al. (1999) Proc Natl Acad Sci USA 96:6171-6176; Fang et al. (1999) J Am Chem Soc 121:2921-2922; Marras et al. (1999) Genet Anal Biomol Eng 14:151-156; and, Vet et al. (1999) Proc Natl Acad Sci USA 96:6394-6399. Additional details regarding MB construction and use are also found in the patent literature, e.g., U.S. Pat. Nos. 5,925,517; 6,150,097; and 6,037,130.

Another real-time detection method is the 5′-exonuclease detection method, also called the TAQMAN™ assay, for example as set forth in U.S. Pat. Nos. 5,804,375; 5,538,848; 5,487,972; and 5,210,015, each of which is hereby incorporated by reference in its entirety. In the TAQMAN™ assay, a modified probe, typically 10-30 nucleotides in length, is employed during PCR which binds intermediate to or between the two members of the amplification primer pair. The modified probe possesses a reporter and a quencher and is designed to generate a detectable signal to indicate that it has hybridized with the target nucleic acid sequence during PCR. As long as both the reporter and the quencher are on the probe, the quencher stops the reporter from emitting a detectable signal. However, as the polymerase extends the primer during amplification, the intrinsic 5′ to 3′ nuclease activity of the polymerase degrades the probe, separating the reporter from the quencher, and enabling the detectable signal to be emitted. Generally, the amount of detectable signal generated during the amplification cycle is proportional to the amount of product generated in each cycle.

It is well known that the efficiency of quenching is a strong function of the proximity of the reporter and the quencher, i.e., as the two molecules get closer, the quenching efficiency increases. As quenching is strongly dependent on the physical proximity of the reporter and quencher, the reporter and the quencher are typically attached to the probe within a few nucleotides of one another, usually within 30 nucleotides of one another, or within 6 to 16 nucleotides. Typically, this separation is achieved by attaching one member of a reporter-quencher pair to the 5′ end of the probe and the other member to a nucleotide about 6 to 16 nucleotides away, in some cases at the 3′ end of the probe.

Separate detection probes can also be omitted in amplification/detection methods, e.g., by performing a real time amplification reaction that detects product formation by modification of the relevant amplification primer upon incorporation into a product, incorporation of labeled nucleotides into an amplicon, or by monitoring changes in molecular rotation properties of amplicons as compared to unamplified precursors (e.g., by fluorescence polarization).

One example of a suitable real-time detection technique that does not use a separate probe that binds intermediate to the two primers is the KASPar detection system/method, which is well-known in the art. In KASPar, two allele specific primers are designed such that the 3′ nucleotide of each primer hybridizes to the polymorphic base. For example, if the SNP is an A/C polymorphism, one of the primers would have an “A” in the 3′ position, while the other primer would have a “C” in the 3′ position. Each of these two allele specific primers also has a unique tail sequence on the 5′ end of the primer. A common reverse primer is employed that amplifies in conjunction with either of the two allele specific primers. Two 5′ fluor-labeled reporter oligos are also included in the reaction mix, one designed to interact with each of the unique tail sequences of the allele-specific primers. Lastly, one quencher oligo is included for each of the two reporter oligos, the quencher oligo being complementary to the reporter oligo and being able to quench the fluor signal when bound to the reporter oligo. During PCR, the allele-specific primers and reverse primers bind to complementary DNA, allowing amplification of the amplicon to take place. During a subsequent cycle, a complementary nucleic acid strand containing a sequence complementary to the unique tail sequence of the allele-specific primer is created. In a further cycle, the reporter oligo interacts with this complementary tail sequence, acting as a labeled primer. Thus, the product created from this cycle of PCR is a fluorescently-labeled nucleic acid strand. Because the label incorporated into this amplification product is specific to the allele specific primer that resulted in the amplification, detecting the specific fluor presenting a signal can be used to determine the SNP allele that was present in the sample.

Further, it will be appreciated that amplification is not a requirement for marker detection, for example, one can directly detect unamplified genomic DNA simply by performing a Southern blot on a sample of genomic DNA. Procedures for performing Southern blotting, amplification e.g., (PCR, LCR, or the like), and many other nucleic acid detection methods are well established and are taught, e.g., in Sambrook et al. Molecular Cloning—A Laboratory Manual (3d ed.) Vol. 1-3, Cold Spring Harbor Laboratory, Cold Spring Harbor, N.Y., 2000 (“Sambrook”); Current Protocols in Molecular Biology, F. M. Ausubel et al., eds., Current Protocols, a joint venture between Greene Publishing Associates, Inc. and John Wiley & Sons, Inc., (supplemented through 2002) (“Ausubel”); and, PCR Protocols A Guide to Methods and Applications (Innis et al., eds) Academic Press Inc. San Diego, Calif. (1990) (“Innis”), additional details regarding detection of nucleic acids in plants can also be found, e.g., in Plant Molecular Biology (1993) Croy (ed.) BIOS Scientific Publishers, Inc., each of these are herein incorporated by reference in their entirety.

Other techniques for detecting SNPs can also be employed, such as allele specific hybridization (ASH) or nucleic acid sequencing techniques. ASH technology is based on the stable annealing of a short, single-stranded, oligonucleotide probe to a completely complementary single-stranded target nucleic acid. Detection is via an isotopic or non-isotopic label attached to the probe. For each polymorphism, two or more different ASH probes are designed to have identical DNA sequences except at the polymorphic nucleotides. Each probe will have exact homology with one allele sequence so that the range of probes can distinguish all the known alternative allele sequences. Each probe is hybridized to the target DNA. With appropriate probe design and hybridization conditions, a single-base mismatch between the probe and target DNA will prevent hybridization.

Isolated polynucleotide or fragments thereof are capable of specifically hybridizing to other nucleic acid molecules under appropriate conditions. In one example, the nucleic acid molecules comprise any one or more of SEQ ID NOs: 1-786, complements thereof and fragments thereof. In another aspect, the nucleic acid molecules of the present invention include nucleic acid molecules that hybridize, for example, under high or low stringency, substantially homologous sequences, or that have both to these molecules. Conventional stringency conditions are described by Sambrook et al. In: Molecular Cloning, A Laboratory Manual, 2nd Edition, Cold Spring Harbor Press, Cold Spring Harbor, N.Y. (1989)), and by Haymes et al. In: Nucleic Acid Hybridization, A Practical Approach, IRL Press, Washington, D.C. (1985). Departures from complete complementarity are therefore permissible, as long as such departures do not completely preclude the capacity of the molecules to form a double-stranded structure. In order for a nucleic acid molecule to serve as a primer or probe it need only be sufficiently complementary in sequence to be able to form a stable double-stranded structure under the particular solvent and salt concentrations employed. Appropriate stringency conditions that promote DNA hybridization are, for example, 6.0× sodium chloride/sodium citrate (SSC) at about 45° C., followed by a wash of 2.0×SSC at 50° C., are known to those skilled in the art or can be found in Current Protocols in Molecular Biology, John Wiley & Sons, N.Y., 1989, 6.3.1-6.3.6. For example, the salt concentration in the wash step can be selected from a low stringency of about 2.0×SSC at 50° C. to a high stringency of about 0.2×SSC at 50° C. In addition, the temperature in the wash step can be increased from low stringency conditions at room temperature, about 22° C., to high stringency conditions at about 65° C. Both temperature and salt may be varied, or either the temperature or the salt concentration may be held constant while the other variable is changed.

In some examples, an a marker locus will specifically hybridize to one or more of the nucleic acid molecules set forth in SEQ ID NOs:1-786 or complements thereof or fragments of either under moderately stringent conditions, for example at about 2.0×SSC and about 65° C. In an aspect, a nucleic acid of the present invention will specifically hybridize to one or more SEQ ID NOs: 1-786 or complements or fragments of either under high stringency conditions.

In some examples, a marker associated with a stem canker tolerance phenotype comprises any one of SEQ ID NOs: 1-786 or complements or fragments thereof. In other examples, a marker has between 80%, 85%, 90%, 91%, 92%, 93%, 94%, 95%, 96%, 97%, 98%, 99%, or 100% sequence identity to any one of SEQ ID NOs: 1-786 or complements or fragments thereof. Unless otherwise stated, percent sequence identity is determined using the GAP program is default parameters for nucleic acid alignment (Accelrys, San Diego, Calif., USA).

Real-time amplification assays, including MB or TAQMAN™ based assays, are especially useful for detecting SNP alleles. In such cases, probes are typically designed to bind to the amplicon region that includes the SNP locus, with one allele-specific probe being designed for each possible SNP allele. For instance, if there are two known SNP alleles for a particular SNP locus, “A” or “C,” then one probe is designed with an “A” at the SNP position, while a separate probe is designed with a “C” at the SNP position. While the probes are typically identical to one another other than at the SNP position, they need not be. For instance, the two allele-specific probes could be shifted upstream or downstream relative to one another by one or more bases. However, if the probes are not otherwise identical, they should be designed such that they bind with approximately equal efficiencies, which can be accomplished by designing under a strict set of parameters that restrict the chemical properties of the probes. Further, a different detectable label, for instance a different reporter-quencher pair, is typically employed on each different allele-specific probe to permit differential detection of each probe. In certain examples, each allele-specific probe for a certain SNP locus is 13-18 nucleotides in length, dual-labeled with a florescence quencher at the 3′ end and either the 6-FAM™ (6-carboxyfluorescein) or VIC™ (4,7,2′-trichloro-7′-phenyl-6-carboxyfluorescein) fluorophore at the 5′ end. By detecting signal for each label employed and determining which detectable label(s) demonstrated an increased signal, a determination can be made of which allele-specific probe(s) bound to the amplicon and, thus, which SNP allele(s) the amplicon possessed. For instance, when 6-FAM™- and VIC™-labeled probes are employed, the distinct emission wavelengths of 6-FAM™ (518 nm) and VIC™ (554 nm) can be captured. A sample that is homozygous for one allele will have fluorescence from only the respective 6-FAM™ or VIC™ fluorophore, while a sample that is heterozygous at the analyzed locus will have both 6-FAM™ and VIC™ fluorescence.

Introgression of stem canker tolerance into less tolerant soybean germplasm is provided. Any method for introgressing a QTL or marker into soybean plants known to one of skill in the art can be used. Typically, a first soybean germplasm having tolerance to stem canker based on a particular locus, marker, polymorphism, haplotype, and/or marker profile and a second soybean germplasm that lacks such tolerance are provided. The first soybean germplasm may be crossed with the second soybean germplasm to provide progeny soybean germplasm. These progeny germplasm are screened to determine the presence of stem canker tolerance derived from the locus, marker, polymorphism, haplotype, and/or marker profile, and progeny that tests positive for the presence of tolerance derived from the locus, marker, polymorphism, haplotype, and/or marker profile are selected as being soybean germplasm into which the marker or haplotype has been introgressed. Methods for performing such screening are well known in the art and any suitable method can be used, including but not limited to the methods taught in Keeling (1982) Phytopathology 72:807-809, herein incorporated by reference in its entirety.

One application of MAS is to use the tolerance markers or haplotypes to increase the efficiency of an introgression or backcrossing effort aimed at introducing a tolerance trait into a desired (typically high yielding) background. In marker assisted backcrossing of specific markers from a donor source, e.g., to an elite genetic background, one selects among backcross progeny for the donor trait and then uses repeated backcrossing to the elite line to reconstitute as much of the elite background's genome as possible.

Thus, the markers and methods can be utilized to guide marker assisted selection or breeding of soybean varieties with the desired complement (set) of allelic forms of chromosome segments associated with superior agronomic performance (tolerance, along with any other available markers for yield, disease tolerance, etc.). Any of the disclosed marker alleles or haplotypes can be introduced into a soybean line via introgression, by traditional breeding (or introduced via transformation, or both) to yield a soybean plant with superior agronomic performance. The number of alleles associated with tolerance that can be introduced or be present in a soybean plant ranges from 1 to the number of alleles disclosed herein, each integer of which is incorporated herein as if explicitly recited.

This also provides a method of making a progeny soybean plant and these progeny soybean plants, per se. The method comprises crossing a first parent soybean plant with a second soybean plant and growing the female soybean plant under plant growth conditions to yield soybean plant progeny. Methods of crossing and growing soybean plants are well within the ability of those of ordinary skill in the art. Such soybean plant progeny can be assayed for at least one locus, marker, polymorphism, haplotype, and/or marker profile associated with tolerance and, thereby, the desired progeny selected. Such progeny plants or seed can be sold commercially for soybean production, used for food, processed to obtain a desired constituent of the soybean, or further utilized in subsequent rounds of breeding. At least one of the first or second soybean plants is a soybean plant that comprises at least one of the locus, marker, polymorphism, haplotype, and/or marker profile associated with tolerance, such that the progeny are capable of inheriting the locus, marker, polymorphism, haplotype, and/or marker profile.

Often, a method is applied to at least one related soybean plant such as from progenitor or descendant lines in the subject soybean plants pedigree such that inheritance of the desired tolerance can be traced. The number of generations separating the soybean plants being subject to the methods will generally be from 1 to 20, commonly 1 to 5, and typically 1, 2, or 3 generations of separation, and quite often a direct descendant or parent of the soybean plant will be subject to the method (i.e., 1 generation of separation).

Genetic diversity is important for long term genetic gain in any breeding program. With limited diversity, genetic gain will eventually plateau when all of the favorable alleles have been fixed within the elite population. One objective is to incorporate diversity into an elite pool without losing the genetic gain that has already been made and with the minimum possible investment. MAS provides an indication of which genomic regions and which favorable alleles from the original ancestors have been selected for and conserved over time, facilitating efforts to incorporate favorable variation from exotic germplasm sources (parents that are unrelated to the elite gene pool) in the hopes of finding favorable alleles that do not currently exist in the elite gene pool. For example, the markers, haplotypes, primers, and probes can be used for MAS involving crosses of non-elite lines to elite lines or to exotic lines, elite lines to exotic soybean lines (elite X exotic), or any other crossing strategy, by subjecting the segregating progeny to MAS to maintain major yield alleles, along with the tolerance marker alleles herein.

As an alternative to standard breeding methods of introducing traits of interest into soybean (e.g., introgression), transgenic approaches can also be used to create transgenic plants with the desired traits. In these methods, exogenous nucleic acids that encode a desired QTL, marker, or haplotype are introduced into target plants or germplasm. For example, a nucleic acid that codes for a stem canker tolerance trait is cloned, e.g., via positional cloning, and introduced into a target plant or germplasm.

Experienced plant breeders can recognize stem canker tolerant soybean plants in the field, and can select the tolerant individuals or populations for breeding purposes or for propagation. In this context, the plant breeder recognizes tolerant and non-tolerant or susceptible soybean plants. However, plant tolerance is a phenotypic spectrum consisting of extremes in tolerance and susceptibility, as well as a continuum of intermediate tolerance phenotypes. Evaluation of these intermediate phenotypes using reproducible assays are of value to scientists who seek to identify genetic loci that impart tolerance, to conduct marker assisted selection for tolerant populations, and to use introgression techniques to breed a tolerance trait into an elite soybean line, for example.

Phenotypic screening and selection of tolerant and/or susceptible soybean plants may be performed, for example, by exposing plants to a stem canker pathogen, including but not limited to examples such as inoculation, natural exposure, spray tests, dosage tests, leaf painting assays, tissue culture assays, and/or germination assays, and selecting those plants showing tolerance. Any such assay known to the art may be used, e.g., as described in Keeling (1982) Phytopathology 72:807-809 which is incorporated herein by reference in its entirety, or as described herein.

In some examples, a kit or an automated system for detecting one or more locus, marker, polymorphism, haplotype, and/or marker profile, and/or for correlating the locus, marker, polymorphism, haplotype, and/or marker profile with a desired phenotype (e.g., stem canker tolerance), are provided. Thus, a typical kit can include a set of marker probes and/or primers configured to detect at least one favorable allele of one or more marker locus associated with tolerance, improved tolerance, or susceptibility to stem canker. These probes or primers can be configured, for example, to detect the marker alleles noted in the tables and examples herein, e.g., using any available allele detection format, such as solid or liquid phase array based detection, microfluidic-based sample detection, one or more heterologous detectable labels, etc. The kits can further include packaging materials for packaging the probes, or primers, instructions, controls, such as control amplification reactions that include probes, primers, and/or template nucleic acids for amplifications, molecular size markers, buffers, other reagents, containers for mixing and/or reactions, or the like.

A typical system can also include a detector that is configured to detect one or more signal outputs from the set of marker probes or primers, or amplicon thereof, thereby identifying the presence or absence of the allele. A wide variety of signal detection apparatus are available, including photo multiplier tubes, spectrophotometers, CCD arrays, scanning detectors, phototubes and photodiodes, microscope stations, galvo-scans, microfluidic nucleic acid amplification detection appliances, and the like. The precise configuration of the detector will depend, in part, on the type of label used to detect the marker allele, as well as the instrumentation that is most conveniently obtained for the user. Detectors that detect fluorescence, phosphorescence, radioactivity, pH, charge, absorbance, luminescence, temperature, magnetism or the like can be used. Typical detector examples include light (e.g., fluorescence) detectors or radioactivity detectors. For example, detection of a light emission (e.g., a fluorescence emission) or other probe label is indicative of the presence or absence of a marker allele. Fluorescent detection is generally used for detection of amplified nucleic acids (however, upstream and/or downstream operations can also be performed on amplicons, which can involve other detection methods). In general, the detector detects one or more label (e.g., light) emission from a probe label, which is indicative of the presence or absence of a marker allele. The detector(s) optionally monitors one or a plurality of signals from an amplification reaction. For example, the detector can monitor optical signals which correspond to real time amplification assay results.

System or kit instructions that describe how to use the system or kit and/or that correlate the presence or absence of the allele with the predicted tolerance or susceptibility phenotype are also provided. For example, the instructions can include at least one look-up table that includes a correlation between the presence or absence of one or more of the favorable allele(s) or polymorphisms and the predicted tolerance or improved tolerance. The precise form of the instructions can vary depending on the components of the system, e.g., they can be present as system software in one or more integrated unit of the system (e.g., a microprocessor, computer or computer readable medium), or can be present in one or more units (e.g., computers or computer readable media) operably coupled to the detector.

Isolated nucleic acids comprising a nucleic acid sequence coding for tolerance or susceptibility to stem canker, or capable of detecting such a phenotypic trait, or sequences complementary thereto, are also included. In certain examples, the isolated nucleic acids are capable of hybridizing under stringent conditions to nucleic acids of a soybean cultivar displaying tolerance to stem canker, for instance to particular markers, including but not limited to one or more of a marker locus associated with stem canker tolerance, a marker locus closely linked to any of the marker loci, SEQ ID NOs: 1-786, loci identified and provided in FIG. 1 and/or any one of Tables 1-13, and any combination of thereof. In some examples the isolated nucleic acid has been chemically synthesized in vitro. In some examples the isolated nucleic acid comprises a detectable label or tag. In some examples the detectable label or tag comprises at least one compound selected from the group consisting of a fluorophore, a ligand, an enzyme, a dye, a radioisotope, and a metal.

Vectors comprising such nucleic acids, expression products of such vectors expressed in a host compatible therewith, antibodies to the expression product (both polyclonal and monoclonal), and antisense nucleic acids are also included. In some examples, one or more of these nucleic acids is provided in a kit.

Soybean plants and germplasm disclosed herein or derived therefrom or identified using the methods provided and having marker loci associated with stem canker tolerance may be used as a parental line. Also included are soybean plants produced by any of the foregoing methods. Seed of a soybean germplasm produced by crossing a soybean variety having a locus, a marker, a polymorphism, an allele, a haplotype, and/or a marker profile associated with stem canker tolerance with a soybean variety lacking such locus, marker, polymorphism, allele, haplotype, and/or marker profile and progeny thereof, is also included.

The present invention is illustrated by the following examples. The foregoing and following description of the present invention and the various examples are not intended to be limiting of the invention but rather are illustrative thereof. Hence, it will be understood that the invention is not limited to the specific details of these examples.

### EXAMPLES

### Example 1

### Stem Canker Screen

Any screening protocol known in the art can be used to evaluate the tolerance of a plant or plant variety to stem canker, including but not limited to field screens, greenhouse screens, bioassays, and the like.

A. Controlled Environment

Soybean varieties were planted in a controlled environment and screened for tolerance to stem canker pathogen using a toothpick inoculation method essentially as described by Keeling (1982) Phytopathology 72:807-809. Plants are inoculated with Diaporthe phaseolorum var. meridionalis at the V3 growth stage. Watering is discontinued 10 days before the target evaluation date. Plants are evaluated for stem canker lesions approximately 30 days after inoculation. Optionally one or more susceptible and/or resistant check variety can be included in each experiment.

In some experiments, plants are pulled, leaves removed, each stem split longitudinally to measure any internal lesions. Infection types are recorded and grouped into four classes:

Alive, plants have no symptoms or lesions (resistant)

Lightly infected, plants with small stem lesions

Heavily infected, plants with large stem lesions

Dead, whole plant died (susceptible)

These classes represent scores of 1 (susceptible), 3, 6, or 9 (resistant) on a 1-9 scoring scale.

Alternatively, or additionally, a survival rate can be calculated and translated to a 1-9 scoring scale where 1 (susceptible) shows 19% or less surviving plants and 9 (resistant) shows 90% or greater surviving plants.

B. Field Screen

Soybean varieties were screened for tolerance to stem canker pathogen using a toothpick inoculation method essentially as described by Keeling (1982) Phytopathology 72:807-809. Plants are inoculated with Diaporthe phaseolorum var. meridionalis at the V3 to V4 growth stage. Plants are evaluated for both external and internal stem canker lesions approximately 60 days after inoculation. Optionally one or more susceptible and/or resistant check variety can be included in each experiment.

Individual plants are scored for External lesions and classified using a 1 to 9 scale as follows:

1=Foliar symptoms or plant death (susceptible)

3=Lesion greater than 2 inches, no foliar symptoms

5=Lesion between 0.5 to 2 inches, no foliar symptoms

7=Lesion less than 0.5 inches, no foliar symptoms

9=No lesion (resistant)

Individual plants are scored for Internal lesions after splitting the stem longitudinally. Plants are classified as resistant (RES) or susceptible (SUS) base on the presence or absence of lesions.

The External and Internal scores are combined and translated to a 1 to 9 scale to provide an Overall stem canker score, where 1 is susceptible and 9 is resistant.

### Example 2

### Case Control Association Study

Using a case-control association analysis, a previously identified QTL on LG B2

(Gm14), which conditions variation in stem canker resistance, was fine-mapped between 1697331-2019929 bp on the Williams82 soybean reference genome. A set of 647 SNPs, including the current Pioneer Production SNPs S08231-1 and S08255-3, that perfectly differentiate resistant from susceptible lines were identified in this region. These SNPs are ideal candidates for marker-assisted selection of resistance to stem canker. Phenotype scores covered the low and the high ends of the range from the most susceptible (score=1) to the most tolerance (score=9), wherein the susceptible group had scores ranging from 1-3.82 (11 lines), and the resistant group scored from 7.71-9.0 (17 lines).

DNA was prepped using standard Illumina TruSeq Chemistry and lines were sequenced to ˜0.5-40× genome coverage on an Illumina HiSeq2000. SNPs were called using proprietary software. The publicly available software Haploview (Barrett et al. (2005) Bioinformatics 21:263-265) was used to conduct a case-control association analysis on a set of 7426 SNPs identified in the region from 97,600-2,500,000 bp on Gm14. The case group comprised 17 proprietary soybean lines resistant to stem canker, and the control group comprised 11 proprietary susceptible lines (Table 1). Following Haploview filtering using the settings noted below, 6473 SNPs remained in the analysis. Physical positions are based on the Glymal Williams82 soybean reference assembly from JGI.

### Haploview Settings:

Do Association Test
Case/Control Data
Ignore Pairwise comparisons of markers >100 kb apart
Exclude individuals with >50% missing genotypes
HW p-value cutoff: 0.0
Min genotype % 50
Max # mendel errors: 1
Minimum minor allele freq. 0.04

The chi square values from the case-control association analysis against the physical positions of 61911 SNPs reveals a peak of SNP to trait association between 1697331-2019929 bp on Gm14 (LG B2), suggesting a locus conditioning stem canker tolerance is in this region (Glymal Williams82 soybean reference assembly from JGI, described in Schmutz et al. (2010) Nature 463:178-183). 647 SNPs have a perfect association between 11 susceptible (control) and 17 resistant (case) lines, and comprise ideal targets for marker assay design and marker assisted selection. Numerous additional SNPs identified here that are linked to the region but are not highly associated with the trait could be very informative markers when used in select germplasm.

Table 2 summarizes the case control results and associated information for SNPs having perfect association between 11 susceptible (control) and 17 tolerant (case) lines. The naming convention indicates chromosome and physical position (e.g., Gm14:1706695). Pioneer markers for MAS of stem canker, S08231-3 and S08255-3 (Gm14:1727084 and Gm14:1755250 bp respectively), are formatted in bold.

TABLE 2

| | | Case, | | | |
|---|---|---|---|---|---|
| | | Control | | | |
| | Allele | Ratio | Control | Chi | |
| SNP | (R/S) | counts | Frequencies | Square | P-value |
| Gm14:1706695 | A/T | 32:0, 0:20 | 1.000, 0.000 | 52 | 5.55E−13 |
| Gm14:1723656 | C/T | 32:0, 0:20 | 1.000, 0.000 | 52 | 5.55E−13 |
| Gm14:1723831 | G/A | 32:0, 0:20 | 1.000, 0.000 | 52 | 5.55E−13 |
| Gm14:1724319 | G/A | 32:0, 0:20 | 1.000, 0.000 | 52 | 5.55E−13 |
| Gm14:1738483 | G/A | 32:0, 0:20 | 1.000, 0.000 | 52 | 5.55E−13 |
| Gm14:1739929 | A/G | 32:0, 0:20 | 1.000, 0.000 | 52 | 5.55E−13 |
| Gm14:1850843 | A/G | 32:0, 0:20 | 1.000, 0.000 | 52 | 5.55E−13 |
| Gm14:1853044 | T/G | 32:0, 0:20 | 1.000, 0.000 | 52 | 5.55E−13 |
| Gm14:1863371 | G/A | 32:0, 0:20 | 1.000, 0.000 | 52 | 5.55E−13 |
| Gm14:1863436 | G/A | 32:0, 0:20 | 1.000, 0.000 | 52 | 5.55E−13 |
| Gm14:1863868 | C/T | 32:0, 0:20 | 1.000, 0.000 | 52 | 5.55E−13 |
| Gm14:1914305 | C/T | 32:0, 0:20 | 1.000, 0.000 | 52 | 5.55E−13 |
| Gm14:1936105 | T/C | 32:0, 0:20 | 1.000, 0.000 | 52 | 5.55E−13 |
| Gm14:1939210 | A/G | 32:0, 0:20 | 1.000, 0.000 | 52 | 5.55E−13 |
| Gm14:1940046 | C/T | 32:0, 0:20 | 1.000, 0.000 | 52 | 5.55E−13 |
| Gm14:1942461 | C/T | 32:0, 0:20 | 1.000, 0.000 | 52 | 5.55E−13 |
| Gm14:1942681 | T/C | 32:0, 0:20 | 1.000, 0.000 | 52 | 5.55E−13 |
| Gm14:1700735 | T/C | 32:0, 0:20 | 1.000, 0.000 | 50 | 1.54E−12 |
| Gm14:1702258 | G/A | 32:0, 0:20 | 1.000, 0.000 | 50 | 1.54E−12 |
| Gm14:1715352 | C/T | 32:0, 0:20 | 1.000, 0.000 | 50 | 1.54E−12 |
| Gm14:1718565 | A/C | 32:0, 0:20 | 1.000, 0.000 | 50 | 1.54E−12 |
| Gm14:1722078 | C/T | 32:0, 0:20 | 1.000, 0.000 | 50 | 1.54E−12 |
| Gm14:1722192 | T/C | 32:0, 0:20 | 1.000, 0.000 | 50 | 1.54E−12 |
| Gm14:1723045 | G/C | 32:0, 0:20 | 1.000, 0.000 | 50 | 1.54E−12 |
| Gm14:1724087 | A/G | 32:0, 0:20 | 1.000, 0.000 | 50 | 1.54E−12 |
| Gm14:1724245 | A/G | 32:0, 0:20 | 1.000, 0.000 | 50 | 1.54E−12 |
| Gm14:1724558 | T/A | 32:0, 0:20 | 1.000, 0.000 | 50 | 1.54E−12 |
| Gm14:1724603 | T/C | 32:0, 0:20 | 1.000, 0.000 | 50 | 1.54E−12 |
| Gm14:1725318 | A/G | 32:0, 0:20 | 1.000, 0.000 | 50 | 1.54E−12 |
| Gm14:1726899 | A/T | 32:0, 0:20 | 1.000, 0.000 | 50 | 1.54E−12 |
| Gm14:1738054 | T/G | 32:0, 0:20 | 1.000, 0.000 | 50 | 1.54E−12 |
| Gm14:1784545 | A/G | 32:0, 0:20 | 1.000, 0.000 | 50 | 1.54E−12 |
| Gm14:1785276 | C/T | 32:0, 0:20 | 1.000, 0.000 | 50 | 1.54E−12 |
| Gm14:1793750 | C/T | 32:0, 0:20 | 1.000, 0.000 | 50 | 1.54E−12 |
| Gm14:1842226 | T/C | 32:0, 0:20 | 1.000, 0.000 | 50 | 1.54E−12 |
| Gm14:1850134 | T/C | 32:0, 0:20 | 1.000, 0.000 | 50 | 1.54E−12 |
| Gm14:1850654 | A/G | 32:0, 0:20 | 1.000, 0.000 | 50 | 1.54E−12 |
| Gm14:1850808 | C/T | 32:0, 0:20 | 1.000, 0.000 | 50 | 1.54E−12 |
| Gm14:1851180 | C/T | 32:0, 0:20 | 1.000, 0.000 | 50 | 1.54E−12 |
| Gm14:1851369 | G/C | 32:0, 0:20 | 1.000, 0.000 | 50 | 1.54E−12 |
| Gm14:1851467 | A/G | 32:0, 0:20 | 1.000, 0.000 | 50 | 1.54E−12 |
| Gm14:1851793 | C/T | 32:0, 0:20 | 1.000, 0.000 | 50 | 1.54E−12 |
| Gm14:1852388 | G/T | 32:0, 0:20 | 1.000, 0.000 | 50 | 1.54E−12 |
| Gm14:1852579 | C/T | 32:0, 0:20 | 1.000, 0.000 | 50 | 1.54E−12 |
| Gm14:1855401 | A/T | 32:0, 0:20 | 1.000, 0.000 | 50 | 1.54E−12 |
| Gm14:1867651 | A/G | 32:0, 0:20 | 1.000, 0.000 | 50 | 1.54E−12 |
| Gm14:1867913 | A/G | 32:0, 0:20 | 1.000, 0.000 | 50 | 1.54E−12 |
| Gm14:1867927 | C/G | 32:0, 0:20 | 1.000, 0.000 | 50 | 1.54E−12 |
| Gm14:1910547 | C/A | 32:0, 0:20 | 1.000, 0.000 | 50 | 1.54E−12 |
| Gm14:1910746 | T/A | 32:0, 0:20 | 1.000, 0.000 | 50 | 1.54E−12 |
| Gm14:1910787 | C/G | 32:0, 0:20 | 1.000, 0.000 | 50 | 1.54E−12 |
| Gm14:1911679 | A/T | 32:0, 0:20 | 1.000, 0.000 | 50 | 1.54E−12 |
| Gm14:1916729 | A/G | 32:0, 0:20 | 1.000, 0.000 | 50 | 1.54E−12 |
| Gm14:1916742 | T/A | 32:0, 0:20 | 1.000, 0.000 | 50 | 1.54E−12 |
| Gm14:1919121 | T/C | 32:0, 0:20 | 1.000, 0.000 | 50 | 1.54E−12 |
| Gm14:1934770 | C/T | 32:0, 0:20 | 1.000, 0.000 | 50 | 1.54E−12 |
| Gm14:1935561 | C/T | 32:0, 0:20 | 1.000, 0.000 | 50 | 1.54E−12 |
| Gm14:1935695 | G/A | 32:0, 0:20 | 1.000, 0.000 | 50 | 1.54E−12 |
| Gm14:1935949 | A/G | 32:0, 0:20 | 1.000, 0.000 | 50 | 1.54E−12 |
| Gm14:1936112 | T/A | 32:0, 0:20 | 1.000, 0.000 | 50 | 1.54E−12 |
| Gm14:1936619 | A/G | 32:0, 0:20 | 1.000, 0.000 | 50 | 1.54E−12 |
| Gm14:1936750 | T/A | 32:0, 0:20 | 1.000, 0.000 | 50 | 1.54E−12 |
| Gm14:1936804 | A/G | 32:0, 0:20 | 1.000, 0.000 | 50 | 1.54E−12 |
| Gm14:1936827 | G/A | 32:0, 0:20 | 1.000, 0.000 | 50 | 1.54E−12 |
| Gm14:1937070 | T/A | 32:0, 0:20 | 1.000, 0.000 | 50 | 1.54E−12 |
| Gm14:1937136 | C/T | 32:0, 0:20 | 1.000, 0.000 | 50 | 1.54E−12 |
| Gm14:1937495 | C/A | 32:0, 0:20 | 1.000, 0.000 | 50 | 1.54E−12 |
| Gm14:1938061 | A/G | 32:0, 0:20 | 1.000, 0.000 | 50 | 1.54E−12 |
| Gm14:1940079 | C/T | 32:0, 0:20 | 1.000, 0.000 | 50 | 1.54E−12 |
| Gm14:1940534 | T/C | 32:0, 0:20 | 1.000, 0.000 | 50 | 1.54E−12 |
| Gm14:1940728 | G/A | 32:0, 0:20 | 1.000, 0.000 | 50 | 1.54E−12 |
| Gm14:1940825 | C/T | 32:0, 0:20 | 1.000, 0.000 | 50 | 1.54E−12 |
| Gm14:1941047 | G/A | 32:0, 0:20 | 1.000, 0.000 | 50 | 1.54E−12 |
| Gm14:1941105 | G/T | 32:0, 0:20 | 1.000, 0.000 | 50 | 1.54E−12 |
| Gm14:1944642 | A/T | 32:0, 0:20 | 1.000, 0.000 | 50 | 1.54E−12 |
| Gm14:1700746 | T/C | 32:0, 0:20 | 1.000, 0.000 | 48 | 4.26E−12 |
| Gm14:1706616 | G/A | 32:0, 0:20 | 1.000, 0.000 | 48 | 4.26E−12 |
| Gm14:1706705 | G/A | 32:0, 0:20 | 1.000, 0.000 | 48 | 4.26E−12 |
| Gm14:1706782 | A/G | 32:0, 0:20 | 1.000, 0.000 | 48 | 4.26E−12 |
| Gm14:1708836 | C/T | 32:0, 0:20 | 1.000, 0.000 | 48 | 4.26E−12 |
| Gm14:1709816 | T/A | 32:0, 0:20 | 1.000, 0.000 | 48 | 4.26E−12 |
| Gm14:1712174 | T/A | 32:0, 0:20 | 1.000, 0.000 | 48 | 4.26E−12 |
| Gm14:1717270 | A/G | 32:0, 0:20 | 1.000, 0.000 | 48 | 4.26E−12 |
| Gm14:1722544 | C/T | 32:0, 0:20 | 1.000, 0.000 | 48 | 4.26E−12 |
| Gm14:1723756 | T/A | 32:0, 0:20 | 1.000, 0.000 | 48 | 4.26E−12 |
| Gm14:1725153 | T/C | 32:0, 0:20 | 1.000, 0.000 | 48 | 4.26E−12 |
| Gm14:1725269 | C/A | 32:0, 0:20 | 1.000, 0.000 | 48 | 4.26E−12 |
| Gm14:1726477 | G/C | 32:0, 0:20 | 1.000, 0.000 | 48 | 4.26E−12 |
| Gm14:1727084 | C/G | 32:0, 0:20 | 1.000, 0.000 | 48 | 4.26E−12 |
| Gm14:1731690 | C/T | 32:0, 0:20 | 1.000, 0.000 | 48 | 4.26E−12 |
| Gm14:1738538 | T/C | 32:0, 0:20 | 1.000, 0.000 | 48 | 4.26E−12 |
| Gm14:1739491 | T/G | 32:0, 0:20 | 1.000, 0.000 | 48 | 4.26E−12 |
| Gm14:1739824 | G/C | 32:0, 0:20 | 1.000, 0.000 | 48 | 4.26E−12 |
| Gm14:1785457 | G/C | 32:0, 0:20 | 1.000, 0.000 | 48 | 4.26E−12 |
| Gm14:1787260 | T/G | 32:0, 0:20 | 1.000, 0.000 | 48 | 4.26E−12 |
| Gm14:1796497 | T/G | 32:0, 0:20 | 1.000, 0.000 | 48 | 4.26E−12 |
| Gm14:1840314 | G/T | 32:0, 0:20 | 1.000, 0.000 | 48 | 4.26E−12 |
| Gm14:1842837 | A/G | 32:0, 0:20 | 1.000, 0.000 | 48 | 4.26E−12 |
| Gm14:1850412 | C/A | 32:0, 0:20 | 1.000, 0.000 | 48 | 4.26E−12 |
| Gm14:1850433 | T/A | 32:0, 0:20 | 1.000, 0.000 | 48 | 4.26E−12 |
| Gm14:1850907 | T/A | 32:0, 0:20 | 1.000, 0.000 | 48 | 4.26E−12 |
| Gm14:1851535 | T/A | 32:0, 0:20 | 1.000, 0.000 | 48 | 4.26E−12 |
| Gm14:1852210 | A/G | 32:0, 0:20 | 1.000, 0.000 | 48 | 4.26E−12 |
| Gm14:1859255 | C/T | 32:0, 0:20 | 1.000, 0.000 | 48 | 4.26E−12 |
| Gm14:1860411 | G/A | 32:0, 0:20 | 1.000, 0.000 | 48 | 4.26E−12 |
| Gm14:1909564 | T/C | 32:0, 0:20 | 1.000, 0.000 | 48 | 4.26E−12 |
| Gm14:1910968 | G/T | 32:0, 0:20 | 1.000, 0.000 | 48 | 4.26E−12 |
| Gm14:1935936 | A/G | 32:0, 0:20 | 1.000, 0.000 | 48 | 4.26E−12 |
| Gm14:1936214 | A/G | 32:0, 0:20 | 1.000, 0.000 | 48 | 4.26E−12 |
| Gm14:1936876 | G/A | 32:0, 0:20 | 1.000, 0.000 | 48 | 4.26E−12 |
| Gm14:1939030 | C/T | 32:0, 0:20 | 1.000, 0.000 | 48 | 4.26E−12 |
| Gm14:1939031 | T/A | 32:0, 0:20 | 1.000, 0.000 | 48 | 4.26E−12 |
| Gm14:1939081 | A/G | 32:0, 0:20 | 1.000, 0.000 | 48 | 4.26E−12 |
| Gm14:1940392 | A/C | 32:0, 0:20 | 1.000, 0.000 | 48 | 4.26E−12 |
| Gm14:1941517 | A/G | 32:0, 0:20 | 1.000, 0.000 | 48 | 4.26E−12 |
| Gm14:1989692 | G/A | 32:0, 0:20 | 1.000, 0.000 | 48 | 4.26E−12 |
| Gm14:1989717 | C/T | 32:0, 0:20 | 1.000, 0.000 | 48 | 4.26E−12 |
| Gm14:1992951 | T/A | 32:0, 0:20 | 1.000, 0.000 | 48 | 4.26E−12 |
| Gm14:2003498 | A/G | 32:0, 0:20 | 1.000, 0.000 | 48 | 4.26E−12 |
| Gm14:2004242 | A/G | 32:0, 0:20 | 1.000, 0.000 | 48 | 4.26E−12 |
| Gm14:2004409 | T/C | 32:0, 0:20 | 1.000, 0.000 | 48 | 4.26E−12 |
| Gm14:2006356 | A/T | 32:0, 0:20 | 1.000, 0.000 | 48 | 4.26E−12 |
| Gm14:1698254 | A/G | 32:0, 0:20 | 1.000, 0.000 | 46 | 1.18E−11 |
| Gm14:1698262 | T/C | 32:0, 0:20 | 1.000, 0.000 | 46 | 1.18E−11 |
| Gm14:1698269 | C/T | 32:0, 0:20 | 1.000, 0.000 | 46 | 1.18E−11 |
| Gm14:1701170 | T/A | 32:0, 0:20 | 1.000, 0.000 | 46 | 1.18E−11 |
| Gm14:1701174 | G/A | 32:0, 0:20 | 1.000, 0.000 | 46 | 1.18E−11 |
| Gm14:1701472 | T/A | 32:0, 0:20 | 1.000, 0.000 | 46 | 1.18E−11 |
| Gm14:1701825 | G/T | 32:0, 0:20 | 1.000, 0.000 | 46 | 1.18E−11 |
| Gm14:1702477 | A/T | 32:0, 0:20 | 1.000, 0.000 | 46 | 1.18E−11 |
| Gm14:1702552 | G/A | 32:0, 0:20 | 1.000, 0.000 | 46 | 1.18E−11 |
| Gm14:1702622 | T/C | 32:0, 0:20 | 1.000, 0.000 | 46 | 1.18E−11 |
| Gm14:1704204 | C/A | 32:0, 0:20 | 1.000, 0.000 | 46 | 1.18E−11 |
| Gm14:1705803 | A/G | 32:0, 0:20 | 1.000, 0.000 | 46 | 1.18E−11 |
| Gm14:1706201 | T/G | 32:0, 0:20 | 1.000, 0.000 | 46 | 1.18E−11 |
| Gm14:1706361 | C/T | 32:0, 0:20 | 1.000, 0.000 | 46 | 1.18E−11 |
| Gm14:1706585 | A/G | 32:0, 0:20 | 1.000, 0.000 | 46 | 1.18E−11 |
| Gm14:1706915 | T/C | 32:0, 0:20 | 1.000, 0.000 | 46 | 1.18E−11 |
| Gm14:1708746 | A/G | 32:0, 0:20 | 1.000, 0.000 | 46 | 1.18E−11 |
| Gm14:1713290 | A/T | 32:0, 0:20 | 1.000, 0.000 | 46 | 1.18E−11 |
| Gm14:1718056 | A/G | 32:0, 0:20 | 1.000, 0.000 | 46 | 1.18E−11 |
| Gm14:1718944 | A/G | 32:0, 0:20 | 1.000, 0.000 | 46 | 1.18E−11 |
| Gm14:1719373 | T/C | 32:0, 0:20 | 1.000, 0.000 | 46 | 1.18E−11 |
| Gm14:1723066 | A/C | 32:0, 0:20 | 1.000, 0.000 | 46 | 1.18E−11 |
| Gm14:1726434 | T/A | 32:0, 0:20 | 1.000, 0.000 | 46 | 1.18E−11 |
| Gm14:1726867 | G/A | 32:0, 0:20 | 1.000, 0.000 | 46 | 1.18E−11 |
| Gm14:1727235 | A/T | 32:0, 0:20 | 1.000, 0.000 | 46 | 1.18E−11 |
| Gm14:1728343 | G/A | 32:0, 0:20 | 1.000, 0.000 | 46 | 1.18E−11 |
| Gm14:1730044 | A/G | 32:0, 0:20 | 1.000, 0.000 | 46 | 1.18E−11 |
| Gm14:1731518 | T/G | 32:0, 0:20 | 1.000, 0.000 | 46 | 1.18E−11 |
| Gm14:1736053 | C/T | 32:0, 0:20 | 1.000, 0.000 | 46 | 1.18E−11 |
| Gm14:1738509 | C/T | 32:0, 0:20 | 1.000, 0.000 | 46 | 1.18E−11 |
| Gm14:1738914 | A/G | 32:0, 0:20 | 1.000, 0.000 | 46 | 1.18E−11 |
| Gm14:1739468 | C/T | 32:0, 0:20 | 1.000, 0.000 | 46 | 1.18E−11 |
| Gm14:1739718 | C/T | 32:0, 0:20 | 1.000, 0.000 | 46 | 1.18E−11 |
| Gm14:1739772 | T/G | 32:0, 0:20 | 1.000, 0.000 | 46 | 1.18E−11 |
| Gm14:1749899 | C/T | 32:0, 0:20 | 1.000, 0.000 | 46 | 1.18E−11 |
| Gm14:1785516 | A/G | 32:0, 0:20 | 1.000, 0.000 | 46 | 1.18E−11 |
| Gm14:1785559 | G/C | 32:0, 0:20 | 1.000, 0.000 | 46 | 1.18E−11 |
| Gm14:1786034 | T/G | 32:0, 0:20 | 1.000, 0.000 | 46 | 1.18E−11 |
| Gm14:1786259 | A/C | 32:0, 0:20 | 1.000, 0.000 | 46 | 1.18E−11 |
| Gm14:1793453 | A/G | 32:0, 0:20 | 1.000, 0.000 | 46 | 1.18E−11 |
| Gm14:1796469 | A/C | 32:0, 0:20 | 1.000, 0.000 | 46 | 1.18E−11 |
| Gm14:1797188 | A/G | 32:0, 0:20 | 1.000, 0.000 | 46 | 1.18E−11 |
| Gm14:1798987 | G/A | 32:0, 0:20 | 1.000, 0.000 | 46 | 1.18E−11 |
| Gm14:1800055 | A/G | 32:0, 0:20 | 1.000, 0.000 | 46 | 1.18E−11 |
| Gm14:1842374 | T/A | 32:0, 0:20 | 1.000, 0.000 | 46 | 1.18E−11 |
| Gm14:1850066 | A/G | 32:0, 0:20 | 1.000, 0.000 | 46 | 1.18E−11 |
| Gm14:1850741 | A/G | 32:0, 0:20 | 1.000, 0.000 | 46 | 1.18E−11 |
| Gm14:1850928 | C/T | 32:0, 0:20 | 1.000, 0.000 | 46 | 1.18E−11 |
| Gm14:1852469 | T/A | 32:0, 0:20 | 1.000, 0.000 | 46 | 1.18E−11 |
| Gm14:1854015 | G/A | 32:0, 0:20 | 1.000, 0.000 | 46 | 1.18E−11 |
| Gm14:1854023 | A/C | 32:0, 0:20 | 1.000, 0.000 | 46 | 1.18E−11 |
| Gm14:1859810 | T/A | 32:0, 0:20 | 1.000, 0.000 | 46 | 1.18E−11 |
| Gm14:1860271 | G/A | 32:0, 0:20 | 1.000, 0.000 | 46 | 1.18E−11 |
| Gm14:1862541 | G/A | 32:0, 0:20 | 1.000, 0.000 | 46 | 1.18E−11 |
| Gm14:1867332 | A/T | 32:0, 0:20 | 1.000, 0.000 | 46 | 1.18E−11 |
| Gm14:1868414 | C/A | 32:0, 0:20 | 1.000, 0.000 | 46 | 1.18E−11 |
| Gm14:1919937 | C/T | 32:0, 0:20 | 1.000, 0.000 | 46 | 1.18E−11 |
| Gm14:1935444 | T/C | 32:0, 0:20 | 1.000, 0.000 | 46 | 1.18E−11 |
| Gm14:1936433 | T/C | 32:0, 0:20 | 1.000, 0.000 | 46 | 1.18E−11 |
| Gm14:1937170 | G/A | 32:0, 0:20 | 1.000, 0.000 | 46 | 1.18E−11 |
| Gm14:1937923 | T/A | 32:0, 0:20 | 1.000, 0.000 | 46 | 1.18E−11 |
| Gm14:1938818 | T/C | 32:0, 0:20 | 1.000, 0.000 | 46 | 1.18E−11 |
| Gm14:1939343 | T/C | 32:0, 0:20 | 1.000, 0.000 | 46 | 1.18E−11 |
| Gm14:1939364 | T/C | 32:0, 0:20 | 1.000, 0.000 | 46 | 1.18E−11 |
| Gm14:1939647 | A/G | 32:0, 0:20 | 1.000, 0.000 | 46 | 1.18E−11 |
| Gm14:1939662 | T/A | 32:0, 0:20 | 1.000, 0.000 | 46 | 1.18E−11 |
| Gm14:1939746 | C/T | 32:0, 0:20 | 1.000, 0.000 | 46 | 1.18E−11 |
| Gm14:1939749 | G/A | 32:0, 0:20 | 1.000, 0.000 | 46 | 1.18E−11 |
| Gm14:1940207 | A/T | 32:0, 0:20 | 1.000, 0.000 | 46 | 1.18E−11 |
| Gm14:1940573 | A/G | 32:0, 0:20 | 1.000, 0.000 | 46 | 1.18E−11 |
| Gm14:1941546 | T/A | 32:0, 0:20 | 1.000, 0.000 | 46 | 1.18E−11 |
| Gm14:1945962 | A/T | 32:0, 0:20 | 1.000, 0.000 | 46 | 1.18E−11 |
| Gm14:1946355 | A/G | 32:0, 0:20 | 1.000, 0.000 | 46 | 1.18E−11 |
| Gm14:1990996 | A/G | 32:0, 0:20 | 1.000, 0.000 | 46 | 1.18E−11 |
| Gm14:1991421 | C/T | 32:0, 0:20 | 1.000, 0.000 | 46 | 1.18E−11 |
| Gm14:2001506 | T/C | 32:0, 0:20 | 1.000, 0.000 | 46 | 1.18E−11 |
| Gm14:2001882 | T/C | 32:0, 0:20 | 1.000, 0.000 | 46 | 1.18E−11 |
| Gm14:2003462 | G/A | 32:0, 0:20 | 1.000, 0.000 | 46 | 1.18E−11 |
| Gm14:2003672 | T/C | 32:0, 0:20 | 1.000, 0.000 | 46 | 1.18E−11 |
| Gm14:1697331 | C/T | 32:0, 0:20 | 1.000, 0.000 | 44 | 3.28E−11 |
| Gm14:1698694 | G/A | 32:0, 0:20 | 1.000, 0.000 | 44 | 3.28E−11 |
| Gm14:1700508 | C/T | 32:0, 0:20 | 1.000, 0.000 | 44 | 3.28E−11 |
| Gm14:1701739 | G/C | 32:0, 0:20 | 1.000, 0.000 | 44 | 3.28E−11 |
| Gm14:1701858 | G/T | 32:0, 0:20 | 1.000, 0.000 | 44 | 3.28E−11 |
| Gm14:1702110 | A/T | 32:0, 0:20 | 1.000, 0.000 | 44 | 3.28E−11 |
| Gm14:1702378 | C/T | 32:0, 0:20 | 1.000, 0.000 | 44 | 3.28E−11 |
| Gm14:1706884 | T/C | 32:0, 0:20 | 1.000, 0.000 | 44 | 3.28E−11 |
| Gm14:1706973 | T/C | 32:0, 0:20 | 1.000, 0.000 | 44 | 3.28E−11 |
| Gm14:1708902 | G/C | 32:0, 0:20 | 1.000, 0.000 | 44 | 3.28E−11 |
| Gm14:1711952 | T/A | 32:0, 0:20 | 1.000, 0.000 | 44 | 3.28E−11 |
| Gm14:1712050 | C/T | 32:0, 0:20 | 1.000, 0.000 | 44 | 3.28E−11 |
| Gm14:1712345 | A/C | 32:0, 0:20 | 1.000, 0.000 | 44 | 3.28E−11 |
| Gm14:1717261 | G/T | 32:0, 0:20 | 1.000, 0.000 | 44 | 3.28E−11 |
| Gm14:1722228 | T/C | 32:0, 0:20 | 1.000, 0.000 | 44 | 3.28E−11 |
| Gm14:1722277 | C/T | 32:0, 0:20 | 1.000, 0.000 | 44 | 3.28E−11 |
| Gm14:1723101 | C/T | 32:0, 0:20 | 1.000, 0.000 | 44 | 3.28E−11 |
| Gm14:1724793 | T/A | 32:0, 0:20 | 1.000, 0.000 | 44 | 3.28E−11 |
| Gm14:1725740 | G/A | 32:0, 0:20 | 1.000, 0.000 | 44 | 3.28E−11 |
| Gm14:1726334 | T/A | 32:0, 0:20 | 1.000, 0.000 | 44 | 3.28E−11 |
| Gm14:1727170 | T/C | 32:0, 0:20 | 1.000, 0.000 | 44 | 3.28E−11 |
| Gm14:1727625 | T/C | 32:0, 0:20 | 1.000, 0.000 | 44 | 3.28E−11 |
| Gm14:1730199 | C/T | 32:0, 0:20 | 1.000, 0.000 | 44 | 3.28E−11 |
| Gm14:1736329 | G/C | 32:0, 0:20 | 1.000, 0.000 | 44 | 3.28E−11 |
| Gm14:1736463 | G/A | 32:0, 0:20 | 1.000, 0.000 | 44 | 3.28E−11 |
| Gm14:1736832 | T/C | 32:0, 0:20 | 1.000, 0.000 | 44 | 3.28E−11 |
| Gm14:1737771 | G/C | 32:0, 0:20 | 1.000, 0.000 | 44 | 3.28E−11 |
| Gm14:1738824 | G/A | 32:0, 0:20 | 1.000, 0.000 | 44 | 3.28E−11 |
| Gm14:1738960 | A/C | 32:0, 0:20 | 1.000, 0.000 | 44 | 3.28E−11 |
| Gm14:1743818 | G/A | 32:0, 0:20 | 1.000, 0.000 | 44 | 3.28E−11 |
| Gm14:1756757 | G/T | 32:0, 0:20 | 1.000, 0.000 | 44 | 3.28E−11 |
| Gm14:1760506 | C/T | 32:0, 0:20 | 1.000, 0.000 | 44 | 3.28E−11 |
| Gm14:1778710 | T/A | 32:0, 0:20 | 1.000, 0.000 | 44 | 3.28E−11 |
| Gm14:1783716 | T/C | 32:0, 0:20 | 1.000, 0.000 | 44 | 3.28E−11 |
| Gm14:1784221 | T/A | 32:0, 0:20 | 1.000, 0.000 | 44 | 3.28E−11 |
| Gm14:1786098 | G/A | 32:0, 0:20 | 1.000, 0.000 | 44 | 3.28E−11 |
| Gm14:1797150 | A/T | 32:0, 0:20 | 1.000, 0.000 | 44 | 3.28E−11 |
| Gm14:1797155 | G/A | 32:0, 0:20 | 1.000, 0.000 | 44 | 3.28E−11 |
| Gm14:1799977 | T/G | 32:0, 0:20 | 1.000, 0.000 | 44 | 3.28E−11 |
| Gm14:1811618 | G/T | 32:0, 0:20 | 1.000, 0.000 | 44 | 3.28E−11 |
| Gm14:1812051 | C/G | 32:0, 0:20 | 1.000, 0.000 | 44 | 3.28E−11 |
| Gm14:1824464 | A/G | 32:0, 0:20 | 1.000, 0.000 | 44 | 3.28E−11 |
| Gm14:1839360 | T/A | 32:0, 0:20 | 1.000, 0.000 | 44 | 3.28E−11 |
| Gm14:1843408 | G/C | 32:0, 0:20 | 1.000, 0.000 | 44 | 3.28E−11 |
| Gm14:1850104 | T/C | 32:0, 0:20 | 1.000, 0.000 | 44 | 3.28E−11 |
| Gm14:1851115 | A/G | 32:0, 0:20 | 1.000, 0.000 | 44 | 3.28E−11 |
| Gm14:1851116 | G/A | 32:0, 0:20 | 1.000, 0.000 | 44 | 3.28E−11 |
| Gm14:1855960 | G/T | 32:0, 0:20 | 1.000, 0.000 | 44 | 3.28E−11 |
| Gm14:1858300 | C/T | 32:0, 0:20 | 1.000, 0.000 | 44 | 3.28E−11 |
| Gm14:1860243 | A/G | 32:0, 0:20 | 1.000, 0.000 | 44 | 3.28E−11 |
| Gm14:1860675 | C/A | 32:0, 0:20 | 1.000, 0.000 | 44 | 3.28E−11 |
| Gm14:1866981 | C/T | 32:0, 0:20 | 1.000, 0.000 | 44 | 3.28E−11 |
| Gm14:1869210 | C/A | 32:0, 0:20 | 1.000, 0.000 | 44 | 3.28E−11 |
| Gm14:1872625 | A/G | 32:0, 0:20 | 1.000, 0.000 | 44 | 3.28E−11 |
| Gm14:1902929 | C/T | 32:0, 0:20 | 1.000, 0.000 | 44 | 3.28E−11 |
| Gm14:1913544 | T/C | 32:0, 0:20 | 1.000, 0.000 | 44 | 3.28E−11 |
| Gm14:1914780 | T/C | 32:0, 0:20 | 1.000, 0.000 | 44 | 3.28E−11 |
| Gm14:1935408 | C/T | 32:0, 0:20 | 1.000, 0.000 | 44 | 3.28E−11 |
| Gm14:1936381 | C/T | 32:0, 0:20 | 1.000, 0.000 | 44 | 3.28E−11 |
| Gm14:1936425 | A/G | 32:0, 0:20 | 1.000, 0.000 | 44 | 3.28E−11 |
| Gm14:1939383 | G/A | 32:0, 0:20 | 1.000, 0.000 | 44 | 3.28E−11 |
| Gm14:1988977 | T/A | 32:0, 0:20 | 1.000, 0.000 | 44 | 3.28E−11 |
| Gm14:1989052 | A/T | 32:0, 0:20 | 1.000, 0.000 | 44 | 3.28E−11 |
| Gm14:1990632 | A/T | 32:0, 0:20 | 1.000, 0.000 | 44 | 3.28E−11 |
| Gm14:1990775 | A/G | 32:0, 0:20 | 1.000, 0.000 | 44 | 3.28E−11 |
| Gm14:1991550 | G/A | 32:0, 0:20 | 1.000, 0.000 | 44 | 3.28E−11 |
| Gm14:1999220 | C/T | 32:0, 0:20 | 1.000, 0.000 | 44 | 3.28E−11 |
| Gm14:2003981 | G/A | 32:0, 0:20 | 1.000, 0.000 | 44 | 3.28E−11 |
| Gm14:1697620 | G/T | 32:0, 0:20 | 1.000, 0.000 | 42 | 9.13E−11 |
| Gm14:1697978 | A/T | 32:0, 0:20 | 1.000, 0.000 | 42 | 9.13E−11 |
| Gm14:1699410 | A/G | 32:0, 0:20 | 1.000, 0.000 | 42 | 9.13E−11 |
| Gm14:1700636 | G/C | 32:0, 0:20 | 1.000, 0.000 | 42 | 9.13E−11 |
| Gm14:1701683 | C/T | 32:0, 0:20 | 1.000, 0.000 | 42 | 9.13E−11 |
| Gm14:1701935 | T/C | 32:0, 0:20 | 1.000, 0.000 | 42 | 9.13E−11 |
| Gm14:1702661 | T/C | 32:0, 0:20 | 1.000, 0.000 | 42 | 9.13E−11 |
| Gm14:1702755 | A/T | 32:0, 0:20 | 1.000, 0.000 | 42 | 9.13E−11 |
| Gm14:1704651 | C/T | 32:0, 0:20 | 1.000, 0.000 | 42 | 9.13E−11 |
| Gm14:1704664 | C/T | 32:0, 0:20 | 1.000, 0.000 | 42 | 9.13E−11 |
| Gm14:1712038 | A/G | 32:0, 0:20 | 1.000, 0.000 | 42 | 9.13E−11 |
| Gm14:1712320 | A/C | 32:0, 0:20 | 1.000, 0.000 | 42 | 9.13E−11 |
| Gm14:1712713 | A/G | 32:0, 0:20 | 1.000, 0.000 | 42 | 9.13E−11 |
| Gm14:1712865 | T/C | 32:0, 0:20 | 1.000, 0.000 | 42 | 9.13E−11 |
| Gm14:1712866 | A/T | 32:0, 0:20 | 1.000, 0.000 | 42 | 9.13E−11 |
| Gm14:1713999 | T/C | 32:0, 0:20 | 1.000, 0.000 | 42 | 9.13E−11 |
| Gm14:1719022 | T/G | 32:0, 0:20 | 1.000, 0.000 | 42 | 9.13E−11 |
| Gm14:1728216 | C/T | 32:0, 0:20 | 1.000, 0.000 | 42 | 9.13E−11 |
| Gm14:1731767 | T/C | 32:0, 0:20 | 1.000, 0.000 | 42 | 9.13E−11 |
| Gm14:1735093 | C/A | 32:0, 0:20 | 1.000, 0.000 | 42 | 9.13E−11 |
| Gm14:1735102 | T/A | 32:0, 0:20 | 1.000, 0.000 | 42 | 9.13E−11 |
| Gm14:1736945 | A/G | 32:0, 0:20 | 1.000, 0.000 | 42 | 9.13E−11 |
| Gm14:1743592 | G/A | 32:0, 0:20 | 1.000, 0.000 | 42 | 9.13E−11 |
| Gm14:1748064 | C/T | 32:0, 0:20 | 1.000, 0.000 | 42 | 9.13E−11 |
| Gm14:1749126 | C/A | 32:0, 0:20 | 1.000, 0.000 | 42 | 9.13E−11 |
| Gm14:1754686 | G/A | 32:0, 0:20 | 1.000, 0.000 | 42 | 9.13E−11 |
| Gm14:1757996 | C/G | 32:0, 0:20 | 1.000, 0.000 | 42 | 9.13E−11 |
| Gm14:1760483 | C/T | 32:0, 0:20 | 1.000, 0.000 | 42 | 9.13E−11 |
| Gm14:1783231 | T/A | 32:0, 0:20 | 1.000, 0.000 | 42 | 9.13E−11 |
| Gm14:1783538 | G/A | 32:0, 0:20 | 1.000, 0.000 | 42 | 9.13E−11 |
| Gm14:1783756 | T/G | 32:0, 0:20 | 1.000, 0.000 | 42 | 9.13E−11 |
| Gm14:1786013 | G/A | 32:0, 0:20 | 1.000, 0.000 | 42 | 9.13E−11 |
| Gm14:1787773 | A/G | 32:0, 0:20 | 1.000, 0.000 | 42 | 9.13E−11 |
| Gm14:1792218 | C/T | 32:0, 0:20 | 1.000, 0.000 | 42 | 9.13E−11 |
| Gm14:1799327 | A/T | 32:0, 0:20 | 1.000, 0.000 | 42 | 9.13E−11 |
| Gm14:1799390 | C/T | 32:0, 0:20 | 1.000, 0.000 | 42 | 9.13E−11 |
| Gm14:1799540 | T/C | 32:0, 0:20 | 1.000, 0.000 | 42 | 9.13E−11 |
| Gm14:1800012 | G/T | 32:0, 0:20 | 1.000, 0.000 | 42 | 9.13E−11 |
| Gm14:1800319 | T/G | 32:0, 0:20 | 1.000, 0.000 | 42 | 9.13E−11 |
| Gm14:1811113 | T/G | 32:0, 0:20 | 1.000, 0.000 | 42 | 9.13E−11 |
| Gm14:1811977 | G/A | 32:0, 0:20 | 1.000, 0.000 | 42 | 9.13E−11 |
| Gm14:1824284 | C/T | 32:0, 0:20 | 1.000, 0.000 | 42 | 9.13E−11 |
| Gm14:1837872 | C/T | 32:0, 0:20 | 1.000, 0.000 | 42 | 9.13E−11 |
| Gm14:1837888 | G/A | 32:0, 0:20 | 1.000, 0.000 | 42 | 9.13E−11 |
| Gm14:1851947 | G/A | 32:0, 0:20 | 1.000, 0.000 | 42 | 9.13E−11 |
| Gm14:1858277 | G/C | 32:0, 0:20 | 1.000, 0.000 | 42 | 9.13E−11 |
| Gm14:1861083 | A/T | 32:0, 0:20 | 1.000, 0.000 | 42 | 9.13E−11 |
| Gm14:1887021 | T/G | 32:0, 0:20 | 1.000, 0.000 | 42 | 9.13E−11 |
| Gm14:1903293 | C/T | 32:0, 0:20 | 1.000, 0.000 | 42 | 9.13E−11 |
| Gm14:1910033 | T/C | 32:0, 0:20 | 1.000, 0.000 | 42 | 9.13E−11 |
| Gm14:1919915 | A/G | 32:0, 0:20 | 1.000, 0.000 | 42 | 9.13E−11 |
| Gm14:1934737 | A/G | 32:0, 0:20 | 1.000, 0.000 | 42 | 9.13E−11 |
| Gm14:1946125 | A/G | 32:0, 0:20 | 1.000, 0.000 | 42 | 9.13E−11 |
| Gm14:1958086 | T/C | 32:0, 0:20 | 1.000, 0.000 | 42 | 9.13E−11 |
| Gm14:1986602 | T/C | 32:0, 0:20 | 1.000, 0.000 | 42 | 9.13E−11 |
| Gm14:1987892 | A/G | 32:0, 0:20 | 1.000, 0.000 | 42 | 9.13E−11 |
| Gm14:1988001 | G/A | 32:0, 0:20 | 1.000, 0.000 | 42 | 9.13E−11 |
| Gm14:1989951 | A/C | 32:0, 0:20 | 1.000, 0.000 | 42 | 9.13E−11 |
| Gm14:1991474 | A/C | 32:0, 0:20 | 1.000, 0.000 | 42 | 9.13E−11 |
| Gm14:1997517 | C/A | 32:0, 0:20 | 1.000, 0.000 | 42 | 9.13E−11 |
| Gm14:2006319 | C/G | 32:0, 0:20 | 1.000, 0.000 | 42 | 9.13E−11 |
| Gm14:2017162 | C/A | 32:0, 0:20 | 1.000, 0.000 | 42 | 9.13E−11 |
| Gm14:2019929 | A/G | 32:0, 0:20 | 1.000, 0.000 | 42 | 9.13E−11 |
| Gm14:1701692 | C/T | 32:0, 0:20 | 1.000, 0.000 | 40 | 2.54E−10 |
| Gm14:1702315 | A/T | 32:0, 0:20 | 1.000, 0.000 | 40 | 2.54E−10 |
| Gm14:1702956 | C/G | 32:0, 0:20 | 1.000, 0.000 | 40 | 2.54E−10 |
| Gm14:1703980 | A/C | 32:0, 0:20 | 1.000, 0.000 | 40 | 2.54E−10 |
| Gm14:1706523 | A/G | 32:0, 0:20 | 1.000, 0.000 | 40 | 2.54E−10 |
| Gm14:1711087 | C/A | 32:0, 0:20 | 1.000, 0.000 | 40 | 2.54E−10 |
| Gm14:1712377 | C/A | 32:0, 0:20 | 1.000, 0.000 | 40 | 2.54E−10 |
| Gm14:1714965 | G/A | 32:0, 0:20 | 1.000, 0.000 | 40 | 2.54E−10 |
| Gm14:1727031 | G/A | 32:0, 0:20 | 1.000, 0.000 | 40 | 2.54E−10 |
| Gm14:1727851 | A/T | 32:0, 0:20 | 1.000, 0.000 | 40 | 2.54E−10 |
| Gm14:1728741 | G/A | 32:0, 0:20 | 1.000, 0.000 | 40 | 2.54E−10 |
| Gm14:1729293 | A/G | 32:0, 0:20 | 1.000, 0.000 | 40 | 2.54E−10 |
| Gm14:1730480 | A/G | 32:0, 0:20 | 1.000, 0.000 | 40 | 2.54E−10 |
| Gm14:1730798 | A/T | 32:0, 0:20 | 1.000, 0.000 | 40 | 2.54E−10 |
| Gm14:1731840 | G/A | 32:0, 0:20 | 1.000, 0.000 | 40 | 2.54E−10 |
| Gm14:1732232 | G/A | 32:0, 0:20 | 1.000, 0.000 | 40 | 2.54E−10 |
| Gm14:1732358 | G/A | 32:0, 0:20 | 1.000, 0.000 | 40 | 2.54E−10 |
| Gm14:1732363 | T/G | 32:0, 0:20 | 1.000, 0.000 | 40 | 2.54E−10 |
| Gm14:1732403 | T/C | 32:0, 0:20 | 1.000, 0.000 | 40 | 2.54E−10 |
| Gm14:1735755 | T/C | 32:0, 0:20 | 1.000, 0.000 | 40 | 2.54E−10 |
| Gm14:1735945 | T/C | 32:0, 0:20 | 1.000, 0.000 | 40 | 2.54E−10 |
| Gm14:1743425 | A/G | 32:0, 0:20 | 1.000, 0.000 | 40 | 2.54E−10 |
| Gm14:1744356 | T/C | 32:0, 0:20 | 1.000, 0.000 | 40 | 2.54E−10 |
| Gm14:1746573 | T/C | 32:0, 0:20 | 1.000, 0.000 | 40 | 2.54E−10 |
| Gm14:1748959 | A/C | 32:0, 0:20 | 1.000, 0.000 | 40 | 2.54E−10 |
| Gm14:1750564 | G/C | 32:0, 0:20 | 1.000, 0.000 | 40 | 2.54E−10 |
| Gm14:1753875 | G/A | 32:0, 0:20 | 1.000, 0.000 | 40 | 2.54E−10 |
| Gm14:1754007 | A/G | 32:0, 0:20 | 1.000, 0.000 | 40 | 2.54E−10 |
| Gm14:1755671 | C/T | 32:0, 0:20 | 1.000, 0.000 | 40 | 2.54E−10 |
| Gm14:1756046 | G/T | 32:0, 0:20 | 1.000, 0.000 | 40 | 2.54E−10 |
| Gm14:1760299 | T/G | 32:0, 0:20 | 1.000, 0.000 | 40 | 2.54E−10 |
| Gm14:1760329 | A/G | 32:0, 0:20 | 1.000, 0.000 | 40 | 2.54E−10 |
| Gm14:1761362 | G/T | 32:0, 0:20 | 1.000, 0.000 | 40 | 2.54E−10 |
| Gm14:1766162 | T/G | 32:0, 0:20 | 1.000, 0.000 | 40 | 2.54E−10 |
| Gm14:1780165 | G/T | 32:0, 0:20 | 1.000, 0.000 | 40 | 2.54E−10 |
| Gm14:1780408 | G/A | 32:0, 0:20 | 1.000, 0.000 | 40 | 2.54E−10 |
| Gm14:1782983 | C/A | 32:0, 0:20 | 1.000, 0.000 | 40 | 2.54E−10 |
| Gm14:1789475 | G/T | 32:0, 0:20 | 1.000, 0.000 | 40 | 2.54E−10 |
| Gm14:1799494 | C/G | 32:0, 0:20 | 1.000, 0.000 | 40 | 2.54E−10 |
| Gm14:1799548 | C/T | 32:0, 0:20 | 1.000, 0.000 | 40 | 2.54E−10 |
| Gm14:1799571 | A/G | 32:0, 0:20 | 1.000, 0.000 | 40 | 2.54E−10 |
| Gm14:1811318 | A/T | 32:0, 0:20 | 1.000, 0.000 | 40 | 2.54E−10 |
| Gm14:1825691 | T/C | 32:0, 0:20 | 1.000, 0.000 | 40 | 2.54E−10 |
| Gm14:1834023 | G/A | 32:0, 0:20 | 1.000, 0.000 | 40 | 2.54E−10 |
| Gm14:1841281 | G/A | 32:0, 0:20 | 1.000, 0.000 | 40 | 2.54E−10 |
| Gm14:1846850 | T/C | 32:0, 0:20 | 1.000, 0.000 | 40 | 2.54E−10 |
| Gm14:1887108 | G/A | 32:0, 0:20 | 1.000, 0.000 | 40 | 2.54E−10 |
| Gm14:1887251 | T/C | 32:0, 0:20 | 1.000, 0.000 | 40 | 2.54E−10 |
| Gm14:1935369 | C/T | 32:0, 0:20 | 1.000, 0.000 | 40 | 2.54E−10 |
| Gm14:1969791 | A/G | 32:0, 0:20 | 1.000, 0.000 | 40 | 2.54E−10 |
| Gm14:1981574 | G/C | 32:0, 0:20 | 1.000, 0.000 | 40 | 2.54E−10 |
| Gm14:1981644 | G/A | 32:0, 0:20 | 1.000, 0.000 | 40 | 2.54E−10 |
| Gm14:1983907 | G/T | 32:0, 0:20 | 1.000, 0.000 | 40 | 2.54E−10 |
| Gm14:1986614 | C/G | 32:0, 0:20 | 1.000, 0.000 | 40 | 2.54E−10 |
| Gm14:1986675 | G/A | 32:0, 0:20 | 1.000, 0.000 | 40 | 2.54E−10 |
| Gm14:1987587 | A/T | 32:0, 0:20 | 1.000, 0.000 | 40 | 2.54E−10 |
| Gm14:1988422 | T/G | 32:0, 0:20 | 1.000, 0.000 | 40 | 2.54E−10 |
| Gm14:1988424 | C/T | 32:0, 0:20 | 1.000, 0.000 | 40 | 2.54E−10 |
| Gm14:1988520 | T/C | 32:0, 0:20 | 1.000, 0.000 | 40 | 2.54E−10 |
| Gm14:1988558 | G/A | 32:0, 0:20 | 1.000, 0.000 | 40 | 2.54E−10 |
| Gm14:1989403 | G/T | 32:0, 0:20 | 1.000, 0.000 | 40 | 2.54E−10 |
| Gm14:1991457 | C/T | 32:0, 0:20 | 1.000, 0.000 | 40 | 2.54E−10 |
| Gm14:1991910 | T/G | 32:0, 0:20 | 1.000, 0.000 | 40 | 2.54E−10 |
| Gm14:1992830 | T/C | 32:0, 0:20 | 1.000, 0.000 | 40 | 2.54E−10 |
| Gm14:1993931 | T/G | 32:0, 0:20 | 1.000, 0.000 | 40 | 2.54E−10 |
| Gm14:1997124 | A/G | 32:0, 0:20 | 1.000, 0.000 | 40 | 2.54E−10 |
| Gm14:1998879 | C/T | 32:0, 0:20 | 1.000, 0.000 | 40 | 2.54E−10 |
| Gm14:2004939 | G/A | 32:0, 0:20 | 1.000, 0.000 | 40 | 2.54E−10 |
| Gm14:2005896 | C/T | 32:0, 0:20 | 1.000, 0.000 | 40 | 2.54E−10 |
| Gm14:1698816 | A/G | 32:0, 0:20 | 1.000, 0.000 | 38 | 7.07E−10 |
| Gm14:1701991 | G/A | 32:0, 0:20 | 1.000, 0.000 | 38 | 7.07E−10 |
| Gm14:1710294 | G/A | 32:0, 0:20 | 1.000, 0.000 | 38 | 7.07E−10 |
| Gm14:1710303 | C/G | 32:0, 0:20 | 1.000, 0.000 | 38 | 7.07E−10 |
| Gm14:1711007 | G/T | 32:0, 0:20 | 1.000, 0.000 | 38 | 7.07E−10 |
| Gm14:1711575 | C/T | 32:0, 0:20 | 1.000, 0.000 | 38 | 7.07E−10 |
| Gm14:1713816 | G/C | 32:0, 0:20 | 1.000, 0.000 | 38 | 7.07E−10 |
| Gm14:1728293 | G/T | 32:0, 0:20 | 1.000, 0.000 | 38 | 7.07E−10 |
| Gm14:1729743 | A/G | 32:0, 0:20 | 1.000, 0.000 | 38 | 7.07E−10 |
| Gm14:1730281 | A/G | 32:0, 0:20 | 1.000, 0.000 | 38 | 7.07E−10 |
| Gm14:1730795 | G/T | 32:0, 0:20 | 1.000, 0.000 | 38 | 7.07E−10 |
| Gm14:1732205 | G/C | 32:0, 0:20 | 1.000, 0.000 | 38 | 7.07E−10 |
| Gm14:1744432 | A/G | 32:0, 0:20 | 1.000, 0.000 | 38 | 7.07E−10 |
| Gm14:1751350 | G/A | 32:0, 0:20 | 1.000, 0.000 | 38 | 7.07E−10 |
| Gm14:1755250 | C/T | 32:0, 0:20 | 1.000, 0.000 | 38 | 7.07E−10 |
| Gm14:1755398 | T/A | 32:0, 0:20 | 1.000, 0.000 | 38 | 7.07E−10 |
| Gm14:1756188 | G/A | 32:0, 0:20 | 1.000, 0.000 | 38 | 7.07E−10 |
| Gm14:1761081 | A/G | 32:0, 0:20 | 1.000, 0.000 | 38 | 7.07E−10 |
| Gm14:1761387 | A/G | 32:0, 0:20 | 1.000, 0.000 | 38 | 7.07E−10 |
| Gm14:1762867 | A/G | 32:0, 0:20 | 1.000, 0.000 | 38 | 7.07E−10 |
| Gm14:1765302 | G/A | 32:0, 0:20 | 1.000, 0.000 | 38 | 7.07E−10 |
| Gm14:1777323 | T/A | 32:0, 0:20 | 1.000, 0.000 | 38 | 7.07E−10 |
| Gm14:1779323 | A/G | 32:0, 0:20 | 1.000, 0.000 | 38 | 7.07E−10 |
| Gm14:1780397 | A/T | 32:0, 0:20 | 1.000, 0.000 | 38 | 7.07E−10 |
| Gm14:1800359 | C/T | 32:0, 0:20 | 1.000, 0.000 | 38 | 7.07E−10 |
| Gm14:1803234 | A/C | 32:0, 0:20 | 1.000, 0.000 | 38 | 7.07E−10 |
| Gm14:1811158 | A/G | 32:0, 0:20 | 1.000, 0.000 | 38 | 7.07E−10 |
| Gm14:1825653 | G/A | 32:0, 0:20 | 1.000, 0.000 | 38 | 7.07E−10 |
| Gm14:1869500 | G/A | 32:0, 0:20 | 1.000, 0.000 | 38 | 7.07E−10 |
| Gm14:1876026 | T/C | 32:0, 0:20 | 1.000, 0.000 | 38 | 7.07E−10 |
| Gm14:1876675 | G/A | 32:0, 0:20 | 1.000, 0.000 | 38 | 7.07E−10 |
| Gm14:1876844 | G/A | 32:0, 0:20 | 1.000, 0.000 | 38 | 7.07E−10 |
| Gm14:1876847 | G/T | 32:0, 0:20 | 1.000, 0.000 | 38 | 7.07E−10 |
| Gm14:1879162 | A/T | 32:0, 0:20 | 1.000, 0.000 | 38 | 7.07E−10 |
| Gm14:1884603 | T/C | 32:0, 0:20 | 1.000, 0.000 | 38 | 7.07E−10 |
| Gm14:1904675 | A/G | 32:0, 0:20 | 1.000, 0.000 | 38 | 7.07E−10 |
| Gm14:1944708 | T/C | 32:0, 0:20 | 1.000, 0.000 | 38 | 7.07E−10 |
| Gm14:1954056 | C/T | 32:0, 0:20 | 1.000, 0.000 | 38 | 7.07E−10 |
| Gm14:1954062 | T/G | 32:0, 0:20 | 1.000, 0.000 | 38 | 7.07E−10 |
| Gm14:1967108 | A/G | 32:0, 0:20 | 1.000, 0.000 | 38 | 7.07E−10 |
| Gm14:1972107 | C/A | 32:0, 0:20 | 1.000, 0.000 | 38 | 7.07E−10 |
| Gm14:2006215 | C/T | 32:0, 0:20 | 1.000, 0.000 | 38 | 7.07E−10 |
| Gm14:2011465 | G/C | 32:0, 0:20 | 1.000, 0.000 | 38 | 7.07E−10 |
| Gm14:1704689 | G/A | 32:0, 0:20 | 1.000, 0.000 | 36 | 1.97E−10 |
| Gm14:1711569 | G/A | 32:0, 0:20 | 1.000, 0.000 | 36 | 1.97E−10 |
| Gm14:1713323 | A/C | 32:0, 0:20 | 1.000, 0.000 | 36 | 1.97E−10 |
| Gm14:1728052 | G/A | 32:0, 0:20 | 1.000, 0.000 | 36 | 1.97E−10 |
| Gm14:1729537 | T/C | 32:0, 0:20 | 1.000, 0.000 | 36 | 1.97E−10 |
| Gm14:1732261 | T/A | 32:0, 0:20 | 1.000, 0.000 | 36 | 1.97E−10 |
| Gm14:1732593 | A/G | 32:0, 0:20 | 1.000, 0.000 | 36 | 1.97E−10 |
| Gm14:1735227 | A/C | 32:0, 0:20 | 1.000, 0.000 | 36 | 1.97E−10 |
| Gm14:1744452 | A/G | 32:0, 0:20 | 1.000, 0.000 | 36 | 1.97E−10 |
| Gm14:1754156 | A/G | 32:0, 0:20 | 1.000, 0.000 | 36 | 1.97E−10 |
| Gm14:1755877 | A/G | 32:0, 0:20 | 1.000, 0.000 | 36 | 1.97E−10 |
| Gm14:1756355 | C/A | 32:0, 0:20 | 1.000, 0.000 | 36 | 1.97E−10 |
| Gm14:1759702 | A/G | 32:0, 0:20 | 1.000, 0.000 | 36 | 1.97E−10 |
| Gm14:1780316 | A/G | 32:0, 0:20 | 1.000, 0.000 | 36 | 1.97E−10 |
| Gm14:1801585 | G/T | 32:0, 0:20 | 1.000, 0.000 | 36 | 1.97E−10 |
| Gm14:1810099 | A/G | 32:0, 0:20 | 1.000, 0.000 | 36 | 1.97E−10 |
| Gm14:1810126 | A/G | 32:0, 0:20 | 1.000, 0.000 | 36 | 1.97E−10 |
| Gm14:1810421 | C/T | 32:0, 0:20 | 1.000, 0.000 | 36 | 1.97E−10 |
| Gm14:1823776 | C/G | 32:0, 0:20 | 1.000, 0.000 | 36 | 1.97E−10 |
| Gm14:1875278 | T/A | 32:0, 0:20 | 1.000, 0.000 | 36 | 1.97E−10 |
| Gm14:1875874 | T/G | 32:0, 0:20 | 1.000, 0.000 | 36 | 1.97E−10 |
| Gm14:1902961 | G/A | 32:0, 0:20 | 1.000, 0.000 | 36 | 1.97E−10 |
| Gm14:1910054 | A/T | 32:0, 0:20 | 1.000, 0.000 | 36 | 1.97E−10 |
| Gm14:1949216 | T/A | 32:0, 0:20 | 1.000, 0.000 | 36 | 1.97E−10 |
| Gm14:1957357 | G/C | 32:0, 0:20 | 1.000, 0.000 | 36 | 1.97E−10 |
| Gm14:1957358 | A/T | 32:0, 0:20 | 1.000, 0.000 | 36 | 1.97E−10 |
| Gm14:1960447 | A/T | 32:0, 0:20 | 1.000, 0.000 | 36 | 1.97E−10 |
| Gm14:1980728 | C/T | 32:0, 0:20 | 1.000, 0.000 | 36 | 1.97E−10 |
| Gm14:1984129 | G/C | 32:0, 0:20 | 1.000, 0.000 | 36 | 1.97E−10 |
| Gm14:1992331 | C/T | 32:0, 0:20 | 1.000, 0.000 | 36 | 1.97E−10 |
| Gm14:1993147 | T/G | 32:0, 0:20 | 1.000, 0.000 | 36 | 1.97E−10 |
| Gm14:1995593 | G/A | 32:0, 0:20 | 1.000, 0.000 | 36 | 1.97E−10 |
| Gm14:1995600 | T/C | 32:0, 0:20 | 1.000, 0.000 | 36 | 1.97E−10 |
| Gm14:1996952 | G/T | 32:0, 0:20 | 1.000, 0.000 | 36 | 1.97E−10 |
| Gm14:1997412 | G/C | 32:0, 0:20 | 1.000, 0.000 | 36 | 1.97E−10 |
| Gm14:2004591 | T/A | 32:0, 0:20 | 1.000, 0.000 | 36 | 1.97E−10 |
| Gm14:1645762 | G/A | 32:0, 0:20 | 1.000, 0.000 | 34 | 5.51E−09 |
| Gm14:1713775 | T/C | 32:0, 0:20 | 1.000, 0.000 | 34 | 5.51E−09 |
| Gm14:1716505 | C/A | 32:0, 0:20 | 1.000, 0.000 | 34 | 5.51E−09 |
| Gm14:1729186 | A/G | 32:0, 0:20 | 1.000, 0.000 | 34 | 5.51E−09 |
| Gm14:1729798 | A/C | 32:0, 0:20 | 1.000, 0.000 | 34 | 5.51E−09 |
| Gm14:1732529 | A/G | 32:0, 0:20 | 1.000, 0.000 | 34 | 5.51E−09 |
| Gm14:1753922 | T/G | 32:0, 0:20 | 1.000, 0.000 | 34 | 5.51E−09 |
| Gm14:1755572 | A/C | 32:0, 0:20 | 1.000, 0.000 | 34 | 5.51E−09 |
| Gm14:1759619 | C/T | 32:0, 0:20 | 1.000, 0.000 | 34 | 5.51E−09 |
| Gm14:1759635 | C/T | 32:0, 0:20 | 1.000, 0.000 | 34 | 5.51E−09 |
| Gm14:1760144 | A/T | 32:0, 0:20 | 1.000, 0.000 | 34 | 5.51E−09 |
| Gm14:1761168 | T/G | 32:0, 0:20 | 1.000, 0.000 | 34 | 5.51E−09 |
| Gm14:1771612 | C/A | 32:0, 0:20 | 1.000, 0.000 | 34 | 5.51E−09 |
| Gm14:1777105 | T/A | 32:0, 0:20 | 1.000, 0.000 | 34 | 5.51E−09 |
| Gm14:1778517 | T/C | 32:0, 0:20 | 1.000, 0.000 | 34 | 5.51E−09 |
| Gm14:1779288 | T/C | 32:0, 0:20 | 1.000, 0.000 | 34 | 5.51E−09 |
| Gm14:1779568 | A/G | 32:0, 0:20 | 1.000, 0.000 | 34 | 5.51E−09 |
| Gm14:1804682 | A/C | 32:0, 0:20 | 1.000, 0.000 | 34 | 5.51E−09 |
| Gm14:1810135 | A/G | 32:0, 0:20 | 1.000, 0.000 | 34 | 5.51E−09 |
| Gm14:1825225 | G/T | 32:0, 0:20 | 1.000, 0.000 | 34 | 5.51E−09 |
| Gm14:1828925 | G/T | 32:0, 0:20 | 1.000, 0.000 | 34 | 5.51E−09 |
| Gm14:1846994 | G/C | 32:0, 0:20 | 1.000, 0.000 | 34 | 5.51E−09 |
| Gm14:1848374 | A/T | 32:0, 0:20 | 1.000, 0.000 | 34 | 5.51E−09 |
| Gm14:1874941 | T/C | 32:0, 0:20 | 1.000, 0.000 | 34 | 5.51E−09 |
| Gm14:1875261 | G/A | 32:0, 0:20 | 1.000, 0.000 | 34 | 5.51E−09 |
| Gm14:1875470 | A/G | 32:0, 0:20 | 1.000, 0.000 | 34 | 5.51E−09 |
| Gm14:1879189 | C/T | 32:0, 0:20 | 1.000, 0.000 | 34 | 5.51E−09 |
| Gm14:1881168 | G/C | 32:0, 0:20 | 1.000, 0.000 | 34 | 5.51E−09 |
| Gm14:1923548 | A/T | 32:0, 0:20 | 1.000, 0.000 | 34 | 5.51E−09 |
| Gm14:1923935 | T/C | 32:0, 0:20 | 1.000, 0.000 | 34 | 5.51E−09 |
| Gm14:1955048 | G/A | 32:0, 0:20 | 1.000, 0.000 | 34 | 5.51E−09 |
| Gm14:1955118 | C/A | 32:0, 0:20 | 1.000, 0.000 | 34 | 5.51E−09 |
| Gm14:1955144 | T/C | 32:0, 0:20 | 1.000, 0.000 | 34 | 5.51E−09 |
| Gm14:1959610 | T/G | 32:0, 0:20 | 1.000, 0.000 | 34 | 5.51E−09 |
| Gm14:1960454 | A/C | 32:0, 0:20 | 1.000, 0.000 | 34 | 5.51E−09 |
| Gm14:1967167 | T/G | 32:0, 0:20 | 1.000, 0.000 | 34 | 5.51E−09 |
| Gm14:1970908 | C/T | 32:0, 0:20 | 1.000, 0.000 | 34 | 5.51E−09 |
| Gm14:1976442 | G/T | 32:0, 0:20 | 1.000, 0.000 | 34 | 5.51E−09 |
| Gm14:1981936 | C/A | 32:0, 0:20 | 1.000, 0.000 | 34 | 5.51E−09 |
| Gm14:1982632 | C/T | 32:0, 0:20 | 1.000, 0.000 | 34 | 5.51E−09 |
| Gm14:1982756 | C/T | 32:0, 0:20 | 1.000, 0.000 | 34 | 5.51E−09 |
| Gm14:1984276 | G/T | 32:0, 0:20 | 1.000, 0.000 | 34 | 5.51E−09 |
| Gm14:1994907 | A/T | 32:0, 0:20 | 1.000, 0.000 | 34 | 5.51E−09 |
| Gm14:2013156 | G/T | 32:0, 0:20 | 1.000, 0.000 | 34 | 5.51E−09 |
| Gm14:2019411 | A/G | 32:0, 0:20 | 1.000, 0.000 | 34 | 5.51E−09 |
| Gm14:1711379 | G/A | 32:0, 0:20 | 1.000, 0.000 | 32 | 1.54E−09 |
| Gm14:1713140 | T/C | 32:0, 0:20 | 1.000, 0.000 | 32 | 1.54E−09 |
| Gm14:1727997 | G/A | 32:0, 0:20 | 1.000, 0.000 | 32 | 1.54E−09 |
| Gm14:1744286 | A/G | 32:0, 0:20 | 1.000, 0.000 | 32 | 1.54E−09 |
| Gm14:1757864 | T/C | 32:0, 0:20 | 1.000, 0.000 | 32 | 1.54E−09 |
| Gm14:1759363 | C/T | 32:0, 0:20 | 1.000, 0.000 | 32 | 1.54E−09 |
| Gm14:1765933 | A/G | 32:0, 0:20 | 1.000, 0.000 | 32 | 1.54E−09 |
| Gm14:1767525 | C/T | 32:0, 0:20 | 1.000, 0.000 | 32 | 1.54E−09 |
| Gm14:1767582 | C/G | 32:0, 0:20 | 1.000, 0.000 | 32 | 1.54E−09 |
| Gm14:1768845 | C/T | 32:0, 0:20 | 1.000, 0.000 | 32 | 1.54E−09 |
| Gm14:1775564 | T/A | 32:0, 0:20 | 1.000, 0.000 | 32 | 1.54E−09 |
| Gm14:1778106 | G/A | 32:0, 0:20 | 1.000, 0.000 | 32 | 1.54E−09 |
| Gm14:1778621 | T/C | 32:0, 0:20 | 1.000, 0.000 | 32 | 1.54E−09 |
| Gm14:1780281 | T/C | 32:0, 0:20 | 1.000, 0.000 | 32 | 1.54E−09 |
| Gm14:1780494 | G/A | 32:0, 0:20 | 1.000, 0.000 | 32 | 1.54E−09 |
| Gm14:1805372 | C/T | 32:0, 0:20 | 1.000, 0.000 | 32 | 1.54E−09 |
| Gm14:1809090 | A/T | 32:0, 0:20 | 1.000, 0.000 | 32 | 1.54E−09 |
| Gm14:1810313 | G/A | 32:0, 0:20 | 1.000, 0.000 | 32 | 1.54E−09 |
| Gm14:1812109 | T/A | 32:0, 0:20 | 1.000, 0.000 | 32 | 1.54E−09 |
| Gm14:1812114 | A/T | 32:0, 0:20 | 1.000, 0.000 | 32 | 1.54E−09 |
| Gm14:1824651 | A/G | 32:0, 0:20 | 1.000, 0.000 | 32 | 1.54E−09 |
| Gm14:1875656 | A/G | 32:0, 0:20 | 1.000, 0.000 | 32 | 1.54E−09 |
| Gm14:1877489 | G/T | 32:0, 0:20 | 1.000, 0.000 | 32 | 1.54E−09 |
| Gm14:1878068 | T/C | 32:0, 0:20 | 1.000, 0.000 | 32 | 1.54E−09 |
| Gm14:1903061 | G/A | 32:0, 0:20 | 1.000, 0.000 | 32 | 1.54E−09 |
| Gm14:1950278 | A/G | 32:0, 0:20 | 1.000, 0.000 | 32 | 1.54E−09 |
| Gm14:1950577 | C/G | 32:0, 0:20 | 1.000, 0.000 | 32 | 1.54E−09 |
| Gm14:1950969 | G/A | 32:0, 0:20 | 1.000, 0.000 | 32 | 1.54E−09 |
| Gm14:1970110 | T/A | 32:0, 0:20 | 1.000, 0.000 | 32 | 1.54E−09 |
| Gm14:1982411 | C/T | 32:0, 0:20 | 1.000, 0.000 | 32 | 1.54E−09 |
| Gm14:1983489 | T/C | 32:0, 0:20 | 1.000, 0.000 | 32 | 1.54E−09 |
| Gm14:1983507 | T/C | 32:0, 0:20 | 1.000, 0.000 | 32 | 1.54E−09 |
| Gm14:1983593 | T/C | 32:0, 0:20 | 1.000, 0.000 | 32 | 1.54E−09 |
| Gm14:1993730 | G/A | 32:0, 0:20 | 1.000, 0.000 | 32 | 1.54E−09 |
| Gm14:1993788 | T/G | 32:0, 0:20 | 1.000, 0.000 | 32 | 1.54E−09 |
| Gm14:1993806 | A/G | 32:0, 0:20 | 1.000, 0.000 | 32 | 1.54E−09 |
| Gm14:2006229 | C/T | 32:0, 0:20 | 1.000, 0.000 | 32 | 1.54E−09 |
| Gm14:2015853 | C/A | 32:0, 0:20 | 1.000, 0.000 | 32 | 1.54E−09 |
| Gm14:2359579 | G/A | 32:0, 0:20 | 1.000, 0.000 | 32 | 1.54E−09 |
| Gm14:1579650 | G/A | 32:0, 0:20 | 1.000, 0.000 | 30 | 4.32E−08 |
| Gm14:1764334 | T/C | 32:0, 0:20 | 1.000, 0.000 | 30 | 4.32E−08 |
| Gm14:1764392 | G/A | 32:0, 0:20 | 1.000, 0.000 | 30 | 4.32E−08 |
| Gm14:1764403 | T/C | 32:0, 0:20 | 1.000, 0.000 | 30 | 4.32E−08 |
| Gm14:1767878 | C/T | 32:0, 0:20 | 1.000, 0.000 | 30 | 4.32E−08 |
| Gm14:1805366 | T/G | 32:0, 0:20 | 1.000, 0.000 | 30 | 4.32E−08 |
| Gm14:1825173 | A/G | 32:0, 0:20 | 1.000, 0.000 | 30 | 4.32E−08 |
| Gm14:1879313 | C/T | 32:0, 0:20 | 1.000, 0.000 | 30 | 4.32E−08 |
| Gm14:1886238 | G/C | 32:0, 0:20 | 1.000, 0.000 | 30 | 4.32E−08 |
| Gm14:1899408 | G/T | 32:0, 0:20 | 1.000, 0.000 | 30 | 4.32E−08 |
| Gm14:1960137 | G/A | 32:0, 0:20 | 1.000, 0.000 | 30 | 4.32E−08 |
| Gm14:1971009 | G/C | 32:0, 0:20 | 1.000, 0.000 | 30 | 4.32E−08 |
| Gm14:1983348 | T/G | 32:0, 0:20 | 1.000, 0.000 | 30 | 4.32E−08 |
| Gm14:2011607 | C/A | 32:0, 0:20 | 1.000, 0.000 | 30 | 4.32E−08 |
| Gm14:2012105 | C/T | 32:0, 0:20 | 1.000, 0.000 | 30 | 4.32E−08 |
| Gm14:2340746 | A/C | 32:0, 0:20 | 1.000, 0.000 | 30 | 4.32E−08 |
| Gm14:1767671 | C/A | 32:0, 0:20 | 1.000, 0.000 | 28 | 1.21E−07 |
| Gm14:1771502 | A/G | 32:0, 0:20 | 1.000, 0.000 | 28 | 1.21E−07 |
| Gm14:1771578 | A/C | 32:0, 0:20 | 1.000, 0.000 | 28 | 1.21E−07 |
| Gm14:1803995 | G/A | 32:0, 0:20 | 1.000, 0.000 | 28 | 1.21E−07 |
| Gm14:1805322 | G/A | 32:0, 0:20 | 1.000, 0.000 | 28 | 1.21E−07 |
| Gm14:1809988 | C/T | 32:0, 0:20 | 1.000, 0.000 | 28 | 1.21E−07 |
| Gm14:1825310 | C/T | 32:0, 0:20 | 1.000, 0.000 | 28 | 1.21E−07 |
| Gm14:1888310 | G/A | 32:0, 0:20 | 1.000, 0.000 | 28 | 1.21E−07 |
| Gm14:1889307 | A/T | 32:0, 0:20 | 1.000, 0.000 | 28 | 1.21E−07 |
| Gm14:1957202 | A/G | 32:0, 0:20 | 1.000, 0.000 | 28 | 1.21E−07 |
| Gm14:1981029 | A/C | 32:0, 0:20 | 1.000, 0.000 | 28 | 1.21E−07 |
| Gm14:1981073 | A/T | 32:0, 0:20 | 1.000, 0.000 | 28 | 1.21E−07 |
| Gm14:1981255 | T/C | 32:0, 0:20 | 1.000, 0.000 | 28 | 1.21E−07 |
| Gm14:1992432 | C/G | 32:0, 0:20 | 1.000, 0.000 | 28 | 1.21E−07 |
| Gm14:2010834 | C/T | 32:0, 0:20 | 1.000, 0.000 | 28 | 1.21E−07 |
| Gm14:1759440 | C/T | 32:0, 0:20 | 1.000, 0.000 | 26 | 3.41E−07 |
| Gm14:1775298 | A/T | 32:0, 0:20 | 1.000, 0.000 | 26 | 3.41E−07 |
| Gm14:1775605 | C/T | 32:0, 0:20 | 1.000, 0.000 | 26 | 3.41E−07 |
| Gm14:1804116 | C/T | 32:0, 0:20 | 1.000, 0.000 | 26 | 3.41E−07 |
| Gm14:1804868 | T/C | 32:0, 0:20 | 1.000, 0.000 | 26 | 3.41E−07 |
| Gm14:1806330 | C/T | 32:0, 0:20 | 1.000, 0.000 | 26 | 3.41E−07 |
| Gm14:1806377 | T/C | 32:0, 0:20 | 1.000, 0.000 | 26 | 3.41E−07 |
| Gm14:1889158 | T/C | 32:0, 0:20 | 1.000, 0.000 | 26 | 3.41E−07 |
| Gm14:1899428 | T/A | 32:0, 0:20 | 1.000, 0.000 | 26 | 3.41E−07 |
| Gm14:1926218 | T/A | 32:0, 0:20 | 1.000, 0.000 | 26 | 3.41E−07 |
| Gm14:1928522 | C/T | 32:0, 0:20 | 1.000, 0.000 | 26 | 3.41E−07 |
| Gm14:1957206 | T/C | 32:0, 0:20 | 1.000, 0.000 | 26 | 3.41E−07 |
| Gm14:1959033 | A/T | 32:0, 0:20 | 1.000, 0.000 | 26 | 3.41E−07 |
| Gm14:1969518 | C/T | 32:0, 0:20 | 1.000, 0.000 | 26 | 3.41E−07 |
| Gm14:1975722 | A/G | 32:0, 0:20 | 1.000, 0.000 | 26 | 3.41E−07 |
| Gm14:1981293 | C/T | 32:0, 0:20 | 1.000, 0.000 | 26 | 3.41E−07 |
| Gm14:1983189 | T/A | 32:0, 0:20 | 1.000, 0.000 | 26 | 3.41E−07 |
| Gm14:2013786 | T/C | 32:0, 0:20 | 1.000, 0.000 | 26 | 3.41E−07 |
| Gm14:2014487 | G/A | 32:0, 0:20 | 1.000, 0.000 | 26 | 3.41E−07 |
| Gm14:2191538 | A/C | 32:0, 0:20 | 1.000, 0.000 | 26 | 3.41E−07 |
| Gm14:2191541 | C/T | 32:0, 0:20 | 1.000, 0.000 | 26 | 3.41E−07 |
| Gm14:2191563 | T/C | 32:0, 0:20 | 1.000, 0.000 | 26 | 3.41E−07 |
| Gm14:1711534 | C/A | 32:0, 0:20 | 1.000, 0.000 | 24 | 9.63E−07 |
| Gm14:1773993 | C/A | 32:0, 0:20 | 1.000, 0.000 | 24 | 9.63E−07 |
| Gm14:1808347 | G/A | 32:0, 0:20 | 1.000, 0.000 | 24 | 9.63E−07 |
| Gm14:1808348 | C/A | 32:0, 0:20 | 1.000, 0.000 | 24 | 9.63E−07 |
| Gm14:1812356 | T/A | 32:0, 0:20 | 1.000, 0.000 | 24 | 9.63E−07 |
| Gm14:1812358 | C/A | 32:0, 0:20 | 1.000, 0.000 | 24 | 9.63E−07 |
| Gm14:1874583 | G/T | 32:0, 0:20 | 1.000, 0.000 | 24 | 9.63E−07 |
| Gm14:1971096 | C/T | 32:0, 0:20 | 1.000, 0.000 | 24 | 9.63E−07 |
| Gm14:1975723 | G/A | 32:0, 0:20 | 1.000, 0.000 | 24 | 9.63E−07 |
| Gm14:1976101 | G/C | 32:0, 0:20 | 1.000, 0.000 | 24 | 9.63E−07 |
| Gm14:1998648 | T/A | 32:0, 0:20 | 1.000, 0.000 | 24 | 9.63E−07 |
| Gm14:2006929 | T/G | 32:0, 0:20 | 1.000, 0.000 | 24 | 9.63E−07 |
| Gm14:2013850 | T/A | 32:0, 0:20 | 1.000, 0.000 | 24 | 9.63E−07 |
| Gm14:2014479 | T/A | 32:0, 0:20 | 1.000, 0.000 | 24 | 9.63E−07 |
| Gm14:2014481 | T/A | 32:0, 0:20 | 1.000, 0.000 | 24 | 9.63E−07 |
| Gm14:2101794 | A/T | 32:0, 0:20 | 1.000, 0.000 | 24 | 9.63E−07 |
| Gm14:766045 | A/T | 32:0, 0:20 | 1.000, 0.000 | 22 | 2.73E−06 |
| Gm14:1801323 | T/A | 32:0, 0:20 | 1.000, 0.000 | 22 | 2.73E−06 |
| Gm14:1805026 | A/T | 32:0, 0:20 | 1.000, 0.000 | 22 | 2.73E−06 |
| Gm14:1879302 | G/A | 32:0, 0:20 | 1.000, 0.000 | 22 | 2.73E−06 |
| Gm14:1975774 | T/C | 32:0, 0:20 | 1.000, 0.000 | 22 | 2.73E−06 |
| Gm14:2014429 | G/A | 32:0, 0:20 | 1.000, 0.000 | 20 | 7.74E−06 |
| Gm14:2191662 | G/A | 32:0, 0:20 | 1.000, 0.000 | 20 | 7.74E−06 |
| Gm14:584976 | C/T | 32:0, 0:20 | 1.000, 0.000 | 18 | 2.21E−05 |
| Gm14:2007005 | C/A | 32:0, 0:20 | 1.000, 0.000 | 18 | 2.21E−05 |
| Gm14:1650177 | A/C | 32:0, 0:20 | 1.000, 0.000 | 16 | 6.33E−05 |
| Gm14:1899046 | A/G | 32:0, 0:20 | 1.000, 0.000 | 16 | 6.33E−05 |
| Gm14:806705 | C/T | 32:0, 0:20 | 1.000, 0.000 | 14 | 2.00E−04 |
| Gm14:1976057 | G/A | 32:0, 0:20 | 1.000, 0.000 | 14 | 2.00E−04 |
| Gm14:1889837 | A/T | 32:0, 0:20 | 1.000, 0.000 | 12 | 5.00E−04 |

A set of perfectly associated SNPs are predicted to have a non-synonymous variation or premature stop within a predicted encoded amino acid sequence of the gene models as shown in Table 3. These predicted mutations are candidate putative causative variants underlying stem canker trait variation. These overlap with gene models predicted to have a functional effect. Table 3 summarizes SNPs predicted to generate non-synonymous amino acid changes or stop codon within the predicted protein-coding region of the indicated gene model. A marker for the locus S08231-3 targets the polymorphism at Gm14:1727084 in gene model Glyma14g02740.1 is shown in bold in Table 3.

TABLE 3

| | | | Chi | Allele |
|---|---|---|---|---|
| SNP | Gene Model | Change | Value | (R/S) |
| Gm14:1727084 | Glyma14g02740.1 | Exonic; Non-Syn | 48 | C/G |
| Gm14:1731518 | | Exonic; Non-Syn | 46 | T/G |
| Gm14:1731690 | | Exonic; Non-Syn | 48 | C/T |
| Gm14:1731767 | | Exonic; Non-Syn | 42 | T/C |
| Gm14:1739468 | Glyma14g02750.1 | Exonic; Non-Syn | 46 | C/T |
| Gm14:1743592 | | Exonic; Non-Syn | 42 | G/A |
| Gm14:1744286 | | Exonic; Non-Syn | 32 | A/G |
| Gm14:1767525 | Glyma14g02780.1 | Exonic; Non-Syn | 32 | C/T |
| Gm14:1777105 | | Exonic; Non-Syn | 34 | T/A |
| Gm14:1784221 | Glyma14g02800.1 | Exonic; Non-Syn | 44 | T/A |
| Gm14:1792218 | Glyma14g02820.1 | Exonic; Non-Syn | 42 | C/T |
| Gm14:1851947 | Glyma14g02920.1 | Stop | 42 | G/A |
| Gm14:1911679 | Glyma14g03010.1 | Exonic; Non-Syn | 50 | A/T |
| Gm14:1914305 | Glyma14g03030.1 | Exonic; Non-Syn | 52 | C/T |
| Gm14:1935369 | Glyma14g03050.1 | Exonic; Non-Syn | 40 | C/T |
| Gm14:1936433 | Glyma14g03060.1 | Exonic; Non-Syn | 46 | T/C |
| Gm14:1981029 | Glyma14g03100.1 | Exonic; Non-Syn | 28 | A/C |
| Gm14:1981936 | | Exonic; Non-Syn | 34 | C/A |
| Gm14:1706973 | Glyma14g02710.4 | Exonic; Non-Syn | 44 | T/C |
| Gm14:1723656 | Glyma14g02730.1 | Exonic; Non-Syn | 52 | C/T |
| Gm14:1724087 | | Exonic; Non-Syn | 50 | A/G |
| Gm14:1724319 | | Exonic; Non-Syn | 52 | G/A |
| Gm14:1724793 | | Exonic; Non-Syn | 44 | T/A |

### Example 3

### Mapping Population Study

An F2 population of 180 progeny from a cross of a Brazilian soybean line and Pioneer proprietary soybean line EMGOPA313×98R31, segregating for response to southern stem canker, was used for stem canker marker validation and refinement. Parental line 98R31 scores high for stem canker resistance (score=9), while EMGOPA313 is susceptible (score=1) to the pathogen. The population was screened for QTLs for stem canker on LG-B2 or LG-D1b. A total of 15 markers polymorphic between the parental lines on LG-B2 and LG-D1b were utilized to construct the linkage map and perform QTL analysis. One major QTL for stem canker resistance was identified on LG-B2. The QTL spanned interval of marker S04492-1-A (10.27 cM, Gm14:1197243) and marker 500802-1-A (17.87 cM, Gm14:3468738) which includes 501591-1-A (11.17 cM, Gm14:1923247) and was closely linked with marker S04492-1-A (10.27 cM, Gm14:1197243), which explained about 48% of the phenotypic variation.

Phenotypic data for this mapping population was collected from plants and the phenotypic data were scored on a scale of 1 (susceptible), 3, 6, and 9 (resistant).

Markers polymorphic between the two parents (EMGOPA313 and 98R31) in the regions of interest (top of LG-B2, middle of LG-D1b) were identified manually based on a proprietary composite genetic map. A total of 17 polymorphic markers were identified to assay the entire population. One marker was removed due to monomorphism. The allele source was assigned maternal=A, paternal=B, and heterozygous=H. Chi-square analysis revealed one marker exhibited severe segregation distortion, and was removed from any subsequent mapping analyses.

Map Manager QTX.b20 (Manly et al. (2001) Mammalian Genome 12:930-932) was used to construct the linkage map, consisting of 15 markers, and perform the QTL analysis. A 1000 permutation test was used to establish the threshold for statistical significance (LOD ratio statistic—LRS) to declare putative QTL. The mean score was used for QTL mapping. Initial Map Manager Parameters were set to:

1) Linkage Evaluation: Intercross

2) Search Criteria: P=1e-5

3) Map Function: Kosambi

4) Cross Type: Line Cross

Progeny stem canker score distribution was skewed toward the higher scores, but there were sufficient numbers of progeny in each class for analysis. When progeny with scores of 1 and 3 were classified as susceptible, and those with scores of 6 and 9 as resistant, then the p value of chi-square test for the ratio 3:1 was 0.23 (<0.05), which implied that one dominant gene might control the resistance to the disease.

The 15 segregation markers were mapped to the two linkage groups, with 6 markers mapping to LG B2, and 9 markers mapping to LG D1b. The 1000 permutation test set the significance thresholds for stem canker at 10.4 for a p-value of 0.01 (significant), and at 17.6 for a p-value of 0.05 (highly significant).

The marker regression analysis showed that the QTL associated with stem canker resistance could be located on LG-B2. Marker regression data for LG B2 is summarized in Table 4 below.

TABLE 4

| Locus | Genetic (cM) | Physical (bp) | LRS | % PhenoVar | p-value |
|---|---|---|---|---|---|
| S01591-1 | 11.17 | 1923247 | 69.3 | 48 | 0.00000 |
| S04492-1 | 10.27 | 1197243 | 119.0 | 48 | 0.00000 |
| S00802-1 | 17.87 | 3468738 | 71.0 | 33 | 0.00000 |
| S01799-1 | 19.06 | 3727753 | 75.0 | 34 | 0.00000 |
| S03923-1 | 24.86 | 4946161 | 53.3 | 26 | 0.00000 |

This region was investigated further by interval mapping method in Map Manager

QTX.b20 using Kosambi map function with no control for other QTLs. The QTL spanned an interval comprising marker S04492-1-A through marker S00802-1-A and was closely linked with marker S04492-1-A which explained about 48% of the phenotypic variation observed.

### Example 4

### Mapping Population Study

An F2 population of 180 progeny from a cross of a Brazilian soybean line and Pioneer proprietary soybean line IGUACU×98R31, segregating for response to southern stem canker, was used for stem canker marker validation and refinement. Parental line 98R31 scores high for stem canker resistance (score=9), while IGUACU is susceptible to the pathogen (score=1). The population was screened for QTLs for stem canker on LG-B2 or LG-D1b. A total of 13 markers polymorphic between the parental lines on LG-B2 and LG-D1b were utilized to construct the linkage map and perform QTL analysis. One major QTL for stem canker resistance was identified on LG-B2. The QTL spanned interval of marker 504492-1-A (10.27 cM, Gm14:1197243) and marker 504785-1-A (26.08 cM, Gm14:5096522) and closely linked with marker S01799-1-A (19.06 cM, Gm14:3727753), which explained about 38% of the phenotypic variation.

Phenotypic data for this mapping population was collected from plants which were scored on a scale of 1 (susceptible), 3, 6, and 9 (resistant).

Markers polymorphic between the two parents (IGUACU and 98R31) in the regions of interest (top of LG-B2, middle of LG-D1b) were identified manually based on a proprietary composite genetic map. A total of 17 polymorphic markers were identified to assay the entire population. One marker was removed due to monomorphism. The allele source was assigned maternal=A, paternal=B, and heterozygous=H. Chi-square analysis revealed three markers exhibited severe segregation distortion, and these were removed from any subsequent mapping analyses.

Map Manager QTX.b20 (Manly et al. (2001) Mammalian Genome 12:930-932) was used to construct the linkage map, consisting of 13 markers, and perform the QTL analysis. A 1000 permutation test was used to establish the threshold for statistical significance (LOD ratio statistic—LRS) to declare putative QTL. The mean score was used for QTL mapping. Initial Map Manager Parameters were set to:

1) Linkage Evaluation: Intercross

2) Search Criteria: P=1e-5

3) Map Function: Kosambi

4) Cross Type: Line Cross

Progeny stem canker score distribution was somewhat skewed toward the higher score of 6, but there were sufficient numbers of progeny in each class for analysis. When progeny with scores of 1 and 3 were classified as susceptible, and those with scores of 6 and 9 as resistant, then the p value of chi-square test for the ratio 3:1 was 0.28 (<0.05), which implied that one dominant gene might control the resistance to the disease.

The 13 segregation markers were mapped to four linkage groups, 2 each corresponding to LG B2 and to LG D1b. Two sets of 3 markers markers mapped to LG B2, and a set of 5 markers plus a set of 2 markers mapped to LG D1b. The 1000 permutation test set the significance thresholds for stem canker at 16.5 for a p-value of 0.01 (significant), and at 43.9 for a p-value of 0.05 (highly significant).

The marker regression analysis showed that the QTL associated with stem canker resistance could be located on LG-B2 and LG D1b at p-0.001. Marker regression data for LG B2 is summarized in Table 5 below.

TABLE 5

| Locus | Genetic (cM) | Physical (bp) | LRS | % PhenoVar | p-value |
|---|---|---|---|---|---|
| S04785-1 | 26.08 | 5096522 | 59.1 | 29 | 0.00000 |
| S01799-1 | 19.06 | 3727753 | 51.0 | 25 | 0.00000 |
| S04492-1 | 10.27 | 1197243 | 62.4 | 30 | 0.00000 |

This region was investigated further by interval mapping method in Map Manager QTX.b20 using Kosambi map function with no control for other QTLs. A highly significant (LRS=83) QTL spanned an interval comprising marker S04785-1-A through marker S04492-1-A and was closely linked with marker S01799-1-A which explained about 38% of the phenotypic variation observed.

### Example 5

### Mapping Population Study

An F2 population of 180 progeny from a cross of a Brazilian soybean line and Pioneer proprietary soybean line IGUACU×98Y11, segregating for response to southern stem canker, was used for stem canker marker validation and refinement. Parental line 98Y11 scores high for stem canker resistance (score=9), while IGUACU is susceptible to the pathogen (score=1). The population was screened for QTLs for stem canker on LG-B2 or LG-D1b. A total of 10 markers polymorphic between the parental lines on LG-B2 and LG-D1b were utilized to construct the linkage map and perform QTL analysis. One major QTL for stem canker resistance was identified on LG-B2. The QTL spanned interval of marker S01591-1-A (11.17 cM, Gm14:1923247) and marker S01718-1-A (15 cM, Gm14:2747160) and closely linked with marker S01591-1-A (11.17 cM, Gm14:1923247), which explained about 36% of the phenotypic variation.

Phenotypic data for this mapping population was collected from plants which were scored on a scale of 1 (susceptible), 3, 6, and 9 (resistant).

Markers polymorphic between the two parents (IGUACU and 98Y11) in the regions of interest (top of LG-B2, middle of LG-D1b) were identified manually based on a proprietary composite genetic map. A total of 17 polymorphic markers were identified to assay the entire population. Six markers were removed due to monomorphism. The allele source was assigned maternal=A, paternal=B, and heterozygous=H. Chi-square analysis revealed one marker exhibited severe segregation distortion, and this was removed from any subsequent mapping analyses.

Map Manager QTX.b20 (Manly et al. (2001) Mammalian Genome 12:930-932) was used to construct the linkage map, consisting of 13 markers, and perform the QTL analysis. A 1000 permutation test was used to establish the threshold for statistical significance (LOD ratio statistic—LRS) to declare putative QTL. The mean score was used for QTL mapping. Initial Map Manager Parameters were set to:

1) Linkage Evaluation: Intercross

2) Search Criteria: P=1e-5

3) Map Function: Kosambi

4) Cross Type: Line Cross

Progeny stem canker score distribution was somewhat skewed toward the higher scores, but there were sufficient numbers of progeny in each class for analysis. When progeny with scores of 1 and 3 were classified as susceptible, and those with scores of 6 and 9 as resistant, then the p value of chi-square test for the ratio 3:1 was 0.18 (<0.05), which implied that one dominant gene might control the resistance to the disease.

The 10 segregation markers were mapped to three linkage groups, one corresponding to LG B2 and 2 corresponding to LG D1b. Four markers mapped to LG B2, and a set of 2 markers plus a set of 4 markers mapped to LG D1b. The 1000 permutation test set the significance thresholds for stem canker at 9.3 for a p-value of 0.01 (significant), and at 17.7 for a p-value of 0.05 (highly significant).

The marker regression analysis showed that the QTL associated with stem canker resistance could be located on LG-B2. Marker regression data for LG B2 is summarized in Table 6 below.

TABLE 6

| Locus | Genetic (cM) | Physical (bp) | LRS | % PhenoVar | p-value |
|---|---|---|---|---|---|
| S01591-1 | 11.17 | 1923247 | 80.5 | 36 | 0.00000 |
| S02987-1 | 11.74 | 2129691 | 75.8 | 35 | 0.00000 |
| S01718-1 | 15.00 | 2747150 | 63.3 | 30 | 0.00000 |
| S04785-1 | 26.08 | 5096522 | 39.7 | 20 | 0.00000 |

This region was investigated further by interval mapping method in Map Manager QTX.b20 using Kosambi map function with no control for other QTLs. A highly significant (LRS=80) QTL spanned an interval comprising marker S01591-1-A through marker S01718-1-A and was closely linked with marker S01591-1-A which explained about 36% of the phenotypic variation observed.

### Example 6

### Mapping Population Study

An F2 population of 184 progeny from a cross of a two Pioneer proprietary soybean lines 95M60×PHI_Res, segregating for response to southern stem canker, was used for stem canker marker validation and refinement. Parental line PHI_Res scores high for stem canker resistance (score=9), while 95M60 is susceptible to the pathogen (score=1). The population was screened for QTLs for stem canker on LG-B2 or LG-D1b. A total of 21 markers polymorphic between the parental lines on LG-B2 and LG-D1b were utilized to construct the linkage map and perform QTL analysis. One major QTL for stem canker resistance was identified on LG-B2 for all three phenotypic data sets. The QTL was closely linked with marker S03188-1-A (10.17 cM, Gm14:1088724), which explained about 24%, 12%, and 31% of the phenotypic variation observed in the external screen, internal screen, and overall phenotypic data sets respectively.

Phenotypic data for this mapping population were measured by external and internal screens with plants scored as resistant (R), susceptible (S), and heterogeneous (H). These scores were converted to a numerical scale for mapping analysis, with R=9, H=5, and S=1.

Markers polymorphic between the two parents (95M60 and PHI_Res) in the regions of interest (top of LG-B2, middle of LG-D1b) were identified using proprietary software. A total of 22 polymorphic markers were identified to assay the entire population. One marker was removed due to monomorphism. The allele source was assigned codes maternal=A, paternal=B, and heterozygous=H.

Map Manager QTX.b20 (Manly et al. (2001) Mammalian Genome 12:930-932) was used to construct the linkage map and perform the QTL analysis. A 1000 permutation test was used to establish the threshold for statistical significance (LOD ratio statistic—LRS) to declare putative QTL. The mean score was used for QTL mapping. Initial Map Manager Parameters were set to:

1) Linkage Evaluation: Intercross

2) Search Criteria: P=1e-5

3) Map Function: Kosambi

4) Cross Type: Line Cross

Progeny stem canker score distribution showing the number of individuals in each phenotypic class for each screen is summarized in Table 7 below.

TABLE 7

| Data Set | Susceptible | Heterozygous | Resistant |
|---|---|---|---|
| External | 54 | 6 | 121 |
| Internal | 97 | 10 | 74 |
| Overall | 53 | 0 | 73 |

The 21 segregation markers were mapped to three linkage groups, with 9 markers on LG B2 and 12 markers (two sets of 6) on LG D1b. The 1000 permutation test set the significance thresholds for stem canker at 8.1 for a p-value of 0.01 (significant), and at 15.6 for a p-value of 0.05 (highly significant) for the external screen, 7.7 for a p-value of 0.01 (significant) and 16.1 for a p-value of 0.05 (highly significant) for the external screen, and 8.4 for a p-value of 0.01 (significant) and 15.7 for a p-value of 0.05 (highly significant) for the overall screen.

The marker regression analysis showed that the QTL associated with stem canker resistance could be located on LG-B2 for all three phenotypic data sets at the p=0.001 level. Marker regression data for LG B2 using the External phenotypic dataset is summarized in Table 8 below.

TABLE 8

| Locus | Genetic (cM) | Physical (bp) | LRS | % PhenoVar | p-value |
|---|---|---|---|---|---|
| S03188-1 | 10.17 | 1088724 | 44.1 | 23 | 0.00000 |
| S01718-1 | 15.00 | 2747160 | 42.3 | 21 | 0.00000 |
| S00802-1 | 17.87 | 3468738 | 22.4 | 12 | 0.00000 |
| S00777-1 | 19.06 | 3726937 | 26.4 | 14 | 0.00000 |
| S00288-1 | 21.26 | 4204414 | 25.5 | 13 | 0.00000 |
| S00341-1 | 25.61 | 5028737 | 18.3 | 10 | 0.00002 |

Marker regression data for LG B2 using the Internal phenotypic dataset is summarized in Table 9 below.

TABLE 9

| Locus | Genetic (cM) | Physical (bp) | LRS | % PhenoVar | p-value |
|---|---|---|---|---|---|
| S03188-1 | 10.17 | 1088724 | 19.4 | 11 | 0.00001 |
| S01718-1 | 15.00 | 2747160 | 21.8 | 12 | 0.00000 |
| S00802-1 | 17.87 | 3468738 | 11.4 | 6 | 0.00074 |
| S00777-1 | 19.06 | 3726937 | 14.3 | 8 | 0.00015 |
| S00288-1 | 21.26 | 4204414 | 13.7 | 7 | 0.00022 |

Marker regression data for LG B2 using the Overall phenotypic dataset is summarized in Table 10 below.

TABLE 10

| Locus | Genetic (cM) | Physical (bp) | LRS | % PhenoVar | p-value |
|---|---|---|---|---|---|
| S03188-1 | 10.17 | 1088724 | 39.4 | 28 | 0.00000 |
| S01718-1 | 14.89 | 2747160 | 41.4 | 29 | 0.00000 |
| S00802-1 | 17.87 | 3468738 | 21.6 | 16 | 0.00000 |
| S00777-1 | 19.06 | 3726937 | 26.1 | 19 | 0.00000 |
| S00288-1 | 21.26 | 4204414 | 25.1 | 18 | 0.00000 |
| S00341-1 | 25.61 | 5028737 | 18.6 | 14 | 0.00002 |

This region was investigated further by interval mapping method in Map Manager QTX.b20 using Kosambi map function with no control for other QTLs. Using the External screen dataset, a highly significant (LRS=45.6) QTL spanned an interval comprising marker S03188-1-A through marker S01718-1-A and was closely linked with marker S03188-1-A which explained about 24% of the phenotypic variation observed. Using the Internal screen dataset, a highly significant (LRS=21.7) QTL spanned an interval comprising marker S03188-1-A through marker S01718-1-A and was closely linked with marker S03188-1-A which explained about 12% of the phenotypic variation observed. Using the Overall screen dataset, a highly significant (LRS=43.3) QTL spanned an interval comprising marker S03188-1-A through marker S01718-1-A and was closely linked with marker S03188-1-A which explained about 31% of the phenotypic variation observed.

This population was further evaluated with additional markers on LG B2. Genotype data was collected using new markers near the identified region on the top of LG B2. Six markers were monomorphic in this population, and were removed for subsequent analysis, the remaining 17 markers were used for analysis. Map Manager QTX.b20 was used to construct the linkage map and perform the QTL analysis as described above. A1000 permutation test was used to establish the threshold for statistical significance (LOD ratio statistic—LRS) to declare putative QTL.

The 17 markers all mapped as one group to LG B2. The permutation test established the following critical LRS statistic values for the three phenotypic datasets: External screen: LRS 6.5 for a p-value of 0.01 (significant), and at 17.3 for a p-value of 0.05 (highly significant)

Internal screen: LRS 6.6 for a p-value of 0.01 (significant) and 17.3 for a p-value of 0.05 (highly significant)

Overall: 6.8 for a p-value of 0.01 (significant) and 13.6 for a p-value of 0.05 (highly significant)

The marker regression analysis showed that the QTL associated with stem canker resistance could be located on LG-B2 for all three phenotypic data sets. Marker regression data for LG B2 using the External phenotypic dataset is summarized in Table 11 below.

TABLE 11

| Locus | Genetic (cM) | Physical (bp) | LRS | % PhenoVar | p-value |
|---|---|---|---|---|---|
| S08257-1 | 10.74 | 1650065 | 46.6 | 25 | 0.00000 |
| S03188-1 | 10.17 | 1088724 | 43.8 | 23 | 0.00000 |
| S08256-1 | 10.68 | 1594889 | 41.3 | 21 | 0.00000 |
| S08231-3 | 10.83 | 1727084 | 38.1 | 19 | 0.00000 |
| S08231-4 | 10.83 | 1727625 | 33.9 | 17 | 0.00000 |
| S13722-1 | 11.18 | 1925222 | 39.4 | 20 | 0.00000 |
| S13721-2 | 10.89 | 1788456 | 36.2 | 18 | 0.00000 |
| S08255-3 | 10.86 | 1755250 | 36.9 | 18 | 0.00000 |
| S08255-4 | 10.86 | 1755572 | 41.6 | 21 | 0.00000 |
| S01718-1 | 15.00 | 2747160 | 42.5 | 21 | 0.00000 |
| S00802-1 | 17.87 | 3468738 | 22.6 | 12 | 0.00000 |
| S00777-1 | 19.06 | 3726937 | 26.3 | 14 | 0.00000 |
| S00288-1 | 21.26 | 4204414 | 25.2 | 13 | 0.00000 |
| S00341-1 | 25.61 | 5028737 | 18.1 | 10 | 0.00002 |

Marker regression data for LG B2 using the Internal phenotypic dataset is summarized in Table 12 below.

TABLE 12

| Locus | Genetic (cM) | Physical (bp) | LRS | % PhenoVar | p-value |
|---|---|---|---|---|---|
| S08257-1 | 10.74 | 1650065 | 20.8 | 12 | 0.00001 |
| S03188-1 | 10.17 | 1088724 | 19.7 | 11 | 0.00001 |
| S08256-1 | 10.68 | 1594889 | 19.1 | 10 | 0.00001 |
| S08231-3 | 10.83 | 1727084 | 15.8 | 8 | 0.00007 |
| S13722-1 | 11.18 | 1925222 | 16.4 | 9 | 0.00005 |
| S08255-3 | 10.86 | 1755250 | 18.3 | 10 | 0.00002 |
| S08255-4 | 10.86 | 1755572 | 20.0 | 10 | 0.00001 |

Marker regression data for LG B2 using the Overall phenotypic dataset is summarized Table 13 below.

TABLE 13

| Locus | Genetic (cM) | Physical (bp) | LRS | % PhenoVar | p-value |
|---|---|---|---|---|---|
| S08257-1 | 10.74 | 1650065 | 42.3 | 31 | 0.00000 |
| S03188-1 | 10.17 | 1088724 | 39.4 | 28 | 0.00000 |
| S08256-1 | 10.68 | 1594889 | 37.8 | 26 | 0.00000 |
| S08231-3 | 10.83 | 1727084 | 33.7 | 24 | 0.00000 |
| S08231-4 | 10.83 | 1727625 | 28.6 | 20 | 0.00000 |
| S13722-1 | 11.18 | 1925222 | 34.4 | 24 | 0.00000 |
| S13721-2 | 10.89 | 1788456 | 30.9 | 22 | 0.00000 |
| S08255-3 | 10.86 | 1755250 | 34.4 | 24 | 0.00000 |
| S08255-4 | 10.86 | 1755572 | 38.6 | 26 | 0.00000 |
| S01718-1 | 15.00 | 2747160 | 42.2 | 28 | 0.00000 |
| S00802-1 | 17.87 | 3468738 | 22.1 | 16 | 0.00000 |
| S00777-1 | 19.06 | 3726937 | 26.3 | 19 | 0.00000 |
| S00288-1 | 21.26 | 4204414 | 25.1 | 18 | 0.00000 |

This region was investigated further by interval mapping method in Map Manager QTX.b20 using Kosambi map function with no control for other QTLs. Using the External screen dataset, a highly significant (LRS=46.6) QTL spanned an interval comprising marker S08257-1-A through marker S03188-1-A and was closely linked with marker S08257-1-A which explained about 25% of the phenotypic variation observed. Using the Internal screen dataset, a highly significant (LRS=21.8) QTL spanned an interval comprising marker S08257-1-A through marker S03188-1-A and was closely linked with marker S08257-1-A which explained about 12% of the phenotypic variation observed. Using the Overall screen dataset, a highly significant (LRS=42.3) QTL spanned an interval comprising marker S08257-1-A through marker S03188-1-A and was closely linked with marker S08257-1-A which explained about 31% of the phenotypic variation observed.

### Example 7

### Marker Development

Markers were developed to characterize, identify, and/or select resistant or susceptible alleles for stem canker on linkage group B2 (ch 14). Markers were screened and validated against various known resistant and susceptible parents.

Markers to loci S08241-1 (10.85 cM, Gm14:1747540), S08251-2 (10.85 cM, Gm14:1748141), and S08251-4 (10.85 cM, Gm14:1748042) were developed to identify alleles associated with the stem canker resistance phenotype. Consensus sequence from a panel of lines was used for development for markers to identify potential marker loci and to provide information on alleles in the targeted genomic region on LG B2 (ch14). During development, markers were validated and confirmed against a panel of about 30 resistant and susceptible varieties which included proprietary experimental lines, proprietary commercial lines, and public lines. Potential markers were tested against genomic DNA extracted via different methods, such as citrate or urea extraction, as well from different tissues, such as leaf or seed tissue. Further development and testing was done to optimize the marker system for high throughput analysis of soybean using a standard amplification procedure provided in Example 8. From this testing, S08241-1-Q6, S08251-2-Q10, and S08251-4-Q7 were chosen for high throughput analysis needs, but other versions can be used to detect the polymorphism.

### Example 8

### Marker Assay

Any source tissue, nucleic acid isolation, and analysis method or combination thereof may be used to isolate, detect and/or characterize polynucleotides associated with stem canker tolerance. One or more of primers and/or probes may comprise a heterologous detectable label. Exemplary options are provided below.

Samples for DNA preparation are taken by leaf punch and DNA isolated by citrate extraction. Sample replicates of each variety may be used in the analysis. Samples are set up in a 96 well plate, which is replicated 4 times into a 384 plate and dried down.

For the TAQMAN® assay, each reaction mix is as follows:

| | |
|---|---|
| Water | 3.625 μl |
| Hottub Buffer | 0.5 μl |
| dNTP (2.5 mM each) | 0.15 μl |
| Primer1 + Primer2 (10 μM each) | 0.0375 μl |
| Primer3 + Primer3 (10 μM each) | 0.0375 μl |
| Probe 1 (10 μM) | 0.05 μl |
| Probe 2 (10 μM) | 0.05 μl |
| Hottub Enzyme | 0.025 μl |
| Invitrogen ROX ™ dye (50X) | 0.075 μl |
| DNA | 0.05 μl |

DNA is amplified by PCR in a hydrocycler using the following conditions:

| | |
|---|---|
| 94° C. | 10 min 1 cycle, followed by 40 cycles of: |
| 94° C. | 30 sec |
| 60° C. | 60 sec |

An alternative reaction mix which can be amplified using the temperature and cycle conditions provided above is as follows:

DNA (dried down)

| | |
|---|---|
| Water | 2.42 μl |
| KlearKall Mastermix | 2.5 μl |
| Forward Primer (100 μm) | 0.0375 μl |
| Reverse Primer (100 μm) | 0.0375 μl |
| Probe 1 (100 μm) | 0.005 μl |
| Probe 2 (100 μm) | 0.005 μl |
| Total | 5 μl |

The markers provided and exemplified herein could be useful, for example, for detecting and/or selecting soybean plants with improved tolerance to stem canker. The physical position of each locus and each SNP has been provided. Any marker capable of detecting a polymorphism at one of these physical positions, or a marker closely linked thereto, could also be useful, for example, for detecting and/or selecting soybean plants with improved stem canker tolerance. In some examples, the SNP allele present in the tolerant parental line could be used as a favorable allele to detect or select plants with improved tolerance. In other examples, the SNP allele present in the susceptible parent line could be used as an unfavorable allele to detect or select plants without improved tolerance.

What is claimed is:

1. A method of identifying a first soybean plant or germplasm having improved tolerance to stem canker, said method comprising detecting in the first soybean plant or germplasm at least one allele of a quantitative trait locus that is associated with stem canker resistance, wherein said quantitative trait locus is localized to a chromosomal interval flanked by and including markers BARC-013365-00489 and BARCSOYSSR_14_0281 on linkage group B2 (ch 14).

2. The method of claim 1, wherein the chromosomal interval is flanked by and includes markers BARC-013365-00489 and S04785-1 on linkage group B2 (ch 14).

3. The method of claim 1, wherein the chromosomal interval is flanked by and includes markers BARCSOYSSR_14_0036 and BARCSOYSSR_14_0281 on linkage group B2 (ch 14).

4. The method of claim 1, wherein the chromosomal interval is flanked by and includes markers S03188-1 and S04785-1 on linkage group B2 (ch 14).

5. The method of claim 1, wherein the chromosomal interval is flanked by and includes markers S03188-1 and S02987-1 on linkage group B2 (ch 14).

6. The method of claim 1, wherein the chromosomal interval is flanked by and includes markers Gm14:584976 and Gm14:2359579 on linkage group B2 (ch 14).

7. The method of claim 1, wherein the chromosomal interval is flanked by and includes markers Gm14:1697331 and Gm14:2019929 on linkage group B2 (ch 14).

8. The method of claim 1, wherein the chromosomal interval is flanked by and includes markers Gm14:1706695 and Gm14:1942681 on linkage group B2 (ch 14).

9. The method of claim 1, wherein detecting comprises detecting an allele of one or more marker locus selected from the group consisting of:
(a) a marker that detects a polymorphism at S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14);
(b) a marker locus comprising S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14);
(c) S03188-1-A, S04492-1-A, S08256-1-01, S08257-1-01, S08231-3-01, S08231-4-01, S08241-1-06, S08251-4-07, S08251-2-010, S08255-3-03, S08255-4-Q1, S13721-2-01, S01591-1-A, S13722-1-01, S02987-1-A, S00802-1-A, S00777-1-A, S01799-1-A, S00288-1-A, S03923-1-A, S00341-1-A, S01718-1-A, and S04785-1-A on LG B2 (ch 14);
(d) a marker locus comprising Gm14:1088724, Gm14:1197243, Gm14:1594889, Gm14:1650065, Gm14:1727084, Gm14:1727625, Gm14:1747540, Gm14:1748042, Gm14:1748141, Gm14:1755250, Gm14:1755572, Gm14:1788456, Gm14:1923247, Gm14:1925222, Gm14:2129691, Gm14:3468738, Gm14:3726937, Gm14:3727753, Gm14:4204414, Gm14:4946161, Gm14:5028737, Gm14:5096522, Gm14:1706695, Gm14:1723656, Gm14:1723831, Gm14:1724319, Gm14:1738483, Gm14:1739929, Gm14:1850843, Gm14:1853044, Gm14:1863371, Gm14:1863436, Gm14:1863868, Gm14:1914305, Gm14:1936105, Gm14:1939210, Gm14:1940046, Gm14:1942461, Gm14:1942681, Gm14:1700735, Gm14:1702258, Gm14:1715352, Gm14:1718565, Gm14:1722078, Gm14:1722192, Gm14:1723045, Gm14:1724087, Gm14:1724245, Gm14:1724558, Gm14:1724603, Gm14:1725318, Gm14:1726899, Gm14:1738054, Gm14:1784545, Gm14:1785276, Gm14:1793750, Gm14:1842226, Gm14:1850134, Gm14:1850654, Gm14:1850808, Gm14:1851180, Gm14:1851369, Gm14:1851467, Gm14:1851793, Gm14:1852388, Gm14:1852579, Gm14:1855401, Gm14:1867651, Gm14:1867913, Gm14:1867927, Gm14:1910547, Gm14:1910746, Gm14:1910787, Gm14:1911679, Gm14:1916729, Gm14:1916742, Gm14:1919121, Gm14:1934770, Gm14:1935561, Gm14:1935695, Gm14:1935949, Gm14:1936112, Gm14:1936619, Gm14:1936750, Gm14:1936804, Gm14:1936827, Gm14:1937070, Gm14:1937136, Gm14:1937495, Gm14:1938061, Gm14:1940079, Gm14:1940534, Gm14:1940728, Gm14:1940825, Gm14:1941047, Gm14:1941105, Gm14:1944642, Gm14:1700746, Gm14:1706616, Gm14:1706705, Gm14:1706782, Gm14:1708836, Gm14:1709816, Gm14:1712174, Gm14:1717270, Gm14:1722544, Gm14:1723756, Gm14:1725153, Gm14:1725269, Gm14:1726477, Gm14:1731690, Gm14:1738538, Gm14:1739491, Gm14:1739824, Gm14:1785457, Gm14:1787260, Gm14:1796497, Gm14:1840314, Gm14:1842837, Gm14:1850412, Gm14:1850433, Gm14:1850907, Gm14:1851535, Gm14:1852210, Gm14:1859255, Gm14:1860411, Gm14:1909564, Gm14:1910968, Gm14:1935936, Gm14:1936214, Gm14:1936876, Gm14:1939030, Gm14:1939031, Gm14:1939081, Gm14:1940392, Gm14:1941517, Gm14:1989692, Gm14:1989717, Gm14:1992951, Gm14:2003498, Gm14:2004242, Gm14:2004409, Gm14:2006356, Gm14:1698254, Gm14:1698262, Gm14:1698269, Gm14:1701170, Gm14:1701174, Gm14:1701472, Gm14:1701825, Gm14:1702477, Gm14:1702552, Gm14:1702622, Gm14:1704204, Gm14:1705803, Gm14:1706201, Gm14:1706361, Gm14:1706585, Gm14:1706915, Gm14:1708746, Gm14:1713290, Gm14:1718056, Gm14:1718944, Gm14:1719373, Gm14:1723066, Gm14:1726434, Gm14:1726867, Gm14:1727235, Gm14:1728343, Gm14:1730044, Gm14:1731518, Gm14:1736053, Gm14:1738509, Gm14:1738914, Gm14:1739468, Gm14:1739718, Gm14:1739772, Gm14:1749899, Gm14:1785516, Gm14:1785559, Gm14:1786034, Gm14:1786259, Gm14:1793453, Gm14:1796469, Gm14:1797188, Gm14:1798987, Gm14:1800055, Gm14:1842374, Gm14:1850066, Gm14:1850741, Gm14:1850928, Gm14:1852469, Gm14:1854015, Gm14:1854023, Gm14:1859810, Gm14:1860271, Gm14:1862541, Gm14:1867332, Gm14:1868414, Gm14:1919937, Gm14:1935444, Gm14:1936433, Gm14:1937170, Gm14:1937923, Gm14:1938818, Gm14:1939343, Gm14:1939364, Gm14:1939647, Gm14:1939662, Gm14:1939746, Gm14:1939749, Gm14:1940207, Gm14:1940573, Gm14:1941546, Gm14:1945962, Gm14:1946355, Gm14:1990996, Gm14:1991421, Gm14:2001506, Gm14:2001882, Gm14:2003462, Gm14:2003672, Gm14:1697331, Gm14:1698694, Gm14:1700508, Gm14:1701739, Gm14:1701858, Gm14:1702110, Gm14:1702378, Gm14:1706884, Gm14:1706973, Gm14:1708902, Gm14:1711952, Gm14:1712050, Gm14:1712345, Gm14:1717261, Gm14:1722228, Gm14:1722277, Gm14:1723101, Gm14:1724793, Gm14:1725740, Gm14:1726334, Gm14:1727170, Gm14:1730199, Gm14:1736329, Gm14:1736463, Gm14:1736832, Gm14:1737771, Gm14:1738824, Gm14:1738960, Gm14:1743818, Gm14:1756757, Gm14:1760506, Gm14:1778710, Gm14:1783716, Gm14:1784221, Gm14:1786098, Gm14:1797150, Gm14:1797155, Gm14:1799977, Gm14:1811618, Gm14:1812051, Gm14:1824464, Gm14:1839360, Gm14:1843408, Gm14:1850104, Gm14:1851115, Gm14:1851116, Gm14:1855960, Gm14:1858300, Gm14:1860243, Gm14:1860675, Gm14:1866981, Gm14:1869210, Gm14:1872625, Gm14:1902929, Gm14:1913544, Gm14:1914780, Gm14:1935408, Gm14:1936381, Gm14:1936425, Gm14:1939383, Gm14:1988977, Gm14:1989052, Gm14:1990632, Gm14:1990775, Gm14:1991550, Gm14:1999220, Gm14:2003981, Gm14:1697620, Gm14:1697978, Gm14:1699410, Gm14:1700636, Gm14:1701683, Gm14:1701935, Gm14:1702661, Gm14:1702755, Gm14:1704651, Gm14:1704664, Gm14:1712038, Gm14:1712320, Gm14:1712713, Gm14:1712865, Gm14:1712866, Gm14:1713999, Gm14:1719022, Gm14:1728216, Gm14:1731767, Gm14:1735093, Gm14:1735102, Gm14:1736945, Gm14:1743592, Gm14:1748064, Gm14:1749126, Gm14:1754686, Gm14:1757996, Gm14:1760483, Gm14:1783231, Gm14:1783538, Gm14:1783756, Gm14:1786013, Gm14:1787773, Gm14:1792218, Gm14:1799327, Gm14:1799390, Gm14:1799540, Gm14:1800012, Gm14:1800319, Gm14:1811113, Gm14:1811977, Gm14:1824284, Gm14:1837872, Gm14:1837888, Gm14:1851947, Gm14:1858277, Gm14:1861083, Gm14:1887021, Gm14:1903293, Gm14:1910033, Gm14:1919915, Gm14:1934737, Gm14:1946125, Gm14:1958086, Gm14:1986602, Gm14:1987892, Gm14:1988001, Gm14:1989951, Gm14:1991474, Gm14:1997517, Gm14:2006319, Gm14:2017162, Gm14:2019929, Gm14:1701692, Gm14:1702315, Gm14:1702956, Gm14:1703980, Gm14:1706523, Gm14:1711087, Gm14:1712377, Gm14:1714965, Gm14:1727031, Gm14:1727851, Gm14:1728741, Gm14:1729293, Gm14:1730480, Gm14:1730798, Gm14:1731840, Gm14:1732232, Gm14:1732358, Gm14:1732363, Gm14:1732403, Gm14:1735755, Gm14:1735945, Gm14:1743425, Gm14:1744356, Gm14:1746573, Gm14:1748959, Gm14:1750564, Gm14:1753875, Gm14:1754007, Gm14:1755671, Gm14:1756046, Gm14:1760299, Gm14:1760329, Gm14:1761362, Gm14:1766162, Gm14:1780165, Gm14:1780408, Gm14:1782983, Gm14:1789475, Gm14:1799494, Gm14:1799548, Gm14:1799571, Gm14:1811318, Gm14:1825691, Gm14:1834023, Gm14:1841281, Gm14:1846850, Gm14:1887108, Gm14:1887251, Gm14:1935369, Gm14:1969791, Gm14:1981574, Gm14:1981644, Gm14:1983907, Gm14:1986614, Gm14:1986675, Gm14:1987587, Gm14:1988422, Gm14:1988424, Gm14:1988520, Gm14:1988558, Gm14:1989403, Gm14:1991457, Gm14:1991910, Gm14:1992830, Gm14:1993931, Gm14:1997124, Gm14:1998879, Gm14:2004939, Gm14:2005896, Gm14:1698816, Gm14:1701991, Gm14:1710294, Gm14:1710303, Gm14:1711007, Gm14:1711575, Gm14:1713816, Gm14:1728293, Gm14:1729743, Gm14:1730281, Gm14:1730795, Gm14:1732205, Gm14:1744432, Gm14:1751350, Gm14:1755398, Gm14:1756188, Gm14:1761081, Gm14:1761387, Gm14:1762867, Gm14:1765302, Gm14:1777323, Gm14:1779323, Gm14:1780397, Gm14:1800359, Gm14:1803234, Gm14:1811158, Gm14:1825653, Gm14:1869500, Gm14:1876026, Gm14:1876675, Gm14:1876844, Gm14:1876847, Gm14:1879162, Gm14:1884603, Gm14:1904675, Gm14:1944708, Gm14:1954056, Gm14:1954062, Gm14:1967108, Gm14:1972107, Gm14:2006215, Gm14:2011465, Gm14:1704689, Gm14:1711569, Gm14:1713323, Gm14:1728052, Gm14:1729537, Gm14:1732261, Gm14:1732593, Gm14:1735227, Gm14:1744452, Gm14:1754156, Gm14:1755877, Gm14:1756355, Gm14:1759702, Gm14:1780316, Gm14:1801585, Gm14:1810099, Gm14:1810126, Gm14:1810421, Gm14:1823776, Gm14:1875278, Gm14:1875874, Gm14:1902961, Gm14:1910054, Gm14:1949216, Gm14:1957357, Gm14:1957358, Gm14:1960447, Gm14:1980728, Gm14:1984129, Gm14:1992331, Gm14:1993147, Gm14:1995593, Gm14:1995600, Gm14:1996952, Gm14:1997412, Gm14:2004591, Gm14:1645762, Gm14:1713775, Gm14:1716505, Gm14:1729186, Gm14:1729798, Gm14:1732529, Gm14:1753922, Gm14:1759619, Gm14:1759635, Gm14:1760144, Gm14:1761168, Gm14:1771612, Gm14:1777105, Gm14:1778517, Gm14:1779288, Gm14:1779568, Gm14:1804682, Gm14:1810135, Gm14:1825225, Gm14:1828925, Gm14:1846994, Gm14:1848374, Gm14:1874941, Gm14:1875261, Gm14:1875470, Gm14:1879189, Gm14:1881168, Gm14:1923548, Gm14:1923935, Gm14:1955048, Gm14:1955118, Gm14:1955144, Gm14:1959610, Gm14:1960454, Gm14:1967167, Gm14:1970908, Gm14:1976442, Gm14:1981936, Gm14:1982632, Gm14:1982756, Gm14:1984276, Gm14:1994907, Gm14:2013156, Gm14:2019411, Gm14:1711379, Gm14:1713140, Gm14:1727997, Gm14:1744286, Gm14:1757864, Gm14:1759363, Gm14:1765933, Gm14:1767525, Gm14:1767582, Gm14:1768845, Gm14:1775564, Gm14:1778106, Gm14:1778621, Gm14:1780281, Gm14:1780494, Gm14:1805372, Gm14:1809090, Gm14:1810313, Gm14:1812109, Gm14:1812114, Gm14:1824651, Gm14:1875656, Gm14:1877489, Gm14:1878068, Gm14:1903061, Gm14:1950278, Gm14:1950577, Gm14:1950969, Gm14:1970110, Gm14:1982411, Gm14:1983489, Gm14:1983507, Gm14:1983593, Gm14:1993730, Gm14:1993788, Gm14:1993806, Gm14:2006229, Gm14:2015853, Gm14:2359579, Gm14:1579650, Gm14:1764334, Gm14:1764392, Gm14:1764403, Gm14:1767878, Gm14:1805366, Gm14:1825173, Gm14:1879313, Gm14:1886238, Gm14:1899408, Gm14:1960137, Gm14:1971009, Gm14:1983348, Gm14:2011607, Gm14:2012105, Gm14:2340746, Gm14:1767671, Gm14:1771502, Gm14:1771578, Gm14:1803995, Gm14:1805322, Gm14:1809988, Gm14:1825310, Gm14:1888310, Gm14:1889307, Gm14:1957202, Gm14:1981029, Gm14:1981073, Gm14:1981255, Gm14:1992432, Gm14:2010834, Gm14:1759440, Gm14:1775298, Gm14:1775605, Gm14:1804116, Gm14:1804868, Gm14:1806330, Gm14:1806377, Gm14:1889158, Gm14:1899428, Gm14:1926218, Gm14:1928522, Gm14:1957206, Gm14:1959033, Gm14:1969518, Gm14:1975722, Gm14:1981293, Gm14:1983189, Gm14:2013786, Gm14:2014487, Gm14:2191538, Gm14:2191541, Gm14:2191563, Gm14:1711534, Gm14:1773993, Gm14:1808347, Gm14:1808348, Gm14:1812356, Gm14:1812358, Gm14:1874583, Gm14:1971096, Gm14:1975723, Gm14:1976101, Gm14:1998648, Gm14:2006929, Gm14:2013850, Gm14:2014479, Gm14:2014481, Gm14:2101794, Gm14:766045, Gm14:1801323, Gm14:1805026, Gm14:1879302, Gm14:1975774, Gm14:2014429, Gm14:2191662, Gm14:584976, Gm14:2007005, Gm14:1650177, Gm14:1899046, Gm14:806705, Gm14:1976057, Gm14:2747160, or Gm14:1889837;
(e) a marker that detects a polymorphism at Gm14:1088724, Gm14:1197243, Gm14:1594889, Gm14:1650065, Gm14:1727084, Gm14:1727625, Gm14:1747540, Gm14:1748042, Gm14:1748141, Gm14:1755250, Gm14:1755572, Gm14:1788456, Gm14:1923247, Gm14:1925222, Gm14:2129691, Gm14:3468738, Gm14:3726937, Gm14:3727753, Gm14:4204414, Gm14:4946161, Gm14:5028737, Gm14:5096522, Gm14:1706695, Gm14:1723656, Gm14:1723831, Gm14:1724319, Gm14:1738483, Gm14:1739929, Gm14:1850843, Gm14:1853044, Gm14:1863371, Gm14:1863436, Gm14:1863868, Gm14:1914305, Gm14:1936105, Gm14:1939210, Gm14:1940046, Gm14:1942461, Gm14:1942681, Gm14:1700735, Gm14:1702258, Gm14:1715352, Gm14:1718565, Gm14:1722078, Gm14:1722192, Gm14:1723045, Gm14:1724087, Gm14:1724245, Gm14:1724558, Gm14:1724603, Gm14:1725318, Gm14:1726899, Gm14:1738054, Gm14:1784545, Gm14:1785276, Gm14:1793750, Gm14:1842226, Gm14:1850134, Gm14:1850654, Gm14:1850808, Gm14:1851180, Gm14:1851369, Gm14:1851467, Gm14:1851793, Gm14:1852388, Gm14:1852579, Gm14:1855401, Gm14:1867651, Gm14:1867913, Gm14:1867927, Gm14:1910547, Gm14:1910746, Gm14:1910787, Gm14:1911679, Gm14:1916729, Gm14:1916742, Gm14:1919121, Gm14:1934770, Gm14:1935561, Gm14:1935695, Gm14:1935949, Gm14:1936112, Gm14:1936619, Gm14:1936750, Gm14:1936804, Gm14:1936827, Gm14:1937070, Gm14:1937136, Gm14:1937495, Gm14:1938061, Gm14:1940079, Gm14:1940534, Gm14:1940728, Gm14:1940825, Gm14:1941047, Gm14:1941105, Gm14:1944642, Gm14:1700746, Gm14:1706616, Gm14:1706705, Gm14:1706782, Gm14:1708836, Gm14:1709816, Gm14:1712174, Gm14:1717270, Gm14:1722544, Gm14:1723756, Gm14:1725153, Gm14:1725269, Gm14:1726477, Gm14:1731690, Gm14:1738538, Gm14:1739491, Gm14:1739824, Gm14:1785457, Gm14:1787260, Gm14:1796497, Gm14:1840314, Gm14:1842837, Gm14:1850412, Gm14:1850433, Gm14:1850907, Gm14:1851535, Gm14:1852210, Gm14:1859255, Gm14:1860411, Gm14:1909564, Gm14:1910968, Gm14:1935936, Gm14:1936214, Gm14:1936876, Gm14:1939030, Gm14:1939031, Gm14:1939081, Gm14:1940392, Gm14:1941517, Gm14:1989692, Gm14:1989717, Gm14:1992951, Gm14:2003498, Gm14:2004242, Gm14:2004409, Gm14:2006356, Gm14:1698254, Gm14:1698262, Gm14:1698269, Gm14:1701170, Gm14:1701174, Gm14:1701472, Gm14:1701825, Gm14:1702477, Gm14:1702552, Gm14:1702622, Gm14:1704204, Gm14:1705803, Gm14:1706201, Gm14:1706361, Gm14:1706585, Gm14:1706915, Gm14:1708746, Gm14:1713290, Gm14:1718056, Gm14:1718944, Gm14:1719373, Gm14:1723066, Gm14:1726434, Gm14:1726867, Gm14:1727235, Gm14:1728343, Gm14:1730044, Gm14:1731518, Gm14:1736053, Gm14:1738509, Gm14:1738914, Gm14:1739468, Gm14:1739718, Gm14:1739772, Gm14:1749899, Gm14:1785516, Gm14:1785559, Gm14:1786034, Gm14:1786259, Gm14:1793453, Gm14:1796469, Gm14:1797188, Gm14:1798987, Gm14:1800055, Gm14:1842374, Gm14:1850066, Gm14:1850741, Gm14:1850928, Gm14:1852469, Gm14:1854015, Gm14:1854023, Gm14:1859810, Gm14:1860271, Gm14:1862541, Gm14:1867332, Gm14:1868414, Gm14:1919937, Gm14:1935444, Gm14:1936433, Gm14:1937170, Gm14:1937923, Gm14:1938818, Gm14:1939343, Gm14:1939364, Gm14:1939647, Gm14:1939662, Gm14:1939746, Gm14:1939749, Gm14:1940207, Gm14:1940573, Gm14:1941546, Gm14:1945962, Gm14:1946355, Gm14:1990996, Gm14:1991421, Gm14:2001506, Gm14:2001882, Gm14:2003462, Gm14:2003672, Gm14:1697331, Gm14:1698694, Gm14:1700508, Gm14:1701739, Gm14:1701858, Gm14:1702110, Gm14:1702378, Gm14:1706884, Gm14:1706973, Gm14:1708902, Gm14:1711952, Gm14:1712050, Gm14:1712345, Gm14:1717261, Gm14:1722228, Gm14:1722277, Gm14:1723101, Gm14:1724793, Gm14:1725740, Gm14:1726334, Gm14:1727170, Gm14:1730199, Gm14:1736329, Gm14:1736463, Gm14:1736832, Gm14:1737771, Gm14:1738824, Gm14:1738960, Gm14:1743818, Gm14:1756757, Gm14:1760506, Gm14:1778710, Gm14:1783716, Gm14:1784221, Gm14:1786098, Gm14:1797150, Gm14:1797155, Gm14:1799977, Gm14:1811618, Gm14:1812051, Gm14:1824464, Gm14:1839360, Gm14:1843408, Gm14:1850104, Gm14:1851115, Gm14:1851116, Gm14:1855960, Gm14:1858300, Gm14:1860243, Gm14:1860675, Gm14:1866981, Gm14:1869210, Gm14:1872625, Gm14:1902929, Gm14:1913544, Gm14:1914780, Gm14:1935408, Gm14:1936381, Gm14:1936425, Gm14:1939383, Gm14:1988977, Gm14:1989052, Gm14:1990632, Gm14:1990775, Gm14:1991550, Gm14:1999220, Gm14:2003981, Gm14:1697620, Gm14:1697978, Gm14:1699410, Gm14:1700636, Gm14:1701683, Gm14:1701935, Gm14:1702661, Gm14:1702755, Gm14:1704651, Gm14:1704664, Gm14:1712038, Gm14:1712320, Gm14:1712713, Gm14:1712865, Gm14:1712866, Gm14:1713999, Gm14:1719022, Gm14:1728216, Gm14:1731767, Gm14:1735093, Gm14:1735102, Gm14:1736945, Gm14:1743592, Gm14:1748064, Gm14:1749126, Gm14:1754686, Gm14:1757996, Gm14:1760483, Gm14:1783231, Gm14:1783538, Gm14:1783756, Gm14:1786013, Gm14:1787773, Gm14:1792218, Gm14:1799327, Gm14:1799390, Gm14:1799540, Gm14:1800012, Gm14:1800319, Gm14:1811113, Gm14:1811977, Gm14:1824284, Gm14:1837872, Gm14:1837888, Gm14:1851947, Gm14:1858277, Gm14:1861083, Gm14:1887021, Gm14:1903293, Gm14:1910033, Gm14:1919915, Gm14:1934737, Gm14:1946125, Gm14:1958086, Gm14:1986602, Gm14:1987892, Gm14:1988001, Gm14:1989951, Gm14:1991474, Gm14:1997517, Gm14:2006319, Gm14:2017162, Gm14:2019929, Gm14:1701692, Gm14:1702315, Gm14:1702956, Gm14:1703980, Gm14:1706523, Gm14:1711087, Gm14:1712377, Gm14:1714965, Gm14:1727031, Gm14:1727851, Gm14:1728741, Gm14:1729293, Gm14:1730480, Gm14:1730798, Gm14:1731840, Gm14:1732232, Gm14:1732358, Gm14:1732363, Gm14:1732403, Gm14:1735755, Gm14:1735945, Gm14:1743425, Gm14:1744356, Gm14:1746573, Gm14:1748959, Gm14:1750564, Gm14:1753875, Gm14:1754007, Gm14:1755671, Gm14:1756046, Gm14:1760299, Gm14:1760329, Gm14:1761362, Gm14:1766162, Gm14:1780165, Gm14:1780408, Gm14:1782983, Gm14:1789475, Gm14:1799494, Gm14:1799548, Gm14:1799571, Gm14:1811318, Gm14:1825691, Gm14:1834023, Gm14:1841281, Gm14:1846850, Gm14:1887108, Gm14:1887251, Gm14:1935369, Gm14:1969791, Gm14:1981574, Gm14:1981644, Gm14:1983907, Gm14:1986614, Gm14:1986675, Gm14:1987587, Gm14:1988422, Gm14:1988424, Gm14:1988520, Gm14:1988558, Gm14:1989403, Gm14:1991457, Gm14:1991910, Gm14:1992830, Gm14:1993931, Gm14:1997124, Gm14:1998879, Gm14:2004939, Gm14:2005896, Gm14:1698816, Gm14:1701991, Gm14:1710294, Gm14:1710303, Gm14:1711007, Gm14:1711575, Gm14:1713816, Gm14:1728293, Gm14:1729743, Gm14:1730281, Gm14:1730795, Gm14:1732205, Gm14:1744432, Gm14:1751350, Gm14:1755398, Gm14:1756188, Gm14:1761081, Gm14:1761387, Gm14:1762867, Gm14:1765302, Gm14:1777323, Gm14:1779323, Gm14:1780397, Gm14:1800359, Gm14:1803234, Gm14:1811158, Gm14:1825653, Gm14:1869500, Gm14:1876026, Gm14:1876675, Gm14:1876844, Gm14:1876847, Gm14:1879162, Gm14:1884603, Gm14:1904675, Gm14:1944708, Gm14:1954056, Gm14:1954062, Gm14:1967108, Gm14:1972107, Gm14:2006215, Gm14:2011465, Gm14:1704689, Gm14:1711569, Gm14:1713323, Gm14:1728052, Gm14:1729537, Gm14:1732261, Gm14:1732593, Gm14:1735227, Gm14:1744452, Gm14:1754156, Gm14:1755877, Gm14:1756355, Gm14:1759702, Gm14:1780316, Gm14:1801585, Gm14:1810099, Gm14:1810126, Gm14:1810421, Gm14:1823776, Gm14:1875278, Gm14:1875874, Gm14:1902961, Gm14:1910054, Gm14:1949216, Gm14:1957357, Gm14:1957358, Gm14:1960447, Gm14:1980728, Gm14:1984129, Gm14:1992331, Gm14:1993147, Gm14:1995593, Gm14:1995600, Gm14:1996952, Gm14:1997412, Gm14:2004591, Gm14:1645762, Gm14:1713775, Gm14:1716505, Gm14:1729186, Gm14:1729798, Gm14:1732529, Gm14:1753922, Gm14:1759619, Gm14:1759635, Gm14:1760144, Gm14:1761168, Gm14:1771612, Gm14:1777105, Gm14:1778517, Gm14:1779288, Gm14:1779568, Gm14:1804682, Gm14:1810135, Gm14:1825225, Gm14:1828925, Gm14:1846994, Gm14:1848374, Gm14:1874941, Gm14:1875261, Gm14:1875470, Gm14:1879189, Gm14:1881168, Gm14:1923548, Gm14:1923935, Gm14:1955048, Gm14:1955118, Gm14:1955144, Gm14:1959610, Gm14:1960454, Gm14:1967167, Gm14:1970908, Gm14:1976442, Gm14:1981936, Gm14:1982632, Gm14:1982756, Gm14:1984276, Gm14:1994907, Gm14:2013156, Gm14:2019411, Gm14:1711379, Gm14:1713140, Gm14:1727997, Gm14:1744286, Gm14:1757864, Gm14:1759363, Gm14:1765933, Gm14:1767525, Gm14:1767582, Gm14:1768845, Gm14:1775564, Gm14:1778106, Gm14:1778621, Gm14:1780281, Gm14:1780494, Gm14:1805372, Gm14:1809090, Gm14:1810313, Gm14:1812109, Gm14:1812114, Gm14:1824651, Gm14:1875656, Gm14:1877489, Gm14:1878068, Gm14:1903061, Gm14:1950278, Gm14:1950577, Gm14:1950969, Gm14:1970110, Gm14:1982411, Gm14:1983489, Gm14:1983507, Gm14:1983593, Gm14:1993730, Gm14:1993788, Gm14:1993806, Gm14:2006229, Gm14:2015853, Gm14:2359579, Gm14:1579650, Gm14:1764334, Gm14:1764392, Gm14:1764403, Gm14:1767878, Gm14:1805366, Gm14:1825173, Gm14:1879313, Gm14:1886238, Gm14:1899408, Gm14:1960137, Gm14:1971009, Gm14:1983348, Gm14:2011607, Gm14:2012105, Gm14:2340746, Gm14:1767671, Gm14:1771502, Gm14:1771578, Gm14:1803995, Gm14:1805322, Gm14:1809988, Gm14:1825310, Gm14:1888310, Gm14:1889307, Gm14:1957202, Gm14:1981029, Gm14:1981073, Gm14:1981255, Gm14:1992432, Gm14:2010834, Gm14:1759440, Gm14:1775298, Gm14:1775605, Gm14:1804116, Gm14:1804868, Gm14:1806330, Gm14:1806377, Gm14:1889158, Gm14:1899428, Gm14:1926218, Gm14:1928522, Gm14:1957206, Gm14:1959033, Gm14:1969518, Gm14:1975722, Gm14:1981293, Gm14:1983189, Gm14:2013786, Gm14:2014487, Gm14:2191538, Gm14:2191541, Gm14:2191563, Gm14:1711534, Gm14:1773993, Gm14:1808347, Gm14:1808348, Gm14:1812356, Gm14:1812358, Gm14:1874583, Gm14:1971096, Gm14:1975723, Gm14:1976101, Gm14:1998648, Gm14:2006929, Gm14:2013850, Gm14:2014479, Gm14:2014481, Gm14:2101794, Gm14:766045, Gm14:1801323, Gm14:1805026, Gm14:1879302, Gm14:1975774, Gm14:2014429, Gm14:2191662, Gm14:584976, Gm14:2007005, Gm14:1650177, Gm14:1899046, Gm14:806705, Gm14:1976057, Gm14:2747160, or Gm14:1889837;
(f) a marker locus comprising Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, or Glyma14g02730.1;
(g) a marker that detects a polymorphism in Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, and Glyma14g02730.1;
(h) a marker that detects a polymorphism in one or more or SEQ ID NOs: 1-786;
(i) a marker that detects a favorable allele of (a), (b), (c), (d), (e), (f), or (g); and,
(j) a marker locus closely linked to a marker locus of (a), (b), (c), (d), (e), (f), (g), (h), or (i).

10. The method of claim 9, comprising detecting two or more marker loci of (a)-(k).

11. The method of claim 1, wherein the at least one allele is a favorable allele that positively correlates with improved stem canker resistance when compared to a soybean plant lacking the favorable allele, wherein the at least one favorable allele selected from the group consisting of allele A of S03188-1, allele A of S04492-1, allele A of S08256-1, allele T of S08257-1, allele C of S08231-3, allele T of S08231-4, allele T of S08241-1, allele C of S08251-4, allele G of S08251-2, allele C of S08255-3, allele A of S08255-4, allele T of S13721-2, allele A of S01591-1, allele G of S13722-1, allele G of S02987-1, allele G of S00802-1, allele A of S00777-1, allele C of S01799-1, allele A of S00288-1, allele A of S03923-1, allele T of S00341-1, allele T of S01718-1, allele T of S04785-1, allele A of Gm14:1088724, allele A of Gm14:1197243, allele A of Gm14:1594889, allele T of Gm14:1650065, allele C of Gm14:1727084, allele T of Gm14:1727625, allele T of Gm14:1747540, allele C of Gm14:1748042, allele G of Gm14:1748141, allele C of Gm14:1755250, allele A of Gm14:1755572, allele T of Gm14:1788456, allele A of Gm14:1923247, allele G of Gm14:1925222, allele G of Gm14:2129691, allele G of Gm14:3468738, allele A of Gm14:3726937, allele C of Gm14:3727753, allele A of Gm14:4204414, allele A of Gm14:4946161, allele T of Gm14:5028737, allele T of Gm14:5096522, allele A of Gm14:1706695, allele C of Gm14:1723656, allele G of Gm14:1723831, allele G of Gm14:1724319, allele G of Gm14:1738483, allele A of Gm14:1739929, allele A of Gm14:1850843, allele T of Gm14:1853044, allele G of Gm14:1863371, allele G of Gm14:1863436, allele C of Gm14:1863868, allele C of Gm14:1914305, allele T of Gm14:1936105, allele A of Gm14:1939210, allele C of Gm14:1940046, allele C of Gm14:1942461, allele T of Gm14:1942681, allele T of Gm14:1700735, allele G of Gm14:1702258, allele C of Gm14:1715352, allele A of Gm14:1718565, allele C of Gm14:1722078, allele T of Gm14:1722192, allele G of Gm14:1723045, allele A of Gm14:1724087, allele A of Gm14:1724245, allele T of Gm14:1724558, allele T of Gm14:1724603, allele A of Gm14:1725318, allele A of Gm14:1726899, allele T of Gm14:1738054, allele A of Gm14:1784545, allele C of Gm14:1785276, allele C of Gm14:1793750, allele T of Gm14:1842226, allele T of Gm14:1850134, allele A of Gm14:1850654, allele C of Gm14:1850808, allele C of Gm14:1851180, allele G of Gm14:1851369, allele A of Gm14:1851467, allele C of Gm14:1851793, allele G of Gm14:1852388, allele C of Gm14:1852579, allele A of Gm14:1855401, allele A of Gm14:1867651, allele A of Gm14:1867913, allele C of Gm14:1867927, allele C of Gm14:1910547, allele T of Gm14:1910746, allele C of Gm14:1910787, allele A of Gm14:1911679, allele A of Gm14:1916729, allele T of Gm14:1916742, allele T of Gm14:1919121, allele C of Gm14:1934770, allele C of Gm14:1935561, allele G of Gm14:1935695, allele A of Gm14:1935949, allele T of Gm14:1936112, allele A of Gm14:1936619, allele T of Gm14:1936750, allele A of Gm14:1936804, allele G of Gm14:1936827, allele T of Gm14:1937070, allele C of Gm14:1937136, allele C of Gm14:1937495, allele A of Gm14:1938061, allele C of Gm14:1940079, allele T of Gm14:1940534, allele G of Gm14:1940728, allele C of Gm14:1940825, allele G of Gm14:1941047, allele G of Gm14:1941105, allele A of Gm14:1944642, allele T of Gm14:1700746, allele G of Gm14:1706616, allele G of Gm14:1706705, allele A of Gm14:1706782, allele C of Gm14:1708836, allele T of Gm14:1709816, allele T of Gm14:1712174, allele A of Gm14:1717270, allele C of Gm14:1722544, allele T of Gm14:1723756, allele T of Gm14:1725153, allele C of Gm14:1725269, allele G of Gm14:1726477, allele C of Gm14:1731690, allele T of Gm14:1738538, allele T of Gm14:1739491, allele G of Gm14:1739824, allele G of Gm14:1785457, allele T of Gm14:1787260, allele T of Gm14:1796497, allele G of Gm14:1840314, allele A of Gm14:1842837, allele C of Gm14:1850412, allele T of Gm14:1850433, allele T of Gm14:1850907, allele T of Gm14:1851535, allele A of Gm14:1852210, allele C of Gm14:1859255, allele G of Gm14:1860411, allele T of Gm14:1909564, allele G of Gm14:1910968, allele A of Gm14:1935936, allele A of Gm14:1936214, allele G of Gm14:1936876, allele C of Gm14:1939030, allele T of Gm14:1939031, allele A of Gm14:1939081, allele A of Gm14:1940392, allele A of Gm14:1941517, allele G of Gm14:1989692, allele C of Gm14:1989717, allele T of Gm14:1992951, allele A of Gm14:2003498, allele A of Gm14:2004242, allele T of Gm14:2004409, allele A of Gm14:2006356, allele A of Gm14:1698254, allele T of Gm14:1698262, allele C of Gm14:1698269, allele T of Gm14:1701170, allele G of Gm14:1701174, allele T of Gm14:1701472, allele G of Gm14:1701825, allele A of Gm14:1702477, allele G of Gm14:1702552, allele T of Gm14:1702622, allele C of Gm14:1704204, allele A of Gm14:1705803, allele T of Gm14:1706201, allele C of Gm14:1706361, allele A of Gm14:1706585, allele T of Gm14:1706915, allele A of Gm14:1708746, allele A of Gm14:1713290, allele A of Gm14:1718056, allele A of Gm14:1718944, allele T of Gm14:1719373, allele A of Gm14:1723066, allele T of Gm14:1726434, allele G of Gm14:1726867, allele A of Gm14:1727235, allele G of Gm14:1728343, allele A of Gm14:1730044, allele T of Gm14:1731518, allele C of Gm14:1736053, allele C of Gm14:1738509, allele A of Gm14:1738914, allele C of Gm14:1739468, allele C of Gm14:1739718, allele T of Gm14:1739772, allele C of Gm14:1749899, allele A of Gm14:1785516, allele G of Gm14:1785559, allele T of Gm14:1786034, allele A of Gm14:1786259, allele A of Gm14:1793453, allele A of Gm14:1796469, allele A of Gm14:1797188, allele G of Gm14:1798987, allele A of Gm14:1800055, allele T of Gm14:1842374, allele A of Gm14:1850066, allele A of Gm14:1850741, allele C of Gm14:1850928, allele T of Gm14:1852469, allele G of Gm14:1854015, allele A of Gm14:1854023, allele T of Gm14:1859810, allele G of Gm14:1860271, allele G of Gm14:1862541, allele A of Gm14:1867332, allele C of Gm14:1868414, allele C of Gm14:1919937, allele T of Gm14:1935444, allele T of Gm14:1936433, allele G of Gm14:1937170, allele T of Gm14:1937923, allele T of Gm14:1938818, allele T of Gm14:1939343, allele T of Gm14:1939364, allele A of Gm14:1939647, allele T of Gm14:1939662, allele C of Gm14:1939746, allele G of Gm14:1939749, allele A of Gm14:1940207, allele A of Gm14:1940573, allele T of Gm14:1941546, allele A of Gm14:1945962, allele A of Gm14:1946355, allele A of Gm14:1990996, allele C of Gm14:1991421, allele T of Gm14:2001506, allele T of Gm14:2001882, allele G of Gm14:2003462, allele T of Gm14:2003672, allele C of Gm14:1697331, allele G of Gm14:1698694, allele C of Gm14:1700508, allele G of Gm14:1701739, allele G of Gm14:1701858, allele A of Gm14:1702110, allele C of Gm14:1702378, allele T of Gm14:1706884, allele T of Gm14:1706973, allele G of Gm14:1708902, allele T of Gm14:1711952, allele C of Gm14:1712050, allele A of Gm14:1712345, allele G of Gm14:1717261, allele T of Gm14:1722228, allele C of Gm14:1722277, allele C of Gm14:1723101, allele T of Gm14:1724793, allele G of Gm14:1725740, allele T of Gm14:1726334, allele T of Gm14:1727170, allele C of Gm14:1730199, allele G of Gm14:1736329, allele G of Gm14:1736463, allele T of Gm14:1736832, allele G of Gm14:1737771, allele G of Gm14:1738824, allele A of Gm14:1738960, allele G of Gm14:1743818, allele G of Gm14:1756757, allele C of Gm14:1760506, allele T of Gm14:1778710, allele T of Gm14:1783716, allele T of Gm14:1784221, allele G of Gm14:1786098, allele A of Gm14:1797150, allele G of Gm14:1797155, allele T of Gm14:1799977, allele G of Gm14:1811618, allele C of Gm14:1812051, allele A of Gm14:1824464, allele T of Gm14:1839360, allele G of Gm14:1843408, allele T of Gm14:1850104, allele A of Gm14:1851115, allele G of Gm14:1851116, allele G of Gm14:1855960, allele C of Gm14:1858300, allele A of Gm14:1860243, allele C of Gm14:1860675, allele C of Gm14:1866981, allele C of Gm14:1869210, allele A of Gm14:1872625, allele C of Gm14:1902929, allele T of Gm14:1913544, allele T of Gm14:1914780, allele C of Gm14:1935408, allele C of Gm14:1936381, allele A of Gm14:1936425, allele G of Gm14:1939383, allele T of Gm14:1988977, allele A of Gm14:1989052, allele A of Gm14:1990632, allele A of Gm14:1990775, allele G of Gm14:1991550, allele C of Gm14:1999220, allele G of Gm14:2003981, allele G of Gm14:1697620, allele A of Gm14:1697978, allele A of Gm14:1699410, allele G of Gm14:1700636, allele C of Gm14:1701683, allele T of Gm14:1701935, allele T of Gm14:1702661, allele A of Gm14:1702755, allele C of Gm14:1704651, allele C of Gm14:1704664, allele A of Gm14:1712038, allele A of Gm14:1712320, allele A of Gm14:1712713, allele T of Gm14:1712865, allele A of Gm14:1712866, allele T of Gm14:1713999, allele T of Gm14:1719022, allele C of Gm14:1728216, allele T of Gm14:1731767, allele C of Gm14:1735093, allele T of Gm14:1735102, allele A of Gm14:1736945, allele G of Gm14:1743592, allele C of Gm14:1748064, allele C of Gm14:1749126, allele G of Gm14:1754686, allele C of Gm14:1757996, allele C of Gm14:1760483, allele T of Gm14:1783231, allele G of Gm14:1783538, allele T of Gm14:1783756, allele G of Gm14:1786013, allele A of Gm14:1787773, allele C of Gm14:1792218, allele A of Gm14:1799327, allele C of Gm14:1799390, allele T of Gm14:1799540, allele G of Gm14:1800012, allele T of Gm14:1800319, allele T of Gm14:1811113, allele G of Gm14:1811977, allele C of Gm14:1824284, allele C of Gm14:1837872, allele G of Gm14:1837888, allele G of Gm14:1851947, allele G of Gm14:1858277, allele A of Gm14:1861083, allele T of Gm14:1887021, allele C of Gm14:1903293, allele T of Gm14:1910033, allele A of Gm14:1919915, allele A of Gm14:1934737, allele A of Gm14:1946125, allele T of Gm14:1958086, allele T of Gm14:1986602, allele A of Gm14:1987892, allele G of Gm14:1988001, allele A of Gm14:1989951, allele A of Gm14:1991474, allele C of Gm14:1997517, allele C of Gm14:2006319, allele C of Gm14:2017162, allele A of Gm14:2019929, allele C of Gm14:1701692, allele A of Gm14:1702315, allele C of Gm14:1702956, allele A of Gm14:1703980, allele A of Gm14:1706523, allele C of Gm14:1711087, allele C of Gm14:1712377, allele G of Gm14:1714965, allele G of Gm14:1727031, allele A of Gm14:1727851, allele G of Gm14:1728741, allele A of Gm14:1729293, allele A of Gm14:1730480, allele A of Gm14:1730798, allele G of Gm14:1731840, allele G of Gm14:1732232, allele G of Gm14:1732358, allele T of Gm14:1732363, allele T of Gm14:1732403, allele T of Gm14:1735755, allele T of Gm14:1735945, allele A of Gm14:1743425, allele T of Gm14:1744356, allele T of Gm14:1746573, allele A of Gm14:1748959, allele G of Gm14:1750564, allele G of Gm14:1753875, allele A of Gm14:1754007, allele C of Gm14:1755671, allele G of Gm14:1756046, allele T of Gm14:1760299, allele A of Gm14:1760329, allele G of Gm14:1761362, allele T of Gm14:1766162, allele G of Gm14:1780165, allele G of Gm14:1780408, allele C of Gm14:1782983, allele G of Gm14:1789475, allele C of Gm14:1799494, allele C of Gm14:1799548, allele A of Gm14:1799571, allele A of Gm14:1811318, allele T of Gm14:1825691, allele G of Gm14:1834023, allele G of Gm14:1841281, allele T of Gm14:1846850, allele G of Gm14:1887108, allele T of Gm14:1887251, allele C of Gm14:1935369, allele A of Gm14:1969791, allele G of Gm14:1981574, allele G of Gm14:1981644, allele G of Gm14:1983907, allele C of Gm14:1986614, allele G of Gm14:1986675, allele A of Gm14:1987587, allele T of Gm14:1988422, allele C of Gm14:1988424, allele T of Gm14:1988520, allele G of Gm14:1988558, allele G of Gm14:1989403, allele C of Gm14:1991457, allele T of Gm14:1991910, allele T of Gm14:1992830, allele T of Gm14:1993931, allele A of Gm14:1997124, allele C of Gm14:1998879, allele G of Gm14:2004939, allele C of Gm14:2005896, allele A of Gm14:1698816, allele G of Gm14:1701991, allele G of Gm14:1710294, allele C of Gm14:1710303, allele G of Gm14:1711007, allele C of Gm14:1711575, allele G of Gm14:1713816, allele G of Gm14:1728293, allele A of Gm14:1729743, allele A of Gm14:1730281, allele G of Gm14:1730795, allele G of Gm14:1732205, allele A of Gm14:1744432, allele G of Gm14:1751350, allele T of Gm14:1755398, allele G of Gm14:1756188, allele A of Gm14:1761081, allele A of Gm14:1761387, allele A of Gm14:1762867, allele G of Gm14:1765302, allele T of Gm14:1777323, allele A of Gm14:1779323, allele A of Gm14:1780397, allele C of Gm14:1800359, allele A of Gm14:1803234, allele A of Gm14:1811158, allele G of Gm14:1825653, allele G of Gm14:1869500, allele T of Gm14:1876026, allele G of Gm14:1876675, allele G of Gm14:1876844, allele G of Gm14:1876847, allele A of Gm14:1879162, allele T of Gm14:1884603, allele A of Gm14:1904675, allele T of Gm14:1944708, allele C of Gm14:1954056, allele T of Gm14:1954062, allele A of Gm14:1967108, allele C of Gm14:1972107, allele C of Gm14:2006215, allele G of Gm14:2011465, allele G of Gm14:1704689, allele G of Gm14:1711569, allele A of Gm14:1713323, allele G of Gm14:1728052, allele T of Gm14:1729537, allele T of Gm14:1732261, allele A of Gm14:1732593, allele A of Gm14:1735227, allele A of Gm14:1744452, allele A of Gm14:1754156, allele A of Gm14:1755877, allele C of Gm14:1756355, allele A of Gm14:1759702, allele A of Gm14:1780316, allele G of Gm14:1801585, allele A of Gm14:1810099, allele A of Gm14:1810126, allele C of Gm14:1810421, allele C of Gm14:1823776, allele T of Gm14:1875278, allele T of Gm14:1875874, allele G of Gm14:1902961, allele A of Gm14:1910054, allele T of Gm14:1949216, allele G of Gm14:1957357, allele A of Gm14:1957358, allele A of Gm14:1960447, allele C of Gm14:1980728, allele G of Gm14:1984129, allele C of Gm14:1992331, allele T of Gm14:1993147, allele G of Gm14:1995593, allele T of Gm14:1995600, allele G of Gm14:1996952, allele G of Gm14:1997412, allele T of Gm14:2004591, allele G of Gm14:1645762, allele T of Gm14:1713775, allele C of Gm14:1716505, allele A of Gm14:1729186, allele A of Gm14:1729798, allele A of Gm14:1732529, allele T of Gm14:1753922, allele C of Gm14:1759619, allele C of Gm14:1759635, allele A of Gm14:1760144, allele T of Gm14:1761168, allele C of Gm14:1771612, allele T of Gm14:1777105, allele T of Gm14:1778517, allele T of Gm14:1779288, allele A of Gm14:1779568, allele A of Gm14:1804682, allele A of Gm14:1810135, allele G of Gm14:1825225, allele G of Gm14:1828925, allele G of Gm14:1846994, allele A of Gm14:1848374, allele T of Gm14:1874941, allele G of Gm14:1875261, allele A of Gm14:1875470, allele C of Gm14:1879189, allele G of Gm14:1881168, allele A of Gm14:1923548, allele T of Gm14:1923935, allele G of Gm14:1955048, allele C of Gm14:1955118, allele T of Gm14:1955144, allele T of Gm14:1959610, allele A of Gm14:1960454, allele T of Gm14:1967167, allele C of Gm14:1970908, allele G of Gm14:1976442, allele C of Gm14:1981936, allele C of Gm14:1982632, allele C of Gm14:1982756, allele G of Gm14:1984276, allele A of Gm14:1994907, allele G of Gm14:2013156, allele A of Gm14:2019411, allele G of Gm14:1711379, allele T of Gm14:1713140, allele G of Gm14:1727997, allele A of Gm14:1744286, allele T of Gm14:1757864, allele C of Gm14:1759363, allele A of Gm14:1765933, allele C of Gm14:1767525, allele C of Gm14:1767582, allele C of Gm14:1768845, allele T of Gm14:1775564, allele G of Gm14:1778106, allele T of Gm14:1778621, allele T of Gm14:1780281, allele G of Gm14:1780494, allele C of Gm14:1805372, allele A of Gm14:1809090, allele G of Gm14:1810313, allele T of Gm14:1812109, allele A of Gm14:1812114, allele A of Gm14:1824651, allele A of Gm14:1875656, allele G of Gm14:1877489, allele T of Gm14:1878068, allele G of Gm14:1903061, allele A of Gm14:1950278, allele C of Gm14:1950577, allele G of Gm14:1950969, allele T of Gm14:1970110, allele C of Gm14:1982411, allele T of Gm14:1983489, allele T of Gm14:1983507, allele T of Gm14:1983593, allele G of Gm14:1993730, allele T of Gm14:1993788, allele A of Gm14:1993806, allele C of Gm14:2006229, allele C of Gm14:2015853, allele G of Gm14:2359579, allele G of Gm14:1579650, allele T of Gm14:1764334, allele G of Gm14:1764392, allele T of Gm14:1764403, allele C of Gm14:1767878, allele T of Gm14:1805366, allele A of Gm14:1825173, allele C of Gm14:1879313, allele G of Gm14:1886238, allele G of Gm14:1899408, allele G of Gm14:1960137, allele G of Gm14:1971009, allele T of Gm14:1983348, allele C of Gm14:2011607, allele C of Gm14:2012105, allele A of Gm14:2340746, allele C of Gm14:1767671, allele A of Gm14:1771502, allele A of Gm14:1771578, allele G of Gm14:1803995, allele G of Gm14:1805322, allele C of Gm14:1809988, allele C of Gm14:1825310, allele G of Gm14:1888310, allele A of Gm14:1889307, allele A of Gm14:1957202, allele A of Gm14:1981029, allele A of Gm14:1981073, allele T of Gm14:1981255, allele C of Gm14:1992432, allele C of Gm14:2010834, allele C of Gm14:1759440, allele A of Gm14:1775298, allele C of Gm14:1775605, allele C of Gm14:1804116, allele T of Gm14:1804868, allele C of Gm14:1806330, allele T of Gm14:1806377, allele T of Gm14:1889158, allele T of Gm14:1899428, allele T of Gm14:1926218, allele C of Gm14:1928522, allele T of Gm14:1957206, allele A of Gm14:1959033, allele C of Gm14:1969518, allele A of Gm14:1975722, allele C of Gm14:1981293, allele T of Gm14:1983189, allele T of Gm14:2013786, allele G of Gm14:2014487, allele A of Gm14:2191538, allele C of Gm14:2191541, allele T of Gm14:2191563, allele C of Gm14:1711534, allele C of Gm14:1773993, allele G of Gm14:1808347, allele C of Gm14:1808348, allele T of Gm14:1812356, allele C of Gm14:1812358, allele G of Gm14:1874583, allele C of Gm14:1971096, allele G of Gm14:1975723, allele G of Gm14:1976101, allele T of Gm14:1998648, allele T of Gm14:2006929, allele T of Gm14:2013850, allele T of Gm14:2014479, allele T of Gm14:2014481, allele A of Gm14:2101794, allele A of Gm14:766045, allele T of Gm14:1801323, allele A of Gm14:1805026, allele G of Gm14:1879302, allele T of Gm14:1975774, allele G of Gm14:2014429, allele G of Gm14:2191662, allele C of Gm14:584976, allele C of Gm14:2007005, allele A of Gm14:1650177, allele A of Gm14:1899046, allele C of Gm14:806705, allele G of Gm14:1976057, allele T of Gm14:2747160, and allele A of Gm14:1889837.

12. A kit for characterizing at least one soybean plant, germplasm or seed, the kit comprising:
(a) primers or probes for detecting one or more marker loci selected from the group consisting of the marker loci of claim 1, and markers closely linked thereto; and
(b) instructions for using the primers or probes to detect the one or more marker loci and for correlating the detected marker loci with predicted tolerance to stem canker.

13. The kit of claim 12, wherein one or more of marker locus S08231-3, S08241-1, S08251-2, S08255-3, or S08251-4 is detected.

14. The kit of claim 12, wherein the primers or probes comprise one or more of SEQ ID NOs: 1-786.

15. An isolated polynucleotide that detects a marker locus, said isolated polynucleotide comprising at least one detectable label, wherein said marker locus is selected from the group consisting of:
(a) a polymorphism in S03188-1, S04492-1, S08256-1, S08257-1, S08231-3, S08231-4, S08241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14);
(b) a marker locus comprising S03188-1, S04492-1, S08256-1, S08257-1, S08231-3, S08231-4, 508241-1, S08251-4, S08251-2, S08255-3, S08255-4, S13721-2, S01591-1, S13722-1, S02987-1, S00802-1, S00777-1, S01799-1, S00288-1, S03923-1, S00341-1, S01718-1, and S04785-1 on LG B2 (ch 14);
(c) S03188-1-A, S04492-1-A, S08256-1-01, S08257-1-01, S08231-3-01, S08231-4-Q1, S08241-1-06, S08251-4-07, S08251-2-Q10, S08255-3-03, S08255-4-Q1, S13721-2-Q1, 501591-1-A, 513722-1-01, 502987-1-A, S00802-1-A, S00777-1-A, S01799-1-A, S00288-1-A, S03923-1-A, S00341-1-A, S01718-1-A, and S04785-1-A;
(d) a marker locus comprising Gm14:1088724, Gm14:1197243, Gm14:1594889, Gm14:1650065, Gm14:1727084, Gm14:1727625, Gm14:1747540, Gm14:1748042, Gm14:1748141, Gm14:1755250, Gm14:1755572, Gm14:1788456, Gm14:1923247, Gm14:1925222, Gm14:2129691, Gm14:3468738, Gm14:3726937, Gm14:3727753, Gm14:4204414, Gm14:4946161, Gm14:5028737, Gm14:5096522, Gm14:1706695, Gm14:1723656, Gm14:1723831, Gm14:1724319, Gm14:1738483, Gm14:1739929, Gm14:1850843, Gm14:1853044, Gm14:1863371, Gm14:1863436, Gm14:1863868, Gm14:1914305, Gm14:1936105, Gm14:1939210, Gm14:1940046, Gm14:1942461, Gm14:1942681, Gm14:1700735, Gm14:1702258, Gm14:1715352, Gm14:1718565, Gm14:1722078, Gm14:1722192, Gm14:1723045, Gm14:1724087, Gm14:1724245, Gm14:1724558, Gm14:1724603, Gm14:1725318, Gm14:1726899, Gm14:1738054, Gm14:1784545, Gm14:1785276, Gm14:1793750, Gm14:1842226, Gm14:1850134, Gm14:1850654, Gm14:1850808, Gm14:1851180, Gm14:1851369, Gm14:1851467, Gm14:1851793, Gm14:1852388, Gm14:1852579, Gm14:1855401, Gm14:1867651, Gm14:1867913, Gm14:1867927, Gm14:1910547, Gm14:1910746, Gm14:1910787, Gm14:1911679, Gm14:1916729, Gm14:1916742, Gm14:1919121, Gm14:1934770, Gm14:1935561, Gm14:1935695, Gm14:1935949, Gm14:1936112, Gm14:1936619, Gm14:1936750, Gm14:1936804, Gm14:1936827, Gm14:1937070, Gm14:1937136, Gm14:1937495, Gm14:1938061, Gm14:1940079, Gm14:1940534, Gm14:1940728, Gm14:1940825, Gm14:1941047, Gm14:1941105, Gm14:1944642, Gm14:1700746, Gm14:1706616, Gm14:1706705, Gm14:1706782, Gm14:1708836, Gm14:1709816, Gm14:1712174, Gm14:1717270, Gm14:1722544, Gm14:1723756, Gm14:1725153, Gm14:1725269, Gm14:1726477, Gm14:1731690, Gm14:1738538, Gm14:1739491, Gm14:1739824, Gm14:1785457, Gm14:1787260, Gm14:1796497, Gm14:1840314, Gm14:1842837, Gm14:1850412, Gm14:1850433, Gm14:1850907, Gm14:1851535, Gm14:1852210, Gm14:1859255, Gm14:1860411, Gm14:1909564, Gm14:1910968, Gm14:1935936, Gm14:1936214, Gm14:1936876, Gm14:1939030, Gm14:1939031, Gm14:1939081, Gm14:1940392, Gm14:1941517, Gm14:1989692, Gm14:1989717, Gm14:1992951, Gm14:2003498, Gm14:2004242, Gm14:2004409, Gm14:2006356, Gm14:1698254, Gm14:1698262, Gm14:1698269, Gm14:1701170, Gm14:1701174, Gm14:1701472, Gm14:1701825, Gm14:1702477, Gm14:1702552, Gm14:1702622, Gm14:1704204, Gm14:1705803, Gm14:1706201, Gm14:1706361, Gm14:1706585, Gm14:1706915, Gm14:1708746, Gm14:1713290, Gm14:1718056, Gm14:1718944, Gm14:1719373, Gm14:1723066, Gm14:1726434, Gm14:1726867, Gm14:1727235, Gm14:1728343, Gm14:1730044, Gm14:1731518, Gm14:1736053, Gm14:1738509, Gm14:1738914, Gm14:1739468, Gm14:1739718, Gm14:1739772, Gm14:1749899, Gm14:1785516, Gm14:1785559, Gm14:1786034, Gm14:1786259, Gm14:1793453, Gm14:1796469, Gm14:1797188, Gm14:1798987, Gm14:1800055, Gm14:1842374, Gm14:1850066, Gm14:1850741, Gm14:1850928, Gm14:1852469, Gm14:1854015, Gm14:1854023, Gm14:1859810, Gm14:1860271, Gm14:1862541, Gm14:1867332, Gm14:1868414, Gm14:1919937, Gm14:1935444, Gm14:1936433, Gm14:1937170, Gm14:1937923, Gm14:1938818, Gm14:1939343, Gm14:1939364, Gm14:1939647, Gm14:1939662, Gm14:1939746, Gm14:1939749, Gm14:1940207, Gm14:1940573, Gm14:1941546, Gm14:1945962, Gm14:1946355, Gm14:1990996, Gm14:1991421, Gm14:2001506, Gm14:2001882, Gm14:2003462, Gm14:2003672, Gm14:1697331, Gm14:1698694, Gm14:1700508, Gm14:1701739, Gm14:1701858, Gm14:1702110, Gm14:1702378, Gm14:1706884, Gm14:1706973, Gm14:1708902, Gm14:1711952, Gm14:1712050, Gm14:1712345, Gm14:1717261, Gm14:1722228, Gm14:1722277, Gm14:1723101, Gm14:1724793, Gm14:1725740, Gm14:1726334, Gm14:1727170, Gm14:1730199, Gm14:1736329, Gm14:1736463, Gm14:1736832, Gm14:1737771, Gm14:1738824, Gm14:1738960, Gm14:1743818, Gm14:1756757, Gm14:1760506, Gm14:1778710, Gm14:1783716, Gm14:1784221, Gm14:1786098, Gm14:1797150, Gm14:1797155, Gm14:1799977, Gm14:1811618, Gm14:1812051, Gm14:1824464, Gm14:1839360, Gm14:1843408, Gm14:1850104, Gm14:1851115, Gm14:1851116, Gm14:1855960, Gm14:1858300, Gm14:1860243, Gm14:1860675, Gm14:1866981, Gm14:1869210, Gm14:1872625, Gm14:1902929, Gm14:1913544, Gm14:1914780, Gm14:1935408, Gm14:1936381, Gm14:1936425, Gm14:1939383, Gm14:1988977, Gm14:1989052, Gm14:1990632, Gm14:1990775, Gm14:1991550, Gm14:1999220, Gm14:2003981, Gm14:1697620, Gm14:1697978, Gm14:1699410, Gm14:1700636, Gm14:1701683, Gm14:1701935, Gm14:1702661, Gm14:1702755, Gm14:1704651, Gm14:1704664, Gm14:1712038, Gm14:1712320, Gm14:1712713, Gm14:1712865, Gm14:1712866, Gm14:1713999, Gm14:1719022, Gm14:1728216, Gm14:1731767, Gm14:1735093, Gm14:1735102, Gm14:1736945, Gm14:1743592, Gm14:1748064, Gm14:1749126, Gm14:1754686, Gm14:1757996, Gm14:1760483, Gm14:1783231, Gm14:1783538, Gm14:1783756, Gm14:1786013, Gm14:1787773, Gm14:1792218, Gm14:1799327, Gm14:1799390, Gm14:1799540, Gm14:1800012, Gm14:1800319, Gm14:1811113, Gm14:1811977, Gm14:1824284, Gm14:1837872, Gm14:1837888, Gm14:1851947, Gm14:1858277, Gm14:1861083, Gm14:1887021, Gm14:1903293, Gm14:1910033, Gm14:1919915, Gm14:1934737, Gm14:1946125, Gm14:1958086, Gm14:1986602, Gm14:1987892, Gm14:1988001, Gm14:1989951, Gm14:1991474, Gm14:1997517, Gm14:2006319, Gm14:2017162, Gm14:2019929, Gm14:1701692, Gm14:1702315, Gm14:1702956, Gm14:1703980, Gm14:1706523, Gm14:1711087, Gm14:1712377, Gm14:1714965, Gm14:1727031, Gm14:1727851, Gm14:1728741, Gm14:1729293, Gm14:1730480, Gm14:1730798, Gm14:1731840, Gm14:1732232, Gm14:1732358, Gm14:1732363, Gm14:1732403, Gm14:1735755, Gm14:1735945, Gm14:1743425, Gm14:1744356, Gm14:1746573, Gm14:1748959, Gm14:1750564, Gm14:1753875, Gm14:1754007, Gm14:1755671, Gm14:1756046, Gm14:1760299, Gm14:1760329, Gm14:1761362, Gm14:1766162, Gm14:1780165, Gm14:1780408, Gm14:1782983, Gm14:1789475, Gm14:1799494, Gm14:1799548, Gm14:1799571, Gm14:1811318, Gm14:1825691, Gm14:1834023, Gm14:1841281, Gm14:1846850, Gm14:1887108, Gm14:1887251, Gm14:1935369, Gm14:1969791, Gm14:1981574, Gm14:1981644, Gm14:1983907, Gm14:1986614, Gm14:1986675, Gm14:1987587, Gm14:1988422, Gm14:1988424, Gm14:1988520, Gm14:1988558, Gm14:1989403, Gm14:1991457, Gm14:1991910, Gm14:1992830, Gm14:1993931, Gm14:1997124, Gm14:1998879, Gm14:2004939, Gm14:2005896, Gm14:1698816, Gm14:1701991, Gm14:1710294, Gm14:1710303, Gm14:1711007, Gm14:1711575, Gm14:1713816, Gm14:1728293, Gm14:1729743, Gm14:1730281, Gm14:1730795, Gm14:1732205, Gm14:1744432, Gm14:1751350, Gm14:1755398, Gm14:1756188, Gm14:1761081, Gm14:1761387, Gm14:1762867, Gm14:1765302, Gm14:1777323, Gm14:1779323, Gm14:1780397, Gm14:1800359, Gm14:1803234, Gm14:1811158, Gm14:1825653, Gm14:1869500, Gm14:1876026, Gm14:1876675, Gm14:1876844, Gm14:1876847, Gm14:1879162, Gm14:1884603, Gm14:1904675, Gm14:1944708, Gm14:1954056, Gm14:1954062, Gm14:1967108, Gm14:1972107, Gm14:2006215, Gm14:2011465, Gm14:1704689, Gm14:1711569, Gm14:1713323, Gm14:1728052, Gm14:1729537, Gm14:1732261, Gm14:1732593, Gm14:1735227, Gm14:1744452, Gm14:1754156, Gm14:1755877, Gm14:1756355, Gm14:1759702, Gm14:1780316, Gm14:1801585, Gm14:1810099, Gm14:1810126, Gm14:1810421, Gm14:1823776, Gm14:1875278, Gm14:1875874, Gm14:1902961, Gm14:1910054, Gm14:1949216, Gm14:1957357, Gm14:1957358, Gm14:1960447, Gm14:1980728, Gm14:1984129, Gm14:1992331, Gm14:1993147, Gm14:1995593, Gm14:1995600, Gm14:1996952, Gm14:1997412, Gm14:2004591, Gm14:1645762, Gm14:1713775, Gm14:1716505, Gm14:1729186, Gm14:1729798, Gm14:1732529, Gm14:1753922, Gm14:1759619, Gm14:1759635, Gm14:1760144, Gm14:1761168, Gm14:1771612, Gm14:1777105, Gm14:1778517, Gm14:1779288, Gm14:1779568, Gm14:1804682, Gm14:1810135, Gm14:1825225, Gm14:1828925, Gm14:1846994, Gm14:1848374, Gm14:1874941, Gm14:1875261, Gm14:1875470, Gm14:1879189, Gm14:1881168, Gm14:1923548, Gm14:1923935, Gm14:1955048, Gm14:1955118, Gm14:1955144, Gm14:1959610, Gm14:1960454, Gm14:1967167, Gm14:1970908, Gm14:1976442, Gm14:1981936, Gm14:1982632, Gm14:1982756, Gm14:1984276, Gm14:1994907, Gm14:2013156, Gm14:2019411, Gm14:1711379, Gm14:1713140, Gm14:1727997, Gm14:1744286, Gm14:1757864, Gm14:1759363, Gm14:1765933, Gm14:1767525, Gm14:1767582, Gm14:1768845, Gm14:1775564, Gm14:1778106, Gm14:1778621, Gm14:1780281, Gm14:1780494, Gm14:1805372, Gm14:1809090, Gm14:1810313, Gm14:1812109, Gm14:1812114, Gm14:1824651, Gm14:1875656, Gm14:1877489, Gm14:1878068, Gm14:1903061, Gm14:1950278, Gm14:1950577, Gm14:1950969, Gm14:1970110, Gm14:1982411, Gm14:1983489, Gm14:1983507, Gm14:1983593, Gm14:1993730, Gm14:1993788, Gm14:1993806, Gm14:2006229, Gm14:2015853, Gm14:2359579, Gm14:1579650, Gm14:1764334, Gm14:1764392, Gm14:1764403, Gm14:1767878, Gm14:1805366, Gm14:1825173, Gm14:1879313, Gm14:1886238, Gm14:1899408, Gm14:1960137, Gm14:1971009, Gm14:1983348, Gm14:2011607, Gm14:2012105, Gm14:2340746, Gm14:1767671, Gm14:1771502, Gm14:1771578, Gm14:1803995, Gm14:1805322, Gm14:1809988, Gm14:1825310, Gm14:1888310, Gm14:1889307, Gm14:1957202, Gm14:1981029, Gm14:1981073, Gm14:1981255, Gm14:1992432, Gm14:2010834, Gm14:1759440, Gm14:1775298, Gm14:1775605, Gm14:1804116, Gm14:1804868, Gm14:1806330, Gm14:1806377, Gm14:1889158, Gm14:1899428, Gm14:1926218, Gm14:1928522, Gm14:1957206, Gm14:1959033, Gm14:1969518, Gm14:1975722, Gm14:1981293, Gm14:1983189, Gm14:2013786, Gm14:2014487, Gm14:2191538, Gm14:2191541, Gm14:2191563, Gm14:1711534, Gm14:1773993, Gm14:1808347, Gm14:1808348, Gm14:1812356, Gm14:1812358, Gm14:1874583, Gm14:1971096, Gm14:1975723, Gm14:1976101, Gm14:1998648, Gm14:2006929, Gm14:2013850, Gm14:2014479, Gm14:2014481, Gm14:2101794, Gm14:766045, Gm14:1801323, Gm14:1805026, Gm14:1879302, Gm14:1975774, Gm14:2014429, Gm14:2191662, Gm14:584976, Gm14:2007005, Gm14:1650177, Gm14:1899046, Gm14:806705, Gm14:1976057, Gm14:2747160, or Gm14:1889837;
(e) Gm14:1088724, Gm14:1197243, Gm14:1594889, Gm14:1650065, Gm14:1727084, Gm14:1727625, Gm14:1747540, Gm14:1748042, Gm14:1748141, Gm14:1755250, Gm14:1755572, Gm14:1788456, Gm14:1923247, Gm14:1925222, Gm14:2129691, Gm14:3468738, Gm14:3726937, Gm14:3727753, Gm14:4204414, Gm14:4946161, Gm14:5028737, Gm14:5096522, Gm14:1706695, Gm14:1723656, Gm14:1723831, Gm14:1724319, Gm14:1738483, Gm14:1739929, Gm14:1850843, Gm14:1853044, Gm14:1863371, Gm14:1863436, Gm14:1863868, Gm14:1914305, Gm14:1936105, Gm14:1939210, Gm14:1940046, Gm14:1942461, Gm14:1942681, Gm14:1700735, Gm14:1702258, Gm14:1715352, Gm14:1718565, Gm14:1722078, Gm14:1722192, Gm14:1723045, Gm14:1724087, Gm14:1724245, Gm14:1724558, Gm14:1724603, Gm14:1725318, Gm14:1726899, Gm14:1738054, Gm14:1784545, Gm14:1785276, Gm14:1793750, Gm14:1842226, Gm14:1850134, Gm14:1850654, Gm14:1850808, Gm14:1851180, Gm14:1851369, Gm14:1851467, Gm14:1851793, Gm14:1852388, Gm14:1852579, Gm14:1855401, Gm14:1867651, Gm14:1867913, Gm14:1867927, Gm14:1910547, Gm14:1910746, Gm14:1910787, Gm14:1911679, Gm14:1916729, Gm14:1916742, Gm14:1919121, Gm14:1934770, Gm14:1935561, Gm14:1935695, Gm14:1935949, Gm14:1936112, Gm14:1936619, Gm14:1936750, Gm14:1936804, Gm14:1936827, Gm14:1937070, Gm14:1937136, Gm14:1937495, Gm14:1938061, Gm14:1940079, Gm14:1940534, Gm14:1940728, Gm14:1940825, Gm14:1941047, Gm14:1941105, Gm14:1944642, Gm14:1700746, Gm14:1706616, Gm14:1706705, Gm14:1706782, Gm14:1708836, Gm14:1709816, Gm14:1712174, Gm14:1717270, Gm14:1722544, Gm14:1723756, Gm14:1725153, Gm14:1725269, Gm14:1726477, Gm14:1731690, Gm14:1738538, Gm14:1739491, Gm14:1739824, Gm14:1785457, Gm14:1787260, Gm14:1796497, Gm14:1840314, Gm14:1842837, Gm14:1850412, Gm14:1850433, Gm14:1850907, Gm14:1851535, Gm14:1852210, Gm14:1859255, Gm14:1860411, Gm14:1909564, Gm14:1910968, Gm14:1935936, Gm14:1936214, Gm14:1936876, Gm14:1939030, Gm14:1939031, Gm14:1939081, Gm14:1940392, Gm14:1941517, Gm14:1989692, Gm14:1989717, Gm14:1992951, Gm14:2003498, Gm14:2004242, Gm14:2004409, Gm14:2006356, Gm14:1698254, Gm14:1698262, Gm14:1698269, Gm14:1701170, Gm14:1701174, Gm14:1701472, Gm14:1701825, Gm14:1702477, Gm14:1702552, Gm14:1702622, Gm14:1704204, Gm14:1705803, Gm14:1706201, Gm14:1706361, Gm14:1706585, Gm14:1706915, Gm14:1708746, Gm14:1713290, Gm14:1718056, Gm14:1718944, Gm14:1719373, Gm14:1723066, Gm14:1726434, Gm14:1726867, Gm14:1727235, Gm14:1728343, Gm14:1730044, Gm14:1731518, Gm14:1736053, Gm14:1738509, Gm14:1738914, Gm14:1739468, Gm14:1739718, Gm14:1739772, Gm14:1749899, Gm14:1785516, Gm14:1785559, Gm14:1786034, Gm14:1786259, Gm14:1793453, Gm14:1796469, Gm14:1797188, Gm14:1798987, Gm14:1800055, Gm14:1842374, Gm14:1850066, Gm14:1850741, Gm14:1850928, Gm14:1852469, Gm14:1854015, Gm14:1854023, Gm14:1859810, Gm14:1860271, Gm14:1862541, Gm14:1867332, Gm14:1868414, Gm14:1919937, Gm14:1935444, Gm14:1936433, Gm14:1937170, Gm14:1937923, Gm14:1938818, Gm14:1939343, Gm14:1939364, Gm14:1939647, Gm14:1939662, Gm14:1939746, Gm14:1939749, Gm14:1940207, Gm14:1940573, Gm14:1941546, Gm14:1945962, Gm14:1946355, Gm14:1990996, Gm14:1991421, Gm14:2001506, Gm14:2001882, Gm14:2003462, Gm14:2003672, Gm14:1697331, Gm14:1698694, Gm14:1700508, Gm14:1701739, Gm14:1701858, Gm14:1702110, Gm14:1702378, Gm14:1706884, Gm14:1706973, Gm14:1708902, Gm14:1711952, Gm14:1712050, Gm14:1712345, Gm14:1717261, Gm14:1722228, Gm14:1722277, Gm14:1723101, Gm14:1724793, Gm14:1725740, Gm14:1726334, Gm14:1727170, Gm14:1730199, Gm14:1736329, Gm14:1736463, Gm14:1736832, Gm14:1737771, Gm14:1738824, Gm14:1738960, Gm14:1743818, Gm14:1756757, Gm14:1760506, Gm14:1778710, Gm14:1783716, Gm14:1784221, Gm14:1786098, Gm14:1797150, Gm14:1797155, Gm14:1799977, Gm14:1811618, Gm14:1812051, Gm14:1824464, Gm14:1839360, Gm14:1843408, Gm14:1850104, Gm14:1851115, Gm14:1851116, Gm14:1855960, Gm14:1858300, Gm14:1860243, Gm14:1860675, Gm14:1866981, Gm14:1869210, Gm14:1872625, Gm14:1902929, Gm14:1913544, Gm14:1914780, Gm14:1935408, Gm14:1936381, Gm14:1936425, Gm14:1939383, Gm14:1988977, Gm14:1989052, Gm14:1990632, Gm14:1990775, Gm14:1991550, Gm14:1999220, Gm14:2003981, Gm14:1697620, Gm14:1697978, Gm14:1699410, Gm14:1700636, Gm14:1701683, Gm14:1701935, Gm14:1702661, Gm14:1702755, Gm14:1704651, Gm14:1704664, Gm14:1712038, Gm14:1712320, Gm14:1712713, Gm14:1712865, Gm14:1712866, Gm14:1713999, Gm14:1719022, Gm14:1728216, Gm14:1731767, Gm14:1735093, Gm14:1735102, Gm14:1736945, Gm14:1743592, Gm14:1748064, Gm14:1749126, Gm14:1754686, Gm14:1757996, Gm14:1760483, Gm14:1783231, Gm14:1783538, Gm14:1783756, Gm14:1786013, Gm14:1787773, Gm14:1792218, Gm14:1799327, Gm14:1799390, Gm14:1799540, Gm14:1800012, Gm14:1800319, Gm14:1811113, Gm14:1811977, Gm14:1824284, Gm14:1837872, Gm14:1837888, Gm14:1851947, Gm14:1858277, Gm14:1861083, Gm14:1887021, Gm14:1903293, Gm14:1910033, Gm14:1919915, Gm14:1934737, Gm14:1946125, Gm14:1958086, Gm14:1986602, Gm14:1987892, Gm14:1988001, Gm14:1989951, Gm14:1991474, Gm14:1997517, Gm14:2006319, Gm14:2017162, Gm14:2019929, Gm14:1701692, Gm14:1702315, Gm14:1702956, Gm14:1703980, Gm14:1706523, Gm14:1711087, Gm14:1712377, Gm14:1714965, Gm14:1727031, Gm14:1727851, Gm14:1728741, Gm14:1729293, Gm14:1730480, Gm14:1730798, Gm14:1731840, Gm14:1732232, Gm14:1732358, Gm14:1732363, Gm14:1732403, Gm14:1735755, Gm14:1735945, Gm14:1743425, Gm14:1744356, Gm14:1746573, Gm14:1748959, Gm14:1750564, Gm14:1753875, Gm14:1754007, Gm14:1755671, Gm14:1756046, Gm14:1760299, Gm14:1760329, Gm14:1761362, Gm14:1766162, Gm14:1780165, Gm14:1780408, Gm14:1782983, Gm14:1789475, Gm14:1799494, Gm14:1799548, Gm14:1799571, Gm14:1811318, Gm14:1825691, Gm14:1834023, Gm14:1841281, Gm14:1846850, Gm14:1887108, Gm14:1887251, Gm14:1935369, Gm14:1969791, Gm14:1981574, Gm14:1981644, Gm14:1983907, Gm14:1986614, Gm14:1986675, Gm14:1987587, Gm14:1988422, Gm14:1988424, Gm14:1988520, Gm14:1988558, Gm14:1989403, Gm14:1991457, Gm14:1991910, Gm14:1992830, Gm14:1993931, Gm14:1997124, Gm14:1998879, Gm14:2004939, Gm14:2005896, Gm14:1698816, Gm14:1701991, Gm14:1710294, Gm14:1710303, Gm14:1711007, Gm14:1711575, Gm14:1713816, Gm14:1728293, Gm14:1729743, Gm14:1730281, Gm14:1730795, Gm14:1732205, Gm14:1744432, Gm14:1751350, Gm14:1755398, Gm14:1756188, Gm14:1761081, Gm14:1761387, Gm14:1762867, Gm14:1765302, Gm14:1777323, Gm14:1779323, Gm14:1780397, Gm14:1800359, Gm14:1803234, Gm14:1811158, Gm14:1825653, Gm14:1869500, Gm14:1876026, Gm14:1876675, Gm14:1876844, Gm14:1876847, Gm14:1879162, Gm14:1884603, Gm14:1904675, Gm14:1944708, Gm14:1954056, Gm14:1954062, Gm14:1967108, Gm14:1972107, Gm14:2006215, Gm14:2011465, Gm14:1704689, Gm14:1711569, Gm14:1713323, Gm14:1728052, Gm14:1729537, Gm14:1732261, Gm14:1732593, Gm14:1735227, Gm14:1744452, Gm14:1754156, Gm14:1755877, Gm14:1756355, Gm14:1759702, Gm14:1780316, Gm14:1801585, Gm14:1810099, Gm14:1810126, Gm14:1810421, Gm14:1823776, Gm14:1875278, Gm14:1875874, Gm14:1902961, Gm14:1910054, Gm14:1949216, Gm14:1957357, Gm14:1957358, Gm14:1960447, Gm14:1980728, Gm14:1984129, Gm14:1992331, Gm14:1993147, Gm14:1995593, Gm14:1995600, Gm14:1996952, Gm14:1997412, Gm14:2004591, Gm14:1645762, Gm14:1713775, Gm14:1716505, Gm14:1729186, Gm14:1729798, Gm14:1732529, Gm14:1753922, Gm14:1759619, Gm14:1759635, Gm14:1760144, Gm14:1761168, Gm14:1771612, Gm14:1777105, Gm14:1778517, Gm14:1779288, Gm14:1779568, Gm14:1804682, Gm14:1810135, Gm14:1825225, Gm14:1828925, Gm14:1846994, Gm14:1848374, Gm14:1874941, Gm14:1875261, Gm14:1875470, Gm14:1879189, Gm14:1881168, Gm14:1923548, Gm14:1923935, Gm14:1955048, Gm14:1955118, Gm14:1955144, Gm14:1959610, Gm14:1960454, Gm14:1967167, Gm14:1970908, Gm14:1976442, Gm14:1981936, Gm14:1982632, Gm14:1982756, Gm14:1984276, Gm14:1994907, Gm14:2013156, Gm14:2019411, Gm14:1711379, Gm14:1713140, Gm14:1727997, Gm14:1744286, Gm14:1757864, Gm14:1759363, Gm14:1765933, Gm14:1767525, Gm14:1767582, Gm14:1768845, Gm14:1775564, Gm14:1778106, Gm14:1778621, Gm14:1780281, Gm14:1780494, Gm14:1805372, Gm14:1809090, Gm14:1810313, Gm14:1812109, Gm14:1812114, Gm14:1824651, Gm14:1875656, Gm14:1877489, Gm14:1878068, Gm14:1903061, Gm14:1950278, Gm14:1950577, Gm14:1950969, Gm14:1970110, Gm14:1982411, Gm14:1983489, Gm14:1983507, Gm14:1983593, Gm14:1993730, Gm14:1993788, Gm14:1993806, Gm14:2006229, Gm14:2015853, Gm14:2359579, Gm14:1579650, Gm14:1764334, Gm14:1764392, Gm14:1764403, Gm14:1767878, Gm14:1805366, Gm14:1825173, Gm14:1879313, Gm14:1886238, Gm14:1899408, Gm14:1960137, Gm14:1971009, Gm14:1983348, Gm14:2011607, Gm14:2012105, Gm14:2340746, Gm14:1767671, Gm14:1771502, Gm14:1771578, Gm14:1803995, Gm14:1805322, Gm14:1809988, Gm14:1825310, Gm14:1888310, Gm14:1889307, Gm14:1957202, Gm14:1981029, Gm14:1981073, Gm14:1981255, Gm14:1992432, Gm14:2010834, Gm14:1759440, Gm14:1775298, Gm14:1775605, Gm14:1804116, Gm14:1804868, Gm14:1806330, Gm14:1806377, Gm14:1889158, Gm14:1899428, Gm14:1926218, Gm14:1928522, Gm14:1957206, Gm14:1959033, Gm14:1969518, Gm14:1975722, Gm14:1981293, Gm14:1983189, Gm14:2013786, Gm14:2014487, Gm14:2191538, Gm14:2191541, Gm14:2191563, Gm14:1711534, Gm14:1773993, Gm14:1808347, Gm14:1808348, Gm14:1812356, Gm14:1812358, Gm14:1874583, Gm14:1971096, Gm14:1975723, Gm14:1976101, Gm14:1998648, Gm14:2006929, Gm14:2013850, Gm14:2014479, Gm14:2014481, Gm14:2101794, Gm14:766045, Gm14:1801323, Gm14:1805026, Gm14:1879302, Gm14:1975774, Gm14:2014429, Gm14:2191662, Gm14:584976, Gm14:2007005, Gm14:1650177, Gm14:1899046, Gm14:806705, Gm14:1976057, Gm14:2747160, and Gm14:1889837;
(f) a marker locus comprising Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, or Glyma14g02730.1;
(g) a polymorphism in Glyma14g02740.1, Glyma14g02750.1, Glyma14g02780.1, Glyma14g02800.1, Glyma14g02820.1, Glyma14g02920.1, Glyma14g03010.1, Glyma14g03030.1, Glyma14g03050.1, Glyma14g03060.1, Glyma14g03100.1, Glyma14g02710.4, or Glyma14g02730.1;
(h) an interval flanked by and including BARC-013365-00489 and BARCSOYSSR_14_0281;
(i) an interval flanked by and including S03188-1 and S04785-1 on LG B2;
(j) a marker that detects a favorable allele of (a), (b), (c), (d), (e), (f), or (g);
(k) a marker locus closely linked to a marker locus of (a), (b), (c), (d), (e), (f), (g), (h), (i), or (j); and,
(l) a nucleotide sequence selected from the group consisting of SEQ ID NOs: 1-786.

16. An elite soybean plant, germplasm or seed identified by the method of claim 1, said plant, germplasm or seed comprising at least one quantitative trait locus associated with improved tolerance to stem canker in its genome, wherein said plant or germplasm has improved stem canker resistance when compared to a soybean plant or germplasm lacking said quantitative trait locus in its genome, and wherein said quantitative trait locus is localized to a chromosomal interval flanked by and including markers BARC-013365-00489 and BARCSOYSSR_14_0281 on linkage group B2 (ch 14).

17. The elite soybean plant, germplasm or seed of claim 16 further comprising resistance to a herbicidal formulation comprising a compound selected from the group consisting of a sulfonylurea, a hydroxyphenylpyruvatedioxygenase inhibitor, a glyphosate, a sulfonamide, an imidazolinone, a bialaphos, a phosphinothricin, a mesotrione, an isoxaflutole, an azafenidin, a butafenacil, a sulfosate, a glufosinate, a dicamba, a 2,4-D, a metribuzin, and a protox inhibitor.

18. The elite soybean plant, germplasm or seed of claim 17, wherein resistance to the herbicidal formulation is conferred by a transgene.

19. The elite soybean plant, germplasm or seed of claim 16 further comprising a trait selected from the group consisting of drought tolerance, stress tolerance, disease resistance, enhanced yield, modified oil, modified protein, tolerance to chlorotic conditions, and insect resistance.

20. The elite soybean plant, germplasm or seed of claim 19, wherein the trait is selected from the group consisting of brown stem rot resistance, charcoal rot drought complex resistance, Fusarium resistance, Phytophthora resistance, Soybean Mosaic virus resistance, carlavirus resistance, sudden death syndrome resistance, Sclerotinia resistance, Cercospora resistance, target spot resistance, frogeye leaf spot resistance, soybean cyst nematode resistance, root knot nematode resistance, rust resistance, high oleic, low linolenic, aphid resistance, stink bug resistance, and iron chlorosis deficiency tolerance.
